(19) United States
(12) Patent Application Publication
(10) Pub. No.: US 2010/0056524 A1
(43) Pub. Date: Mar. 4, 2010

(54) Compound

(76) Inventors: Edward Giles Mciver, London (GB); Justin Stephen Bryans, London (GB); Ela Smiljanic, London (GB); Stephen John Lewis, London (GB); Joanne Hough, London (GB); Thomas Drake, London (GB)

Correspondence Address: LAHIVE & COCKFIELD, LLP;FLOOR 30, SUITE 3000, ONE POST OFFICE SQUARE, BOSTON, MA, 02109, US

(21) Appl. No.: 12/384,477

(22) Filed: Apr. 2, 2009

Related U.S. Application Data
(60) Provisional application No. 61/072,764, filed on Apr. 2, 2008.

(30) Foreign Application Priority Data
Apr. 2, 2008 (GB) 0806005.5
Jul. 9, 2008 (GB) 0812580.9
Dec. 11, 2008 (GB) 0822635.9

(51) Int. Cl. A61K 31/5377; C07D 239/02; C07D 403/12; C07D 413/12; A61K 31/505; A61K 31/506

(52) U.S. Cl. 514/235.8; 544/297; 544/122; 514/275

(57) ABSTRACT

A first aspect of the invention relates to a compound of formula (I), or a pharmaceutically acceptable salt or ester thereof,

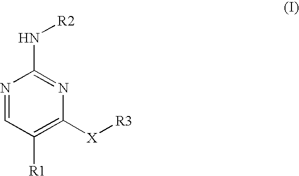

wherein:
R¹ is C₃₋₈-cycloalkyl; X is O, NR⁷ or C₃₋₆-heterocycloalkyl;
R² is aryl, heteroaryl, fused or unfused aryl-C₃₋₆-heterocycloalkyl or fused or unfused heteroaryl-C₃₋₆-heterocycloalkyl, each of which is optionally substituted by one or more substitutents selected from aryl, heteroaryl, C₁₋₆-alkyl, C₃₋₇-cycloalkyl and a group A, wherein said C₁₋₆-alkyl group is optionally substituted by one or more substituents selected from aryl, heteroaryl, R¹⁰ and a group A, said heteroaryl group is optionally substituted by one or more R¹⁰ groups; and wherein said C₃₋₆-heterocycloalkyl group optionally contains one or more groups selected from oxygen, sulfur, nitrogen and CO;
R³ is C₁₋₆-alkyl optionally substituted by one or more substituents selected from aryl, heteroaryl, —NR⁴R⁵, —OR⁶, —NR⁷(CO)R⁶, —NR⁷(CO)NR⁴R⁵, —NR⁷SO₂R⁶, —NR⁷COOR⁷, —CONR⁴R⁵, C₃₋₆-heterocycloalkyl and

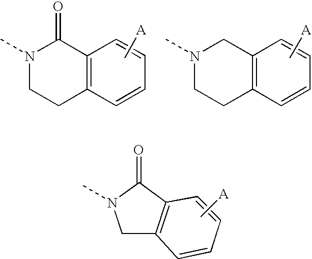

wherein R⁴⁻⁷ and A are as defined in the claims.

Further aspects relate to the use of said compounds in the treatment of various therapeutic disorders, and more particularly as inhibitors of one or more kinases.

### RELATED APPLICATION INFORMATION

This application claims the benefit under 35 USC 119(e) to provisional application 61/072,764 filed Apr. 2, 2008; and claims priority to United Kingdom Application No. 0806005.5 files Apr. 2, 2008, United Kingdom Application No. 0812580.9 files Jul. 9, 2009, and United Kingdom Application No. 0822635.5 filed Dec. 11, 2008. The entire contents of each of these application is incorporated herein by reference in their entirety.

The present invention relates to pyrimidine compounds that are capable of inhibiting one or more kinases. The compounds find applications in the treatment of a variety of disorders, including cancer, septic shock, neurodegenerative diseases, Alzheimer's disease, primary open angle glaucoma (POAG), hyperplasia, rheumatoid arthritis, psoriasis, artherosclerosis, retinopathy, osteoarthritis, endometriosis and/or chronic inflammation.

### BACKGROUND TO THE INVENTION

Pyrimidines and analogues thereof are already described as active ingredients, such as, for example, the 2-anilino-pyrimidines as fungicides (DE-A-4029650) or substituted pyrimidine derivatives for treating neurological or neurodegenerative diseases (WO 99/19305). As CDK inhibitors, the most varied pyrimidine derivatives are described, for example, bis(anilino)-pyrimidine derivatives (WO 00/12486), 2-amino-4-substituted pyrimidines (WO 01/14375), purines (WO 99/02162), 5-cyano-pyrimidines (WO 02/04429), anilinopyrimidines (WO 00/12486) and 2-N-dimethylaminopropoxy-pyrimidines (WO 00/39101).

It is amongst the objects of the present invention to provide compounds which display a high degree of activity and/or specificity to particular kinases and may therefore serve as drug candidates or as starting points for further derivatisation and kinase inhibition studies.

It is a further object of the present invention to provide compounds for potential use as drug candidates for treating cancer, septic shock, neurodegenerative diseases, Alzheimer's disease, inflammatory disease and/or primary open angle glaucoma (POAG).

It is a further object to provide compounds which display a significant inhibitory effect on one or more of the following kinases: TBK1, MKK1, ERK8, RSK1, RSK2, PDK1, S6K1, MNK2, PHK, CHK1, CHK2, GSK3beta, CDK2, MARK3, MELK, IRR, VEG-FR, and/or IKKepsilon.

### STATEMENT OF INVENTION

A first aspect of the invention relates to a compound of formula (I), or a pharmaceutically acceptable salt or ester thereof,

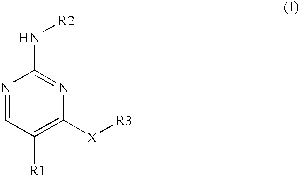

wherein:
R¹ is C₃₋₈-cycloalkyl;
X is O, NR⁷ or C₃₋₆-heterocycloalkyl;
R² is aryl, heteroaryl, fused or unfused aryl-C₃₋₆-heterocycloalkyl or fused or unfused heteroaryl-C₃₋₆-heterocycloalkyl, each of which is optionally substituted by one or more substitutents selected from aryl, heteroaryl, C₁₋₆-alkyl, C₃₋₇-cycloalkyl and a group A, wherein said C₁₋₆-alkyl group is optionally substituted by one or more substituents selected from aryl, heteroaryl, R¹⁰ and a group A, said heteroaryl group is optionally substituted by one or more R¹⁰ groups; and wherein said C₃₋₆-heterocycloalkyl group optionally contains one or more groups selected from oxygen, sulfur, nitrogen and CO;
R³ is C₁₋₆-alkyl optionally substituted by one or more substituents selected from aryl, heteroaryl, —NR⁴R⁵, —OR⁶, —NR⁷(CO)R⁶, —NR⁷(CO)NR⁴R⁵, —NR⁷SO₂R⁶, —NR⁷COOR⁷, —CONR⁴R⁵, C₃₋₆-heterocycloalkyl and

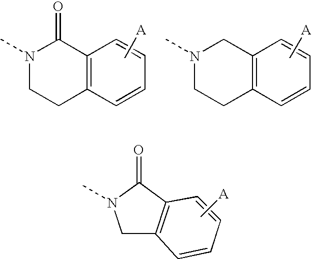

wherein said aryl, heteroaryl and C₃₋₆-heterocycloalkyl groups are each optionally substituted by one or more substituents selected from —C₁₋₆-alkyl and a group A, wherein said —C₁₋₆-alkyl group is optionally substituted by one or more substituents selected from aryl, heteroaryl and a group A;
A is selected from halogen, hydroxyl, cyano, trifluoromethyl, alkoxy, —NO₂, —NH₂, —NR⁴R⁵, —OR⁶, —NR⁷(CO)R⁶, —NR⁷(CO)NR⁴R⁵, —NR⁷COOR⁷, —NR⁷(SO₂)R⁶, CO₂H, —NR⁷(SO₂)NR⁴R⁵, —COOR⁷, —CONR⁴R⁵, COR⁶, SO₂NR⁴R⁵ and —SO₂R⁶;
each R⁴ and R⁵ is independently selected from hydrogen, C₃₋₇-cycloalkyl, aryl, heteroaryl, C₁₋₆-alkyl and a C₃₋₆-heterocycloalkyl ring optionally further containing one or more groups selected from oxygen, sulfur, nitrogen and CO and optionally substituted by one or more R¹⁰ groups, wherein said C₁₋₆-alkyl is optionally substituted by one or more substituents selected from halogen, cyano, hydroxyl, aryl, heteroaryl, —NR⁸R⁹, —NR⁷(CO)R⁶, —NR⁷COOR⁶, —NR⁷(SO₂)R⁶, —COOR⁶, —CONR⁸R⁹, OR¹⁰, —SO₂R⁶ and a C₃₋₆-heterocycloalkyl ring optionally further containing one or more groups selected from oxygen, sulfur, nitrogen and CO and optionally substituted by one or more or R¹⁰ groups; or
R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring may be saturated or unsaturated and is optionally substituted with one or more groups selected from NR⁸R⁹ and R¹⁰ groups;
each R⁶ is independently selected from C₁₋₆-alkyl, C₃₋₇ cycloalkyl, C₄₋₇-heterocycloalkyl, aryl and heteroaryl, each of which may be optionally substituted by one or more substituents selected from halogen, R¹⁰ and —NR⁸R⁹;
each R⁷ is selected from hydrogen, C₁₋₆-alkyl and C₃₋₇-cycloalkyl, wherein said C₁₋₆-alkyl is optionally substituted by one or more halogens;
each of R⁸ and R⁹ is independently selected from hydrogen and C₁₋₆-alkyl, wherein said C₁₋₆-alkyl group is optionally substituted by one or more halogens; or
R⁸ and R⁹ together with the N to which they are attached form a C₄₋₆-heterocycloalkyl ring optionally further containing one or more heteroatoms selected from oxygen and sulfur, wherein said C₄₋₆-heterocycloalkyl ring is optionally substituted by one or more R¹⁰ groups; and
each R¹⁰ is selected from halogen, C₃₋₇-cycloalkyl and C₁₋₆-alkyl optionally substituted by one or more halogens, wherein where R¹⁰ is C₁₋₆-alkyl and two or more R¹⁰ groups are attached to the same carbon atom, the R¹⁰ groups may be linked to form a spiroalkyl group.

As is demonstrated in Examples section that follows, representative compounds of the present invention were tested for their kinase inhibition activity and showed significant potency to TBK1 and/or MKK1, ERK8, RSK1, RSK2, PDK1, S6K1, MNK2, PHK, CHK1, CHK2, GSK3beta, CDK2, MARK3, MELK, IRR, VEG-FR, and/or IKKepsilon. These compounds can therefore efficiently serve for treating diseases or disorders in which inhibiting the activity of one or more of these kinases, would be beneficial. When the kinase is PDK1, the present invention relates to the treatment of diseases where it would be desirable to inhibit PDK1 and at least one other identified kinase.

A second aspect of the invention relates to a pharmaceutical composition comprising at least one compound as described above and a pharmaceutically acceptable carrier, diluent or excipient.

A third aspect of the invention relates to a compound as described above for use in medicine.

A fourth aspect of the invention relates to a compound as described above for use in treating a disorder selected from cancer, septic shock, neurodegenerative diseases, Alzheimer's disease, Primary open Angle Glaucoma (POAG), hyperplasia, rheumatoid arthritis, psoriasis, artherosclerosis, retinopathy, osteoarthritis, endometriosis and chronic inflammation.

A fifth aspect of the invention relates to the use of a compound as described above in the preparation of a medicament for treating or preventing a disorder selected from cancer, septic shock, neurodegenerative diseases, Alzheimer's disease, primary open angle glaucoma (POAG), hyperplasia, rheumatoid arthritis, psoriasis, artherosclerosis, retinopathy, osteoarthritis, endometriosis and chronic inflammation.

A sixth aspect of the invention relates to the use of a compound as described above in the preparation of a medicament for the prevention or treatment of a disorder caused by, associated with or accompanied by any abnormal kinase activity, wherein the kinase is selected from TBK1, MKK1, ERK8, RSK1, RSK2, PDK1, S6K1, MNK2, PHK, CHK1, CHK2, GSK3beta, CDK2, MARK3, MELK, IRR, VEG-FR, IKKepsilon and combinations thereof.

A seventh aspect of the invention relates to a method of treating a mammal having a disease state alleviated by the inhibition of a kinase selected from TBK1, MKK1, ERK8, RSK1, RSK2, PDK1, S6K1, MNK2, PHK, CHK1, CHK2, GSK3beta, CDK2, MARK3, MELK, IRR, VEG-FR, and IKKepsilon, wherein the method comprises administering to a mammal a therapeutically effective amount of a compound as described above.

An eighth aspect of the invention relates to the use of a compound as described above in an assay for identifying further candidate compounds capable of inhibiting one or more kinases selected from TBK1, MKK1, ERK8, RSK1, RSK2, PDK1, S6K1, MNK2, PHK, CHK1, CHK2, GSK3beta, CDK2, MARK3, MELK, IRR, VEG-FR, and IKKepsilon.

A ninth aspect of the invention relates to a process for preparing a compound of formula IV, wherein R¹, R², R³ and R⁷ are as defined above, said process comprising the steps of:

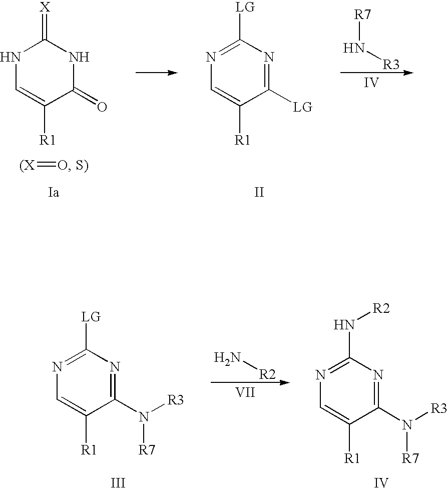

(i) converting a compound of formula Ia to a compound of formula II, where each LG is independently a leaving group;
(ii) reacting said compound of formula II with an amine of formula IV to form a compound of formula III;
(iii) reacting said compound of formula III with an amine of formula VII to form a compound of formula IV.

A tenth aspect of the invention relates to a process for preparing a compound of formula XIX, wherein R¹, R² and R³ are as defined above, said process comprising the steps of:

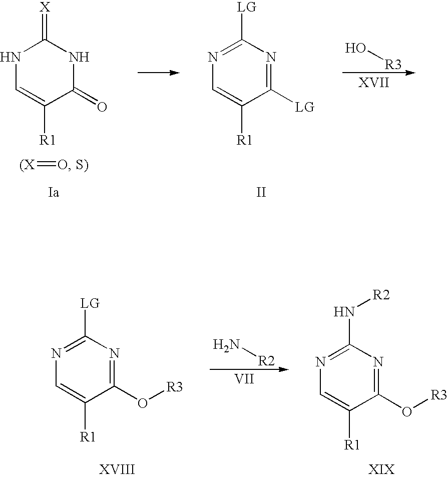

(i) converting a compound of formula Ia to a compound of formula II, where each LG is independently a leaving group;
(ii) reacting said compound of formula II with an amine of formula XVII to form a compound of formula XVIII;
(iii) reacting said compound of formula XVIII with an amine of formula VII to form a compound of formula XIX.

### DETAILED DESCRIPTION

“Alkyl” is defined herein as a straight-chain or branched alkyl radical, for example, methyl, ethyl, propyl, isopropyl, butyl, isobutyl, tert-butyl, pentyl, hexyl. Preferably, the alkyl group is a C₁₋₆ alkyl group, more preferably a C₁₋₄ group.

“Cycloalkyl” is defined herein as a monocyclic alkyl ring, such as, cyclopropyl, cyclobutyl, cyclopentyl, cyclohexyl or cycloheptyl. Preferably, the cycloalkyl group is a C₃₋₁₂ cycloalkyl group, more preferably a C₃₋₆ cycloalkyl group.

“Halogen” is defined herein as chloro, fluoro, bromo or iodo.

As used herein, the term “aryl” refers to a C₆₋₁₂ aromatic group, which may be benzocondensed, for example, phenyl or naphthyl.

“Heteroaryl” is defined herein as a monocyclic or bicyclic C₂₋₁₂ aromatic ring comprising one or more heteroatoms (that may be the same or different), such as oxygen, nitrogen or sulfur. Examples of suitable heteroaryl groups include thienyl, furanyl, pyrrolyl, pyridinyl, oxazolyl, thiazolyl, imidazolyl, pyrazolyl, isoxazolyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc.

“Heterocycloalkyl” refers to a cyclic aliphatic group containing one or more heteroatoms selected from nitrogen, oxygen and sulfur, which is optionally interrupted by one or more —(CO)— groups in the ring and/or which optionally contains one or more double bonds in the ring. Preferably, the heterocycloalkyl group comprises 3-6 carbon atoms and is fully saturated. Preferred heterocycloalkyl groups include piperidinyl, pyrrolidinyl, piperazinyl, thiomorpholinyl and morpholinyl. More preferably, the heterocycloalkyl group is selected from N-piperidinyl, N-pyrrolidinyl, N-piperazinyl, N-thiomorpholinyl and N-morpholinyl.

In one preferred embodiment of the invention, R¹ is cyclopropyl or cyclobutyl.

More preferably, R¹ is cyclopropyl.

In one preferred embodiment of the invention, X is O or NR⁷, more preferably, O, NMe or NH. Even more preferably, X is NH.

In one preferred embodiment, A is selected from halogen, hydroxyl, cyano, trifluoromethyl, alkoxy, —NO₂, —NH₂, —NR⁴R⁵, —OR⁶, —NR⁷(CO)R⁶, —NR⁷(CO)NR⁴R⁵, —NR⁷COOR⁷, —NR⁷(SO₂)R⁶, CO₂H, —NR⁷(SO₂)NR⁴R⁵, —COOR⁷, —CONR⁴R⁵, COR⁶ and —SO₂CH₃.

In one preferred embodiment of the invention, R² is an optionally substituted aryl or heteroaryl group selected from the following:

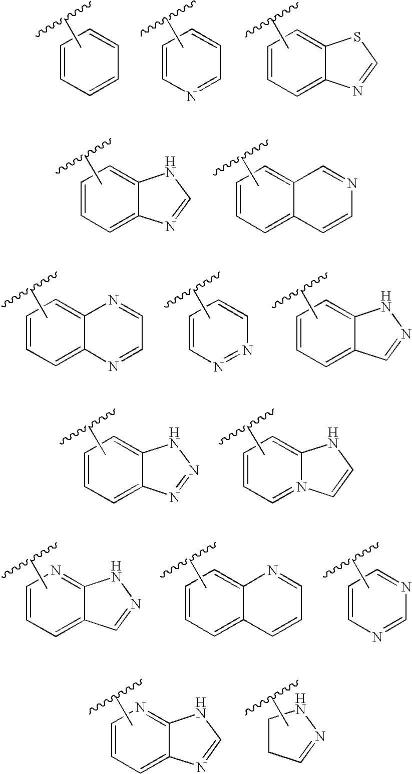

Preferably, the substituent attached to the aryl or heteroaryl group is selected from C₁₋₆-alkyl, C₃₋₇-cycloalkyl and a group A, wherein said C₁₋₆-alkyl group is in turn optionally substituted by one or more substituents selected from aryl, heteroaryl, R¹⁰ and a group A.

In one particularly preferred embodiment of the invention, R² is an optionally substituted aryl or heteroaryl group selected from the following:

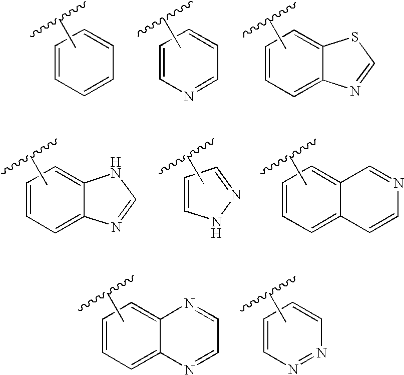

In a more preferred embodiment, R² is an aryl or heteroaryl group each of which is optionally substituted by one or more substituents selected from C₁₋₆-alkyl and a group A, wherein said C₁₋₆-alkyl group is optionally substituted by one or more substituents selected from aryl, heteroaryl and a group A, and wherein A is selected from halogen, OH, CN, CF₃, —NH₂, —NR⁴R⁵, —OR⁶, NR⁷(CO)R⁶, —NR⁷COOR⁷, —NR⁷(SO₂)R⁶, —COOH, —COOR⁷ and CONR⁴R⁵.

In an even more preferred embodiment, R² is an aryl or heteroaryl group each of which is optionally substituted by one or more substituents selected from C₁₋₆-alkyl, halogen, CN, NHCO—C₁₋₆-alkyl, CF₃, COOH, CONH₂, OH, NH₂, NHSO₂—C₁₋₆alkyl, O—CF₃, —NHCOO—C₁₋₆-alkyl, —CO₂—C₁₋₆-alkyl, —N(C₁₋₆-alkyl)₂, 4-methylpiperazin-1-yl, (4-methylpiperazin-1-yl)-CO—, (N-morpholinyl)-(CH₂)ₚ(O)_{q}—, (imidazol-1-yl)-(CH₂)ₚ— where q is 0, 1, 2 or 3 and each p is independently 1, 2 or 3 and NR⁴R⁵, wherein R⁴ and R⁵ and the nitrogen to which they are attached form a C₃₋₆-heterocycloalkyl ring optionally containing a CO group.

More preferably, R² is an aryl or heteroaryl group each of which is optionally substituted by one or more substituents selected from Me, Cl, F, CN, NHCOMe, CF₃, COOH, CONH₂, OH, NH₂, NHSO₂Me, OCF₃, —NHCOO^{t}Bu, —CO₂Me, —NMe₂, 4-methylpiperazin-1-yl, N-morpholinyl, (4-methylpiperazin-1-yl)-CO—, (N-morpholinyl)-CH₂CH₂O—, (imidazol-1-yl)-CH₂— and

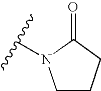

In one preferred embodiment, R² is a phenyl group optionally substituted by one or more groups selected from C₁₋₆-alkyl, heteroaryl and A, wherein said C₁₋₆-alkyl and heteroaryl groups are in turn optionally substituted.

In one preferred embodiment, R² is a phenyl group substituted by one or more A groups. Preferably, the A group is selected from CF₃, halogen, CN, NHSO₂Me, SO₂NR⁴R⁵, NR⁴R⁵, OR⁶, COOR⁷, NR⁷COOR⁷, NR⁷COR⁶, CONR⁴R⁵, NR⁷CONR⁴R⁵ and NR⁷SO₂R⁶.

In one preferred embodiment, R² is a phenyl group substituted by one or more C₁₋₆-alkyl groups, each of which in turn is optionally substituted by one or more groups selected from heteroaryl and A. Preferably, the heteroaryl group is selected from imidazolyl and triazolyl. Preferably, the A group is selected from CONR⁴R⁵, NR⁴R⁵, OR⁶, COOR⁷ and CN.

In one preferred embodiment, R² is a phenyl group substituted by one or more heteroaryl groups. Preferably, the heteroaryl group is selected from pyrimidinyl, tetrazolyl, pyridinyl, pyrazolyl, oxazolyl and triazolyl.

In one preferred embodiment, R² is a pyridyl group optionally substituted by one or more groups selected from C₁₋₆-alkyl, heteroaryl and A, wherein said C₁₋₆-alkyl and heteroaryl groups are in turn optionally substituted.

In one preferred embodiment, R² is a pyridyl group substituted by one or more A groups. Preferably, the A group is selected from NR⁴R⁵, halo and OR⁶.

In one preferred embodiment, R² is a pyridyl group substituted by a heteroaryl group. Preferably, the heteroaryl group is selected from pyrazolyl, pyrimidinyl and pyridinyl.

In one preferred embodiment, R² is a pyridyl group substituted by a C₁₋₆-alkyl group, wherein said C₁₋₆-alkyl group is in turn optionally substituted with one or more substituents selected from NR⁴R⁵ and OR⁶.

In one preferred embodiment, R² is an optionally substituted fused aryl-C₃₋₆-heterocycloalkyl or fused heteroaryl-C₃₋₆-heterocycloalkyl. Preferably, R² is selected from the following:

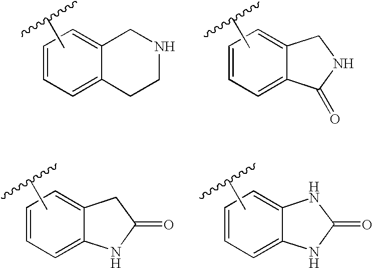

Preferably, the substituent is an optionally substituted C₁₋₆-alkyl group.

In one preferred embodiment of the invention, R³ is C₁₋₄-alkyl optionally substituted by one or more substituents selected from heteroaryl, —NR⁴R⁵, —NR⁷(CO)R⁶, —NR⁷COOR⁷, C₃₋₆-heterocycloalkyl and

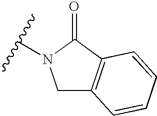

In one highly preferred embodiment, R³ is C₁₋₄-alkyl substituted by —NR⁷(CO)R⁶.

Preferably, for this embodiment, R⁷ is H and R⁶ is selected from C₁₋₆-alkyl, C₃₋₇ cycloalkyl, C₄₋₇-heterocycloalkyl and heteroaryl, each of which may be optionally substituted by one or more substituents selected from halogen, R¹⁰ and —NR⁸R⁹.

In one preferred embodiment, R³ is C₁₋₄-alkyl substituted by —NR⁷(CO)R⁶ and R⁶ is selected from thienyl, cyclopentyl, CH₂-cyclopentyl, methyl, isopropyl, n-propyl, pyrazolyl, cyclohexyl, thiazolyl, oxazolyl, furanyl, imidazolyl, cyclopropyl, CH₂-cyclopropyl, cyclobutyl, triazolyl, pyrrolyl, tetrahydrofuranyl and isoxazolyl, each of which may be substituted by one or more R¹⁰ or —NR⁸R⁹ groups.

In one preferred embodiment, R³ is C₁₋₄-alkyl substituted by —NR⁷(CO)R⁶ and R⁶ is selected from thienyl, cyclopentyl, CH₂-cyclopentyl, methyl, isopropyl, n-propyl, pyrazolyl, cyclohexyl, thiazolyl, oxazolyl, furanyl, imidazolyl, cyclopropyl, CH₂-cyclopropyl, cyclobutyl, triazolyl, pyrrolyl, tetrahydrofuranyl and isoxazolyl, each of which may be substituted by one or more halogen or C₁₋₆-alkyl groups.

Even more preferably, R³ is C₁₋₄-alkyl substituted by —NR⁷(CO)R⁶ and R⁶ is selected from thienyl, cyclopentyl, CH₂-cyclopentyl, methyl, isopropyl, n-propyl, pyrazolyl, cyclohexyl, thiazolyl, oxazolyl, furanyl, CF₃, imidazolyl, cyclopropyl, CH₂-cyclopropyl, cyclobutyl, triazolyl, pyrrolyl, tetrahydrofuranyl, CH₂NMe₂ and isoxazolyl.

In another highly preferred embodiment, R³ is C₁₋₄-alkyl substituted by a heteroaryl group. Preferably, the heteroaryl group is selected from pyrazolyl, tetrazolyl and triazolyl.

In another highly preferred embodiment, R³ is C₁₋₄-alkyl substituted by a —NR⁴R⁵ group. Preferably, R⁴ and R⁵ together with the N to which they are attached form a C₃₋₆-heterocycloalkyl ring optionally further containing one or more CO groups, wherein said C₃₋₆-heterocycloalkyl ring is optionally substituted with one or more R¹⁰ groups. Preferably for this embodiment, R¹⁰ is C₁₋₆-alkyl optionally substituted with one or more halo atoms.

In one preferred embodiment, R³ is C₁₋₄-alkyl substituted by one of the following groups:

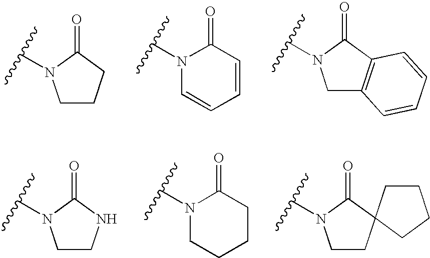

each of which may be optionally substituted with one or more R¹⁰ groups.

In one highly preferred embodiment, R³ is C₁₋₄-alkyl substituted by one of the following groups:

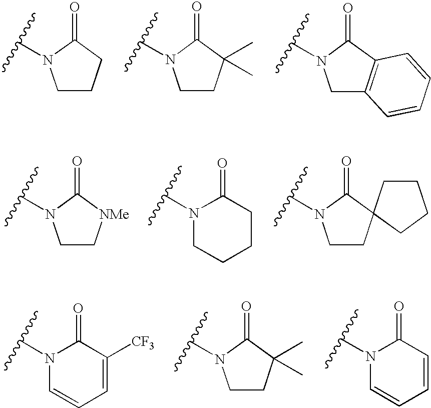

In another highly preferred embodiment, R³ is C₁₋₄-alkyl substituted by a C₃₋₆-heterocycloalkyl group, wherein said C₃₋₆-heterocycloalkyl group is optionally substituted by one or more A groups. Preferably, for this embodiment, the A group is a COR⁶ group.

In another highly preferred embodiment, R³ is C₁₋₄-alkyl substituted by a group selected from:

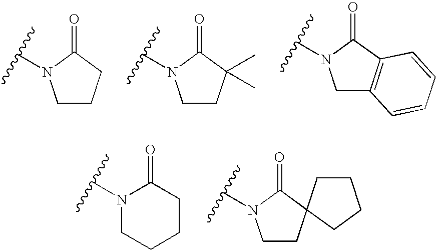

In one especially preferred embodiment of the invention, R³ is an optionally substituted C₃-alkyl group.

In one particularly preferred embodiment, the compound of the invention is selected from the following:

| Structure | Example |
|---|---|
| 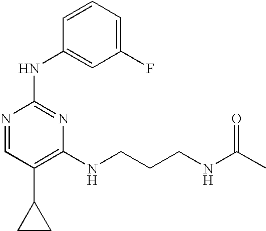 | Example 1 |
| 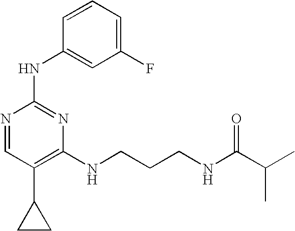 | Example 2 |
| 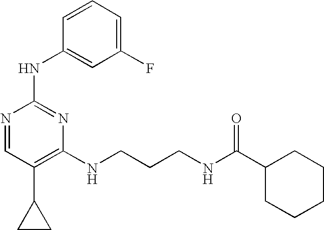 | Example 3 |
| 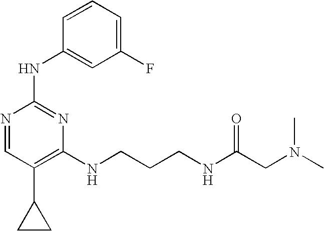 | Example 4 |
| 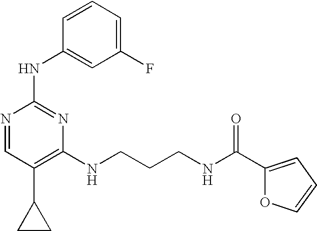 | Example 5 |
| 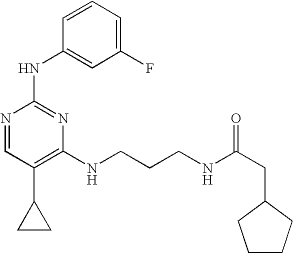 | Example 6 |
| 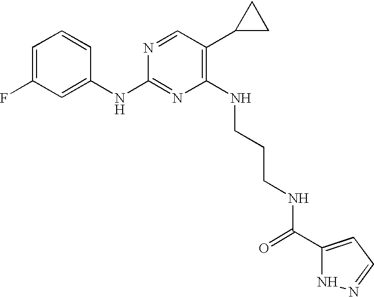 | Example 7 |
| 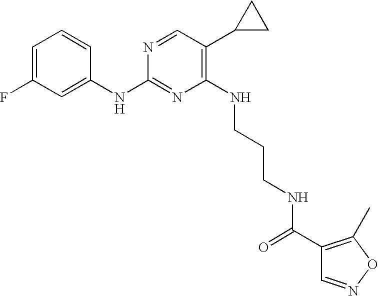 | Example 8 |
| 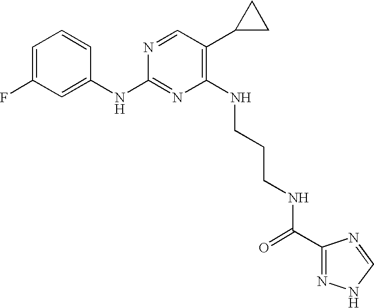 | Example 9 |
| 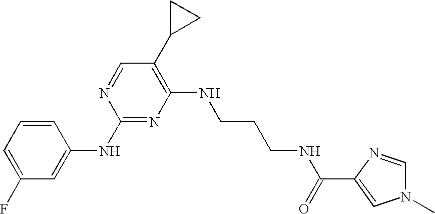 | Example 10 |
| 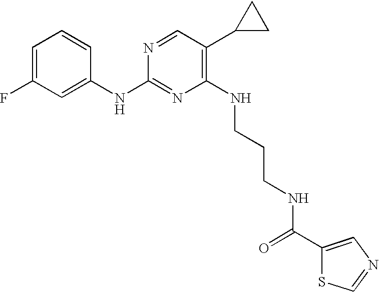 | Example 11 |
| 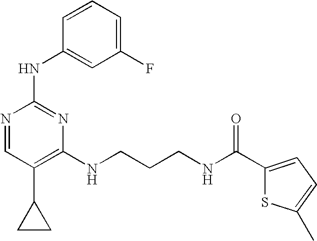 | Example 12 |
| 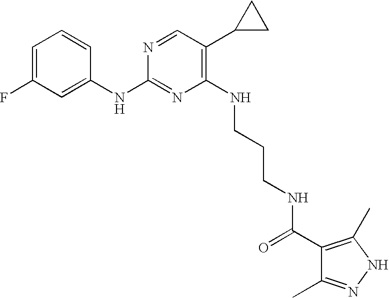 | Example 13 |
| 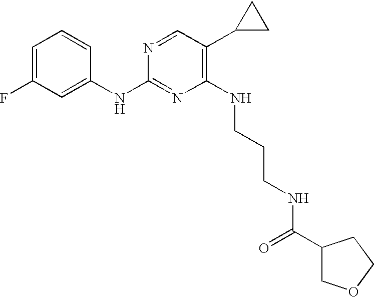 | Example 14 |
| 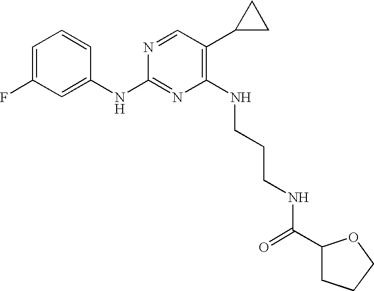 | Example 15 |
| 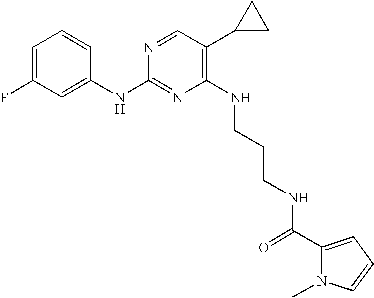 | Example 16 |
| 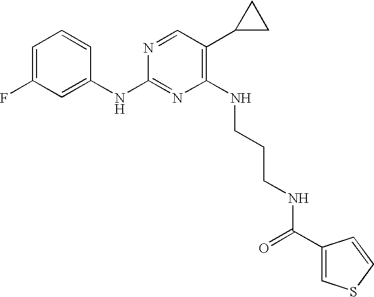 | Example 17 |
| 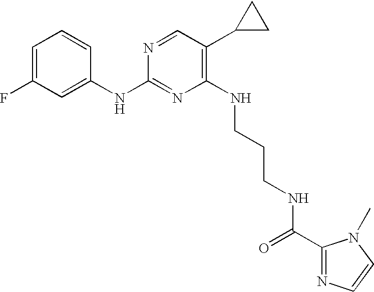 | Example 18 |
| 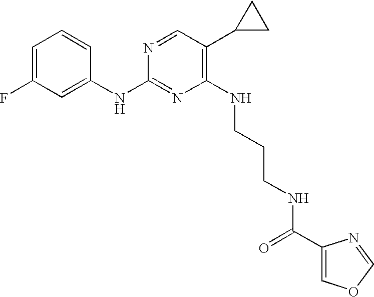 | Example 19 |
| 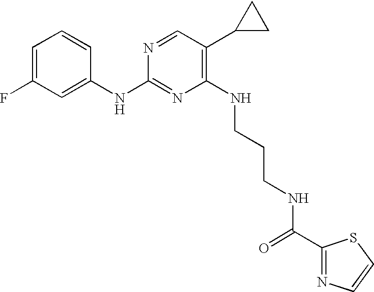 | Example 20 |
| 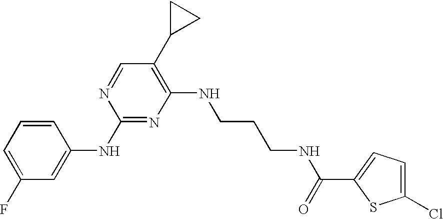 | Example 21 |
| 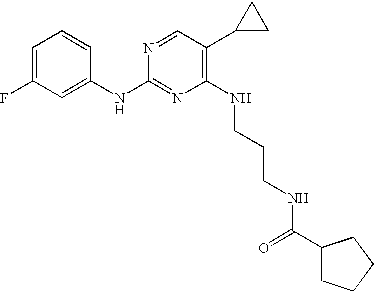 | Example 22 |
| 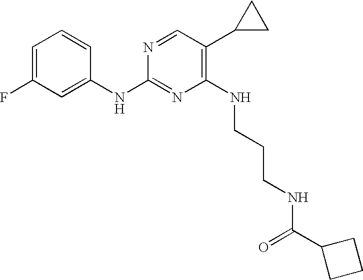 | Example 23 |
| 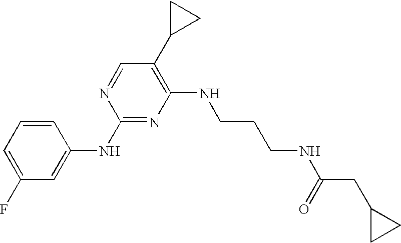 | Example 24 |
| 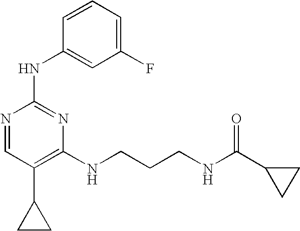 | Example 25 |
| 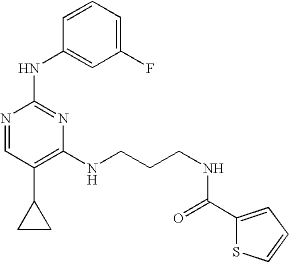 | Example 26 |
| 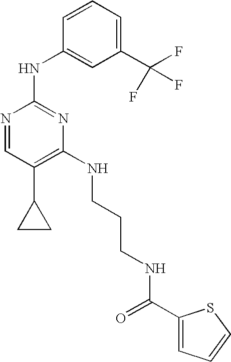 | Example 27 |
| 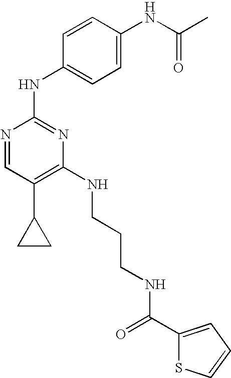 | Example 28 |
| 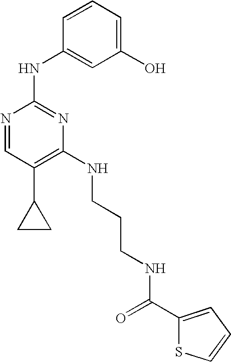 | Example 29 |
| 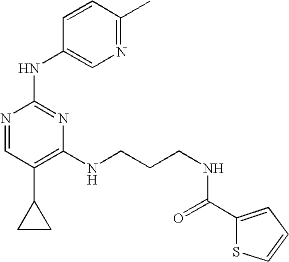 | Example 30 |
| 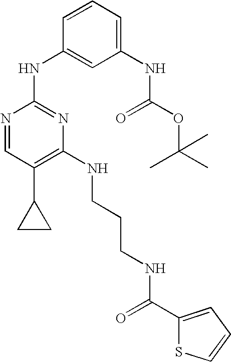 | Example 31 |
| 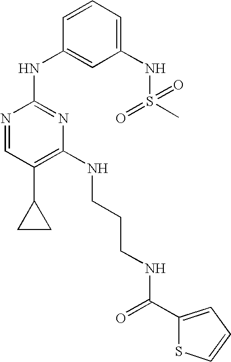 | Example 32 |
| 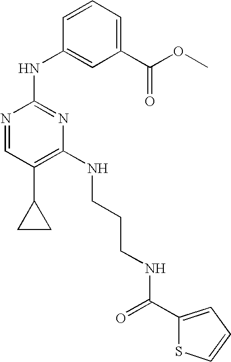 | Example 33 |
| 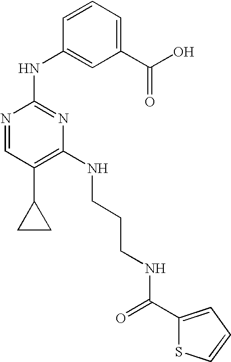 | Example 34 |
| 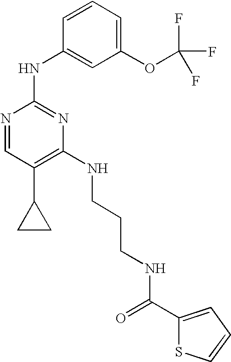 | Example 35 |
| 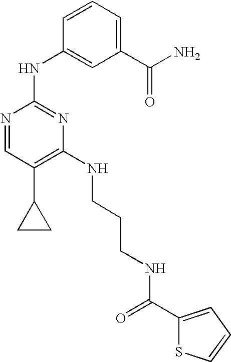 | Example 36 |
| 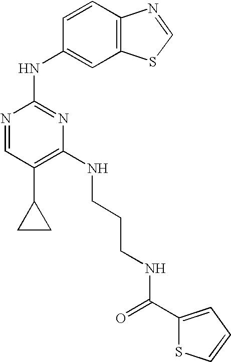 | Example 37 |
| 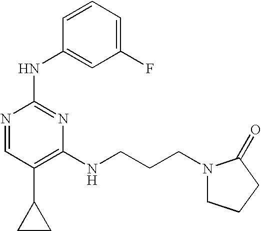 | Example 38 |
| 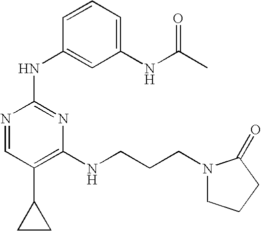 | Example 39 |
| 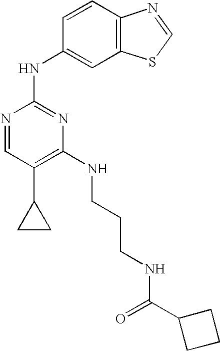 | Example 40 |
| 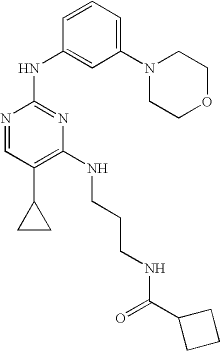 | Example 41 |
| 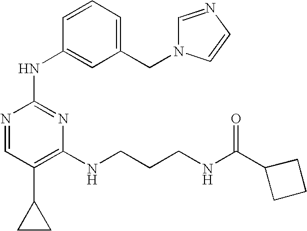 | Example 42 |
| 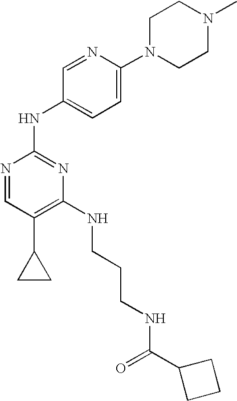 | Example 43 |
| 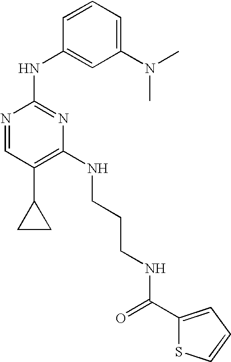 | Example 44 |
| 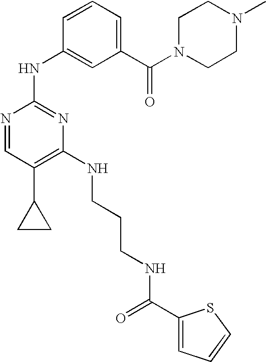 | Example 45 |
| 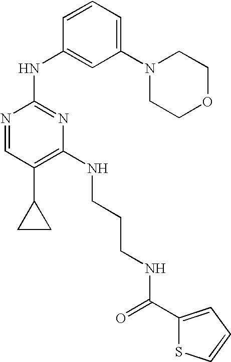 | Example 46 |
| 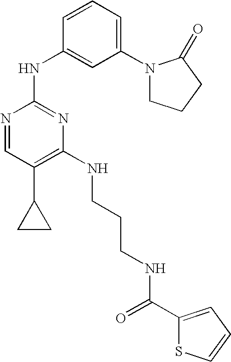 | Example 47 |
| 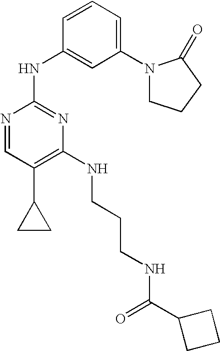 | Example 48 |
| 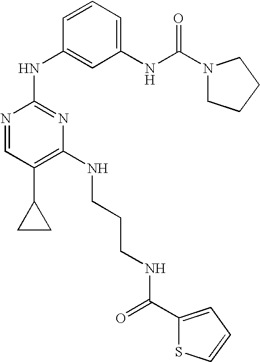 | Example 49 |
| 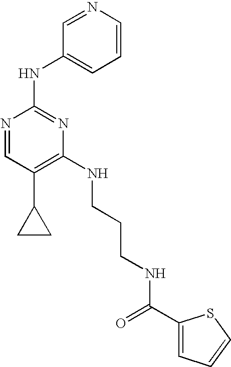 | Example 50 |
| 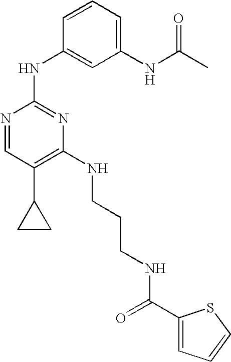 | Example 51 |
| 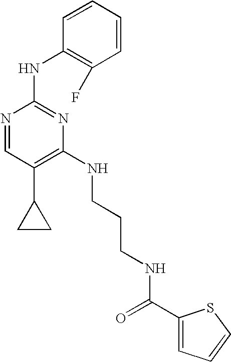 | Example 52 |
| 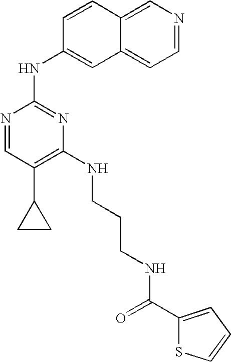 | Example 53 |
| 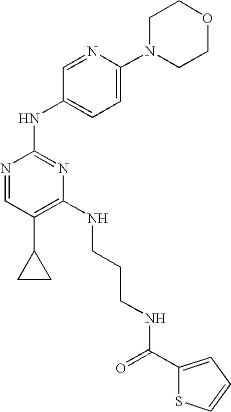 | Example 54 |
| 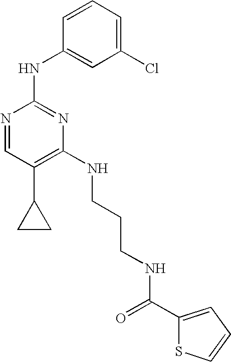 | Example 55 |
| 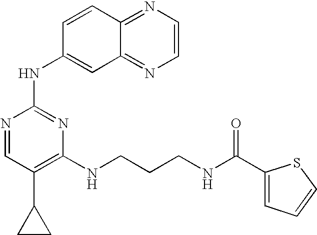 | Example 56 |
| 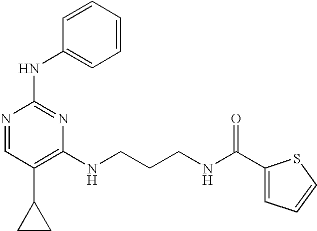 | Example 57 |
| 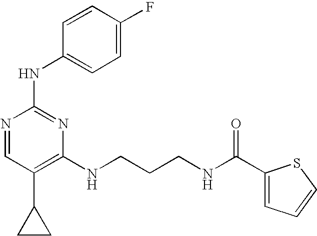 | Example 58 |
| 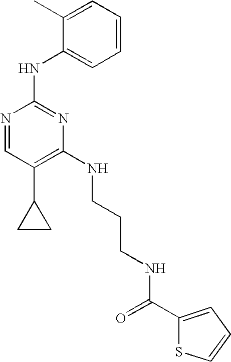 | Example 59 |
| 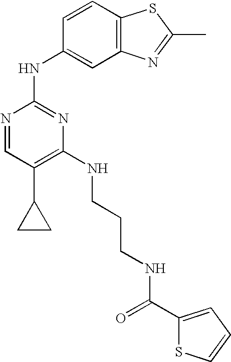 | Example 60 |
| 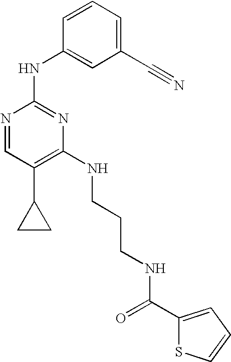 | Example 61 |
| 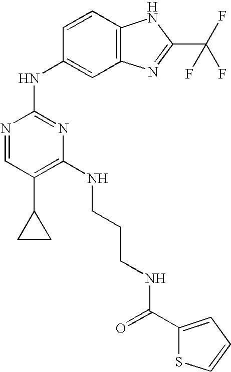 | Example 62 |
| 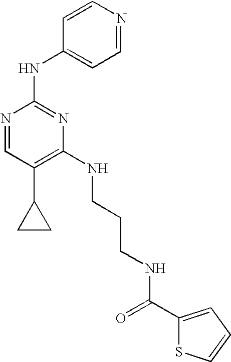 | Example 63 |
| 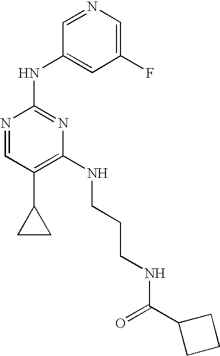 | Example 64 |
| 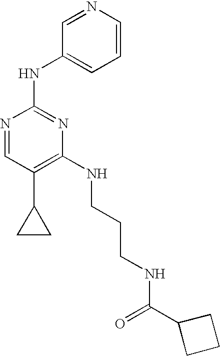 | Example 65 |
| 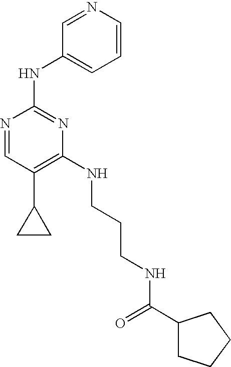 | Example 66 |
| 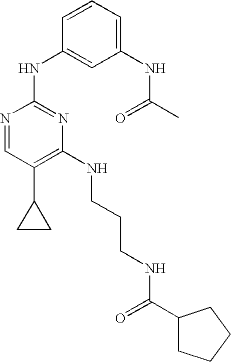 | Example 67 |
| 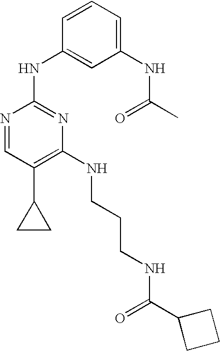 | Example 68 |
| 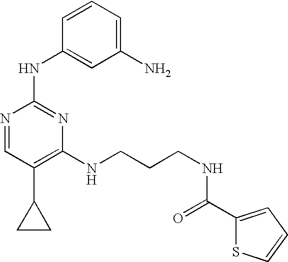 | Example 69 |
| 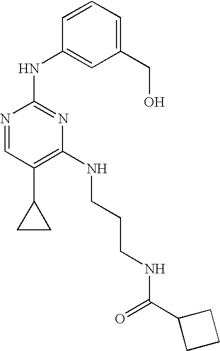 | Example 70 |
| 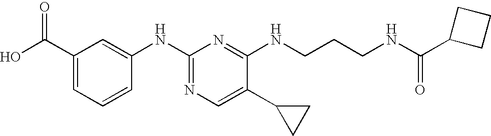 | Example 71 |
| 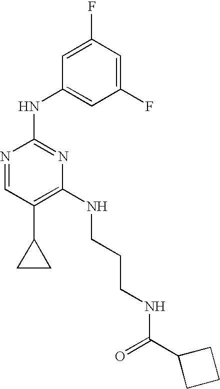 | Example 72 |
| 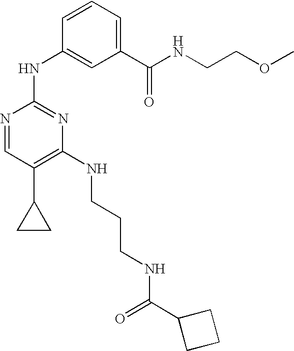 | Example 73 |
| 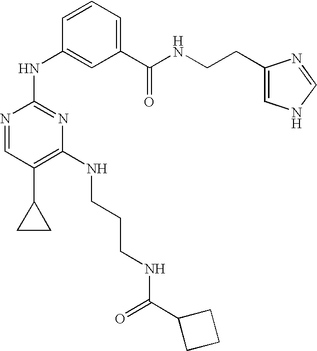 | Example 74 |
| 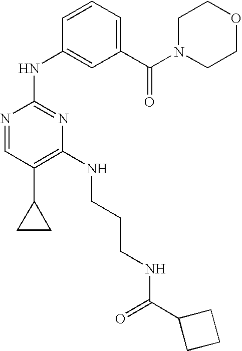 | Example 75 |
| 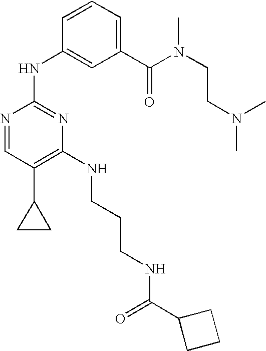 | Example 76 |
| 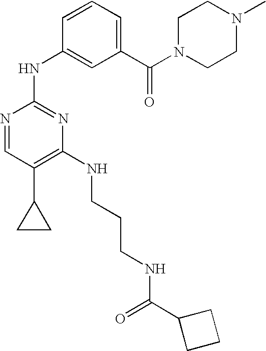 | Example 77 |
| 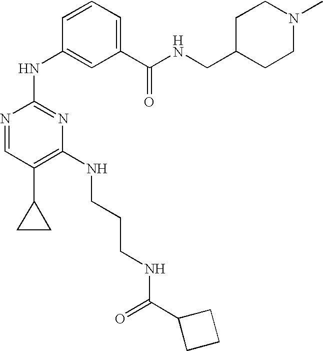 | Example 78 |
| 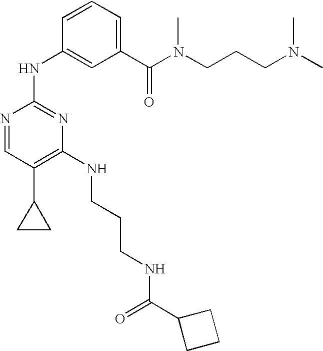 | Example 79 |
| 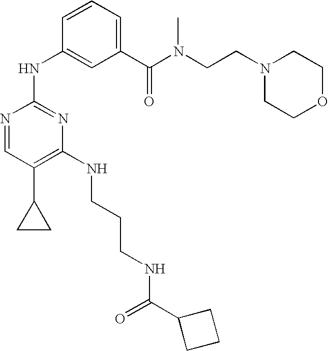 | Example 80 |
| 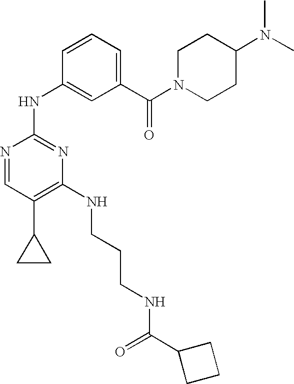 | Example 81 |
| 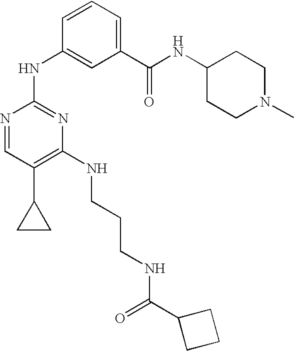 | Example 82 |
| 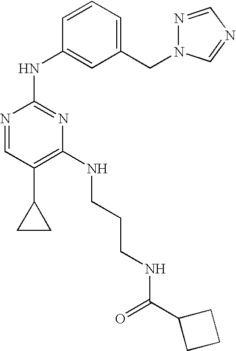 | Example 83 |
| 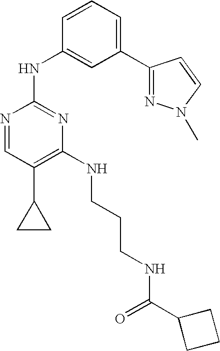 | Example 84 |
| 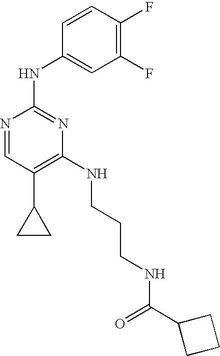 | Example 85 |
| 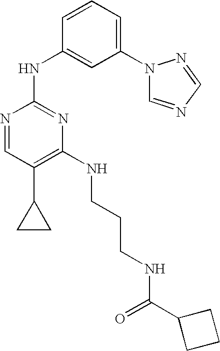 | Example 86 |
| 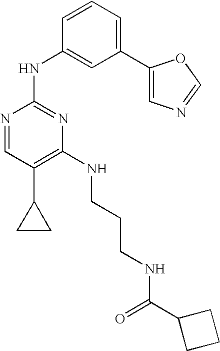 | Example 87 |
| 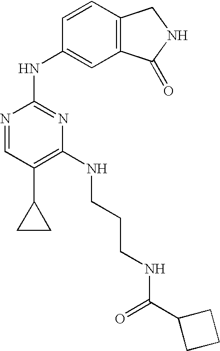 | Example 88 |
| 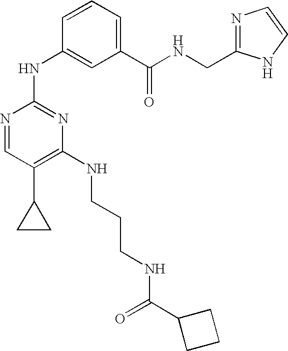 | Example 89 |
| 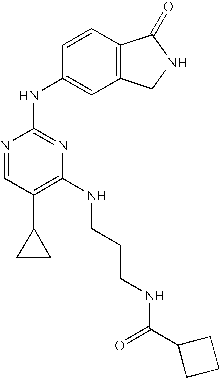 | Example 90 |
| 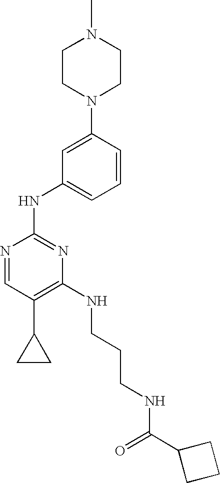 | Example 91 |
| 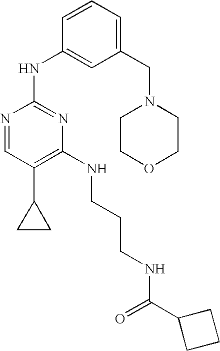 | Example 92 |
| 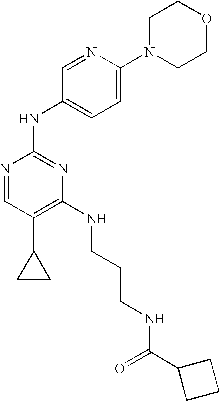 | Example 93 |
| 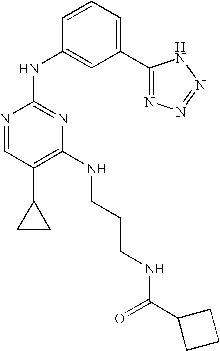 | Example 94 |
| 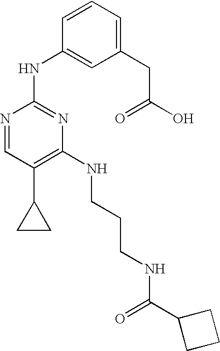 | Example 95 |
| 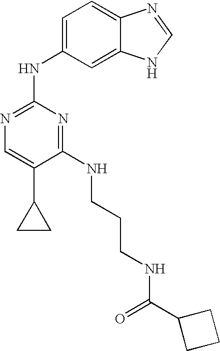 | Example 96 |
| 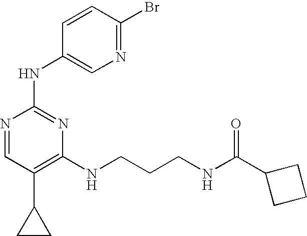 | Example 97 |
| 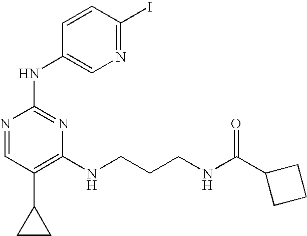 | Example 98 |
| 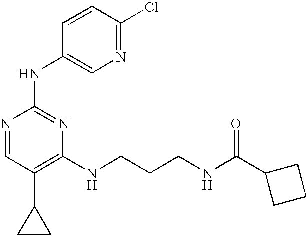 | Example 99 |
| 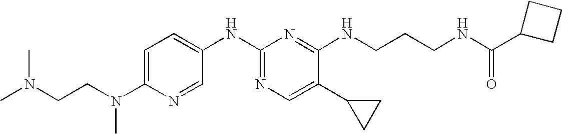 | Example 100 |
| 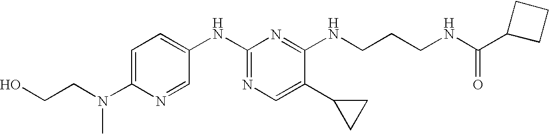 | Example 101 |
| 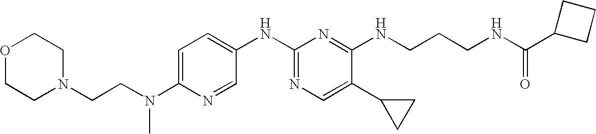 | Example 102 |
| 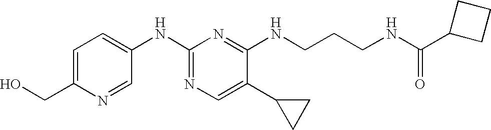 | Example 103 |
| 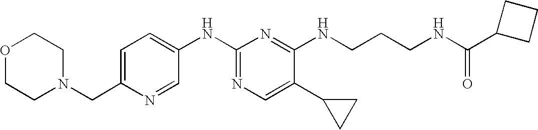 | Example 104 |
| 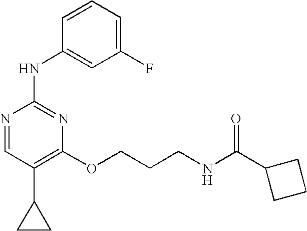 | Example 105 |
| 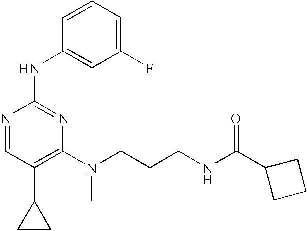 | Example 106 |
| 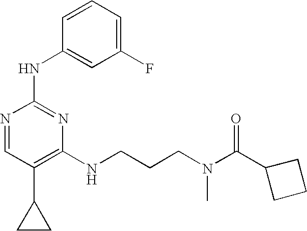 | Example 107 |
| 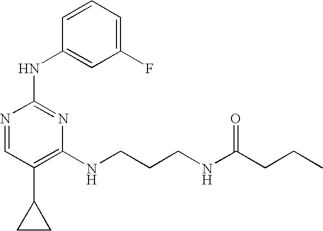 | Example 108 |
| 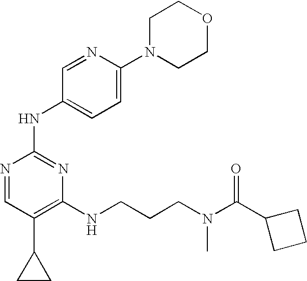 | Example 109 |
| 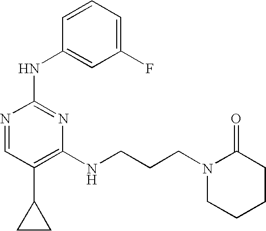 | Example 110 |
| 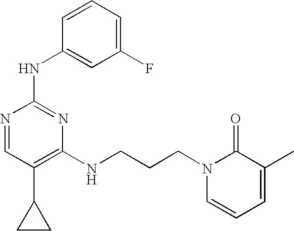 | Example 111 |
| 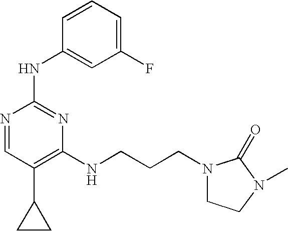 | Example 112 |
| 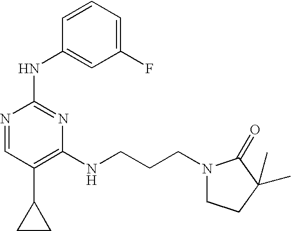 | Example 113 |
| 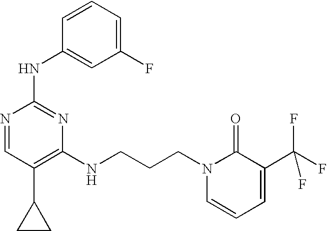 | Example 114 |
| 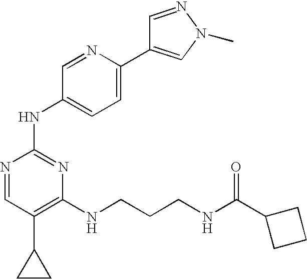 | Example 115 |
| 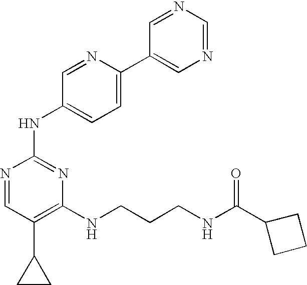 | Example 116 |
| 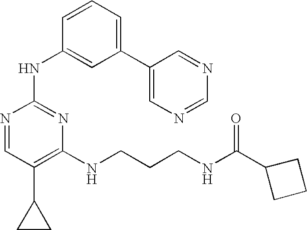 | Example 117 |
| 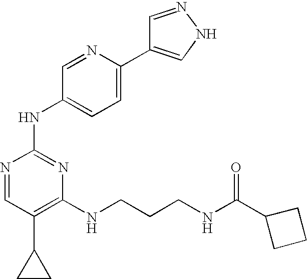 | Example 118 |
| 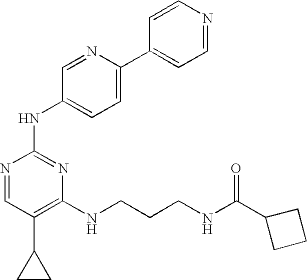 | Example 119 |
| 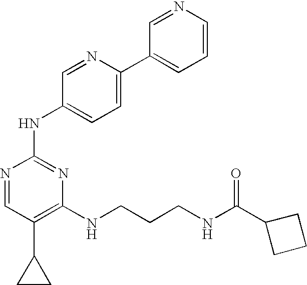 | Example 120 |
| 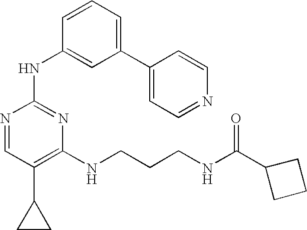 | Example 121 |
| 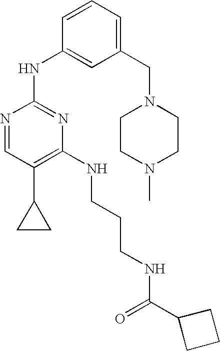 | Example 122 |
| 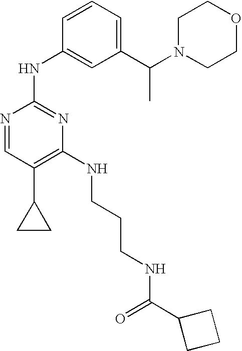 | Example 123 |
| 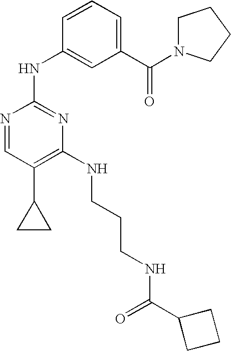 | Example 124 |
| 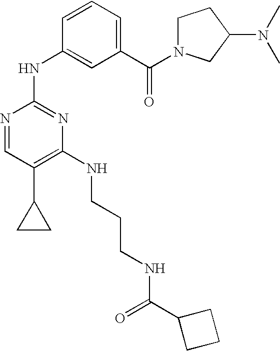 | Example 125 |
| 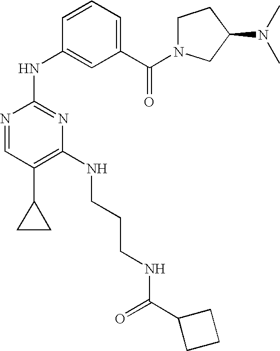 | Example 126 |
| 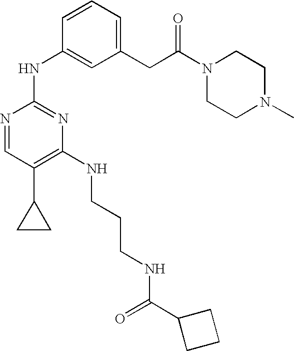 | Example 127 |
| 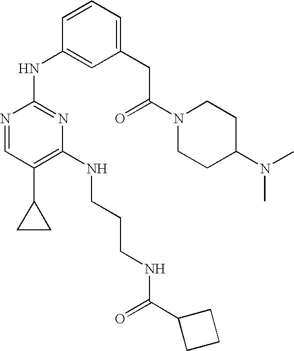 | Example 128 |
| 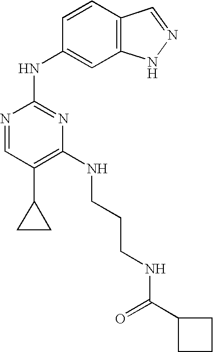 | Example 129 |
| 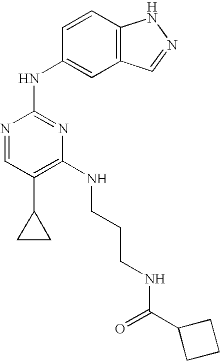 | Example 130 |
| 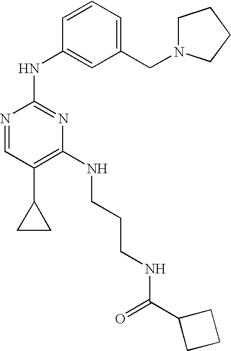 | Example 131 |
| 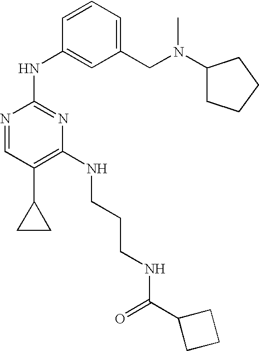 | Example 132 |
| 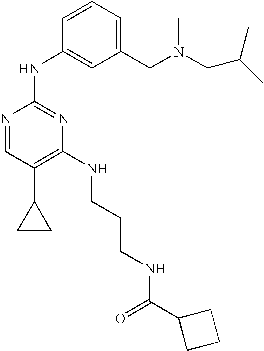 | Example 133 |
| 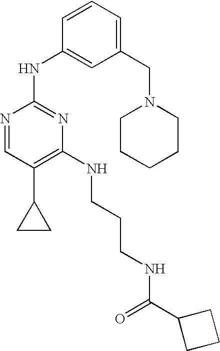 | Example 134 |
| 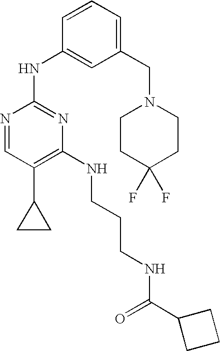 | Example 135 |
| 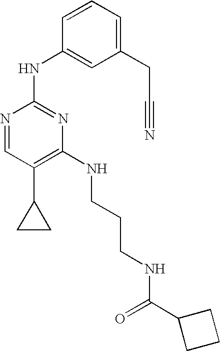 | Example 136 |
| 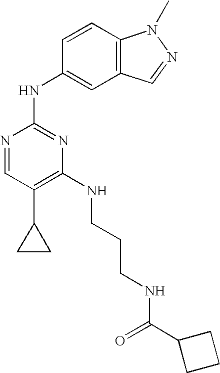 | Example 137 |
| 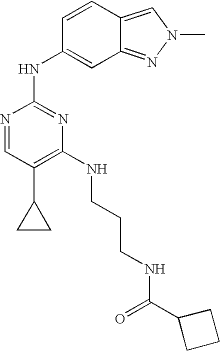 | Example 138 |
| 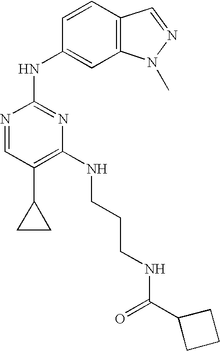 | Example 139 |
| 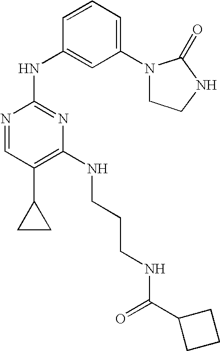 | Example 140 |
| 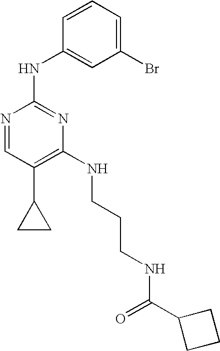 | Example 141 |
| 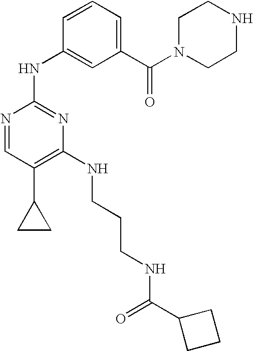 | Example 142 |
| 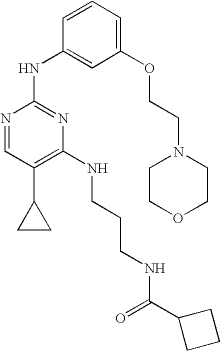 | Example 143 |
| 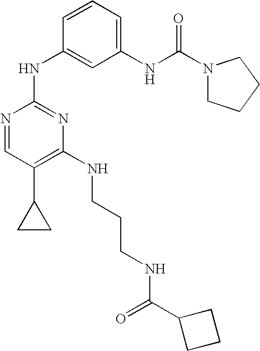 | Example 144 |
| 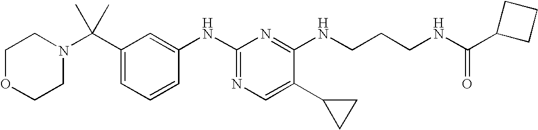 | Example 145 |
| 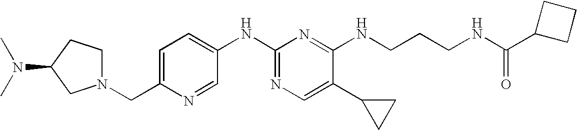 | Example 146 |
| 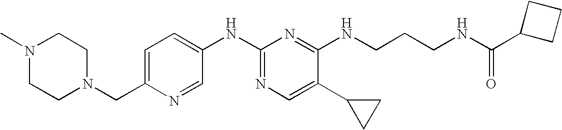 | Example 147 |
| 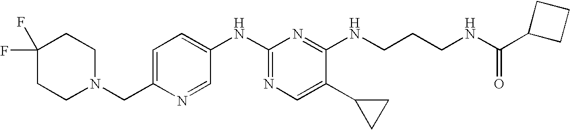 | Example 148 |
| 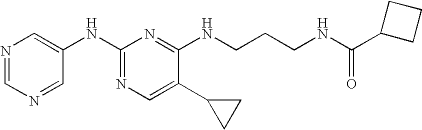 | Example 149 |
| 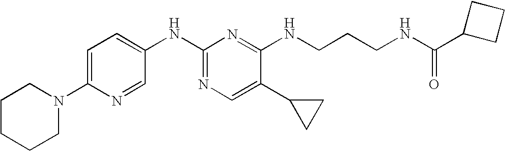 | Example 150 |
| 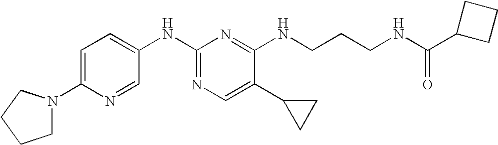 | Example 151 |
| 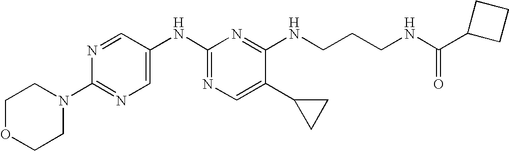 | Example 152 |
| 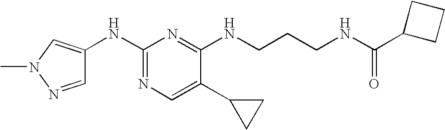 | Example 153 |
| 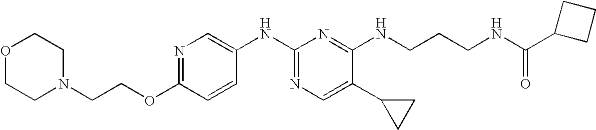 | Example 154 |
| 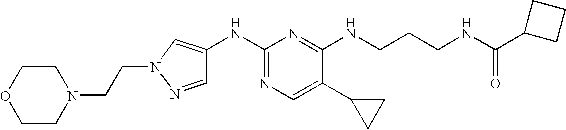 | Example 155 |
| 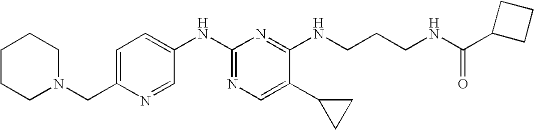 | Example 156 |
| 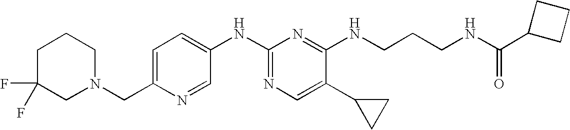 | Example 157 |
| 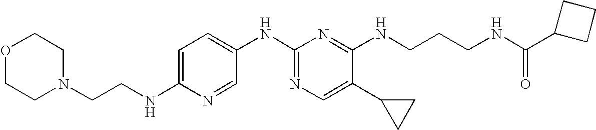 | Example 158 |
| 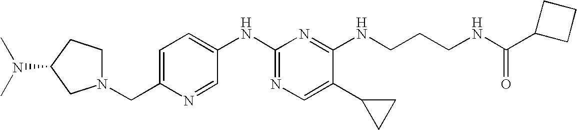 | Example 159 |
| 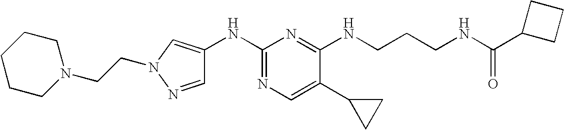 | Example 160 |
| 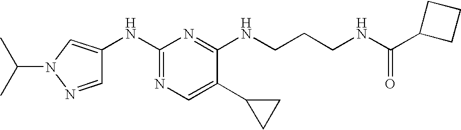 | Example 161 |
| 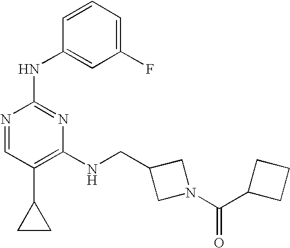 | Example 162 |
| 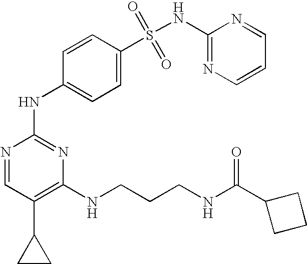 | Example 163 |
| 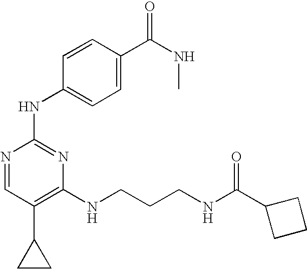 | Example 164 |
| 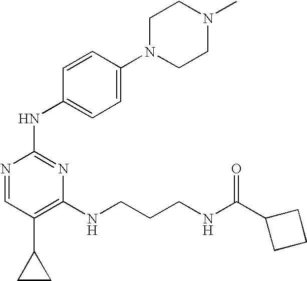 | Example 165 |
| 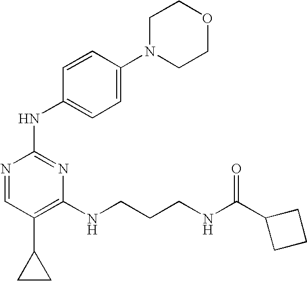 | Example 166 |
| 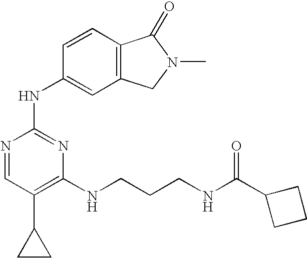 | Example 167 |
| 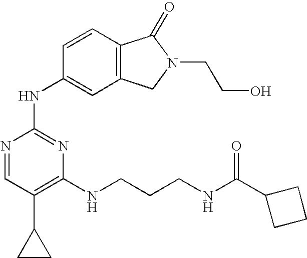 | Example 168 |
| 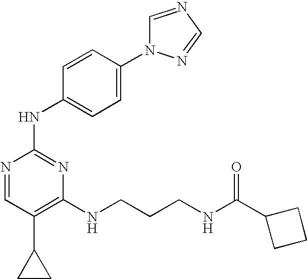 | Example 169 |
| 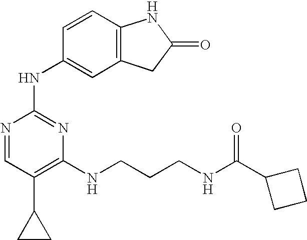 | Example 170 |
| 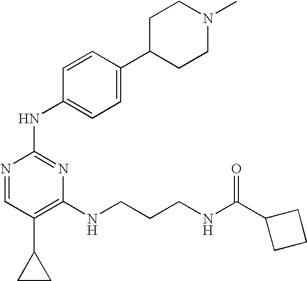 | Example 171 |
| 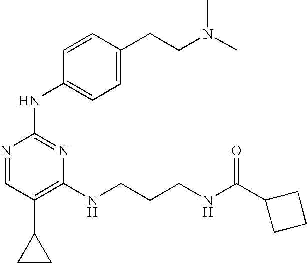 | Example 172 |
| 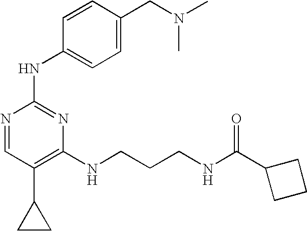 | Example 173 |
| 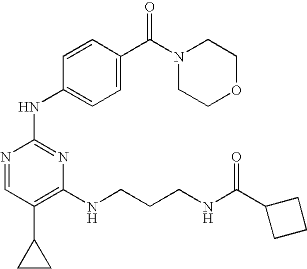 | Example 174 |
| 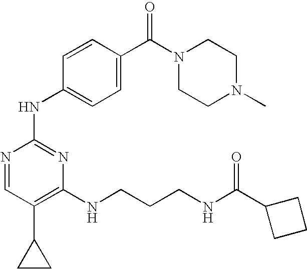 | Example 175 |
| 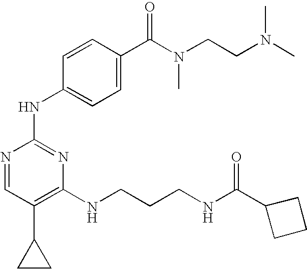 | Example 176 |
| 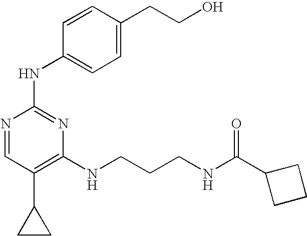 | Example 177 |
| 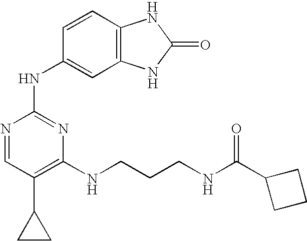 | Example 178 |
| 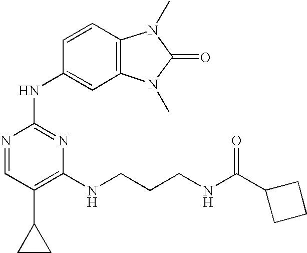 | Example 179 |
| 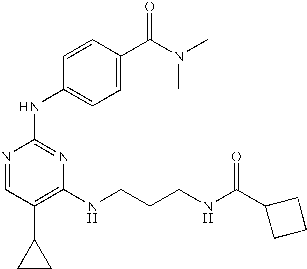 | Example 180 |
| 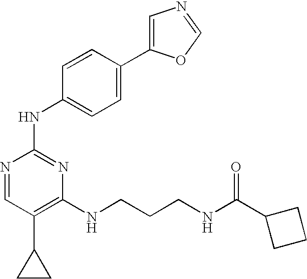 | Example 181 |
| 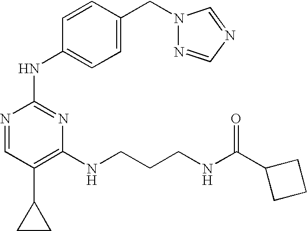 | Example 182 |
| 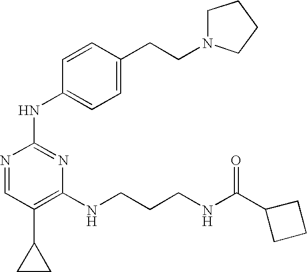 | Example 183 |
| 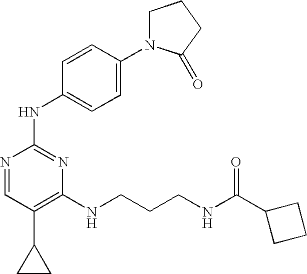 | Example 184 |
| 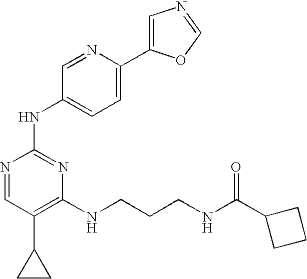 | Example 185 |
| 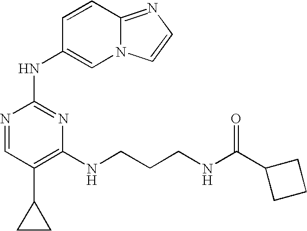 | Example 186 |
| 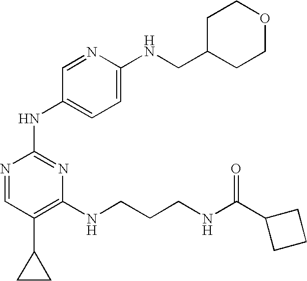 | Example 187 |
| 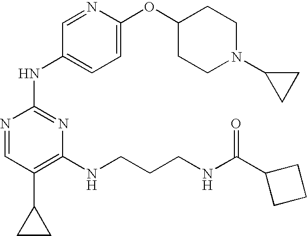 | Example 188 |
| 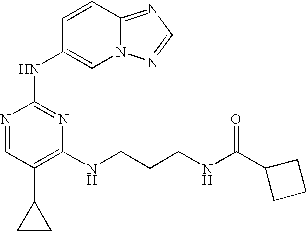 | Example 189 |
| 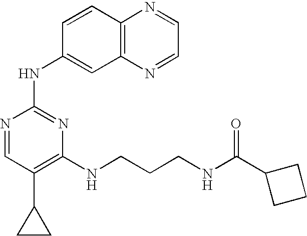 | Example 190 |
| 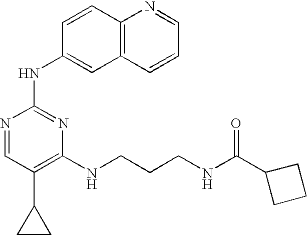 | Example 191 |
| 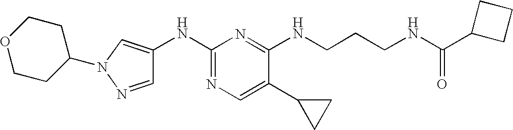 | Example 192 |
| 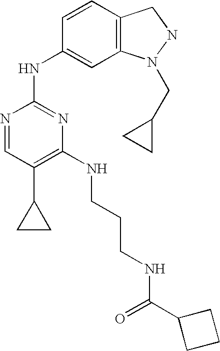 | Example 193 |
| 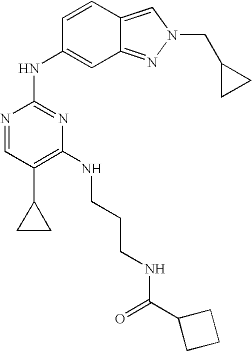 | Example 194 |
| 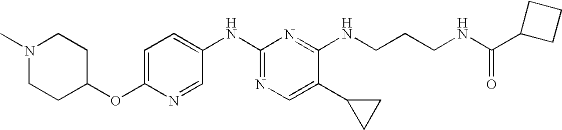 | Example 195 |
| 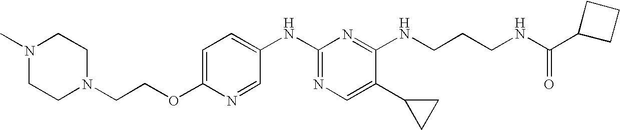 | Example 196 |
| 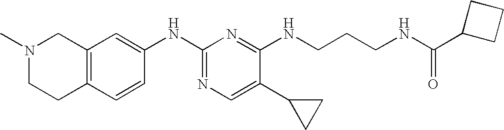 | Example 197 |
| 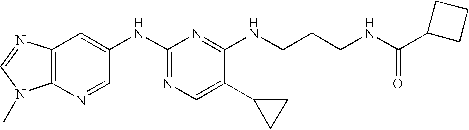 | Example 198 |
| 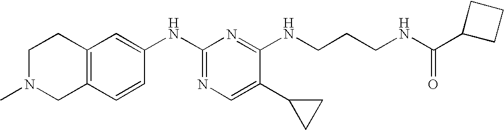 | Example 199 |
| 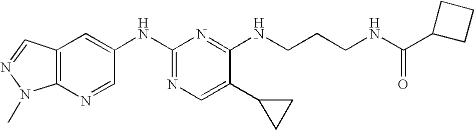 | Example 200 |
| 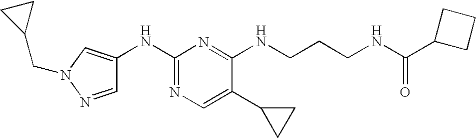 | Example 201 |
| 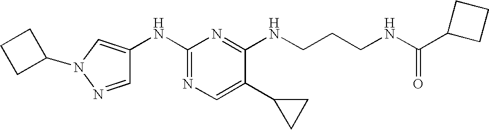 | Example 202 |
| 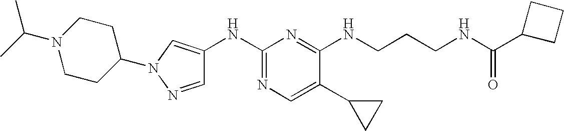 | Example 203 |
| 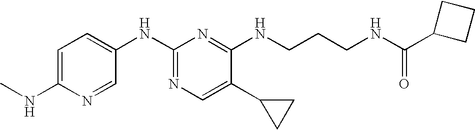 | Example 204 |
| 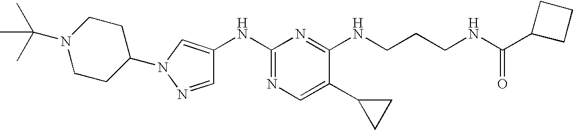 | Example 205 |
| 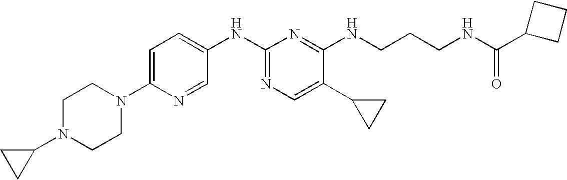 | Example 206 |
| 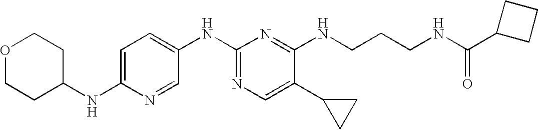 | Example 207 |
| 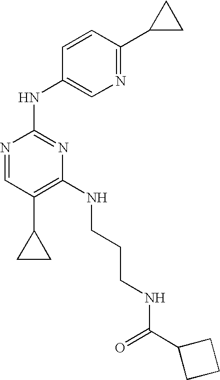 | Example 208 |
| 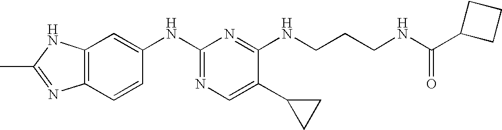 | Example 209 |
| 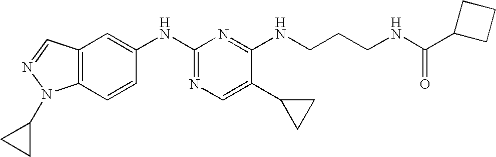 | Example 210 |
| 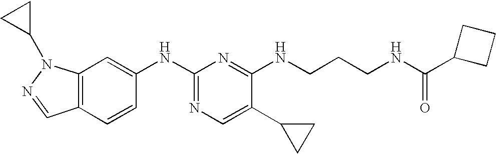 | Example 211 |
| 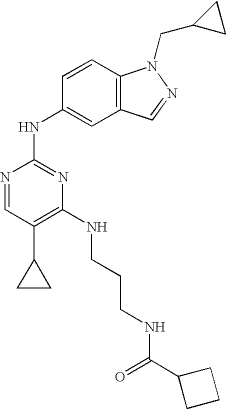 | Example 212 |
| 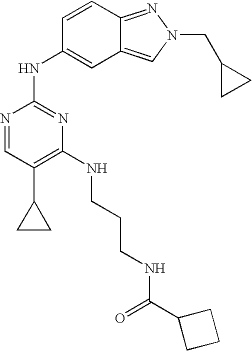 | Example 213 |
| 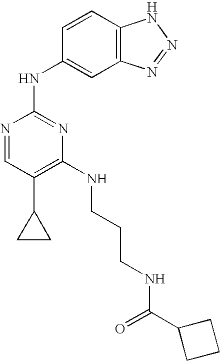 | Example 214 |

In another highly preferred embodiment, the compound of the invention is selected from compounds 1-214 above.

A further aspect of the invention relates to a compound as described above for use in medicine.

### Therapeutic Applications

Another aspect of the invention relates to a compound as described above for use in treating a disorder selected from cancer, septic shock, neurodegenerative diseases, Alzheimer's disease, Primary open Angle Glaucoma (POAG), hyperplasia, rheumatoid arthritis, psoriasis, artherosclerosis, retinopathy, osteoarthritis, endometriosis and chronic inflammation.

Another aspect relates to the use of a compound as described above in the preparation of a medicament for treating or preventing a disorder selected from cancer, septic shock, neurodegenerative diseases, Alzheimer's disease, Primary open Angle Glaucoma (POAG), hyperplasia, rheumatoid arthritis, psoriasis, artherosclerosis, retinopathy, osteoarthritis, endometriosis and chronic inflammation.

Preferably, the compound is administered in an amount sufficient to inhibit a kinase selected from TBK1, MKK1, ERK8, RSK1, RSK2, PDK1, S6K1, MNK2, PHK, CHK1, CHK2, GSK3beta, CDK2, MARK3, MELK, IRR, VEG-FR and IKKepsilon.

Yet another aspect relates to the use of a compound of the invention in the preparation of a medicament for the prevention or treatment of a disorder caused by, associated with or accompanied by any abnormal kinase activity, wherein the kinase is selected from TBK1, MKK1, ERK8, RSK1, RSK2, PDK1, S6K1, MNK2, PHK, CHK1, CHK2, GSK3beta, CDK2, MARK3, MELK, IRR, VEG-FR, IKKepsilon and combinations thereof.

Preferably, the kinase is selected from TBK1, PDK1, ERK8, MARK3, and IKKepsilon and combinations thereof.

More preferably, the kinase is selected from TBK1 and PDK1.

Even more preferably, the kinase is TBK1.

In one preferred embodiment, the compound of the invention is exhibits an IC₅₀ value against TBK1 of from about 1 μM to about 10 μM, more preferably from about 100 nM to about 1 μM, even more preferably, less than about 100 nM.

Preferably, the disorder is selected from cancer, septic shock, neurodegenerative diseases, Alzheimer's disease, diseases of the eye, including Primary open Angle Glaucoma (POAG), hyperplasia, rheumatoid arthritis, autoimmune diseases, artherosclerosis, retinopathy, osteoarthritis, fibrotic diseases, endometriosis and chronic inflammation.

In the innate immune system TBK1 is activated in response to LPS engagement of Toll-like receptor 4 (TLR4) or double-stranded RNA (from double stranded RNA viruses) engagement of TLR3. It is also activated in response to the pro-inflammatory cytokines TNF and IL-1. Once activated TBK1 phosphorylates and activates interferon-regulatory factor 3 (IRF3), a transcription factor that triggers the production of interferon beta and chemokines, like interleukin-8 (IL-8) and RANTES. These substances play a key role in mediating host defence against infection by bacteria and viruses. Mice that do not express interferon beta or IRF3 are resistant to LPS-induced septic shock. These observations suggest that a drug that inhibits TBK1 may have efficacy for the treatment/prevention of septic shock and/or the treatment of inflammatory disease.

TBK1 is also activated in response to hypoxia and stimulates the production of pro-angiogenic factors, such as VEGF and IL-1. The expression of TBK1 rises 2.5-3-fold after 24 h of hypoxia, similar to the increase in expression of VEGF. The hypoxia induced increase in VEGF expression can be abolished by siRNA “knockdown” of TBK1. The level of TBK1 mRNA and protein is elevated in malignant colon and breast cancer cells (see Korherr et al (2006) PNAS 103, 4240-4245 and references therein). TBK1 is also recruited and activated by the RalB/Sec5 effector complex; in cancer cells, constitutive engagement of this pathway via chronic RalB activation, restricts the initiation of apoptotic programmes (Chien et al (2006) Cell 127, 157-170 and references there-in). For these reasons the drugs that inhibit TBK1 may have efficacy for the treatment of cancers. In one preferred embodiment, the compounds of the invention are useful in the treatment of Primary open Angle Glaucoma (POAG).

Primary Open Angle Glaucoma (POAG) is a leading cause of irreversible blindness affecting 35 million people worldwide. The disease is genetically heterogeneous and mutations in the protein optineurin (OPTN) are associated with a form of POAG associated with normal intraocular pressure, termed Normal Tension Glaucoma (NTG) or Low Tension Glaucoma (LTG).^{1,2} A study of 54 families with autosomal dominant adult onset glaucoma, 17% had one of four sequence mutations in OPTN, the most prevalent being the OPTN[E50K] mutant. How this mutation in OPTN might cause POAG is unknown. However, tumour necrosis factor α (TNFα) has been reported to increase the severity of damage in optic nerve heads of POAG and LTG subjects^{3,4}. Moreover, exposure to TNFα¹⁰ induces the de novo expression of optineurin. These observations suggest that some forms of POAG may be caused by an abnormal response to this cytokine.¹¹ The compounds described herein may therefore find use in treating POAG and/or diseases associated with optineurin activity.

Another aspect of the invention relates to a method of treating a TBK1, MKK1, ERK8, RSK1, RSK2, S6K1, MNK2, PHK, CHK1, CHK2, GSK3beta, CDK2, MARK3, MELK, IRR, VEG-FR, and/or IKKepsilon and optionally PDK1 related disease or disorder. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinafter.

Yet another aspect of the invention relates to a method of treating a mammal having a disease state alleviated by the inhibition of a kinase selected from TBK1, MKK1, ERK8, RSK1, RSK2, PDK1, S6K1, MNK2, PHK, CHK1, CHK2, GSK3beta, CDK2, MARK3, MELK, IRR, VEG-FR, and IKKepsilon, wherein the method comprises administering to a mammal a therapeutically effective amount of a compound according to the invention.

Preferably, the disease state is alleviated by the inhibition of TBK1, PDK1, ERK8, MARK3 or IKKepsilon, more preferably TBK1 or IKKepsilon, even more preferably TBK1.

Preferably, the mammal is a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “administering” as used herein refers to a method for bringing a compound of the present invention and a target kinase together in such a manner that the compound can affect the enzyme activity of the kinase either directly; i.e., by interacting with the kinase itself or indirectly; i.e., by interacting with another molecule on which the catalytic activity of the kinase is dependent. As used herein, administration can be accomplished either in vitro, i.e. in a test tube, or in vivo, i.e., in cells or tissues of a living organism.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (see, e.g., Fingl et al., 1975, In: The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/kg/day, commonly from about 100-1000 mg/kg/day, preferably from about 150-700 mg/kg/day and most preferably from about 250-500 mg/kg/day. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, kinase related disease or disorder” refers to a disease or disorder characterized by inappropriate kinase activity or over-activity of a kinase as defined herein. Inappropriate activity refers to either; (i) kinase expression in cells which normally do not express said kinase; (ii) increased kinase expression leading to unwanted cell proliferation, differentiation and/or growth; or, (iii) decreased kinase expression leading to unwanted reductions in cell proliferation, differentiation and/or growth. Over-activity of kinase refers to either amplification of the gene encoding a particular kinase or production of a level of kinase activity, which can correlate with a cell proliferation, differentiation and/or growth disorder (that is, as the level of the kinase increases, the severity of one or more of the symptoms of the cellular disorder increases). Over activity can also be the result of ligand independent or constitutive activation as a result of mutations such as deletions of a fragment of a kinase responsible for ligand binding.

Preferred diseases or disorders that the compounds described herein may be useful in preventing, treating and/or studying are cell proliferative disorders, especially cancer such as, but not limited to, papilloma, blastoglioma, Kaposi's sarcoma, melanoma, lung cancer, ovarian cancer, prostate cancer, squamous cell carcinoma, astrocytoma, head cancer, neck cancer, skin cancer, liver cancer, bladder cancer, breast cancer, lung cancer, uterus cancer, prostate cancer, testis carcinoma, colorectal cancer, thyroid cancer, pancreatic cancer, gastric cancer, hepatocellular carcinoma, leukemia, lymphoma, Hodgkin's disease and Burkitt's disease.

Another condition to which the compounds described herein may be useful in preventing, treating and/or studying is septic shock.

Another condition to which the compounds described herein may be useful in preventing, treating and/or studying is inflammatory disease.

P. Cohen et al. have observed that TBK1 binds in an enhanced manner to the mutant form of optineurin which causes a form of primary open angle glaucoma (POAG).¹¹ The compounds described herein may therefore find use in treating POAG and/or diseases associated with optineurin activity.

A further aspect relates to the use of a compound which is capable of inhibiting the binding of TBK1 to a mutant form of OPTN for the manufacture of a medicament for treating POAG and/or a disease where it would be desirable to inhibit or reduce TBKI binding to mutant form of OPTN. One such mutant is the OPTN (E50K) mutant. Suitable compounds may include the compounds identified herein.

In a further aspect there is provided a method of treating a patient suffering from POAG, comprising the step of administering to the subject an effective amount of a compound which is capable of inhibiting an interaction between TBK1 and a mutant form of OPTN, associated with POAG. Suitable compounds include those according to Formula I.

In a further aspect there is provided a method of treating a patient suffering from a disease associated with abnormal cell proliferation, comprising the step of administering to the subject an effective amount of a compound of the invention.

In a further aspect there is provided a method of treating a patient suffering from septic shock, neurodegenerative diseases, Alzheimer's disease, comprising the step of administering to the subject an effective amount of a compound of the invention.

Thus, the present invention further provides use of compounds as defined herein for the manufacture of medicaments for the treatment of diseases where it is desirable to inhibit TBK1 and/or IKK epsilon. Such diseases include colon and breast cancer, septic shock and/or POAG. A number of papers^{5,6,7} have described that TBK1 and IKKepsilon modulate expression of interferon and interferon inducible genes, without affecting induction of pro-inflammatory cytokines. This indicates that the compounds disclosed herein, may find applications in treating/preventing septic shock or viral infection. Mice that do not express interferon beta or IRF3 are resistant to lipopolysaccharide induced septic shock so that inhibitors of TBK1 should be expected to have a similar effect.

### Pharmaceutical Compostions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectible preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use. Formulations suitable for pulmonary administration via the buccal cavity are presented such that particles containing an active compound and desirably having a diameter in the range of 0.5 to 7 microns are delivered in the bronchial tree of the recipient. As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically or veterinarily acceptable carrier or vehicle.

### Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulfuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art. Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulfur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Prodrugs

The invention further includes the compounds of the present invention in prodrug form, i.e. covalently bonded compounds which release the active parent drug according to general formula (I) in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Solvates

The present invention also includes solvate forms of the compounds of the present invention. The terms used in the claims encompass these forms.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In accordance with this invention, an effective amount of a compound of general formula (I) may be administered to inhibit the kinase implicated with a particular condition or disease. Of course, this dosage amount will further be modified according to the type of administration of the compound. For example, to achieve an “effective amount” for acute therapy, parenteral administration of a compound of general formula (I) is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit a kinase. The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

### Combinations

In a particularly preferred embodiment, the one or more compounds of the invention are administered in combination with one or more other active agents, for example, existing drugs available on the market. In such cases, the compounds of the invention may be administered consecutively, simultaneously or sequentially with the one or more other active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the inhibitory activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

### Assay

A further aspect of the invention relates to the use of a compound as described above in an assay for identifying further candidate compounds capable of inhibiting one or more kinases selected from TBK1, MKK1, ERK8, RSK1, RSK2, PDK1, S6K1, MNK2, PHK, CHK1, CHK2, GSK3beta, CDK2, MARK3, MELK, IRR, VEG-FR, and IKKepsilon.

Preferably, the assay is a competitive binding assay.

More preferably, the competitive binding assay comprises contacting a compound of the invention with a kinase selected from TBK1, MKK1, ERK8, RSK1, RSK2, PDK1, S6K1, MNK2, PHK, CHK1, CHK2, GSK3beta, CDK2, MARK3, MELK, IRR, VEG-FR, and IKKepsilon, and a candidate compound and detecting any change in the interaction between the compound according to the invention and the kinase.

Preferably, the candidate compound is generated by conventional SAR modification of a compound of the invention.

As used herein, the term “conventional SAR modification” refers to standard methods known in the art for varying a given compound by way of chemical derivatisation.

Thus, in one aspect, the identified compound may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through-put screen.

This invention also contemplates the use of competitive drug screening assays in which neutralising antibodies capable of binding a compound specifically compete with a test compound for binding to a compound.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Preferably, the competitive binding assay comprises contacting a compound of the invention with a kinase in the presence of a known substrate of said kinase and detecting any change in the interaction between said kinase and said known substrate.

A further aspect of the invention provides a method of detecting the binding of a ligand to a kinase, said method comprising the steps of:

(i) contacting a ligand with a kinase in the presence of a known substrate of said kinase;
(ii) detecting any change in the interaction between said kinase and said known substrate;
and wherein said ligand is a compound of the invention.

One aspect of the invention relates to a process comprising the steps of:

(a) performing an assay method described hereinabove;
(b) identifying one or more ligands capable of binding to a ligand binding domain; and
(c) preparing a quantity of said one or more ligands.

Another aspect of the invention provides a process comprising the steps of:

(a) performing an assay method described hereinabove;
(b) identifying one or more ligands capable of binding to a ligand binding domain; and
(c) preparing a pharmaceutical composition comprising said one or more ligands.

Another aspect of the invention provides a process comprising the steps of:

(a) performing an assay method described hereinabove;
(b) identifying one or more ligands capable of binding to a ligand binding domain;
(c) modifying said one or more ligands capable of binding to a ligand binding domain;
(d) performing the assay method described hereinabove;
(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of one or more disorders selected from cancer, septic shock, neurodegenerative diseases, Alzheimer's disease, diseases of the eye, including Primary open Angle Glaucoma (POAG), hyperplasia, rheumatoid arthritis, autoimmune diseases, artherosclerosis, retinopathy, osteoarthritis, fibrotic diseases, endometriosis and chronic inflammation.

The above methods may be used to screen for a ligand useful as an inhibitor of one or more kinases.

Compounds of general formula (I) are useful both as laboratory tools and as therapeutic agents. In the laboratory certain compounds of the invention are useful in establishing whether a known or newly discovered kinase contributes a critical or at least significant biochemical function during the establishment or progression of a disease state, a process commonly referred to as ‘target validation’.

### Synthesis

A further aspect of the invention relates to a process for preparing a compound as described above, said process comprising the steps of:

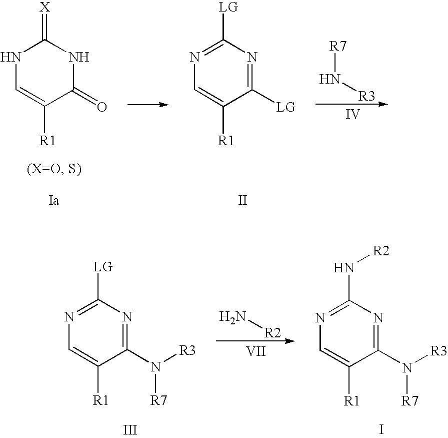

(i) converting a compound of formula Ia to a compound of formula II, where each LG is independently a leaving group;
(ii) reacting said compound of formula II with an amine of formula IV to form a compound of formula III;
(iii) reacting said compound of formula III with an amine of formula VII to form a compound of formula I.

Scheme 1 illustrates the conversion of compounds of formula (Ia) to compounds of formula (II), wherein R1, R3 and R7 are as defined previously.

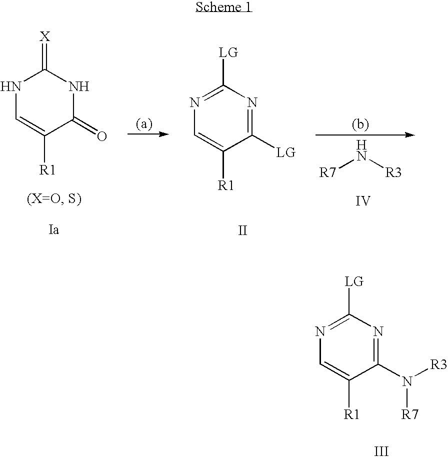

Compounds of formula (Ia) can be obtained from commercial sources or by following known literature procedures; R1, R3 and R7 are as defined previously; LG refers to a leaving group such as halogen, methoxy, thiomethyl, triflate, tosylate or the group —SO₂Me (but is preferably chlorine). Compounds with formula (IV) are commercially available, known in the literature or are readily obtainable by the skilled person by following standard chemical procedures.

### Step (a)

Compounds of formula (Ia) can be converted to compounds of formula (II) by treatment with POCl₃ in the presence of an additive, such as N,N-diisopropylethylamine at temperatures in the range of 50° C. to reflux for reaction times up to 24 h.

Preferred conditions: 1 eq. of formula 1, 1 eq of N,N-diisopropylethylamine, 7.8 eq. of POCl₃ heating at 90° C. for 2 h.

### Step (b)

This step involves the displacement of a leaving group (LG), preferably chlorine, in formula (II) with an amino group of formula (IV) in a suitable solvent (such as isopropanol or dioxane) in the presence of an organic base (such as N,N-diisopropylethylamine) at temperatures in the range of 0-80° C. for reaction times of up to 24 h.

Preferred conditions: 1 eq. of formula (II), 1 eq. of formula (IV), 3 eq. of N—N-diisopropylethylamine in isopropanol at room temperature overnight.

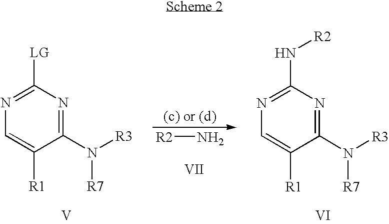

Scheme 2 illustrates the conversion of compounds with formula (V) to compounds with formula (VI), wherein R1, R2, R3, R7 and LG are as defined previously.

### Step (c)

Compounds of formula (V) can be converted to compounds with formula (VI) by displacement of a leaving group (preferably chlorine) in formula (V) with an amine with formula (VII) in an appropriate solvent (e.g. acetonitrile or n-butanol) in the presence of a suitable organic acid or mineral acid (such as acetic acid, HCl, H₂SO₄, p-toluenesulfonic acid). The reaction can be carried out at temperatures ranging from 50° C. to 200° C. by convectional heating or microwave heating.

Preferred method: 1 eq. of formula (V), 1 eq. of acetic acid, 3 eq. of formula (VII) in n-butanol heated to 150° C. in the microwave for 40 minutes.

### Step (d)

Alternatively, compounds of formula (VI) can be prepared from compounds with formula (V) (where LG is chlorine) by reaction with amine (VII) in the presence of a palladium source (e.g. Pd(OAc)₂ or Pd₂(dba)₃), a suitable ligand (e.g. bis(diphenylphosphino)-9,9-dimethylxanthene) and a suitable base (e.g. Cs₂CO₃ or sodium tert-butoxide) in a suitable solvent (e.g. dioxane). The reaction is generally carried out at around the reflux temperature of the solvent under an inert atmosphere.

Preferred method: 1 eq. of formula (V), 1.2 eq. of amine (VII), 0.06 eq. of Pd₂(dba)₃, 0.12 eq. of bis(diphenylphosphino)-9,9-dimethylxanthene, 3 eq. of sodium tert-butoxide in dioxane at 105° C. for 18 h.

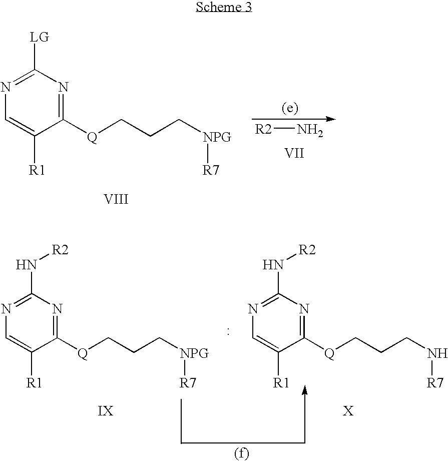

Compounds of formula (IX) and formula (X) can be prepared from compounds of formula (VIII) according to scheme 3, wherein R1, R2, R7 and LG are as defined previously; Q is NR7 or oxygen and PG represents a suitable protecting group (such as tert-butoxycarbonyl or benzyloxycarbonyl). Compounds of formula (VIII) wherein Q=NH, can be prepared according to step (b) Scheme 1; compounds of formula (VIII) wherein Q=oxygen can be prepared according to Scheme 7.

### Step (e)

Compounds of formula (IX) and formula (X) can be prepared from compounds of formula (VIII) by displacement of a leaving group (preferably chlorine) in formula (VIII) with an amine with formula (VII) in an appropriate solvent (e.g. acetonitrile or n-butanol) in the presence of a suitable organic acid or mineral acid (such as acetic acid, HCl, H₂SO₄, p-toluenesulfonic acid). The reaction can be carried out at temperatures typically ranging from 50° C. to 200° C., either by convectional heating or microwave heating. It will be understood by a skilled person that certain protecting groups are sensitive to the reaction conditions and may therefore give the product in the unprotected form (formula X), or indeed give mixtures of both formula (IX) and formula (X).

Preferred conditions: 1 eq of formula (VIII), 1.3 eq of formula (VII), 1.45 eq of 4M HCl in dioxane, in aqueous acetonitrile at 50° C. overnight.

### Step (f)

Compounds of formula (X) can be prepared from compounds of formula (IX) using standard methods for amine deprotection known to a skilled person. For example, where PG is tert-butoxycarbonyl reaction of formula (IX) with a suitable acid (such as HCl or trifluoroacetic acid) in a suitable solvent (such as dioxane or DCM) at temperatures typically in the range of 0° C. to the reflux temperature of the solvent enables this transformation to occur.

Preferred conditions: Formula (IX) is stirred in 4M HCl in dioxane at room temperature for 2 h.

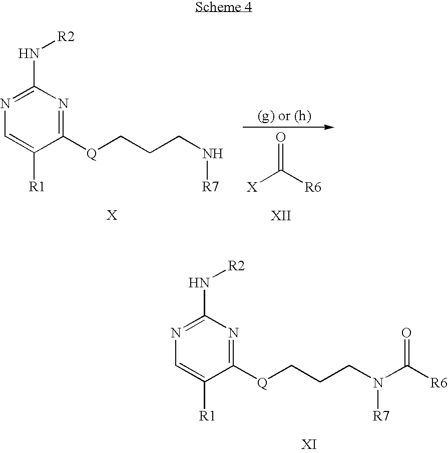

Compounds with formula (XI) can be prepared according to Scheme 4, wherein R1, R2, R6, R7 and Q are as defined previously, X refers to either OH or chlorine.

### Step (g)

Compounds with formula (XI) can be prepared from compounds of formula (X) by treatment with formula (XII), where X is chlorine. The reaction is carried out in the presence of a suitable base (such as N,N-diisopropylethylamine or triethylamine) in a suitable solvent (such as DCM or dioxane) at temperatures ranging from 0° C. to the reflux temperature of the solvent.

Preferred conditions: 1 eq of formula (X), 1.02 eq of formula (XII), 2.2 eq of triethylamine in DCM at room temperature for 2 h.

### Step (h)

Alternatively, compounds with formula (XI) can be prepared from compounds of formula (X) by treatment with formula (XII), where X is hydroxy. The reaction is carried out in the presence of a suitable amine coupling reagent known to the skilled person, (such as but not limited to HATU, DCC, EDCI), optionally in the presence of a suitable base (such as N,N-diisopropylethylamine or triethylamine), optionally in the presence of an additive (such as but not limited to, 1-hydroxybenzotriazole) in a suitable solvent (such as DMF or pyridine) at temperatures ranging from 0° C. to the reflux temperature of the solvent.

Preferred conditions: 1 eq of formula (X), 1.6 eq of formula (XII), 1.7 eq of HATU, 6.2 eq of N,N-diisopropylethylamine in DMF at room temperature overnight.

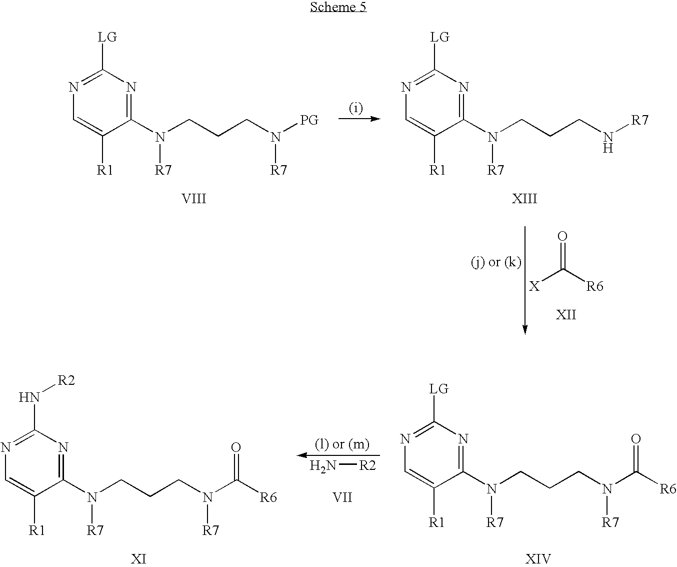

Alternatively, compounds with formula (XI) can be prepared according to Scheme 5 wherein R1, R2, R6 and R7, LG are all as defined previously; X is chlorine or hydroxyl.

### Step (i)

Compounds with formula (VIII), wherein PG is tert-butoxycarbonyl and LG is preferably chlorine are converted to formula (XIII) on treatment with a suitable acid (such as HCl or trifluoroacetic acid) in a suitable solvent (such as dioxane or DCM) at temperatures typically in the range of 0° C. to the reflux temperature of the solvent. Typical reaction times are in the range of 1 h to 24 h.

Preferred conditions: Formula (VIII) is stirred in 4M HCl in dioxane at room temperature for 1 h.

### Step (j)

Compounds with formula (XIV) can be prepared from compounds of formula (XIII) by treatment with formula (XII), wherein X is chlorine. The reaction is carried out in the presence of a suitable base (such as N,N-diisopropylethylamine or triethylamine) in a suitable solvent (such as DCM or dioxane) at temperatures ranging from 0° C. to the reflux temperature of the solvent.

Preferred conditions: 1 eq of formula (XIII), 1.02 eq of formula (XII), 2.2 eq of triethylamine in DCM at room temperature for 2 h.

### Step (k)

Alternatively, compounds with formula (XIV) can be prepared from compounds of formula (XIII) by treatment with formula (XII), wherein X is hydroxyl. The reaction is carried out in the presence of a suitable amine coupling reagent known to the skilled person, (such as but not limited to HATU, DCC, EDCI), optionally in the presence of a suitable base (such as N,N-diisopropylethylamine or triethylamine), optionally in the presence of an additive (such as but not limited to, hydroxybenzotriazole) in a suitable solvent (such as DMF or pyridine) at temperatures ranging from 0° C. to the reflux temperature of the solvent.

Preferred conditions: 1 eq of formula (XIII), 1.6 eq of formula (XII), 1.7 eq of HATU, 6.2 eq of N,N-diisopropylethylamine in DMF at room temperature overnight.

### Step (l)

Compounds of formula (XIV) can be converted to compounds with formula (XI) by displacement of a leaving group (preferably chlorine) in formula (XIV) with an amine with formula (VII) in an appropriate solvent (e.g. acetonitrile or n-butanol) in the presence of a suitable organic acid or mineral acid (such as acetic acid, HCl, H₂SO₄, p-toluenesulfonic acid). The reaction can be carried out at temperatures typically ranging from 50° C. to 200° C., by either convectional heating or optionally with microwave heating.

Preferred method: 1 eq. of formula (XIV), 1 eq. of acetic acid, 3 eq. of formula (VII) in n-butanol heated to 150° C. in the microwave for 40 minutes.

### Step (m)

Alternatively, compounds of formula (XI) can be prepared from compounds with formula (XIV) (wherein LG is chlorine) by reaction with amine (VII) in the presence of a palladium source (e.g. Pd(OAc)₂ or Pd₂(dba)₃), a suitable ligand (e.g. bis(diphenylphosphino)-9,9-dimethylxanthene) and a suitable base (e.g. Cs₂CO₃ or sodium tert-butoxide) in a suitable solvent (e.g. dioxane). The reaction is generally carried out at around the reflux temperature of the solvent under an inert atmosphere.

Preferred method: 1 eq. of formula (XIV), 1.2 eq. of amine (VII), 0.06 eq. of Pd₂(dba)₃, 0.12 eq. of bis(diphenylphosphino)-9,9-dimethylxanthene, 3 eq. of sodium tert-butoxide in dioxane at 105° C. for 18 h.

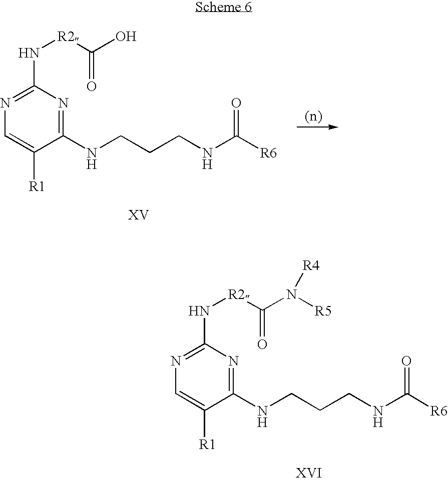

Compounds with formula (XVI) can be prepared from compounds of formula (XV) according to Scheme 6, wherein R1, R2, R4, R5 and R6 are as defined previously. Compounds of formula (XV) can be prepared according to step (c), scheme 2.

### Step (n)

Compounds of with formula (XVI) can be prepared from compounds of formula (XV) by treatment with a suitable amine with the formula NHR4R5. The reaction is carried out in the presence of a suitable amine coupling reagent known to the skilled person, (such as but not limited to HATU, DCC, EDCI), optionally in the presence of a suitable base (such as N,N-diisopropylethylamine or triethylamine), optionally in the presence of an additive (such as but not limited to, 1-hydroxybenzotriazole) in a suitable solvent (such as DMF or pyridine) at temperatures ranging from 0° C. to the reflux temperature of the solvent.

Preferred conditions: 1 eq of formula (XV), 1.5 eq of amine, 1.6 eq of HATU, 6 eq of N,N-diisopropylethylamine in DMF at room temperature overnight.

A further aspect of the invention relates to a process for preparing a compound of formula XIX, wherein R¹, R² and R³ are as defined above, said process comprising the steps of:

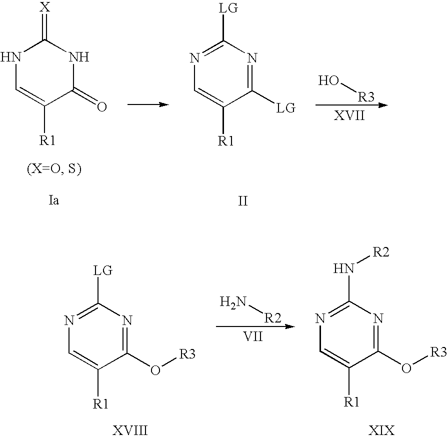

(i) converting a compound of formula Ia to a compound of formula II, where each LG is independently a leaving group;
(ii) reacting said compound of formula II with an alcohol of formula XVII to form a compound of formula XVIII;
(iii) reacting said compound of formula XVIII with an amine of formula VII to form a compound of formula XIX.

Compounds with formula (XIX) can be prepared according to scheme 7, wherein LG, R1, R2 and R3 are as defined previously.

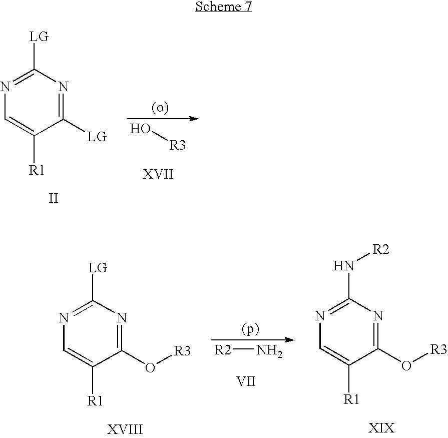

### Step (o)

Compounds with formula XVIII can be prepared by reacting compounds with formula (XVII) with compounds with formula (II) in a suitable solvent (such as THF) in the presence of a strong base, such as sodium hydride at temperatures ranging from 0° C. to the reflux temperature of the solvent.

Preferred conditions: 1 eq. of formula (XVII) is deprotonated with 1 eq. of sodium hydride in THF at 0° C., followed by treatment with 1 eq. of formula (II) at room temperature overnight.

### Step (p)

Compounds of formula (XVIII) can be converted to compounds with formula (XIX) by displacement of a leaving group (preferably chlorine) in formula (XVIII) with an amine with formula (VII) in an appropriate solvent (e.g. n-butanol) in the presence of a suitable organic acid or mineral acid (such as acetic acid, HCl, H₂SO₄, p-toluenesulfonic acid). The reaction can be carried out at temperatures typically ranging from 50° C. to 200° C., by either convectional heating or optionally with microwave heating.

Preferred method: 1 eq. of formula (XVIII), 0.2 eq. of acetic acid, 2 eq. of formula (IV) in n-butanol heated to 150° C. in the microwave for 15 minutes.

The present invention is further described by way of the following non-limiting examples.

### EXAMPLES

### Materials and Methods

### Source and Purification of Kinases

All protein kinases were of human origin and encoded full length proteins, unless indicated otherwise. They were either expressed as glutathione S-transferase (GST) fusion proteins in Escherichia Coli or as hexahistidine (His6)-tagged proteins in insect Sf21 cells. GST fusion proteins were purified by affinity chromatography on glutathione-Sepharose, and His6-tagged proteins on nickel/nitriloacetate-agarose. The procedures for expressing some of the protein kinases used herein have been detailed previously.^{8,9} The following sections outline the DNA vectors synthesised and the procedures used to express and purifying protein kinases that have not been reported previously.

Expression in E. coli

The following proteins were expressed in E. coli: —CHK2[5-543], cyclin-dependent protein kinase 2 (CDK2), MAP kinase-interacting kinase 2 (MNK2), extra-cellular signal-regulated kinase 1 (ERK1).

### Expression in Sf21 Cells

The following kinases were expressed in Sf21 cells: Aurora B and Aurora C, extra-cellular signal-regulated kinase 8 (ERK8), microtubule affinity regulating kinase 3 (MARK3), protein kinase Bα[118-480][S473D], protein kinase Bβ (PKBβ[120-481][S474D], 3-phosphoinositide-dependent protein kinase-1 [52-556]

### (PDK1[52-556], IKKε, TBK1,

### Activation of Protein Kinases

In order to produce activated forms of Aurora B and Aurora C, insect Sf21 cells were incubated for 1 h with the protein phosphatase inhibitor okadaic acid (50 nM). JNK isoforms were activated with MKK4 and MKK7, MNK2 with p38α MAPkinase; PKBα, PKBβ, with PDK1, and ERK1 with MKK1. To activate CDK2, bacterial pellets expressing cyclin A2 and CDK2 were mixed together, lyse and purified on glutathione Sepharose. The GST-tags were removed by cleavage with PreScission protease and the CDK2-cyclin A2 complex purified by chromatography on SP-Sepharose. It was then activated with CAK1/CDK7 followed by chromatography on nickel-nitrilotriacetate agarose to remove CAK1/CDK7, which binds to this column by virtue of its C-terminal His6 tag. All the other protein kinases were active as expressed.

### Protein Kinase Assays

All assays (25.5 μl) were carried out at room temperature (21° C.) and were linear with respect to time and enzyme concentration under the conditions used. Assays were performed for 30 min using Multidrop Micro reagent dispensers (Thermo Electron Corporation, Waltham, Mass. 02454, USA) in a 96-well format. The concentration of magnesium acetate in the assays was 10 mM and the [γ-33P] ATP (800 cpm/pmol) was used at 5, 20 or 50 μM as indicated, in order to be at or below the Km for ATP for each enzyme. Protein kinases assayed at 5 μM ATP were: —ERK1, ERK8, PKBα, MARK3, Aurora C. Protein kinases assayed at 20 μM ATP were: —JNK1, PDK1, CHK1, CHK2, CDK2 and Aurora B. Protein kinases assayed at 50 μM ATP were: —MNK2, IKKepsilon and TBK1.

The assays were initiated with MgATP, stopped by addition of 5 μl of 0.5 M orthophosphoric acid and spotted on to P81 filterplates using a unifilter harvester (PerkinElmer, Boston, Mass. 02118, USA). The IC50 values of inhibitors were determined after carrying out assays at 10 different concentrations of each compound.

ERK1 and ERK8 were both assayed against myelin basic protein (MBP, 0.33 mg/ml). MARK3 was assayed against the peptide KKKVSRSGLYRSPSMPENLNRPR (300 μM), MNK2 against the eIF4E protein (0.5 mg/ml). PKBβ was assayed against the peptide GRPRTSSFAEGKK (30 μM). TBK1 were assayed against (AKPKGNKDYHLQTCCGSLAYRRR) (300 μM). The substrates used for other protein kinases were described previously.^{8,9}

Unless stated otherwise, enzymes were diluted in 50 mM Tris/HCl pH 7.5, 0.1 mM EGTA, 1 mg/ml BSA, 0.1% (v/v) 2-mercaptoethanol and assayed in 50 mM Tris/HCl pH 7.5, 0.1 mM EGTA, 0.1% (v/v) 2-mercaptoethanol.

### General Procedures for Synthesis of Compounds

### Chromatography

Preparative high pressure liquid chromatography was carried out using apparatus made by Agilent. The apparatus is constructed such that the chromatography (column: either a 30×100 mm (10 μm) C-18 Phenomenex Gemini column at a flow rate of 50 ml/min, or a 21.2×100 mm (5 μm) C-18 Phenomenex Gemini column at a flow rate of 20 ml/min) is monitored by a multi-wavelength UV detector (G1365B manufactured by Agilent) and an MM-ES+APCI mass spectrometer (G-1956A, manufactured by Agilent) connected in series, and if the appropriate criteria are met the sample is collected by an automated fraction collector (G1364B manufactured by Agilent). Collection can be triggered by any combination of UV or mass spectrometry or can be based on time. Typical conditions for the separation process are as follows: The gradient is run over a 10 minute period (gradient at start: 10% methanol and 90% water, gradient at finish: 100% methanol and 0% water; as buffer: either 0.1% trifluoroacetic acid is added to the water (low pH buffer), or ammonium bicarbonate (10 mmol/l) and 35% ammonium hydroxide (1.6 ml/l) is added to the water (high pH buffer). It will be appreciated by those skilled in the art that it may be necessary or desirable to modify the conditions for each specific compound, for example by changing the solvent composition at the start or at the end, modifying the solvents or buffers, changing the run time or changing the flow rate.

Flash chromatography refers to silica gel chromatography and carried out using an SP4MPLC system (manufactured by Biotage); pre-packed silica gel cartridges (supplied by Biotage); or using conventional glass column chromatography.

### Analytical Methods

¹H Nuclear magnetic resonance (NMR) spectroscopy was carried out using an ECX400 spectrometer (manufactured by JEOL) in the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; br, broad. Mass spectra were recorded using a MM-ES+APCI mass spectrometer (G-1956A, manufactured by Agilent). Where thin layer chromatography (TLC) has been used it refers to silica gel TLC using silica gel MK6F 60 Å plates, R_{f} is the distance traveled by the compound divided by the distance traveled by the solvent on a TLC plate.

### Compound Preparation

Where the preparation of starting materials is not described, these are commercially available, known in the literature, or readily obtainable by those skilled in the art using standard procedures. Where it is stated that compounds were prepared analogously to earlier or later examples or intermediates, it will be appreciated by the skilled person that the reaction time, number of equivalents of reagents and temperature can be modified for each specific reaction and that it may be necessary or desirable to employ different work-up or purification techniques. Where reactions are carried out using microwave irradiation, the microwave used is an Initiator 60 supplied by Biotage. The actual power supplied varies during the course of the reaction in order to maintain a constant temperature.

### ABBREVIATIONS

### DCM=Dichloromethane

### DMF=N,N-Dimethylformamide

### THF=Tetrahydrofuran

### MeOH=Methanol

TFA=Trifluoroacetic acid
Xantphos=4,5-Bis(diphenylphosphino)-9,9-dimethylxanthene
HATU=N,N,N′,N′-Tetramethyl-O-(7-azabenzotriazol-1-yl)uronium-hexafluorophospate
EDCI=1,3-Propanediamine, N3-(ethylcarbonimidoyl)-N1,N1-dimethyl-, hydrochloride

### DCC=1,3-Dicyclohexylcarbodiimide

Pd₂(dba)₃=tris(dibenzylideneacetone)dipalladium(0)

### TEA=Triethylamine

rm=Reaction mixture
rt=Room temperature
AcOH=Acetic acid

### IPA=Isopropanol

### DIPEA=N,N-diisopropylethylamine

TBSMSCl=Tertiarybutyldimethylsilyl chloride

### MeCN=Acetonitrile

### NH3=Ammonia

### EtOH=Ethanol

### EtOAc=Ethyl Acetate

LCMS=Mass spectrometry directed high pressure liquid chromatography

### UV=Ultraviolet

SCX=Strong cation exchange

### EXAMPLES

### Intermediate 1

### 2,4-Dichloro-5-cyclopropyl-pyrimidine

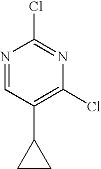

To a suspension of 5-cyclopropyl-2-thioxo-2,3-dihydro-1H-pyrimidin-4-one [Prepared according to J. Org. Chem. 1994, 4791-4799] (2.3 g, 13.7 mmol) in phosphorus oxychloride (10 ml, 107 mmol) was added N,N-diisopropylethylamine (3.5 ml, 13.8 mmol) dropwise at 0° C. The resulting mixture was stirred at 90° C. for 2 h to give complete reaction. The mixture was then concentrated to dryness, diluted with DCM (50 ml) and poured into ice water (50 ml). The organic layer was separated, the aqueous phase was extracted with DCM (×2) and the combined organic phases were dried (MgSO₄) and evaporated to dryness. The crude yellow oil was purified by flash chromatography on the Biotage SP4, eluting with 0 to 30% ethyl acetate/petroleum ether gradient to give a pale yellow oil which solidified on standing to an off-white solid (2.32 g, 90% yield). ¹H NMR (400 MHz, DMSO-d₆) δ ppm 0.91 (m, 2H), 1.07 (m, 2H), 1.97-2.05 (m, 1H), 8.49 (s, 1H). R_{f} (10% ethyl acetate in petroleum ether)=0.75.

### Intermediate 2

### [3-(2-Chloro-5-cyclopropyl-pyrimidin-4-ylamino)-propyl]-carbamic acid tert-butyl ester

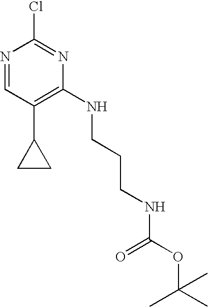

Intermediate 1 (5.84 g, 30.9 mmol), 3-aminopropyl)carbamic acid tert-butyl ester (5.38 g, 30.9 mmol) and N,N-diisopropylethylamine (16.5 ml, 92.7 mmol) were combined in isopropyl alcohol (150 ml) at 0° C. and then stirred at this temperature for 1 hour. After this time, the reaction mixture was warmed up to room temperature and stirring was continued overnight. The volatiles were evaporated and the crude material dissolved in ethyl acetate, washed with water, followed by brine, dried (MgSO₄) and solvents evaporated to dryness. The crude product was purified by flash chromatography using a Biotage SP4 (ethyl acetate/petroleum ether gradient) to give the product as a white solid (7.1 g, 70%). ¹H NMR (400 MHz, DMSO-d₆) δ ppm 0.53-0.59 (m, 2H), 0.84-0.92 (m, 2H), 1.37 (s, 9H), 1.47-1.55 (m, 1H), 1.60-1.69 (m, 2H), 2.93-3.02 (m, 2H), 3.33-3.41 (m, 2H), 6.87 (t, J=5.7 Hz, 1H), 7.41 (t, J=5.7 Hz, 1H), 7.69 (s, 1H); m/z (ES+APCI)⁺ 327 [M+H]⁺.

### Intermediate 3

### N4-(3-Amino-propyl)-5-cyclopropyl-N2-(3-fluoro-phenyl)-pyrimidine-2,4-diamine hydrochloride

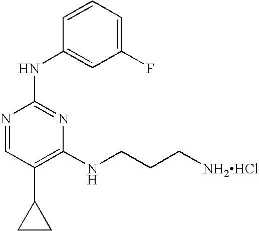

To a solution of Intermediate 2 (2 g, 6.1 mmol) and 3-fluoroaniline (766 μl, 8.0 mmol) in 14:1 acetonitrile:water (53.6 ml) was added 4M HCl in dioxane (2.22 ml, 8.9 mmol). The reaction mixture was heated to 50° C. and stirred at this temperature overnight. The white precipitate that formed was filtered, washed with diethyl ether and dried under vacuum to give a white solid (1.3 g, 63%). ¹H NMR (400 MHz, DMSO-d₆) δ ppm 0.54-0.62 (m, 2H), 0.86-0.96 (m, 2H), 1.53-1.63 (m, 1H), 1.87-1.99 (m, 2H), 2.78-2.90 (m, 2H), 3.49-3.63 (m, 2H), 6.98 (td, J=8.5, 1.8 Hz, 1H), 7.30 (d, J=9.2 Hz, 1H), 7.43 (dd, J=8.2, 6.9 Hz, 1H), 7.64 (dt, J=11.8, 2.2, 2.1 Hz, 1H), 7.71 (s, 1H), 8.02 (br., 3H), 8.90 (br. s, 1H), 10.83 (br. s, 1H); m/z (ES+APCI)⁺: 302 [M+H]⁺.

### Intermediate 4

### N1-(2-Chloro-5-cyclopropyl-pyrimidin-4-yl)-propane-1,3-diamine dihydrochloride

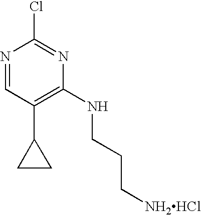

A suspension of Intermediate 2 (2 g, 6.13 mmol) in 4M HCl in dioxane (15 ml, 60 mmol) was stirred vigorously for 1 hour at room temperature The reaction mixture was evaporated to dryness, azeotroped with toluene (25 ml) followed by DCM (40 ml) to give an off-white solid dihydrochloride salt 100% (1.61 g, 100%). ¹H NMR (400 MHz, DMSO-d₆) δ ppm 0.55-0.61 (2H, m), 0.91 (2H, m), 1.58 (1H, m), 1.83-1.92 (2H, m), 2.78-2.87 (2H, m), 3.47 (2H, m), 7.50 (1H, br. s), 7.73 (1H, s), 7.98 (1H, t, J=5.72 Hz), 8.08 (2H, br. s); m/z (ES+APCI)⁺; 227 302 [M+H]⁺.

### Intermediate 5

### N-{3-[4-(3-Amino-propylamino)-5-cyclopropyl-pyrimidin-2-ylamino]-phenyl}-acetamide dihydrochloride

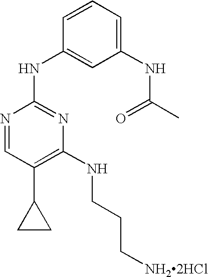

### Step 1

### {3-[2-(3-Acetylamino-phenylamino)-5-cyclopropyl-pyrimidin-4-ylamino]-propyl}-carbamic acid tert-butyl ester

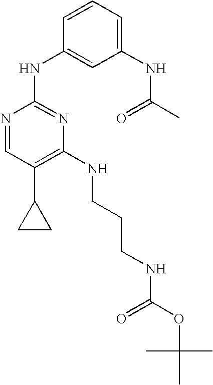

Intermediate 2 (200 mg, 0.61 mmol), N-(3-aminophenyl)-acetamide (184 mg, 1.22 mmol) and glacial acetic acid (7 μl, 0.12 mmol) were dissolved in n-butanol (3 ml). The reaction mixture was irradiated at 140° C. for 15 minutes in a Biotage I-60 microwave reactor. The reaction was evaporated to dryness, dissolved in DCM (10 ml), partitioned with saturated sodium bicarbonate solution (10 ml) and separated. The aqueous was extracted with DCM (20 ml) and the combined organic layers were dried (MgSO₄) and evaporated to dryness. The crude product was subjected to repeated flash chromatography on a Biotage SP4 (DCM/methanol gradient) to give an off-white solid (0.167 g). ¹H NMR data are consistent with the desired product, but indicate the presence of approximately 20% of unreacted N-(3-Aminophenyl)-acetamide starting material impurity. m/z (ES+APCI)⁺: 441 [M+H]⁺. This product was used in the next step without further purification.

### Step 2

### N-{3-[4-(3-Amino-propylamino)-5-cyclopropyl-pyrimidin-2-ylamino]-phenyl}-acetamide dihydrochloride

A suspension of {3-[2-(3-Acetylamino-phenylamino)-5-cyclopropyl-pyrimidin-4-ylamino]-propyl}-carbamic acid tert-butyl ester prepared in step 1 (167 mg, 0.38 mmol) in 4M hydrogen chloride in dioxane (1 ml) was stirred vigorously for 2 h at room temperature. The reaction was evaporated to dryness, triturated with DCM, filtered and dried to give the title compound as an off-white solid (169 mg, 74% over 2 steps). ¹H NMR (400 MHz, DMSO-d₆) δ ppm 0.53-0.61 (m, 2H), 0.84-0.96 (m, 2H), 1.53-1.62 (m, 1H), 1.85-1.95 (m, 2H), 2.07 (s, 3H), 2.78-2.87 (m, 2H), 3.60-3.67 (m, 2H), 7.18-7.23 (m, 2H), 7.27-7.33 (m, 1H), 7.67 (s, 1H), 7.98 (br. s, 2H), 8.12 (s, 1H), 8.76-8.82 (m, 1H), 10.16 (s, 1H), 10.52 (s, 1H); m/z (ES+APCI)⁺: 341 [M+H]⁺.

### Intermediate 6

### Thiophene-2-carboxylic acid [3-(2-chloro-5-cyclopropyl-pyrimidin-4-ylamino)-propyl]-amide

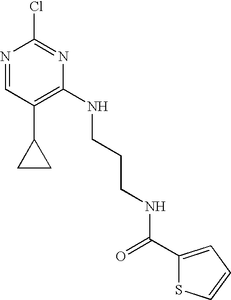

To an ice cooled solution of Intermediate 4 (2.45 g, 9.3 mmol) and triethylamine (2.8 ml, 20.45 mmol) in DCM was added thiophene-2-carbonyl chloride (1.02 ml, 9.5 mmol) dropwise. The reaction was stirred at room temperature for 2.5 h and then quenched with water (50 ml), and the aqueous layer was extracted with DCM (2×50 ml). The combined organic layers were washed with brine (50 ml), dried (MgSO₄) and evaporated to dryness. The crude product was subjected to flash chromatography on the Biotage SP4 (gradient elution from 0-6% methanol in DCM) to give a foam, which was triturated with petroleum ether to give a white solid (1.56 g, 50%). ¹H NMR (400 MHz, DMSO-d₆) δ ppm 0.52-0.59 (m, 2H), 0.84-0.93 (m, 2H), 1.47-1.56 (m, 1H), 1.74-1.84 (m, 2H), 3.26-3.33 (m, 2H), 3.39-3.46 (m, 2H), 7.14 (dd, J=4.8, 3.9 Hz, 1H), 7.45-7.51 (m, 1H), 7.70 (s, 1H), 7.71-7.76 (m, 2H), 8.52-8.57 (m, 1H); m/z (ES+APCI)⁺: 337 [M+H]⁺.

### Intermediate 7

### 1-[3-(2-Chloro-5-cyclopropyl-pyrimidin-4-ylamino)-propyl]-pyrrolidin-2-one

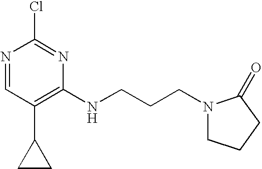

Intermediate 1 (200 mg, 1.06 mmol), N-(3-aminopropyl)-2-pyrrolidinone (148 μl, 1.06 mmol) and N,N-diisopropylethylamine (564 μl, 3.17 mmol) were combined in isopropyl alcohol (1 ml) at 0° C. and then stirred at this temperature for 1 hour. After this time, the reaction mixture was warmed up to room temperature and stirring continued overnight. The reaction mixture was partitioned between water and ethyl acetate, the aqueous phase was extracted with ethyl acetate (×2), the combined organic extracts washed with water, followed by brine, dried over MgSO₄ and solvents evaporated to dryness. The crude product was purified by flash chromatography on the Biotage SP4 (gradient elution from 0-10% methanol in ethyl acetate) to give a brown oil (197 mg, 63%). ¹H NMR (400 MHz, DMSO-d₆) δ ppm 0.50-0.56 (m, 2H), 0.80-0.88 (m, 2H), 1.43-1.51 (m, 1H), 1.60-1.72 (m, 2H), 1.85-1.95 (m, 2H), 2.14-2.23 (m, 2H), 3.20 (t, J=6.9 Hz, 2H), 3.33 (m, 4H), 7.44 (t, J=5.7 Hz, 1H), 7.66 (s, 1H); m/z (ES+APCI)⁺: 295 [M+H]⁺.

### Intermediate 8

Cyclobutanecarboxylic acid [3-(2-chloro-5-cyclopropyl-pyrimidin-4-ylamino)-propyl]-amide

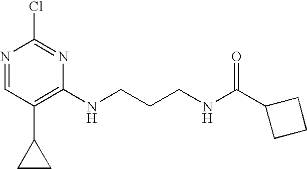

Prepared analogously to Intermediate 6 from Intermediate 4 and cyclobutane carbonyl chloride to give desired product as a colourless oil (0.83 g, 35%). ¹H NMR (400 MHz, DMSO-d₆) δ ppm 0.50-0.61 (m, 2H), 0.82-0.94 (m, 2H), 1.45-1.57 (m, 1H), 1.60-1.68 (m, 2H), 1.68-1.78 (m, 1H), 1.80-1.93 (m, 1H), 1.94-2.04 (m, 2H), 2.05-2.17 (m, 2H), 2.92-3.03 (m, 1H), 3.05-3.12 (m, 2H), 3.29-3.40 (m, 2H), 7.44-7.50 (m, 1H), 7.66-7.74 (m, 2H). R_{f} (5% MeOH in DCM)=0.4.

Alternatively intermediate 8 can be prepared according to the following method:

A solution of Intermediate 11 below (5.16 g, 19.04 mmol) in IPA (20 ml) was added to a stirred solution of intermediate 1 (3.6 g, 19.04 mmol) and DIPEA (13.2 ml, 72.2 mmol) in IPA (80 ml) with ice-cooling. The mixture was allowed to warm to rt and then heated at 55° C. overnight. The mixture was allowed to cool to rt and concentrated. The residue was dispersed into ethyl acetate and water. The organic phase was dried and concentrated to give an oil. The crude oil was purified by flash column chromatography on silica gel (200 g) eluting with 30:1 DCM:MeOH to afford a white solid (4.3 g, 73%).

### Intermediate 9

Cyclopentanecarboxylic acid [3-(2-chloro-5-cyclopropyl-pyrimidin-4-ylamino)-propyl]-amide

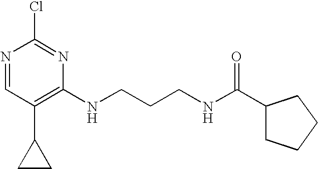

To a solution of Intermediate 4 (0.2 g, 0.76 mmol) in DMF (2 ml) was added cyclopentane carboxylic acid (0.124 ml, 1.14 mmol), HATU (0.46 g, 1.22 mmol) and N,N-diisopropylethylamine (0.79 ml, 4.56 mmol). The reaction mixture was stirred at room temperature for 18 hours. The mixture was evaporated and filtered through a 1 g Isolute-NH₂ cartridge, eluting with 9:1 DCM: methanol. Purification by flash chromatography on the Biotage SP4 (gradient elution from 0 to 5% methanol in DCM) gave the desired product as a colourless oil (45 mg, 18%). ¹H NMR (400 MHz, DMSO-d₆) δ ppm 0.53-0.58 (m, 2H), 0.86-0.92 (m, 2H), 1.46-1.54 (m, 3H), 1.54-1.76 (m, 9H), 3.06-3.13 (m, 2H), 3.29-3.40 (m, 2H), 7.44-7.49 (m, 1H), 7.69 (s, 1H), 7.79-7.84 (m, 1H); m/z (ES+APCI)⁺: 323 [M+H]⁺.

### Intermediate 10

### Pyrrolidine-1-carboxylic acid (3-amino-phenyl)-amide

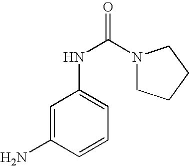

### Step 1

### N-(3-nitrophenyl)pyrrolidine-1-carboxamide

To a stirred solution of 3-nitrophenylisocyanate (10 g, 60.9 mmol) in dry THF (100 ml) at 0° C., was added pyrrolidine (6.04 ml, 73.1 mmol), and the resulting solution was heated to reflux overnight. The solvent was removed under reduced pressure and the resulting yellow solid was triturated with ethyl acetate, filtered, washed with 1:1 ethyl acetate—petroleum ether and dried under vacuum. The product was isolated as a pale yellow solid (12.8 g, 89%). ¹H NMR (400 MHz, DMSO-d₆) δ ppm 1.81-1.91 (m, 4H), 3.35-3.43 (m, 4H), 7.50 (t, J=8.28 Hz, 1H), 7.76 (ddd, J=8.28, 2.76, 0.92 Hz, 1H), 7.96 (ddd, J=8.28, 1.8, 0.92 Hz, 1H), 8.55 (t, J=1.8 Hz, 1H), 8.64 (br s, 1H); m/z (ES+APCI)⁻ 235 [M−H].

### Step 2

### Pyrrolidine-1-carboxylic acid (3-amino-phenyl)-amide

To a solution of N-(3-nitrophenyl)pyrrolidine-1-carboxamide prepared in step 1 (10 g, 42.6 mmol) in ethyl acetate (150 ml) and ethanol (10 ml) was added 10% Pd on activated carbon (1 g). The reaction was stirred under an atmosphere of hydrogen at room temperature for 18 h. More 10% Pd on activated carbon (0.4 g) was added and the reaction was complete after stirring for a further 4 hours. The mixture was filtered through Celite®, washing with ethanol, and then evaporated to give a pink foam, which was triturated with petroleum ether—EtOAc (10:1). The resultant solid was filtered and dried under vacuum to give the desired product as a pale pink solid (4.68 g, 54%). ¹H NMR (400 MHz, DMSO-d₆) δ ppm 1.78-1.88 (m, 4H), 3.25-3.39 (m, 4H), 4.88 (br. s, 2H), 6.14 (ddd, J=7.0, 1.1, 1.0 Hz, 1H), 6.61 (ddd, J=7.0, 1.1, 1.0 Hz, 1H), 6.76-6.89 (m, 2H), 7.75 (br. s, 1H). R_{f} (6% methanol: 94% DCM)=0.35.

### Intermediate 11

### Cyclobutanecarboxylic acid (3-amino-propyl)-amide trifluoroacetate salt

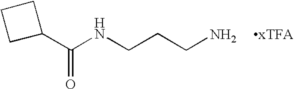

### Step 1

### [3-(Cyclobutanecarbonyl-amino)-propyl]-carbamic acid tert-butyl ester

A solution of cyclobutanecarbonyl chloride (6.7 ml, 58.6 mmol) in DCM (20 ml) was added slowly to a stirred solution of (3-amino-propyl)-carbamic acid tert-butyl ester (10.0 g, 57.5 mmol) and TEA (12.0 ml, 86.2 mmol) in DCM (180 ml) with ice cooling. The RM was then stirred at rt for 2 hours. After this time the mixture was washed with 10% aqueous citric acid and water. The organic phase was separated, dried and concentrated to provide a white solid (14.3 g, 97%). ¹H NMR (400 MHz, DMSO-d₆) δ ppm 1.37 (9H, m), 1.40-1.55 (2H, m), 1.62-2.30 (6H, m), 2.75-3.12 (5H, m), 6.78 (1H, t, J=5.50 Hz), 7.61 (1H, t, J=5.50 Hz)

### Step 2

### Cyclobutanecarboxylic acid (3-amino-propyl)-amide trifluoroacetate salt

[3-(Cyclobutanecarbonyl-amino)-propyl]-carbamic acid tert-butyl ester (11.3 g, 44.0 mmol) in TFA (20 ml) and DCM (60 ml) was stirred at rt for 5 hours. The mixture was then concentrated to dryness. Toluene was added to the residue and the mixture concentrated to dryness again to provide a viscous oil (22.2 g). ¹H NMR (400 MHz, DMSO-d₆) δ ppm 1.59-2.18 (8H, m), 2.60-2.85 (2H, m), 2.85-3.14 (3H, m), 7.60-7.91 (3H, m); m/z (ES+APCI)⁺: 157 [M+H]⁺.

### Intermediate 12

### N,N,N′-Trimethyl-N′-(5-nitro-pyridin-2-yl)-ethane-1,2-diamine

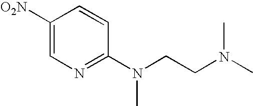

N,N,N′-Trimethylethylenediamine (798 μl, 6.29 mmol) was added to 2-chloro-5-nitropyridine (1.0 g, 6.29 mmol) and DIPEA (1.09 ml, 6.29 mmol) in MeCN (20 ml). The mixture was stirred at rt for 4 hours and then concentrated to dryness. The residue was dispersed into DCM and saturated aqueous sodium carbonate solution. The organic phase was dried and concentrated to give an orange coloured oily solid (1.3 g, 92%). ¹H NMR (400 MHz, CHLOROFORM-d) δ ppm 2.30 (6H, s), 2.49-2.57 (2H, m), 3.19 (3H, s), 3.79 (2H, br. s.), 6.46 (1H, d, J=9.62 Hz), 8.12-8.22 (1H, m), 8.92-9.08 (1H, m).

### Intermediate 13

### N²-(2-Dimethylamino-ethyl)-N²-methyl-pyridine-2,5-diamine

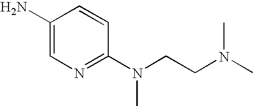

A mixture of Intermediate 12 (1.3 g, 5.8 mmol) and 10% Pd/C (130 mg) in EtOH (40 ml) were stirred at rt under an atmosphere of hydrogen for 4 hours. The mixture was then filtered through Celite and the filtrate concentrated to dryness to give a deep purple coloured oil. The crude oil was purified by flash column chromatography on silica gel (60 g) eluting with 10:1 DCM:2M NH₃ in MeOH to provide a deep purple coloured oil (470 mg, 42%). ¹H NMR (400 MHz, DMSO-d₆) δ ppm 2.14 (6H, s), 2.27-2.34 (2H, m), 2.84 (3H, s), 3.41-3.49 (2H, m), 4.35 (2H, br. s.), 6.39 (1H, d, J=8.70 Hz), 6.88 (1H, dd, J=8.93, 2.98 Hz), 7.54 (1H, d, J=2.29 Hz); m/z (ES+APCI)⁺: 195 [M+H]⁺.

### Intermediate 14

### 2-[Methyl-(5-nitro-pyridin-2-yl)-amino]-ethanol

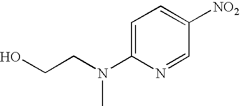

Prepared analogously to Intermediate 12 from 2-chloro-5-nitropyridine and 2-methylamino-ethanol to give an orange coloured oil (1.32 g, 106%). ¹H NMR (400 MHz, DMSO-d₆) δ ppm 3.19 (3H, s), 3.51-3.83 (4H, m), 4.83 (1H, br. s.), 6.76 (1H, br. s.), 8.19 (1H, dd, J=9.62, 2.29 Hz), 8.95 (1H, d, J=2.75 Hz)

### Intermediate 15

### 2-[(5-Amino-pyridin-2-yl)-methyl-amino]-ethanol

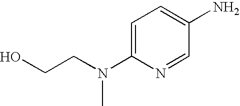

Prepared analogously to Intermediate 13 from Intermediate 14 to give a deep purple coloured oil (912 mg, 83%). ¹H NMR (400 MHz, DMSO-d₆) δ ppm 2.89 (3H, s), 3.37-3.54 (4H, m), 4.36 (2H, s), 4.66 (1H, t, J=5.04 Hz), 6.42 (1H, d, J=8.70 Hz), 6.89 (1H, dd, J=8.93, 2.98 Hz), 7.53 (1H, d, J=2.75 Hz); m/z (ES+APCI)⁺: 168 [M+H]⁺.

### Intermediate 16

### Methyl-(2-morpholin-4-yl-ethyl)-(5-nitro-pyridin-2-yl)-amine

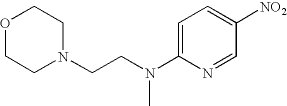

Prepared analogously to Intermediate 12 from 2-chloro-5-nitropyridine and methyl-(2-morpholin-4-yl-ethyl)-amine to give an orange oil (790 mg, 94%). ¹H NMR (400 MHz, CHLOROFORM-d) δ ppm 2.41-2.65 (6H, m), 3.21 (3H, s), 3.70 (4H, m), 3.83 (2H, br. s.), 6.47 (1H, d, J=9.16 Hz), 8.21 (1H, dd, J=9.39, 2.98 Hz), 9.05 (1H, d, J=2.75 Hz)

### Intermediate 17

### N²-Methyl-N²-(2-morpholin-4-yl-ethyl)-pyridine-2,5-diamine

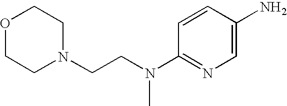

Prepared analogously to Intermediate 13 using Intermediate 16 to give a deep purple coloured oil (352 mg, 50%). ¹H NMR (400 MHz, CHLOROFORM-d) δ ppm 2.41-2.65 (6H, m), 2.99 (3H, s), 3.55-3.75 (6H, m), 6.42 (1H, d, J=9.62 Hz), 6.99 (1H, dd, J=8.70, 2.75 Hz), 7.76 (1H, d, J=2.29 Hz); m/z (ES+APCI)⁺: 237 [M+H]⁺.

### Intermediate 18

### 5-Amino-pyridine-2-carboxylic acid ethyl ester

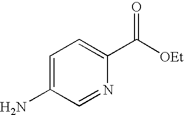

Thionyl chloride (5.2 ml, 71.3 mmol) was added dropwise to 5-amino-pyridine-2-carboxylic acid (4.0 g, 29.0 mmol) in EtOH (30 ml) whilst maintaining the temperature of the mixture below 10° C. During the addition the mixture became very thick and more EtOH (20 ml) was added. The mixture was then stirred and heated at 95° C. overnight. Next day the temperature was increased to 110° C. and the mixture was heated at this temperature overnight. On cooling to rt the mixture was concentrated to give a solid. Saturated aqueous sodium hydrogen carbonate solution was added followed by saturated aqueous sodium carbonate solution. Ethyl acetate and water were then added. The organic phase was washed with brine, dried and concentrated to give an off white solid (4.0 g, 83%). ¹H NMR (400 MHz, DMSO-d₆) δ ppm 1.27 (3H, t, J=7.10 Hz), 4.22 (2H, q, J=6.87 Hz), 6.19 (2H, s), 6.91 (1H, dd, J=8.70, 2.75 Hz), 7.74 (1H, d, J=8.70 Hz), 7.97 (1H, d, J=2.29 Hz).

### Intermediate 19

### (5-Amino-pyridin-2-yl)-methanol

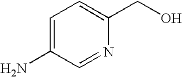

Lithium aluminium hydride, 2 M in THF (29.8 ml, 59.6 mmol) was added slowly to a solution of Intermediate 18 (3.3 g, 19.9 mmol) in THF (120 ml) with ice-cooling. The mixture was stirred at rt for 4 hours. Water (2.3 ml) was then added dropwise whilst maintaining the temperature of the mixture below 10° C. An aqueous solution of sodium hydroxide, 15% w/v (2.3 ml) was subsequently added followed by an additional quantity of water (6.9 ml). The resulting mixture was filtered and the filtrate concentrated to provide an orange coloured solid (2.59 g, 105%). ¹H NMR (400 MHz, DMSO-d₆) δ ppm 4.35 (2H, d, J=5.50 Hz), 5.04 (1H, m), 5.16 (2H, s), 6.90 (1H, dd, J=8.24, 2.75 Hz), 7.07 (1H, d, J=8.24 Hz), 7.82 (1H, d, J=2.75 Hz).

### Intermediate 20

### 6-(tert-Butyl-dimethyl-silanyloxymethyl)-pyridin-3-ylamine

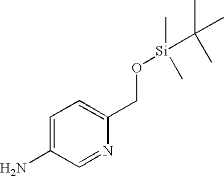

A mixture of Intermediate 19 (1.43 g, 11.5 mmol), TBDMSCl (2.09 g, 13.8 mmol) and imidazole (1.88 g, 27.7 mmol) in DMF (30 ml) were stirred at rt overnight. The mixture was diluted with EtOAc, washed with water (×4) and brine (×1). The organic phase was dried and concentrated. The crude residue was purified by flash column chromatography on silica gel (100 g) eluting with 2:1 ethyl acetate:petrol to give an off-white solid (1.72 g, 63%). ¹H NMR (400 MHz, DMSO-d₆) δ ppm 0.04 (6H, s), 0.88 (9H, s), 4.55 (2H, s), 5.20 (2H, s), 6.91 (1H, dd, J=8.24, 2.75 Hz), 7.06 (1H, d, J=8.24 Hz), 7.83 (1H, d, J=2.75 Hz).

### Intermediate 21

### Cyclobutanecarboxylic acid (3-{2-[6-(tert-butyl-dimethyl-silanyloxymethyl)-pyridin-3-ylamino]-5-cyclopropyl-pyrimidin-4-ylamino}-propyl)-amide

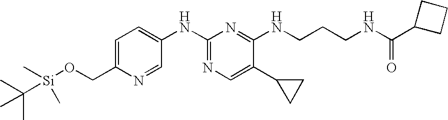

A mixture of Intermediate 20 (1.72 g, 7.23 mmol), Intermediate 8 (1.11 g, 3.61 mmol) and AcOH (206 μl, 3.61 mmol) in n-BuOH (20 ml) were stirred and heated at 115° C. overnight. On cooling to rt the mixture was concentrated to dryness. The residue was taken up in EtOAc and washed with saturated aqueous sodium hydrogen carbonate solution and brine. The organic phase was dried and concentrated. The crude residue was purified by flash column chromatography on silica gel (150 g) eluting with a MeOH-EtOAc gradient (2% to 5% MeOH) to give an off-white solid (590 mg, 32%). ¹H NMR (400 MHz, DMSO-d₆) δ ppm 0.08 (6H, s), 0.45-0.50 (2H, m), 0.81-0.86 (2H, m), 0.91 (9H, s), 1.44-1.51 (1H, m), 1.66-1.78 (3H, m), 1.82-1.90 (1H, m), 1.94-2.03 (2H, m), 2.06-2.17 (2H, m), 2.93-3.00 (1H, m), 3.10-3.17 (2H, m), 3.39-3.46 (2H, m), 4.66 (2H, s), 6.90 (1H, t, J=5.95 Hz), 7.29 (1H, d, J=8.24 Hz), 7.62 (1H, s), 7.69 (1H, t, J=5.72 Hz), 8.21 (1H, dd, J=8.47, 2.52 Hz), 8.81 (1H, d, J=2.29 Hz), 9.08 (1H, s); m/z (ES+APCI)⁺: 511 [M+H]⁺.

### Intermediate 22

### Cyclobutanecarboxylic acid {3-[2-(6-chloromethyl-pyridin-3-ylamino)-5-cyclopropyl-pyrimidin-4-ylamino]-propyl}-amide x.HCl

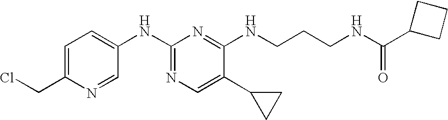

Thionyl chloride (194 μl, 2.66 mmol) was added dropwise to a suspension of Example 103 (211 mg, 0.533 mmol) in DCM (20 ml) with ice-cooling. The mixture was allowed to warm to rt and stirred at this temperature overnight. The mixture was then concentrated to dryness to give an off-white solid as the x.HCl salt (274 mg). ¹H NMR (400 MHz, DMSO-d₆) δ ppm 0.55-0.60 (2H, m), 0.88-0.94 (2H, m), 1.52-1.60 (1H, m), 1.65-1.75 (3H, m), 1.79-1.99 (3H, m), 2.02-2.12 (2H, m), 2.92-2.98 (1H, m), 3.06-3.17 (2H, m), 3.42-3.49 (2H, m), 4.79 (2H, s), 7.61 (1H, d, J=8.70 Hz), 7.66 (1H, s), 7.80 (1H, t, J=5.72 Hz), 8.07 (1H, dd, J=8.47, 2.52 Hz), 8.73-8.79 (2H, m), 10.79 (1H, s); m/z (ES+APCI)⁺: 415/417 [M+H]⁺.

### Intermediate 23

### [3-(2-Chloro-5-cyclopropyl-pyrimidin-4-yloxy)-propyl]-carbamic acid tert-butyl ester

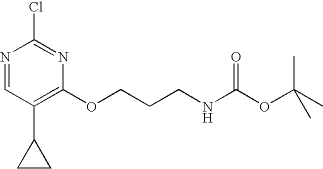

3-(Boc-amino)-1-propanol (278 mg, 1.59 mmol) dissolved in dry THF (1 ml) was added dropwise to a stirred solution of sodium hydride (60% dispersion in mineral oil, 38 mg, 1.59 mmol) in dry THF (1 ml) at 0° C. under nitrogen. The solution was stirred at 0° C. for 30 minutes, then Intermediate 1 (300 mg, 1.59 mmol), in dry THF (1 ml) was added dropwise and stirring continued for further 20 minutes. The reaction mixture was then warmed up to room temperature and stirred at this temperature overnight. The reaction mixture was partitioned between water and DCM, the organic extract was washed with saturated aqueous NaHCO₃, followed by brine, dried (MgSO₄) and solvents removed. Purification by column chromatography using a Biotage SP4 (petroleum ether/ethyl acetate gradient) gave the product as a clear oil (292 mg, 56%). ¹H NMR (400 MHz, CHLOROFORM-d) δ ppm 0.63-0.79 (m, 2H), 0.91-1.07 (m, 2H), 1.44 (s, 9H), 1.82-1.90 (m, 1H), 1.93-2.06 (m, 2H), 3.21-3.37 (m, 2H), 4.51 (t, J=6.0 Hz, 2H), 7.94 (s, 1H). Rf (8:2, petroleum ether/ethyl acetate)=0.24.

### Intermediate 24

### {3-[5-Cyclopropyl-2-(3-fluoro-phenylamino)-pyrimidin-4-yloxy]-propyl}-carbamic acid tert-butyl ester

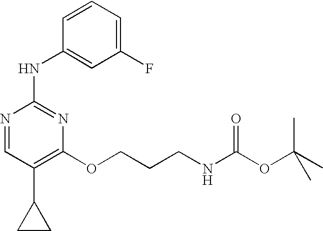

Intermediate 23 (100 mg, 0.31 mmol), 3-fluoroaniline (59 μl, 0.61 mmol) and glacial acetic acid (3.5 μl, 0.06 mmol) were dissolved in n-butanol (1 ml) and irradiated at 150° C. for 15 minutes in a Biotage I-60 microwave reactor. The reaction mixture was concentrated and the crude material purified by column chromatography on Biotage SP4 (petroleum ether/ethyl acetate gradient) to give the product as a white solid (25 mg, 20%). ¹H NMR (400 MHz, DMSO-d₆) δ ppm 0.64-0.69 (m, 2H), 0.79-0.84 (m, 2H), 1.35 (s, 9H), 1.72-1.83 (m, 1H), 1.83-1.93 (m, 2H), 3.04-3.17 (m, 2H), 4.38 (t, J=6.0 Hz, 2H), 6.69 (td, J=8.2, 2.3 Hz, 1H), 6.91 (t, J=5.5 Hz, 1H), 7.22-7.30 (m, 1H), 7.46 (d, J=8.2 Hz, 1H), 7.77 (d, J=12.8 Hz, 1H), 7.94 (s, 1H), 9.60 (s, 1H); m/z (ES+APCI)⁺: 402 [M]⁺.

### Intermediate 25

### [4-(3-Amino-propoxy)-5-cyclopropyl-pyrimidin-2-yl]-(3-fluoro-phenyl)-amine hydrochloride salt

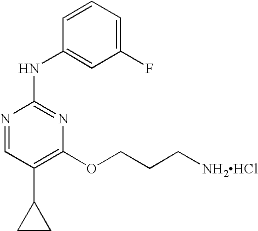

4M HCl in dioxane (1 ml) was added to Intermediate 24 (20 mg, 0.05 mmol) and the reaction mixture stirred at room temperature for 4 hours. The solvents were removed to give the product as a white solid (17 mg, 100%). ¹H NMR (400 MHz, DMSO-d₆) δ ppm 0.65-0.71 (m, 2H), 0.75-0.89 (m, 2H), 1.74-1.82 (m, 1H), 2.03-2.14 (m, 2H), 2.91-3.02 (m, 2H), 4.48 (t, J=6.0 Hz, 2 H), 6.77 (td, J=8.4, 2.1 Hz, 1H), 7.26-7.34 (m, 1H), 7.44 (d, J=8.2 Hz, 1H), 7.73 (dd, J=12.4, 2.3 Hz, 1H), 7.99 (s, 2H), 8.01 (br. s, 2H), 9.98 (br. s, 1H); m/z (ES+APCI)⁺: 303 [M+H]⁺.

### Intermediate 26

Cyclobutanecarboxylic acid (3-methylamino-propyl)-amide hydrochloride salt

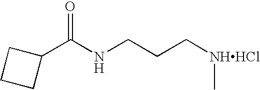

### Step 1

### [3-(Cyclobutanecarbonyl-amino)-propyl]-methyl-carbamic acid tert-butyl ester

To a stirred solution of N-(3-Aminopropyl)N-methylcarbamic acid tert-butylester (600 mg, 3.19 mmol) under nitrogen at 0° C. was added N,N-diisopropylethylamine (610 μl, 3.51 mmol), followed by a dropwise addition of cyclobutanecarbonyl chloride (366 μl, 3.19 mmol). The reaction mixture was stirred at 0° C. for 1 hour, then allowed to warm up to room temperature and stirring continued overnight. The solution was diluted with DCM and washed successively with saturated aqueous NaHCO₃, 1M HCl (aq), water and brine, and the organic phase was dried (MgSO₄) and concentrated. Purification by column chromatography on Biotage SP4 (ethyl acetate/methanol gradient) gave a colourless oil (281 mg, 49%) which was used in Step 2 without further purification.

### Step 2

### Cyclobutanecarboxylic acid (3-methylamino-propyl)-amide hydrochloride salt

4M HCl in dioxane (2 ml) was added to [3-(Cyclobutanecarbonyl-amino)-propyl]-methyl-carbamic acid tert-butyl ester (206 mg, 0.76 mmol) and the reaction mixture stirred for 3 hours at room temperature. The solvents were removed to give the title compound as a white solid (120 mg, 76%). ¹H NMR (400 MHz, DMSO-d₆) δ ppm 1.65-1.79 (m, 3H), 1.83-1.93 (m, 1H), 1.95-2.05 (m, 2H), 2.05-2.17 (m, 2H), 2.73-2.89 (m, 2H), 2.98 (quin, J=8.36, 8.24, 8.01 Hz, 1H), 3.05-3.12 (m, 2H), 7.89 (t, J=5.72 Hz, 1H), 8.75 (br. s, 2H); m/z (ES+APCI)⁺: 171 [M+H]⁺.

### Intermediate 27

Cyclobutanecarboxylic acid {3-[(2-chloro-5-cyclopropyl-pyrimidin-4-yl)-methyl-amino]-propyl}-amide

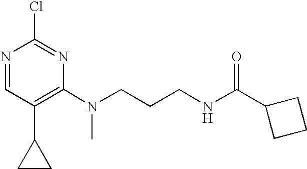

Intermediate 1 (103 mg, 0.54 mmol),

Intermediate 26 (112 mg, 0.54 mmol) and N,N-diisopropylethylamine (289 μl, 1.63 mmol) were combined in isopropanol at 0° C., stirred at this temperature for 45 minutes, warmed up to room temperature, stirred for 10 minutes and then heated to 55° C. overnight. The reaction mixture was allowed to cool to room temperature and partitioned between water and ethyl acetate, extracted twice with ethyl acetate and the combined organic extracts washed with brine, dried (MgSO₄) and solvents removed. Purification by column chromatography on Biotage SP4 (petroleum ether/ethyl acetate gradient) gave the product as a clear oil (120 mg, 69%). ¹H NMR (400 MHz, DMSO-d₆) δ ppm 0.65-0.71 (m, 2H), 0.78-0.96 (m, 2H), 1.63-1.77 (m, 3H), 1.81-2.02 (m, 4H), 2.03-2.14 (m, 2H), 2.94 (quin, J=8.24 Hz, 1H), 3.00-3.08 (m, 2H), 3.12-3.28 (m, 3H), 3.46-3.72 (m, 2H), 7.66 (t, J=5.50 Hz, 1H), 7.88 (s, 1H); m/z (ES+APCI)⁺: 323 [M+H]⁺.

### Intermediate 28

### [3-(2-Chloro-5-cyclopropyl-pyrimidin-4-ylamino)-propyl]-methyl-carbamic acid tert-butyl ester

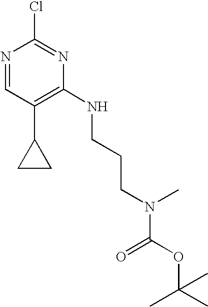

Intermediate 1 (600 mg, 3.2 mmol) was dissolved in isopropyl alcohol and the solution was cooled to 0° C., then N-(3-aminopropyl) carbamic acid tert-butyl ester (450 mg, 3.12 mmol) was added, followed by N,N-diisopropylethylamine (1.69 ml, 9.52 mmol). The solution was stirred for 1 h, warmed to room temperature, stirred for 20 min. and, then warmed to 55° C. and stirred overnight. The reaction mixture was partitioned between ethyl acetate and water, extracted twice with ethyl acetate and the combined organic extracts washed with brine, dried (MgSO₄) and solvents evaporated to dryness. The crude product was purified by flash chromatography using a Biotage SP4 (ethyl acetate/petroleum ether gradient) to give the product as a clear oil (602 mg, 56%). ¹H NMR (400 MHz, DMSO-d₆, 40° C.) δ ppm 0.57-0.62 (m, 2H), 0.87-0.96 (m, 2H), 1.41 (br. s, 9H), 1.48-1.60 (m, 1H), 1.81 (br. s, 2H), 2.82 (s, 3H), 3.14-3.33 (m, 2H), 3.40 (q, J=6.9 Hz, 2H), 7.38 (br. s, 1H), 7.73 (s, 1H); m/z (ES+APCI)⁺: 241 [M−CO₂^{t}Bu+H], 341 [M+H]⁺.

### Intermediate 29

### 5-Cyclopropyl-N2-(3-fluoro-phenyl)-N4-(3-methylamino-propyl)-pyrimidine-2,4-diamine hydrochloride salt

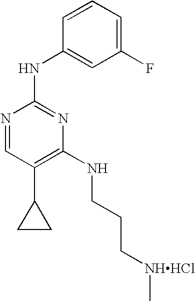

Intermediate 28 (300 mg, 0.9 mmol) was dissolved in 14:1 acetonitrile:water (435 ml) and to it added 3-fluoroaniline (110 μl, 1.2 mmol), followed by 4 M HCl in dioxane (320 μl), and the reaction mixture was heated to 50° C. overnight. The solvents were evaporated and the crude product was purified by flash chromatography using a Biotage SP4 (DCM/3% ammonia in methanol gradient) to give the product as a yellow solid (236 mg, 85%). ¹H NMR (400 MHz, DMSO-d₆) δ ppm 0.60-0.65 (m, 2H), 0.89-1.02 (m, 2H), 1.60-1.67 (m, 1H), 1.96-2.08 (m, 2H), 2.50 (s, 3H, obscured by DMSO peak), 2.81-3.00 (m, 2H), 3.51-3.72 (m, 2H), 6.52-6.65 (m, 1H), 7.03 (td, J=8.2, 2.3 Hz, 1H), 7.29-7.38 (m, 1H), 7.42-7.55 (m, 1H), 7.55-7.71 (m, 1H), 7.76 (s, 1H), 8.92-9.00 (m, 1H), 11.00 (s, 1H); m/z (ES+APCI)⁺: 316 [M+H]⁺.

### Intermediate 30

### 1-(3-Amino-propyl)-piperidin-2-one. hydrochloride salt

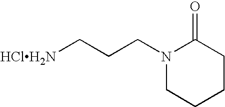

### Step 1

### [3-(2-Oxo-piperidin-1-yl)-propyl]-carbamic acid tert-butyl ester

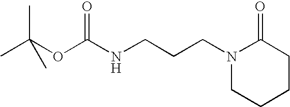

Sodium hydride (60% dispersion in mineral oil; 425 mg, 10.6 mmol) was added to anhydrous THF (10 ml), under nitrogen and the mixture cooled down to 0° C. A solution of valerolactam (500 mg, 5.1 mmol) in anhydrous THF (10 ml) was added dropwise and the reaction mixture stirred at this temperature for 30 min. After this time, a solution of tert-butyl (3-bromopropyl)carbamate (1.44 g, 6.1 mmol) in anhydrous THF (10 ml) was added dropwise, stirring at 0° C. continued for further 15 min., then the solution allowed to warm up to room temperature and stirred overnight. The solution was cooled to 0° C., saturated sodium hydrogen carbonate added and the aqueous phase extracted twice with DCM. The combined organic extracts were washed with brine, dried (MgSO₄) and solvents removed to give the product as a clear oil (960 mg, 74%). The crude material was proceeded through to the next step without further purification. ¹H NMR (400 MHz, DMSO-d₆) δ ppm 1.42 (s, 9H) 1.65-1.83 (m, 4H) 2.12-2.26 (m, 2H) 2.87-2.96 (m, 2H) 3.19-3.35 (m, 4H) 4.14-4.20 (m, 2H) 6.78 (t, J=5.5 Hz, 1H); m/z (ES+APCI)⁺: 157 [M−CO₂^{t}Bu+H].

### Step 2

### 1-(3-Amino-propyl)-piperidin-2-one. hydrochloride salt

4 M HCl in dioxane (6 ml) was added to [3-(2-Oxo-piperidin-1-yl)-propyl]-carbamic acid tert-butyl ester (910 mg, 3.6 mmol) and the reaction mixture was stirred at room temperature for 4 hours. The solvents were evaporated to give the title compound as an oily white solid (984 mg). ¹H NMR (400 MHz, DMSO-d₆) δ ppm 1.76-1.92 (m, 4H), 2.26 (t, J=6.2 Hz, 2H), 2.69-2.80 (m, 2H), 3.14-3.22 (m, 2H), 3.27 (t, J=5.7 Hz, 2H), 3.36 (t, J=6.9 Hz, 2H), 8.03 (br. s, 3H); m/z (ES+APCI)⁺: 157 [M+H]⁺.

### Intermediate 31

### 1-[3-(2-Chloro-5-cyclopropyl-pyrimidin-4-ylamino)-propyl]-piperidin-2-one

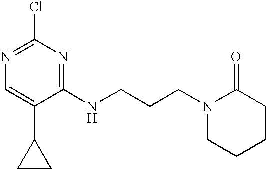

Intermediate 1 (542 mg, 2.9 mmol),

Intermediate 30 (552 mg, 2.9 mmol) and N,N-diisopropylethylamine (1.53 ml, 8.6 mmol) were mixed together in isopropyl alcohol (10 ml) at 0° C., stirred for 1 h, then warmed up to room temperature, stirred for 10 min., then warmed up to 55° C. overnight. The reaction mixture was partitioned between water and ethyl acetate, extracted twice with ethyl acetate, combined organic extracts washed with brine, dried (MgSO₄) and solvents evaporated. The crude product was purified by flash chromatography using a Biotage SP4 (ethyl acetate/methanol gradient) to give the product as a clear oil (401 mg, 45%). ¹H NMR (400 MHz, DMSO-d₆) δ ppm 0.54-0.63 (m, 2H), 0.86-0.99 (m, 2H), 1.50-1.59 (m, 1H), 1.66-1.84 (m, 6H), 2.26 (t, J=6.2 Hz, 2H), 3.30 (t, J=6.0 Hz, 2H), 3.35-3.41 (m, 4H), 7.57 (t, J=6.2 Hz, 1H), 7.74 (s, 1H); m/z (ES+APCI)⁺: 309 [M+H]⁺.

### Intermediate 32

### 1-(3-Amino-propyl)-3-methyl-1H-pyridin-2-one hydrochloride salt

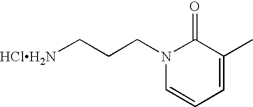

### Step 1

### [3-(3-Methyl-2-oxo-2H-pyridin-1-yl)-propyl]-carbamic acid tert-butyl ester

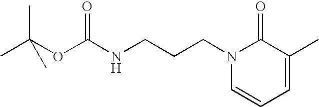

Sodium hydride (60% dispersion in mineral oil; 231 mg, 9.6 mmol) was added to anhydrous THF (10 ml), under nitrogen and the mixture cooled down to 0° C. A solution of 3-methyl-2-pyridone (500 mg, 4.6 mmol) in anhydrous THF (10 ml) was added dropwise and the reaction mixture stirred at this temperature for 30 min. After this time, a solution of tert-butyl (3-bromopropyl)carbamate (1.31 g, 5.5 mmol) in anhydrous THF (10 ml) was added dropwise, and stirring was continued at 0° C. for a further 15 min., and then at room temperature overnight. The solution was cooled to 0° C. and then quenched with saturated sodium hydrogen carbonate(aq) and the aqueous phase extracted twice with DCM. The combined organic extracts were washed with brine and the solution filtered through a phase separation cartridge and solvents evaporated. The crude product was purified by flash chromatography using a Biotage SP4 (ethyl acetate/methanol gradient) to give the product (165 mg, 14%). ¹H NMR (400 MHz, CHLOROFORM-d) δ ppm 1.43 (s, 9H), 1.84-1.93 (m, 2H), 2.16 (s, 3H), 3.04-3.12 (m, 2H), 4.03 (t, J=6.6 Hz, 2H), 6.15 (t, J=6.9 Hz, 1H), 7.22 (d, J=6.4 Hz, 1H), 7.18 (d, J=6.9 Hz, 1H); m/z (ES+APCI)⁺: 167 [M−CO₂^{t}Bu+H]⁺.

### Step 2

4 M HCl in dioxane (1 ml) was added to [3-(3-Methyl-2-oxo-2H-pyridin-1-yl)-propyl]-carbamic acid tert-butyl ester (160 mg, 0.6 mmol) and the reaction mixture stirred at room temperature for 3 hours. The solvents were evaporated to give the product as a brown solid (110 mg, 90%). ¹H NMR (400 MHz, DMSO-d₆) δ ppm 1.94-2.02 (m, 2H), 2.04 (s, 3H), 2.74-2.86 (m, 2H), 4.03 (t, J=6.9 Hz, 2H), 6.23 (t, J=6.9 Hz, 1H), 7.37 (d, J=6.9 Hz, 1H), 7.65 (d, J=5.5 Hz, 1H), 8.11 (br. s, 3H); m/z (ES+APCI)⁺: 167 [M+H]⁺.

### Intermediate 33

### 1-[3-(2-Chloro-5-cyclopropyl-pyrimidin-4-ylamino)-propyl]-3-methyl-1H-pyridin-2-one

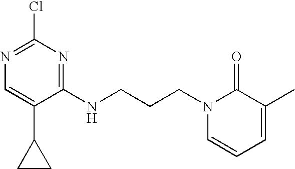

Intermediate 1 (103 mg, 0.5 mmol), Intermediate 32 (110 mg, 0.5 mmol) and N,N-diisopropylethylamine (290 μl, 1.63 mmol) were mixed together in isopropyl alcohol (1 ml) at 0° C., stirred for 30 min., then warmed up to room temperature, stirred for 15 min., then warmed up to 55° C. overnight. The reaction mixture was partitioned between water and ethyl acetate, extracted twice with ethyl acetate and the combined organic extracts were washed with brine, dried (MgSO₄) and solvents evaporated. The crude product was purified by flash chromatography using a Biotage SP4 (petroleum ether/ethyl acetate) to give the product as a clear oil (120 mg, 64%). ¹H NMR (400 MHz, DMSO-d₆) δ ppm 0.55-0.67 (m, 2H), 0.87-0.96 (m, 2H), 1.52-1.59 (m, 1H), 1.89-1.99 (m, 2H), 2.03 (s, 3H), 3.38-3.46 (m, 2H), 3.94-4.03 (m, 2H), 6.19 (t, J=6.9 Hz, 1H), 7.33 (d, J=6.0 Hz, 1H), 7.54-7.63 (m, 2H), 7.74 (s, 1H); m/z (ES+APCI)⁺: 319 [M+H]⁺.

### Intermediate 34

### 1-(3-Amino-propyl)-3-methyl-imidazolidin-2-one

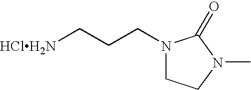

### Step 1

### [3-(3-Methyl-2-oxo-imidazolidin-1-yl)-propyl]-carbamic acid tert-butyl ester

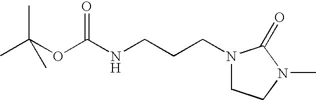

Sodium hydride (60% dispersion in mineral oil; 252 mg, 10.5 mmol) was added to anhydrous THF (10 ml), under nitrogen and the mixture cooled down to 0° C. A solution of 1-methyl-2-imidazolidinone (500 mg, 5.0 mmol) in anhydrous THF (10 ml) was added dropwise and the reaction mixture stirred at this temperature for 30 min. After this time, a solution of tert-butyl (3-bromopropyl)carbamate (1.43 g, 6.0 mmol) in anhydrous THF (10 ml) was added dropwise at 0° C. and stirring at this temperature continued for further 15 min., before allowing to warm up to room temperature overnight. The solution was cooled to 0° C. and quenched with and water. Saturated sodium hydrogen carbonate(aq) was added and the aqueous phase was extracted twice with DCM. The combined organic extracts were washed with brine, dried (MgSO₄) and solvents evaporated. The crude product was purified by flash chromatography using a Biotage SP4 (ethyl acetate/methanol gradient) to give the product as a clear oil (279 mg, 22%). ¹H NMR (400 MHz, CHLOROFORM-d) δ ppm 1.43 (s, 9H), 1.58-1.68 (m, 2H), 2.78 (s, 3H), 3.11 (t, J=6.4 Hz, 2H), 3.24 (t, 2H), 3.29 (s, 4H); m/z (ES+APCI)⁺: 158 [M−CO₂^{t}Bu+H]⁺.

### Step 2

### 1-(3-Amino-propyl)-3-methyl-imidazolidin-2-one

4 M HCl in dioxane (2 ml) was added to [3-(3-Methyl-2-oxo-imidazolidin-1-yl)-propyl]-carbamic acid tert-butyl ester (269 mg, 1.1 mmol) and the reaction mixture stirred at room temperature for 1 h. The solvents were evaporated to give the product as a white solid (206 mg, 100%). ¹H NMR (400 MHz, DMSO-d₆) δ ppm 1.61-1.88 (m, 2H), 2.67 (s, 3H), 2.71-2.81 (m, 2H), 3.15 (t, J=6.9 Hz, 2H), 3.27 (s, 4H), 7.93 (br. s, 3H); m/z (ES+APCI)⁺: 158 [M+H]⁺.

### Intermediate 35

### 1-[3-(2-Chloro-5-cyclopropyl-pyrimidin-4-ylamino)-propyl]-3-methyl-imidazolidin-2-one

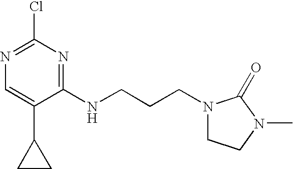

Intermediate 1 (195 mg, 1.0 mmol), Intermediate 34 (200 mg, 1.0 mmol) and N,N-diisopropylethylamine (551 μl, 3.1 mmol) were mixed together in isopropyl alcohol (1 ml) at 0° C., stirred for 30 min., then warmed up to room temperature and stirred for 15 min., then warmed up to 55° C. overnight. The reaction mixture was partitioned between water and ethyl acetate, extracted twice with ethyl acetate and the combined organic extracts were washed with brine, dried (MgSO₄) and solvents evaporated. The crude product was purified by flash chromatography using a Biotage SP4 (petroleum ether/ethyl acetate) to give the product as a white solid (159 mg, 50%). ¹H NMR (400 MHz, DMSO-d₆) δ ppm 0.39-0.68 (m, 2H), 0.88-0.97 (m, 2H), 1.50-1.59 (m, 1H), 1.69-1.80 (m, 2H), 2.68 (s, 3H), 3.17 (t, J=6.9 Hz, 2H), 3.24-3.35 (m, 4H), 3.38-3.43 (m, 2H), 7.52 (t, J=6.0 Hz, 1H), 7.73 (s, 1H); m/z (ES+APCI)⁺: 310 [M+H]⁺.

### Intermediate 36

### 1-(3-Amino-propyl)-3,3-dimethyl-pyrrolidin-2-one hydrochloride salt

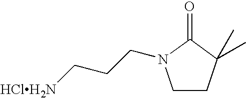

### Step 1

### 2-Oxo-pyrrolidine-1-carboxylic acid tert-butyl ester

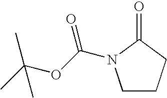

To a solution of 2-pyrrolidinone (3 g, 35.3 mmol) in DCM (80 ml) was added sequentially triethylamine (4.9 ml, 35.3 mmol), di-tert-butyl dicarbonate (15.4 g, 70.6 mmol), and 4-dimethylaminopyridine (4.3 g, 35.3 mmol). The reaction mixture was stirred over 3 days, the solvents removed and the crude material purified by flash chromatography using a Biotage SP4 (ethyl acetate/methanol gradient) to give the product as a yellow oil (5.4 g, 83%). ¹H NMR (400 MHz, CHLOROFORM-d) δ ppm 1.51 (s, 9H), 1.93-2.04 (m, 2H), 2.45-2.54 (m, 2H), 3.68-3.77 (m, 2H); Rf (1:9 MeOH/EtOAc)=0.6.

### Step 2

### 3,3-Dimethyl-2-oxo-pyrrolidine-1-carboxylic acid tert-butyl ester

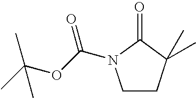

2-Oxo-pyrrolidine-1-carboxylic acid tert-butyl ester (2 g, 10.8 mmol) was dissolved in anhydrous THF (100 ml), under nitrogen, cooled down to −78° C. and to it added LHMDS (1 M solution in THF, 32.4 ml, 32.4 mmol) dropwise. The solution was left to stir at −78° C. for 45 min. prior to dropwise addition of methyl iodide (4.0 ml, 64.9 mmol). The resulting mixture was stirred at −78° C. for a further 30 min. and at then room temperature overnight. The solution was then cooled down to 0° C. then quenched with water, followed by 1M HCl. The aqueous phase was extracted with diethyl ether (×3) and the combined organic extracts washed with saturated sodium hydrogen carbonate, followed by brine, dried (MgSO₄) and the solvent evaporated. The crude material was purified by flash chromatography using a Biotage SP4 (petroleum ether/ethyl acetate gradient) to give the product as a brown solid (1.0 g, 43%). ¹H NMR (400 MHz, CHLOROFORM-d) δ ppm 1.19 (s, 6H), 1.53 (s, 9H), 1.83 (t, J=6.9 Hz, 2H), 3.62-3.69 (m, 2H); m/z (ES+APCI)⁺: 114 [M−CO₂^{t}Bu+H]⁺.

### Step 3

### 3,3-Dimethyl-pyrrolidin-2-one

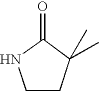

4 M HCl in dioxane (10 ml) was added to 3,3-Dimethyl-2-oxo-pyrrolidine-1-carboxylic acid tert-butyl ester (990 mg, 4.65 mmol) and the reaction mixture stirred at room temperature for 3 h. The solvents were removed to give the product as an oily brown solid (668 mg, 96%). ¹H NMR (400 MHz, DMSO-d₆) δ ppm 1.03 (s, 6H), 1.85 (t, J=6.9 Hz, 2H), 3.15 (t, J=6.4 Hz, 2H), 5.15 (br s, 1H); m/z (ES+APCI)⁺: 114 [M+H]+

### Step 4

### [3-(3,3-Dimethyl-2-oxo-pyrrolidin-1-yl)-propyl]-carbamic acid tert-butyl ester

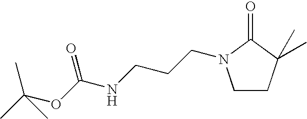

Sodium hydride (60% dispersion in mineral oil; 332 mg, 13.9 mmol) was added to anhydrous THF (10 ml), under nitrogen and the mixture cooled down to 0° C. A solution of 3,3-Dimethyl-pyrrolidin-2-one (668 mg, 4.5 mmol) in anhydrous THF (10 ml) was added dropwise and the reaction mixture stirred at this temperature for 45 min. After this time, a solution of tert-butyl (3-bromopropyl)carbamate (1.28 g, 5.4 mmol) in anhydrous THF (10 ml) was added dropwise and stirring was continued at 0° C. for further 30 min., and then at room temperature overnight. The solution was cooled to 0° C. and then quenched with water, followed by saturated sodium hydrogen carbonate(aq). The aqueous phase was extracted twice with DCM, and the combined organic extracts were washed with brine, dried (MgSO₄) and solvent evaporated. Purification by flash chromatography using a Biotage SP4 (ethyl acetate/methanol gradient) gave a clear oil (657 mg, 54%). ¹H NMR (400 MHz, DMSO-d₆) δ ppm 1.05 (s, 6H), 1.41 (s, 9H), 1.53-1.62 (m, 2H), 1.82 (t, J=6.6 Hz, 2H), 2.87-2.94 (m, 2H), 3.18 (t, J=7.1 Hz, 2H), 3.28 (t, J=6.6 Hz, 2H), 6.70 (m, 1H); m/z (ES+APCI)⁺: 171 [M−CO₂^{t}Bu+H]⁺.

### Step 5

### 1-(3-Amino-propyl)-3,3-dimethyl-pyrrolidin-2-one hydrochloride salt

4 M HCl in dioxane (5 ml) was added to [3-(3,3-Dimethyl-2-oxo-pyrrolidin-1-yl)-propyl]-carbamic acid tert-butyl ester (654 mg, 2.4 mmol) and the reaction mixture stirred at room temperature for 1 h. The solvents were removed to give the title compound as a clear oil (645 mg). ¹H NMR (400 MHz, DMSO-d₆) δ ppm 1.05 (s, 6H), 1.75-1.88 (m, 4H), 2.68-2.78 (m, 2H), 3.24-3.34 (m, 4H), 7.85 (br s, 2H); m/z (ES+APCI)⁺: 171 [M+H]⁺.

### Intermediate 37

### 1-[3-(2-Chloro-5-cyclopropyl-pyrimidin-4-ylamino)-propyl]-3,3-dimethyl-pyrrolidin-2-one

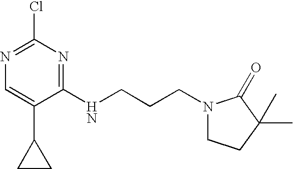

Intermediate 1 (117 mg, 0.6 mmol), Intermediate 36 (128 mg, 0.6 mmol) and N,N-diisopropylethylamine (330 μl, 1.9 mmol) were mixed together in isopropyl alcohol (5 ml) at 0° C., stirred for 30 min., then warmed up to room temperature, stirred for 10 min., then warmed up to 55° C. overnight. The reaction mixture was cooled down and partitioned between water and ethyl acetate, extracted twice with ethyl acetate, and the combined organic extracts were washed with brine, dried (MgSO₄) and solvents evaporated. The crude product was purified by flash chromatography using a Biotage SP4 (petroleum ether/ethyl acetate gradient) to give the product as a colourless oil (86 mg, 43%). ¹H NMR (400 MHz, DMSO-d₆) δ ppm 0.54-0.68 (m, 2H), 0.89-0.96 (m, 2H), 1.08 (s, 6H), 1.51-1.59 (m, 1H), 1.72-1.81 (m, 2H), 1.86 (t, J=6.9 Hz, 2H), 3.28 (t, J=6.6 Hz, 2H), 3.32-3.37 (m, 4H), 7.49-7.55 (m, 1H), 7.74 (s, 1H);); m/z (ES+APCI)⁺: 323 [M+H]⁺.

### Intermediate 38

### 1-(3-Amino-propyl)-3-trifluoromethyl-1H-pyridin-2-one. hydrochloride salt

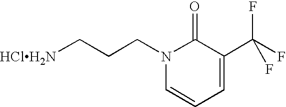

### Step 1

### [3-(2-Oxo-3-trifluoromethyl-2H-pyridin-1-yl)-propyl]-carbamic acid tert-butyl ester

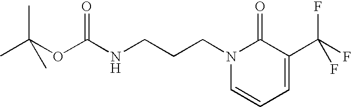

Sodium hydride (60% dispersion in mineral oil, 88 mg, 3.7 mmol) was added to 1:4 anhydrous DMF/1,2-dimethoxyethane (25 ml) under nitrogen. The mixture was cooled down to 0° C., then a solution of 2-hydroxy-3-(trifluoromethyl)pyridine (500 mg, 3.1 mmol) in dry DMF (5 ml) was added dropwise and the resulting mixture was stirred for a further 10 min. Lithium bromide (534 mg, 6.1 mmol) was added and the resulting mixture was stirred for 15 min prior to dropwise addition of tert-butyl (3-bromopropyl)carbamate (876 mg, 3.7 mmol) in DMF (5 ml). After a further 10 min., the reaction mixture was allowed to warm to room temperature and stirring was continued for 3 days. The solution was cooled down to 0° C., quenched with water and the aqueous phase extracted with ethyl acetate (×3). The combined organic extracts were washed with water followed by brine, dried (MgSO₄) and solvents evaporated. The crude product was purified by flash chromatography using a Biotage SP4 (DCM/methanol gradient) to give a yellow oil (111 mg, 11%). ¹H NMR (400 MHz, DMSO-d₆) δ ppm 1.35-1.52 (m, 9H), 1.76-1.88 (m, 2H), 2.94-3.02 (m, 2H), 3.99 (t, J=7.1 Hz, 2H), 6.42 (t, J=6.9 Hz, 1H), 6.95 (t, J=5.7 Hz, 1H), 7.97 (d, J=7.3 Hz, 1H), 8.10 (d, J=6.9 Hz, 1H); m/z (ES+APCI)⁺: 221 [M−CO₂^{t}Bu+H]⁺.

### Step 2

### 1-(3-Amino-propyl)-3-trifluoromethyl-1H-pyridin-2-one. Hydrochloride salt

4 M HCl in dioxane (4 ml) was added to [3-(2-Oxo-3-trifluoromethyl-2H-pyridin-1-yl)-propyl]-carbamic acid tert-butyl ester (107 mg, 0.3 mmol) and the reaction mixture was stirred at room temperature for 3 h. The solvents were removed to give the product as a brown oil (110 mg). ¹H NMR (400 MHz, DMSO-d₆) δ ppm 1.90-2.12 (m, 2H), 2.78-2.87 (m, 2H), 4.09 (t, J=7.1 Hz, 2H), 6.46 (t, J=6.9 Hz, 1H), 7.88-8.07 (m, 4H), 8.15 (d, J=6.9 Hz, 1H); m/z (ES+APCI)⁺: 221 [M+H]⁺

### Intermediate 39

### 1-[3-(2-Chloro-5-cyclopropyl-pyrimidin-4-ylamino)-propyl]-3-trifluoromethyl-1H-pyridin-2-one

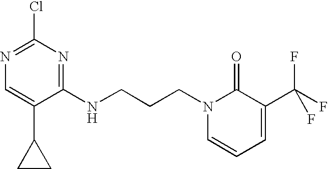

Intermediate 1 (79 mg, 0.4 mmol), Intermediate 38 (107 mg, 0.4 mmol) and N,N-diisopropylethylamine (222 μl, 1.3 mmol) were mixed together in isopropyl alcohol (2 ml) at 0° C., stirred for 30 min., warmed to room temperature and stirred for 15 min. and then warmed to 55° C. and stirred overnight. The reaction mixture was cooled down and partitioned between water and ethyl acetate and the aqueous phase extracted twice with ethyl acetate, the combined organic extracts washed with brine, dried (MgSO₄) and the solvent was evaporated. The crude product was purified by flash chromatography using a Biotage SP4 (petroleum ether/ethyl acetate gradient) to give a white solid (47 mg, 30%). ¹H NMR (400 MHz, DMSO-d₆) δ ppm 0.53-0.66 (m, 2H), 0.88-0.95 (m, 2H), 1.51-1.58 (m, 1H), 1.95-2.03 (m, 2H), 3.39-3.48 (m, 2H), 4.01-4.09 (m, 2H), 6.42 (t, J=6.9 Hz, 1H), 7.53 (t, J=5.7 Hz, 1H), 7.74 (s, 1H), 7.97 (d, J=6.0 Hz, 1H), 8.11-8.15 (m, 1H); m/z (ES+APCI)⁺: 373 [M+H]⁺.

### Intermediate 40

### 5-Cyclopropyl-N4-(3-methylamino-propyl)-N2-(6-morpholin-4-yl-pyridin-3-yl)-pyrimidine-2,4-diamine hydrochloride salt

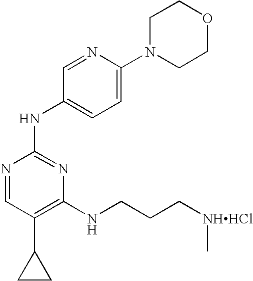

### Step 1

### {3-[5-Cyclopropyl-2-(6-morpholin-4-yl-pyridin-3-ylamino)-pyrimidin-4-ylamino]-propyl}-methyl-carbamic acid tert-butyl ester

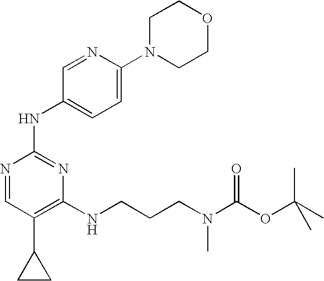

To a stirred solution of Intermediate 28 (194 mg, 0.6 mmol) in 14:1 acetonitrile/water was added 6-morpholinopyridin-3-amine (133 mg, 0.7 mmol) followed by 4M HCl in dioxane (207 μl), and the resulting mixture stirred to 50° C. overnight. The solvents were evaporated and the crude material was purified by preparative LCMS (high pH buffer) to give a purple solid (140 mg, 51%). ¹H NMR (400 MHz, DMSO-d₆, 80° C.) δ ppm 0.47-0.54 (m, 2H), 0.84-0.90 (m, 2H), 1.43 (s, 9H), 1.46-1.55 (m, 1H), 1.81-1.88 (m, 2H), 2.84 (s, 3H), 3.31 (t, J=6.9 Hz, 2H), 3.36-3.41 (m, 4H), 3.43-3.49 (m, 2H), 3.73-3.77 (m, 4H), 6.46-6.51 (m, 1H), 6.78 (d, J=9.2 Hz, 1H), 7.61 (s, 1H), 7.98 (dd, J=8.7, 2.7 Hz, 1H), 8.35 (s, 1H), 8.48 (d, J=2.7 Hz, 1H); m/z (ES+APCI)⁺: 484 [M+H]⁺.

### Step 2

### 5-Cyclopropyl-N4-(3-methylamino-propyl)-N2-(6-morpholin-4-yl-pyridin-3-yl)-pyrimidine-2,4-diamine hydrochloride salt

4 M HCl in dioxane (3 ml) was added to {3-[5-Cyclopropyl-2-(6-morpholin-4-yl-pyridin-3-ylamino)-pyrimidin-4-ylamino]-propyl}-methyl-carbamic acid tert-butyl ester (135 mg, 0.3 mmol) and the reaction mixture was stirred at room temperature for 1.5 h. The volatiles were evaporated to give the product as a purple coloured oil (161 mg). ¹H NMR (400 MHz, DMSO-d₆, 80° C.) δ ppm 0.54-0.68 (m, 2H), 0.88-0.98 (m, 2H), 1.57-1.65 (m, 1H), 1.94-2.03 (m, 2H), 2.57 (s, 3H, obscured by DMSO peak), 2.90-2.99 (m, 2H), 3.51-3.80 (m, 6H, obscured by water peak), 3.74-3.91 (m, 4H), 7.19-7.26 (m, 1H), 7.63 (s, 1H), 7.86-7.93 (m, 1H), 8.42 (s, 1H), 8.86 (br. s, 1H), 9.02 (br. s, 1H), 10.53 (br. s, 1H); m/z (ES+APCI)⁺: 384 [M+H]⁺.

### Intermediate 41

### 6-(1-Methyl-1H-pyrazol-4-yl)-pyridin-3-ylamine

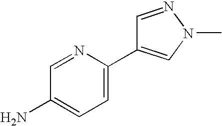

To a solution of 3-amino-6-bromopyridine (400 mg, 2.3 mmol) and 1-methyl-4-(4,4,5,5-tetramethyl-1,3,2-dioxaborolan-2-yl)-1H-pyrazole (721 mg, 3.5 mmol) in 15:1 1,2-dimethoxyethane/water (16 ml), was added potassium carbonate (297 mg, 3.5 mmol), followed by Pd(PPh₃)₄ (267 mg, 0.2 mmol), under nitrogen, and the resulting mixture was heated at 80° C. for 16 h. The mixture was cooled, partitioned between water and ethyl acetate, and the aqueous phase was extracted with ethyl acetate. The combined organic phases were washed with water followed by brine, dried (MgSO₄) and solvents were evaporated. The crude product was purified by flash chromatography using a Biotage SP4 (DCM/methanol gradient) to give a brown solid (45 mg, 11%). ¹H NMR (400 MHz, CHLOROFORM-d) δ ppm 3.65-3.80 (br s, 2H), 3.85 (s, 3H), 6.91-6.95 (m, 1H), 7.20 (d, J=7.8 Hz, 1H), 7.74 (s, 1H), 7.79 (s, 1H), 7.99 (d, J=2.7 Hz, 1H); Rf=0.43 (1:9 MeOH/DCM).

### Intermediate 42

### Cyclobutanecarboxylic acid {3-[2-(3-bromomethyl-phenylamino)-5-cyclopropyl-pyrimidin-4-ylamino]-propyl}-amide

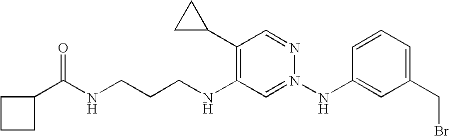

To a solution of Example 70 (120 mg, 0.28 mmol) in glacial acetic acid (10 ml) was added 30% HBr in AcOH (10 ml). The orange solution was allowed to stir at room temperature for 18 h. The reaction mixture was poured into diethyl ether (30 ml), partitioned with water (30 ml), basified with 2M sodium hydroxide solution and the layers separated. The aqueous layer was extracted diethyl ether (30 ml) and DCM (30 ml). The organic phases were combined and washed with brine, dried (MgSO₄) and evaporated. The crude material was purified by flash chromatography on the Biotage SP4, eluting with 0 to 10% Methanol/DCM gradient. This gave the desired product as a white solid. ¹H NMR (400 MHz, DMSO-d₆) δ ppm 0.46-0.52 (m, 2H), 0.81-0.90 (m, 2H), 1.44-1.53 (m, 1H), 1.66-1.78 (m, 3H), 1.79-1.93 (m, 3H), 1.94-2.03 (m, 2H), 2.05-2.17 (m, 2H), 2.90-3.01 (m, 1H), 3.10-3.18 (m, 2H), 3.43-3.51 (m, 2H), 6.93-7.00 (m, 1H), 7.07-7.17 (m, 1H), 7.19-7.25 (m, 1H), 7.52-7.58 (m, 1H), 7.62 (s, 1H), 7.66-7.73 (m, 1H), 7.95-8.01 (m, 1H), 9.14 (br. s, 1H); m/z (ES+APCI)⁺: 458/460 [M+H]⁺.

### Intermediate 43

### 3-(1-Morpholin-4-yl-ethyl)-phenylamine

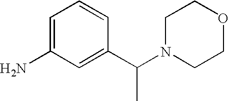

### Step 1

### 1-(1-Chloro-ethyl)-3-nitro-benzene

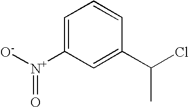

Thionyl chloride (162 ul, 8.9 mmol) was added dropwise to a solution of 1-(3-nitro-phenyl)-ethanol (4.4 mmol) in DCM (50 ml) at 0° C. The reaction was stirred at room temperature for 2 h. Further thionyl chloride (162 μl, 8.9 mmol) was added and the reaction stirred at 35° C. for 24 h. The mixture was evaporated to give the desired product as a yellow oil. NMR (DMSO) shows 70%, together with 3-nitrostyrene impurity. Used in the next step without further purification.

### Step 2

### 4-[1-(3-Nitro-phenyl)-ethyl]-morpholine

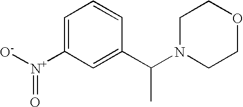

To a solution of the crude 1-(1-chloro-ethyl)-3-nitro-benzene (700 mg, 3.78 mmol) in DCM (20 ml) was added diisopropylethylamine (1.23 ml, 7.57 mmol) and then morpholine (494 μl, 5.68 mmol). The reaction was stirred at 40° C. for 18 h, further diisopropylethylamine (1.23 ml, 7.57 mmol) and morpholine (659 μl, 7.57 mmol) were added and the reaction continued for another 18 h. The reaction was diluted with water (50 ml) and the pH was adjusted to 8 and then extracted twice with DCM (40 ml). The combined organics were washed with saturated sodium chloride solution, dried (MgSO₄) and evaporated. Purification by flash chromatography on the Biotage SP4, eluting with 0 to 50% ethyl acetate/petroleum ether gradient, gave the desired product as a yellow oil (46%).

¹H NMR (400 MHz, DMSO-d₆) δ ppm 1.30 (d, J=6.4 Hz, 3H), 2.23-2.31 (m, 2H), 2.38-2.47 (m, 2H), 3.51-3.60 (m, 5H), 7.61-7.66 (m, 1H), 7.77-7.81 (m, 1H), 8.12 (ddd, J=7.8, 2.3, 1.4 Hz, 1H), 8.14-8.16 (m, 1H); m/z (ES+APCI)⁺: 237 [M+H]⁺.

### Step 3

### 3-(1-Morpholin-4-yl-ethyl)-phenylamine

Iron powder (329 mg, 5.90 mmol) was added to a solution of Step 2 (470 mg, 1.99 mmol) in acetic acid (6.3 ml) and water (3.9 ml). The reaction was stirred at 70° C. for 1 h, cooled to room temperature, partitioned between ethyl acetate and water. The aqueous phase was extracted twice with ethyl acetate (40 ml), the combined organics were dried (MgSO₄) and evaporated to give the title compound as a pale brown oil (88%).

¹H NMR (400 MHz, DMSO-d₆) δ ppm 1.21 (d, J=6.9 Hz, 3H), 2.20-2.29 (m, 2H), 2.30-2.42 (m, 2H), 3.07 (q, J=6.4 Hz, 1H), 3.48-3.59 (m, 4H), 4.98 (br. s, 2H), 6.37-6.44 (m, 2H), 6.49-6.53 (m, 1H), 6.88-6.97 (m, 1H); m/z (ES+APCI)⁺: 207 [M+H]⁺.

### Intermediate 44

### Cyclobutanecarboxylic acid {3-[2-(3-chloromethyl-phenylamino)-5-cyclopropyl-pyrimidin-4-ylamino]-propyl}-amide. Hydrochloride.

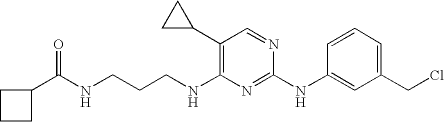

Thionyl chloride (253 μl, 3.5 mmol) was added dropwise to a suspension of Example 70 (300 mg, 0.69 mmol) in DCM (25 ml) at 0° C. The mixture was then allowed to warm to room temperature and stirred for 18 h producing a white precipitate. The reaction was evaporated to give the desired product as a white solid (100%).

¹H NMR (400 MHz, DMSO-d₆) δ ppm 0.55-0.60 (m, 2H), 0.88-0.94 (m, 2H), 1.52-1.60 (m, 1H), 1.66-1.77 (m, 3H), 1.79-1.91 (m, 1H), 1.92-2.01 (m, 2H), 2.03-2.14 (m, 2H), 2.90-2.99 (m, 1H), 3.08-3.15 (m, 2H), 3.47-3.54 (m, 2H), 4.79 (s, 2H), 7.20-7.24 (m, 1H), 7.38-7.43 (m, 1H), 7.46-7.51 (m, 1H), 7.65 (s, 1H), 7.74-7.76 (m, 1H), 7.76-7.81 (m, 1H), 10.53 (br. s, 1H), 12.10 (br. s, 1H); m/z (ES+APCI)⁺: 414 [M+H]⁺.

### Intermediate 45

### (3-Amino-phenyl)-acetonitrile

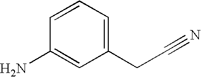

Iron powder (517 mg, 9.27 mmol) was added to a solution of 3-nitrophenylacetonitrile (500 mg, 3.09 mmol) in acetic acid (9.5 ml) and water (6.6 ml). The reaction was stirred at 40° C. for 24 h, then cooled to room temperature. The mixture was diluted with water (20 ml), adjusted to pH 8 with 2N NaOH and then filtered trough Celite®. The aqueous filtrate was extracted with ethyl acetate, the combined organic phases were dried (MgSO₄) and evaporated. Purification by flash chromatography on the Biotage SP4, eluting with 0 to 60% ethyl acetate/petroleum ether gradient, gave the desired product as a colourless oil (51%). ¹H NMR (400 MHz, DMSO-d₆) δ ppm 3.84 (s, 2H), 5.22 (br. s, 2H), 6.40-6.44 (m, 1H), 6.46-6.50 (m, 1H), 6.50-6.53 (m, 1H), 6.96-7.03 (m, 1H); Rf (50% Ethyl acetate/Petroleum ether)=0.5.

### Intermediate 46

### 4-(5-Nitro-pyrimidin-2-yl)-morpholine

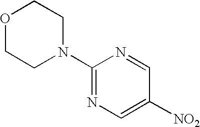

A mixture of DIPEA (1.1 ml, 6.25 mmol) and morpholine (544 μl, 6.25 mmol) in MeCN (5 ml) was added to a stirred solution of 2-chloro-5-nitropyrimidine (1.0 g, 6.25 mmol) in MeCN (15 ml). The mixture was stirred at rt for 3 days. The reaction mixture was concentrated to dryness and diluted with DCM and saturated NaHCO₃ solution (aq). The organic phase was dried and concentrated to dryness to give an orange coloured oily solid (1.25 g, 95%). ¹H NMR (400 MHz, CHLOROFORM-d) δ ppm 3.77-3.86 (m, 4H), 3.97-4.06 (m, 4H), 9.08 (s, 2H).

### Intermediate 47

### 2-Morpholin-4-yl-pyrimidin-5-ylamine

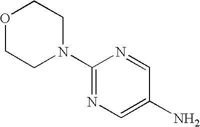

Prepared analogously to Intermediate 13 using Intermediate 46 to afford an orange coloured solid (1.04 g, 97%). ¹H NMR (400 MHz, DMSO-d₆) δ ppm 3.36-3.50 (m, 4H), 3.57-3.68 (m, 4H), 4.66 (s, 2H), 7.90 (s, 2H); m/z (ES+APCI)⁺: 181 [M+H]⁺.

### Intermediate 48

### 4-[2-(5-Nitro-pyridin-2-yloxy)-ethyl]-morpholine

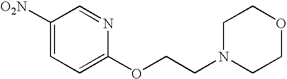

Sodium hydride, 60% dispersion in oil (252 mg, 6.29 mmol) was added portionwise to a solution of 2-chloro-5-nitropyridine (1.0 g, 6.29 mmol) and 2-morpholin-4-yl-ethanol (785 mg, 5.99 mmol) in DMF (20 ml) with ice-cooling. The ice-cooling was removed after 1 hour and the mixture was stirred at rt overnight. The reaction mixture was added to an ice/water mixture and extracted with EtOAc (×2). The combined organic extracts were washed with water (×4) and brine (×1), dried and concentrated to dryness. The residue was purified by flash column chromatography on silica gel (50 g) eluting with 40:1 DCM-MeOH to provide a brown solid (980 mg, 62%). ¹H NMR (400 MHz, DMSO-d₆) δ ppm 2.45 (br. s, 4H), 2.71 (t, J=5.72 Hz, 2H), 3.49-3.61 (m, 4H), 4.52 (t, J=5.72 Hz, 2H), 7.04 (d, J=9.16 Hz, 1H), 8.44-8.49 (m, 1H), 9.08 (d, J=3.66 Hz, 1H); m/z (ES+APCI)⁺: 254 [M+H]⁺.

### Intermediate 49

### 6-(2-Morpholin-4-yl-ethoxy)-pyridin-3-ylamine

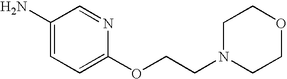

Prepared analogously to Intermediate 13 using Intermediate 48 to give an orange oil (883 mg, 102%). ¹H NMR (400 MHz, DMSO-d₆) δ ppm 2.36-2.48 (m, 4H), 2.60 (t, J=5.95 Hz, 2H), 3.51-3.59 (m, 4H), 4.19 (t, J=5.95 Hz, 2H), 4.73 (s, 2H), 6.52 (d, J=8.70 Hz, 1H), 6.96-7.00 (m, 1H), 7.47 (d, J=3.66 Hz, 1H).

### Intermediate 50

### 4-[2-(4-Nitro-pyrazol-1-yl)-ethyl]-morpholine

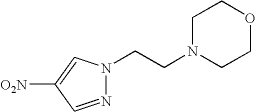

N-(2-chloroethyl)morpholine HCl salt (2.1 g, 11.06 mmol) was added portionwise to a stirred mixture of 4-nitro-1H-pyrazole (1.0 g, 8.85 mmol) and KOH (1.24 g, 22.12 mmol) in EtOH (20 ml). The mixture was heated under reflux for 2 h and allowed to cool to rt. After dilution with EtOAc and water the organic phase was washed with brine, dried and concentrated. The residue was purified by flash column chromatography on silica gel (100 g) eluting with 50:1 DCM-MeOH to provide an orange oil (987 mg, 49%). ¹H NMR (400 MHz, DMSO-d₆) δ ppm 2.30-2.49 (m, 4H), 2.73 (t, J=6.18 Hz, 2H), 3.42-3.61 (m, 4H), 4.30 (t, J=6.18 Hz, 2H), 8.26 (s, 1H), 8.88 (s, 1H); m/z (ES+APCI)⁺: 227 [M+H]⁺.

### Intermediate 51

### 1-(2-Morpholin-4-yl-ethyl)-1H-pyrazol-4-ylamine

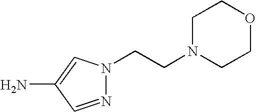

Prepared analogously to Intermediate 13 using Intermediate 50 to provide a red coloured oil (775 mg, 91%). ¹H NMR (400 MHz, DMSO-d₆) δ ppm 2.30-2.44 (m, 4H), 2.61 (t, J=6.64 Hz, 2H), 3.47-3.59 (m, 4H), 3.84 (br. s, 2H), 4.02 (t, J=6.64 Hz, 2H), 6.88 (s, 1H), 7.05 (s, 1H); m/z (ES+APCI)⁺: 197 [M+H]⁺.

### Intermediate 52

### 1-[2-(4-Nitro-pyrazol-1-yl)-ethyl]-piperidine

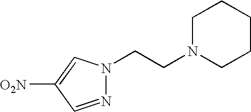

Prepared analogously to Intermediate 50 using N-(2-chloroethyl)piperidine HCl salt and 4-nitro-1H-pyrazole to provide the product as an orange coloured solid (1.94 g, 98%). ¹H NMR (400 MHz, DMSO-d₆) δ ppm 1.27-1.55 (m, 6H), 2.25-2.48 (m, 4H), 2.65 (t, J=6.41 Hz, 2H), 4.26 (t, J=6.41 Hz, 2H), 8.24 (s, 1H), 8.84 (s, 1H); m/z (ES+APCI)⁺: 225 [M+H]⁺.

### Intermediate 53

### 1-(2-Piperidin-1-yl-ethyl)-1H-pyrazol-4-ylamine

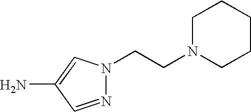

Prepared analogously to Intermediate 13 using Intermediate 52 to afford a red coloured solid (1.64 g, 98%). ¹H NMR (400 MHz, DMSO-d₆) δ ppm 1.19-1.41 (m, 2H), 1.41-1.57 (m, 4H), 2.33 (br. s, 4H), 2.45-2.64 (m, 2H), 3.84 (br. s, 2H), 3.90-4.08 (m, 2H), 6.86 (s, 1H), 7.03 (s, 1H); m/z (ES+APCI)⁺: 195 [M+H]⁺.

### Intermediate 54

### (1-Cyclobutanecarbonyl-azetidin-3-ylmethyl)-carbamic acid tert-butyl ester

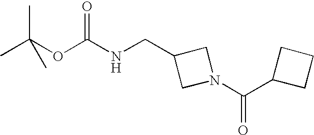

To a solution of azetidin-3-ylmethyl-carbamic acid tert-butyl ester (279 mg, 1.5 mmol) in DCM was added DIPEA (550 μl, 3.15 mmol) followed by cyclobutanecarbonyl chloride (170 μl, 1.5 mmol) dropwise at 0° C. The reaction was stirred for 6 h at RT, quenched with NaHCO₃(aq) and washed with brine. The organic fractions were isolated by phase separation cartridge and concentrated under vacuum to give the desired product. (187 mg, 47%). ¹H NMR (400 MHz, DMSO-d₆) δ ppm 1.37 (s, 9H), 1.66-1.82 (m, 1H), 1.82-1.94 (m, 1H), 1.94-2.05 (m, 1H), 2.05-2.16 (m, 3H), 2.54-2.65 (m, 1H), 2.97-3.07 (m, 1H), 3.05-3.13 (m, 1H), 3.33 (s, 1H), 3.50 (dd, J=9.62, 5.50 Hz, 1H), 3.65 (dd, J=8.70, 5.50 Hz, 1H), 3.75-3.84 (m, 1H), 4.00 (t, J=8.47 Hz, 1H), 7.04 (t, J=5.72 Hz, 1H).

### Intermediate 55

### (3-Aminomethyl-azetidin-1-yl)-cyclobutyl-methanone

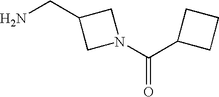

To a solution of Intermediate 54 (187 mg, 0.7 mmol) in MeOH was added 4M HCl in dioxane (1.75 ml, 7 mmol) and stirred overnight at RT. The product was purified on SCX cartridge to give the desired product (66 mg, 57%). ¹H NMR (400 MHz, DMSO-d₆) δ ppm 1.67-1.78 (m, 1H), 1.81-1.94 (m, 1H), 1.94-2.04 (m, 2H), 2.04-2.14 (m, 2H), 2.63-2.69 (m, 1H), 2.96-3.13 (m, 2H), 3.16 (s, 1H), 3.48 (dd, J=9.39, 5.27 Hz, 1H), 3.67 (dd, J8.47, 5.27 Hz, 1H), 3.78 (t, J=8.93 Hz, 1H), 3.99 (t, J=8.24 Hz, 1H); m/z (ES+APCI)⁺: 169 [M+H]⁺.

### Intermediate 56

### {3-[(2-Chloro-5-cyclopropyl-pyrimidin-4-ylamino)-methyl]-azetidin-1-yl}-cyclobutyl-methanone

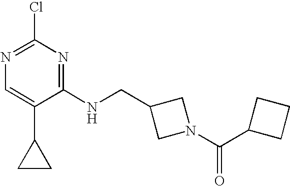

To a solution of Intermediate 1 (75 mg, 0.4 mmol) and DIPEA (155 mg, 1.2 mmol) in IPA was added Intermediate 55 (66 mg, 0.4 mmol) portionwise at 0° C. and stirred overnight at RT. The reaction was concentrated, taken up in DCM, washed with water and brine and purified by Biotage SP4 (ethyl acetate/petroleum ether gradient) to give the desired product as a colourless oil (37 mg, 29%). ¹H NMR (400 MHz, CHLOROFORM-d) δ ppm 0.47-0.59 (m, 2H), 0.88-1.02 (m, 2H), 1.35-1.46 (m, 1H), 1.79-1.92 (m, 1H), 1.92-2.00 (m, 1H), 1.99-2.11 (m, 1H), 2.09-2.23 (m, 2H), 2.24-2.42 (m, 3H), 3.09-3.32 (m, 2H), 3.42-3.54 (m, 1H), 3.54-3.73 (m, 1H), 3.78-3.90 (m, 1H), 4.64-4.83 (m, 1H), 5.43 (d, 6.41 Hz, 1H), 7.84 (d, J=6.41 Hz, 1H).

### Intermediate 57

### 4-{4-[3-(Cyclobutanecarbonyl-amino)-propylamino]-5-cyclopropyl-pyrimidin-2-ylamino}-benzoic acid

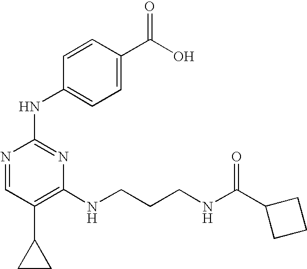

A solution of Intermediate 8 (200 mg, 0.65 mmol), 4-aminobenzoic acid (267 mg, 1.95 mmol) and glacial AcOH (12 mg, 0.2 mmol) in n-butanol was heated at 150° C. in the microwave for 35 min. The resulting precipitate was filtered and dried in a drying pistol to give the desired product (221 mg, 83%). ¹H NMR (400 MHz, DMSO-d₆) δ ppm 0.52-0.64 (m, 2H), 0.87-0.98 (m, 2H), 1.49-1.61 (m, 1H), 1.67-1.78 (m, 4H), 1.78-1.88 (m, 1H), 1.89-2.00 (m, 2H), 2.02-2.14 (m, 2H), 2.94 (t, J=8.01 Hz, 1H), 3.08-3.18 (m, 2H), 3.51 (q, J=6.72 Hz, 2H), 7.67 (s, 1H), 7.73 (d, J=8.70 Hz, 2H), 7.76 (t, J=5.72 Hz, 1H), 7.98 (d, J=9.16 Hz, 2H), 8.62 (br. s, 1H), 10.56 (br. s, 1 H); m/z (ES+APCI)⁺: 410 [M+H]⁺.

### Intermediate 58

### 2-Methyl-4-nitro-benzoic acid methyl ester

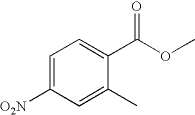

A solution of 2-methyl-4-nitro-benzoic acid (10 g, 55 mmol), MeI (3.8 ml, 61 mmol) and K₂CO₃ (11.4 g, 83 mmol) in DMF was stirred for 3 h at RT after which the reaction was poured onto water and extracted in EtOAc. The organic fractions were washed with water and brine, dried over Na₂SO₄ and concentrated to give the desired product as an orange solid (9.57 g, 89%). ¹H NMR (400 MHz, DMSO-d₆) δ ppm 2.60 (s, 3H), 3.88 (s, 3H), 8.00 (d, J=8.70 Hz, 1H), 8.10-8.16 (m, 1H), 8.21 (d, J=2.29 Hz, 1H).

### Intermediate 59

### 2-Bromomethyl-4-nitro-benzoic acid methyl ester

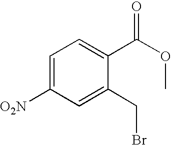

A solution of Intermediate 58 (9.57 g, 49 mmol), NBS (9.6 g, 54 mmol), benzoyl peroxide (121 mg, 0.5 mmol) in carbon tetrachloride was heated at 85° C. for 18 h. The reaction was concentrated under vacuum, analysed by TLC and ¹H NMR and found to have some remaining starting material.

The residues were added to CCl₄ (40 ml) with NBS (4.4 g) and benzoyl peroxide (60 mg) then stirred at 85° C. for 3 h. The reaction was concentrated, taken up in DCM then washed with aqueous NaHCO₃ and brine. The organic phase was dried over Na₂SO₄ and concentrated under vacuum. The residues were purified by flash chromatography using a Biotage SP4. The desired product was isolated as an orange solid (6.58 g, 49%). ¹H NMR (400 MHz, DMSO-d₆) δ ppm 3.93 (s, 3H), 5.10 (s, 2H), 8.08 (d, J=8.24 Hz, 1H), 8.25-8.30 (m, 1H), 8.51 (d, J=2.29 Hz, 1H).

### Intermediate 60

### 2-Methyl-5-nitro-2,3-dihydro-isoindol-1-one

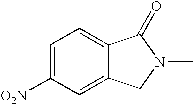

A solution of Intermediate 59 (1 g, 3.7 mmol), methylamine hydrochloride (124 mg, 4 mmol) and Et₃N (404 mg, 4 mmol) were refluxed in MeOH for 24 h under N₂. During this time an additional 0.5 eq of methylamine hydrochloride was added, twice, to compensate for the volatility of methylamine. The mixture was diluted with EtOAc, washed sequentially with 1M HCl and brine, and the organic phase was dried over Na₂SO₄ and concentrated under vacuum. The residues were purified by silica gel chromatography, eluting first with 20% EtOAc in petroleum ether and then with 30% MeOH in DCM. Further purification by preparative LCMS gave the title compound (62 mg, 9%). ¹H NMR (400 MHz, DMSO-d₆) δ ppm 3.12 (s, 3H), 4.60 (s, 2H), 7.90 (d, J=8.24 Hz, 1 H), 8.26-8.36 (m, 1H), 8.51 (s, 1H); m/z (ES+APCI)⁺: 193 [M+H]⁺.

### Intermediate 61

### 2-(2-Hydroxy-ethyl)-5-nitro-2,3-dihydro-isoindol-1-one

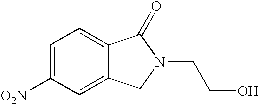

A solution of Intermediate 59 (1 g, 3.7 mmol), 2-hydroxy ethylamine (242 μl, 4 mmol) and Et₃N (404 mg, 4 mmol) were refluxed in MeOH for 24 h under N₂. The mixture was diluted in DCM, washed with 1M HCl and brine. The organic fractions were dried over Na₂SO₄ and concentrated under vacuum. The residues were purified by preparative LCMS to give the title compound (114 mg, 14%). ¹H NMR (400 MHz, DMSO-d₆) δ ppm 3.55-3.69 (m, 4H), 4.68 (s, 2H), 4.86-4.95 (m, 1H), 7.91 (d, J=8.24 Hz, 1H), 8.33 (dd, J=8.47, 2.06 Hz, 1H), 8.52 (s, 1H); m/z (ES+APCI)⁺: 223 [M+H]⁺.

### Intermediate 62

### 5-Amino-2-methyl-2,3-dihydro-isoindol-1-one

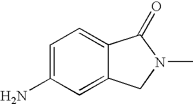

A mixture of Intermediate 60 (68 mg), Fe (180 mg), and HCl (53 μl), in EtOH (3 ml), was refluxed at 95° C. under N₂ for 3 h. The reaction was basified with a few drops of 2M NH₃ in MeOH then concentrated under vacuum. The residues were taken up in 20% MeOH in DCM, filtered twice through a plug of silica gel, and the filtrate was concentrated under vacuum to give the desired product (58 mg, 99%). ¹H NMR (400 MHz, DMSO-d₆) δ ppm 2.96 (s, 3H), 4.24 (s, 2H), 5.75 (br. s, 2H), 6.54-6.64 (m, 2H), 7.27 (d, J=7.78 Hz, 1H); m/z (ES+APCI)⁺: 163 [M+H]⁺.

### Intermediate 63

### 5-Amino-2-(2-hydroxy-ethyl)-2,3-dihydro-isoindol-1-one

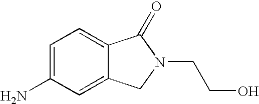

Prepared analogously to Intermediate 62 from Intermediate 61 (114 mg, 0.5 mmol) to give the title compound (87 mg, 89%). ¹H NMR (400 MHz, DMSO-d₆) δ ppm 3.49-3.61 (m, 2H), 4.32 (s, 2H), 4.78 (t, J=5.27 Hz, 1H), 5.69 (s, 2H), 6.51-6.64 (m, 2H), 7.16 (br. s, 2H), 7.28 (d, J=8.24 Hz, 1H): m/z (ES+APCI)⁺: 193 [M+H]⁺.

### Intermediate 64

### 6-Oxazol-5-yl-pyridin-3-ylamine

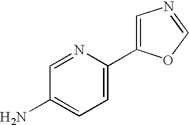

### Step 1

### 5-Nitro-2-oxazol-5-yl-pyridine

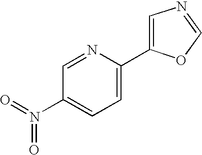

To a solution of 5-nitro-pyridine-2-carbaldehyde (650 mg, 4.3 mmol) in MeOH (30 ml) was added tosyl isocyanide (840 mg, 4.3 mmol) followed by K₂CO₃ and heated with stirring at 70° C. for 2.5 h. The mixture was poured onto ice water and extracted with EtOAc then DCM. The organic components were washed with aqueous HCl (1M) and brine before drying over MgSO₄. The combined organic fractions were concentrated under vacuum to give the desired product as a yellow solid (548 mg, 67%). ¹H NMR (400 MHz, DMSO-d₆) δ ppm 8.04 (d, J=8.70 Hz, 1H), 8.10 (s, 1H), 8.68-8.74 (m, 2H), 9.42 (d, J=2.75 Hz, 1H).

### Step 2

### 6-Oxazol-5-yl-pyridin-3-ylamine

To a solution of 5-nitro-2-oxazol-5-yl-pyridine (545 mg, 2.8 mmol) in EtOH (20 ml) was added Pd/C (30 mg, 0.28 mmol). The flask was evacuated and filled with N₂ three times then evacuated and filled with H₂ three times before stirring overnight at RT under H₂. The mixture was filtered through Celite under N₂, concentrated under reduced pressure and separated on Biotage SP4 (DCM/MeOH gradient) to give the desired product as a yellow solid (106 mg, 24%). ¹H NMR (400 MHz, DMSO-d₆) δ ppm 7.27 (dd, J=8.70, 2.75 Hz, 1H), 7.49-7.57 (m, 1H), 7.59 (d, J=8.24 Hz, 1H), 8.17 (d, J=2.75 Hz, 1H), 8.39-8.45 (m, 1H), 8.70 (d, J=1.83 Hz, 1H), 8.83 (s, 1H); m/z (ES+APCI)⁺: 162 [M+H]⁺.

### Intermediate 65

### Imidazo[1,2-a]pyridin-6-ylamine

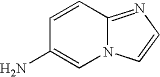

To a solution of 6-nitro-imidazo[1,2-a]pyridine (500 mg, 3 mmol) in EtOH (20 ml) was added Pd/C (30 mg, 0.3 mmol). The flask was evacuated and filled with N₂ three times then evacuated and filled with H₂ three times before stirring overnight at RT under H₂. The mixture was filtered through celite under N₂, concentrated under reduced pressure and run through a short silica column. The eluant was concentrated under vacuum to give the desired product as a dark green solid (295 mg, 74%). ¹H NMR (400 MHz, DMSO-d₆) δ ppm 4.87 (br. s, 2H), 6.78-6.83 (m, 1H), 7.27-7.36 (m, 2H), 7.67 (dd, 1H), 7.69 (s, 1H); m/z (ES+APCI)⁺: 134 [M+H]⁺.

### Intermediate 66

### N²-(Tetrahydro-pyran-4-ylmethyl)-pyridine-2,5-diamine

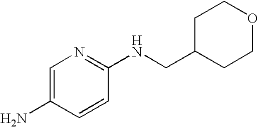

### Step 1

### (5-Nitro-pyridin-2-yl)-(tetrahydro-pyran-4-ylmethyl)-amine

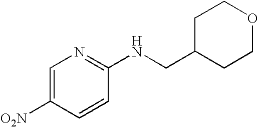

2-Chloro-5-nitro-pyridine (200 mg, 1.3 mmol), 4-aminomethyltetrahydropyran (145 mg, 1.3 mmol) and DIPEA (220 μl, 1.3 mmol) were combined in MeCN (5 ml) and stirred overnight at RT. The resulting precipitate was removed and the filtrate concentrated under vacuum. The filtrate residues were purified on Biotage SP4 (ethyl acetate/petroleum ether gradient) to give the product as a bright green solid (245 mg, 82%). ¹H NMR (400 MHz, DMSO-d₆) δ ppm 1.10-1.32 (m, 2H), 1.60 (d, J=13.28 Hz, 2H), 1.80 (m, 1H), 3.20-3.33 (m, 4H), 3.84 (dd, J=11.22, 2.52 Hz, 2H), 6.57 (d, J=9.16 Hz, 1H), 8.08 (d, J=8.24 Hz, 1H), 8.21 (t, J=5.04 Hz, 1H), 8.90 (d, 1H); m/z (ES+APCI)⁺: 238 [M+H]⁺.

### Step 2

### N²-(Tetrahydro-pyran-4-ylmethyl)-pyridine-2,5-diamine

To a solution of (5-Nitro-pyridin-2-yl)-(tetrahydro-pyran-4-ylmethyl)-amine (240 mg, 1 mmol) in EtOH (20 ml) was added Pd/C (15 mg, 0.15 mmol). The flask was evacuated and filled with N₂ three times then evacuated and filled with H₂ three times before stirring overnight at RT under H₂. The mixture was filtered through Celite under N₂, concentrated under reduced pressure to give the desired product as a red oil (104 mg, 50%). ¹H NMR (400 MHz, DMSO-d₆) δ ppm 1.05-1.26 (m, 2H), 1.61 (d, J=12.82 Hz, 2H), 1.71 (m, 1H), 2.98 (t, J=6.41 Hz, 2H), 3.17-3.30 (m, 2H), 3.83 (dd, J=11.22, 2.52 Hz, 2H), 4.24 (s, 2H), 5.63 (t, J=5.72 Hz, 1H), 6.29 (d, J=8.70 Hz, 1H), 6.79 (dd, J=8.70, 2.75 Hz, 1H), 7.42 (d, 1H); m/z (ES+APCI)⁺: 208 [M+H]⁺.

### Intermediate 67

### 6-(1-Cyclopropyl-piperidin-4-yloxy)-pyridin-3-ylamine

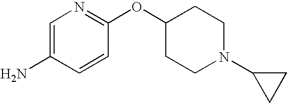

### Step 1

### 1-Cyclopropyl-piperidin-4-ol

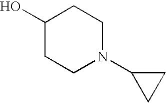

1-Cyclopropyl-piperidin-4-one (500 mg, 3.6 mmol) and NaBH₄ (73 mg, 1.9 mmol) were combined in EtOH and Stirred at RT for 3 h. The reaction was quenched with H₂O and extracted with DCM and 2M NaOH(aq). The organic fractions were dried over MgSO₄ and concentrated under vacuum to give the desired product as a colourless oil (253 mg, 50%). ¹H NMR (400 MHz, DMSO-d₆) δ ppm 0.17-0.29 (m, 2H), 0.32-0.43 (m, 2H), 1.19-1.37 (m, 2H), 1.47-1.58 (m, 1H), 1.59-1.72 (m, 2H), 2.09-2.29 (m, 2H), 2.70-2.86 (m, 2H), 3.43 (dt, J=8.93, 4.69 Hz, 1H), 4.54 (d, 1H); m/z (ES+APCI)⁺: 142 [M+H]⁺.

### Step 2

### 2-(1-Cyclopropyl-piperidin-4-yloxy)-5-nitro-pyridine

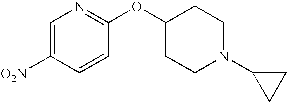

To a solution of 1-cyclopropyl-piperidin-4-ol and 5-nitro-2-chloro pyridine in DMF was added NaH portionwise with ice cooling. The reaction was stirred at 0° C. for 1 h then stirred overnight at RT. The mixture was added to ice water and extracted with EtOAc. The organic extracts were washed with water and brine then dried over MgSO₄ and concentrated under vacuum. The residues were purified by Biotage SP4 (ethyl acetate/petroleum ether gradient) to give the desired product as a yellow solid (472 mg, 19%). ¹H NMR (400 MHz, DMSO-d₆) δ ppm 0.23-0.36 (m, 2H), 0.36-0.47 (m, 2H), 1.54-1.73 (m, 3H), 1.90-2.04 (m, 2H), 2.42 (t, J=9.39 Hz, 2H), 2.78-2.90 (m, 2H), 5.14 (dt, J=8.47, 4.46 Hz, 1H), 7.00 (d, J=9.16 Hz, 1H), 8.45 (dd, J=9.16, 3.21 Hz, 1H), 9.08 (d, 1H).

### Step 3

### 6-(1-Cyclopropyl-piperidin-4-yloxy)-pyridin-3-ylamine

To a solution of 2-(1-cyclopropyl-piperidin-4-yloxy)-5-nitro-pyridine (240 mg, 1 mmol) in EtOH (20 ml) was added Pd/C (15 mg, 0.15 mmol). The flask was evacuated and filled with N₂ three times then evacuated and filled with H₂ three times before stirring overnight at RT under H₂. The mixture was filtered through celite under N₂, concentrated under reduced pressure to give the desired product as a brown oil (104 mg, 50%). ¹H NMR (400 MHz, DMSO-d₆) δ ppm 0.23-0.31 (m, 2H), 0.36-0.45 (m, 2H), 1.43-1.55 (m, 2H), 1.59 (tt, J=6.75, 3.55 Hz, 1H), 1.80-1.92 (m, 2H), 2.28-2.41 (m, 2H), 2.75-2.87 (m, 2H), 4.67-4.81 (m, 3H), 6.49 (d, J=8.70 Hz, 1H), 6.97 (dd, J=8.70, 2.75 Hz, 1H), 7.47 (d, 1H); m/z (ES+APCI)⁺: 233 [M+H]⁺.

### Intermediate 68

### [1, 2, 4]Triazolo[1,5-a]pyridin-6-ylamine

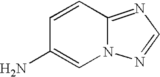

### Step 1

### Benzhydrylidene-[1,2,4]triazolo[1,5-a]pyridin-6-yl-amine

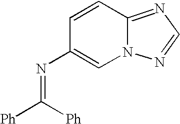

6-Bromo-[1,2,4]triazolo[1,5-a]pyridine (300 mg, 1.5 mmol), bezophenone imine (326 mg, 1.8 mmol), Pd₂(dba)₃ (7 mg, 0.008 mmol), BINAP (14 mg, 0.02 mmol) and sodium tert-butoxide (202 mg, 2.1 mmol) were combined in toluene and heated at 80° C. overnight. The mixture was concentrated under reduced pressure and purified by Biotage SP4 (ethyl acetate/petroleum ether gradient) to give the desired product as a yellow crystalline solid (310 mg, 69%). ¹H NMR (400 MHz, DMSO-d6) δ ppm 7.21 (dd, J=9.39, 2.06 Hz, 1H), 7.24-7.29 (m, 2H), 7.32-7.40 (m, 3H), 7.47-7.54 (m, 2H), 7.55-7.62 (m, 1H), 7.66 (d, J=8.70 Hz, 1H), 7.68-7.74 (m, 2H), 8.35 (s, 1H), 8.41 (d, J=1.83 Hz, 1H); m/z (ES+APCI)⁺: 299 [M+H]⁺.

### Step 2

### [1,2,4]Triazolo[1,5-a]pyridin-6-ylamine

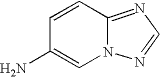

Benzhydrylidene-[1,2,4]triazolo[1,5-a]pyridin-6-yl-amine (260 mg, 0.87 mmol), hydroxylamine hydrochloride (110 mg, 1.6 mmol) and NaOAc (172 mg, 2.1 mmol) were combined in MeOH and stirred at RT overnight. The mixture was concentrated, taken up in DCM, washed with 0.1M NaOH(aq) and dried by phase separator cartridge. The eluant was concentrated under vacuum, taken up in MeOH and run through an SCX cartridge. The filtrate was discarded and the product eluted with 2M NH₃ in methanol, evaporating to give a blue/green crystalline solid (47 mg, 40%). ¹H NMR (400 MHz, DMSO-d₆) δ ppm 5.26 (br. s, 2H), 7.11-7.26 (m, 1H), 7.56 (d, J=10.07 Hz, 1H), 8.02 (d, J=2.75 Hz, 1H), 8.17 (s, 1H); m/z (ES+APCI)⁺: 135 [M+H]⁺.

### Intermediate 69

### 3-(1-Methyl-1-morpholin-4-yl-ethyl)-phenylamine

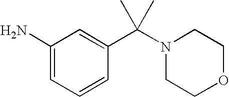

### Step 1

### 4-[1-(3-Bromo-phenyl)-1-methyl-ethyl]-morpholine

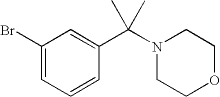

A mixture of 1-(3-bromo-phenyl)-1-methyl-ethylamine (742 mg, 3.47 mmol), 1-bromo-2-(2-bromo-ethoxy)-ethane (522 μl, 4.16 mmol) and potassium carbonate (957 mg, 6.93 mmol) in ethanol (15 ml) was stirred and heated under reflux for 40 hours. The mixture was allowed to cool to rt and diluted with EtOAc and water. The organic phase was washed with brine, dried and concentrated. The crude product was purified by flash column chromatography on silica gel (40 g) eluting with 7:1 petroleum ether:EtOAc to give the product as a colourless oil (585 mg, 59%). ¹H NMR (400 MHz, DMSO-d₆) δ ppm 1.25 (s, 6H), 2.30-2.41 (m, 4H), 3.40-3.62 (m, 4H), 7.26-7.31 (m, 1H), 7.38-7.42 (m, 1H), 7.50 (d, J=7.79 Hz, 1H), 7.58-7.78 (m, 1H).

### Step 2

### Benzhydrylidene-[3-(1-methyl-1-morpholin-4-yl-ethyl)-phenyl]-amine

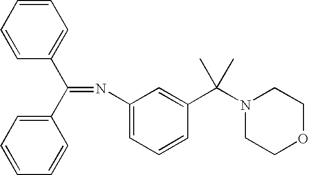

A mixture of 4-[1-(3-bromo-phenyl)-1-methyl-ethyl]-morpholine (585 mg, 2.06 mmol), benzophenone imine (447 mg, 2.47 mmol), sodium tert-butoxide (277 mg, 2.88 mmol), BINAP (49 mg, 0.078 mmol) and Pd₂(dba)₃ (25 mg, 0.027 mmol) in toluene (15 ml) was placed in a round bottomed flask. The mixture was degassed, placed under an atmosphere of nitrogen and stirred and heated at 80° C. overnight. On cooling to rt the mixture was diluted with EtOAc and water. The organic phase was washed with brine, dried and concentrated. The crude product was purified by flash column chromatography on silica gel (50 g) eluting with 3:1 petroleum ether:EtOAc to give the product as a yellow/green coloured oil (730 mg, 92%). ¹H NMR (400 MHz, DMSO-d₆) δ ppm 1.14 (s, 6H), 2.09-2.17 (m, 4H), 3.39-3.48 (m, 4H), 6.63-6.67 (m, 2H), 6.98 (d, J=7.79 Hz, 1 H), 7.06-7.14 (m, 3H), 7.25-7.29 (m, 3H), 7.39-7.57 (m, 3H), 7.66 (d, J=6.87 Hz, 2H).

### Step 3

### 3-(1-Methyl-1-morpholin-4-yl-ethyl)-phenylamine

A mixture of benzhydrylidene-[3-(1-methyl-1-morpholin-4-yl-ethyl)-phenyl]-amine (697 mg, 1.82 mmol), sodium acetate (357 mg, 4.36 mmol) and hydroxylamine hydrochloride (225 mg, 3.27 mmol) in methanol (20 ml) was stirred at rt for 2 hours. The mixture was concentrated to a small volume and diluted with DCM and 0.1M NaOH (aq). The organic phase was dried and concentrated. The crude residue was dissolved in methanol and passed through a SCX cartridge. The product was eluted with 2M ammonia in methanol to provide a yellow solid after concentration of the eluent (314 mg, 79%). ¹H NMR (400 MHz, DMSO-d₆) δ ppm 1.24 (s, 6H), 2.31-2.40 (m, 4H), 3.49-3.58 (m, 4H), 4.95 (s, 2H), 6.38 (d, J=9.16 Hz, 1H), 6.63 (d, J=7.78 Hz, 1H), 6.74 (s, 1H), 6.93 (t, J=7.79 Hz, 1H).

### Intermediate 70

### 1-(Tetrahydro-pyran-4-yl)-1H-pyrazol-4-ylamine

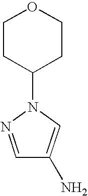

### Step 1

### 4-Nitro-1-(tetrahydro-pyran-4-yl)-1H-pyrazole

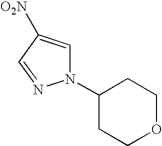

A solution of di-tert-butyl azodicarboxylate (2.6 g, 11.5 mmol) in THF (5 ml) was added to a stirred solution of tetrahydro-pyran-4-ol (903 mg, 8.85 mmol), 4-nitro-1H-pyrazole (1.0 g, 8.85 mmol) and triphenylphosphine (2.8 g, 10.62 mmol) in THF (35 ml) at rt. The mixture was stirred at rt overnight and subsequently concentrated to dryness. The crude product was purified by flash column chromatography on silica gel (250 g) eluting with 4:1 to 1:1 petroleum ether:EtOAc to give the product as a white solid (1.59 g, 91%). ¹H NMR (400 MHz, DMSO-d₆) δ ppm 1.90-2.04 (m, 4H), 3.44 (td, J=11.33, 3.43 Hz, 2H), 3.92-3.99 (m, 2H), 4.46-4.55 (m, 1H), 8.29 (s, 1H), 8.96 (s, 1H).

### Step 2

### 1-(Tetrahydro-pyran-4-yl)-1H-pyrazol-4-ylamine

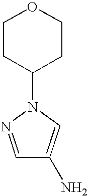

A mixture of 4-nitro-1-(tetrahydro-pyran-4-yl)-1H-pyrazole (1.59 g, 8.07 mmol) and 10% palladium on charcoal in ethanol (30 ml) was stirred at rt overnight under a balloon of hydrogen. The mixture was filtered through Celite and the filtrate was concentrated to give the product as a purple coloured solid (1.33 g, 98%). ¹H NMR (400 MHz, DMSO-d₆) δ ppm 1.76-1.94 (m, 4H), 3.29-3.48 (m, 2H), 3.80 (br. s, 2H), 3.84-4.00 (m, 2H), 4.16 (tt, J=10.42, 5.38 Hz, 1H), 6.90 (s, 1H), 7.06 (s, 1H); m/z (ES+APCI)⁺: 168 [M+H]⁺.

### Intermediate 71 and Intermediate 72

### 1-Cyclopropylmethyl-6-nitro-1H-indazole (Intermediate 71)

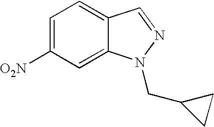

### 2-Cyclopropylmethyl-6-nitro-2H-indazole (Intermediate 72)

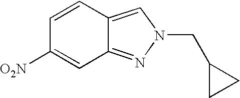

A mixture of 6-nitroindazole (2.0 g, 12.3 mmol), cyclopropylmethyl bromide (1.19 ml, 12.3 mmol) and potassium carbonate (3.39 g, 24.5 mmol) in DMF were stirred at 35° C. overnight. The mixture was cooled to rt and diluted with EtOAc and water. The organic phase was washed with water (×3), washed with brine (×1), dried and concentrated. The crude product was purified by flash column chromatography on silica gel (125 g) eluting with 4:1 to 2:1 petroleum ether:EtOAc to provide to isomeric products. The first eluting product was 1-cyclopropylmethyl-6-nitro-1H-indazole (Intermediate 71) obtained as a yellow solid (1.14 g, 43%). ¹H NMR (400 MHz, CHLOROFORM-d) δ ppm 0.42-0.54 (m, 2H), 0.59-0.72 (m, 2H), 1.33-1.44 (m, 1H), 4.37 (d, J=6.87 Hz, 2H), 7.86 (d, J=8.24 Hz, 1H), 7.99-8.06 (m, 1H), 8.14 (s, 1H), 8.43 (s, 1H).

The second eluting product was 2-cyclopropylmethyl-6-nitro-2H-indazole (Intermediate 72) obtained as an orange solid (1.25 g, 47%). ¹H NMR (400 MHz, CHLOROFORM-d) δ ppm 0.45-0.61 (m, 2H), 0.63-0.82 (m, 2H), 1.43-1.53 (m, 1H), 4.37 (d, J=7.33 Hz, 2H), 7.79 (d, J=9.16 Hz, 1H), 7.92 (dd, J=9.16, 1.83 Hz, 1H), 8.19 (s, 1H), 8.72 (s, 1H); m/z (ES+APCI)⁺: 218 [M+H]⁺.

### Intermediate 73

### 1-Cyclopropylmethyl-1H-indazol-6-ylamine

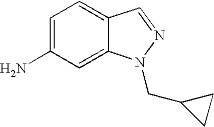

Prepared analogously to Intermediate 70 (Step 2) using 1-cyclopropylmethyl-6-nitro-1H-indazole (Intermediate 71). The product was obtained as a red oil (1.0 g, 102%). ¹H NMR (400 MHz, DMSO-d₆) δ ppm 0.31-0.40 (m, 2H), 0.40-0.50 (m, 2H), 1.15-1.25 (m, 1H), 4.06 (d, J=6.87 Hz, 2H), 5.30 (s, 2H), 6.46-6.52 (m, 2H), 7.35 (d, J=7.78 Hz, 1H), 7.69 (s, 1H); m/z (ES+APCI)⁺: 188 [M+H]⁺.

### Intermediate 74

### 2-Cyclopropylmethyl-2H-indazol-6-ylamine

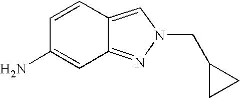

Prepared analogously to Intermediate 70 (Step 2) Intermediate 72. The product was obtained as a brown oil (850 mg, 79%). ¹H NMR (400 MHz, DMSO-d₆) δ ppm 0.31-0.45 (m, 2H), 0.45-0.59 (m, 2H), 1.26-1.37 (m, 1H), 4.09 (d, J=7.33 Hz, 2H), 5.02 (s, 2H), 6.44-6.53 (m, 2H), 7.35 (d, J=8.70 Hz, 1H), 8.06 (s, 1H); m/z (ES+APCI)⁺: 188 [M+H]⁺.

### Intermediate 75

### 1-Cyclopropyl-1H-indazol-6-ylamine

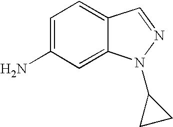

### Step 1

### 1-Cyclopropyl-6-nitro-1H-indazole

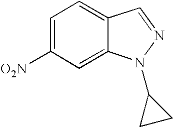

A suspension of copper (II) acetate (1.12 g, 6.13 mmol) and [2,2′]bipyridinyl (957 mg, 6.13 mmol) in hot DCE (40 ml) was added to a suspension of cyclopropylboronic acid (1.06 g, 12.27 mmol), 6-nitroindazole (1.0 g, 6.13 mmol) and sodium carbonate (1.30 g, 12.27 mmol) in DCE (20 ml). The mixture was heated at 70° C. for 3 hours. On cooling to rt saturated ammonium chloride solution (aq) was added followed by the addition of DCM and water. The organic phase was separated and the aqueous phase re-extracted with DCM. The combined organic phases were dried and concentrated. The crude product was purified by flash column chromatography on silica gel (75 g) eluting with 3:1 petroleum ether:EtOAc to give the product as a yellow solid (626 mg, 50%). ¹H NMR (400 MHz, DMSO-d₆) δ ppm 1.12-1.27 (m, 4H), 3.95-4.02 (m, 1H), 7.95-8.04 (m, 2H), 8.27 (s, 1H), 8.63 (s, 1H).

### Step 2

### 1-Cyclopropyl-1H-indazol-6-ylamine

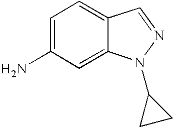

Prepared analogously to Intermediate 70 (Step 2) using 1-cyclopropyl-6-nitro-1H-indazole to provide the product as a brown solid (542 mg, 102%). ¹H NMR (400 MHz, DMSO-d₆) δ ppm 0.95-1.08 (m, 4H), 3.45 (tt, J=6.98, 3.55 Hz, 1H), 5.39 (s, 2H), 6.49 (dd, J=8.24, 1.83 Hz, 1H), 6.59 (s, 1H), 7.33 (d, J=9.16 Hz, 1H), 7.64 (s, 1H); m/z (ES+APCI)⁺: 174 [M+H]⁺.

### Intermediate 76

### 6-Cyclopropyl-pyridin-3-ylamine

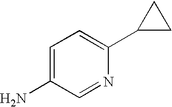

### Step 1

### 2-Cyclopropyl-5-nitro-pyridine

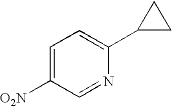

A mixture of 2-chloro-5-nitopyridine (1.0 g, 6.29 mmol), cyclopropylboronic acid (594 mg, 8.36 mmol), palladium (II) acetate (70 mg, 0.314 mmol), tricyclohexylphosphine (176 mg, 0.629 mmol) and potassium phosphate tribasic (4.93 g, 23.3 mmol) in water (0.3 ml) and toluene (6 ml) was placed in a RBF. The mixture was degassed, placed under an atmosphere of nitrogen and subsequently heated at 100° C. overnight. On cooling to rt the mixture was diluted with EtOAc and water and was then filtered through Celite. The organic phase was washed with brine, dried and concentrated. The crude product was purified by flash column chromatography on silica gel (25 g) in 10:1 petroleum ether:EtOAc to give the product as an off-white solid (305 mg, 30%). ¹H NMR (400 MHz, DMSO-d₆) δ ppm 1.02-1.18 (m, 4H), 2.30-2.38 (m, 1H), 7.62 (d, J=8.70 Hz, 1H), 8.44 (dd, J=8.70, 2.75 Hz, 1H), 9.20 (d, J=2.75 Hz, 1H).

### Step 2

### 6-Cyclopropyl-pyridin-3-ylamine

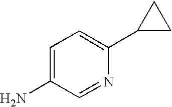

A mixture of 2-cyclopropyl-5-nitro-pyridine (300 mg, 2.24 mmol) and 10% palladium on charcoal (32 mg) in ethanol (15 ml) was stirred under a balloon of hydrogen at rt overnight. The mixture was then filtered through Celite and the filtrate was concentrated. The residue was then treated to identical hydrogenation conditions for a second time and the crude product isolated as previously. The crude material was purified by preparative HPLC (high pH buffer) to provide the product as an orange oil (30 mg, 12%). ¹H NMR (400 MHz, CHLOROFORM-d) δ ppm 0.85-1.06 (m, 4H), 1.98-2.09 (m, 1H), 3.78-4.07 (m, 2H), 6.93 (d, J=7.79 Hz, 1H), 7.01 (dd, J=8.24, 2.75 Hz, 1H), 8.06 (d, J=3.21 Hz, 1H); m/z (ES+APCI)⁺: 135 [M+H]⁺.

### Intermediate 77

### 6-(4-Cyclopropyl-piperazin-1-yl)-pyridin-3-ylamine

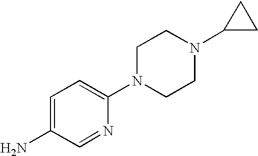

### Step 1

### 1-Cyclopropyl-4-(5-nitro-pyridin-2-yl)-piperazine

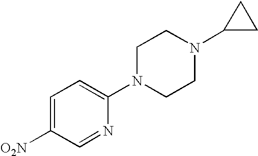

A solution of DIPEA (690 μl, 3.97 mmol) and 1-cyclopropylpiperazine (500 mg, 3.97 mmol) in acetonitrile (5 ml) was added to a solution of 2-chloro-5-nitropyridine (631 mg, 3.97 mmol) in acetonitrile (10 ml). The mixture was stirred at rt for 4 hours and then concentrated to dryness. The residue was diluted with DCM and 2M sodium carbonate solution (aq). The organic phase was dried and concentrated to give a yellow solid (912 mg, 93%). ¹H NMR (400 MHz, DMSO-d₆) δ ppm 0.31-0.49 (m, 4H), 1.65 (tt, J=6.70, 3.38 Hz, 1H), 2.52-2.69 (m, 4H), 3.61-3.82 (m, 4H), 6.95 (d, J=9.62 Hz, 1H), 8.21 (dd, J=9.62, 2.75 Hz, 1H), 8.95 (d, J=2.75 Hz, 1H); m/z (ES+APCI)⁺: 249 [M+H]⁺.

### Step 2

### 6-(4-Cyclopropyl-piperazin-1-yl)-pyridin-3-ylamine

Prepared analogously Intermediate 70 (Step 2) using 1-cyclopropyl-4-(5-nitro-pyridin-2-yl)-piperazine to provide the product as a dark purple coloured oil (678 mg, 85%). ¹H NMR (400 MHz, DMSO-d₆) δ ppm 0.27-0.48 (m, 4H), 1.61 (tt, J=6.75, 3.55 Hz, 1H), 2.54-2.69 (m, 4H), 3.10-3.23 (m, 4H), 4.55 (s, 2H), 6.60 (d, J=8.70 Hz, 1H), 6.89 (dd, J=8.70, 2.75 Hz, 1H), 7.58 (d, J=2.29 Hz, 1H); m/z (ES+APCI)⁺: 219 [M+H]⁺.

### Intermediate 78

### 6-[2-(4-Methyl-piperazin-1-yl)-ethoxy]-pyridin-3-ylamine

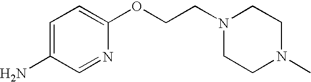

### Step 1

### 1-Methyl-4-[2-(5-nitro-pyridin-2-yloxy)-ethyl]-piperazine

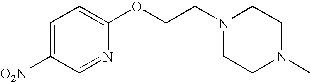

Sodium hydride, 60% dispersion in mineral oil (252 mg, 6.29 mmol) was added, portionwise, to an ice-cooled solution of 2-(4-methyl-piperazin-1-yl)-ethanol (863 mg, 5.99 mmol) and 2-chloro-5-nitropyridine (1.0 g, 6.29 mmol) in DMF (20 ml). The ice-cooling was removed after 1 hour and the mixture was allowed to stir at rt overnight. The reaction mixture was added to ice/water and subsequently extracted with EtOAc (×2). The combined organic extracts were washed with water (×4) and brine (×1), dried and concentrated. The crude product was purified by flash column chromatography on silica gel (60 g) eluting with 10:1 DCM:MeOH to give the product as a brown oil (400 mg, 24%). ¹H NMR (400 MHz, DMSO-d₆) δ ppm 2.13 (s, 3H), 2.21-2.54 (m, 8H), 2.70 (t, J=5.95 Hz, 2H), 4.50 (t, J=5.95 Hz, 2H), 7.04 (d, J=9.16 Hz, 1H), 8.47 (dd, J=9.16, 2.75 Hz, 1 H), 9.07 (d, J=2.29 Hz, 1H); m/z (ES+APCI)⁺: 267 [M+H]⁺.

### Step 2

### 6-[2-(4-Methyl-piperazin-1-yl)-ethoxy]-pyridin-3-ylamine

Prepared analogously to Intermediate 70 (Step 2) using 1-methyl-4-[2-(5-nitro-pyridin-2-yloxy)-ethyl]-piperazine to provide the product as a brown solid (396 mg, 114%). ¹H NMR (400 MHz, DMSO-d₆) δ ppm 2.13 (s, 3H), 2.21-2.51 (m, 8H), 2.58 (t, J=5.95 Hz, 2H), 4.17 (t, J=5.95 Hz, 2H), 4.73 (br. s, 2H), 6.52 (d, J=8.70 Hz, 1H), 6.98 (dd, J=8.70, 2.75 Hz, 1H), 7.47 (d, J=3.66 Hz, 1H); m/z (ES+APCI)⁺: 237 [M+H]⁺.

### Intermediate 79

### 3-Methyl-3H-imidazo[4,5-b]pyridin-6-ylamine

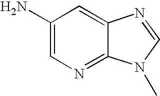

### Step 1

### Benzhydrylidene-(3-methyl-3H-imidazo[4,5-b]pyridin-6-yl)-amine

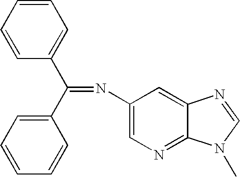

A mixture of 6-bromo-3-methyl-3H-imidazo[4,5-b]pyridine (786 mg, 3.71 mmol), benzophenone imine (805 mg, 4.45 mmol), sodium tert-butoxide (498 mg, 5.19 mmol), BINAP (17 mg, 0.028 mmol) and Pd₂(dba)₃ (8.5 mg, 0.0092 mmol) in toluene (15 ml) was place in a RBF, degassed and placed under an atmosphere of nitrogen. The mixture was stirred and heated at 80° C. overnight. On cooling to rt the mixture was diluted with EtOAc and water. The organic phase was washed with brine, dried and concentrated. The crude product was purified by flash column chromatography on silica gel (100 g) eluting with 40:1 DCM:MeOH to give the product as a yellow solid (663 mg, 57%). ¹H NMR (400 MHz, DMSO-d₆) δ ppm 3.74 (s, 3H), 7.16-7.24 (m, 2H), 7.26-7.42 (m, 4H), 7.42-7.59 (m, 3H), 7.69 (d, J=7.33 Hz, 2H), 7.83 (d, J=2.29 Hz, 1H), 8.28 (s, 1H).

### Step 2

### 3-Methyl-3H-imidazo[4,5-b]pyridin-6-ylamine

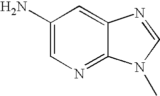

Benzhydrylidene-(3-methyl-3H-imidazo[4,5-b]pyridin-6-yl)-amine (735 mg, 2.36 mmol) in 2M HCl (0.4 ml) and THF (8 ml) was stirred at rt overnight. After stirring overnight more 2M HCl (0.4 ml) was added and stirring was continued at rt for 3 hours. The mixture was diluted with DCM and saturated sodium carbonate solution (aq). The organic phase was dried and concentrated. The crude product was purified by flash column chromatography on silica gel (30 g) eluting with 10:1 DCM:MeOH to give the product as a white solid (65 mg, 19%). ¹H NMR (400 MHz, DMSO-d₆) δ ppm 3.73 (s, 3H), 4.98 (s, 2H), 7.16 (d, J=2.29 Hz, 1H), 7.83 (d, J=2.75 Hz, 1H), 8.13 (s, 1H); m/z (ES+APCI)⁺: 149 [M+H]⁺.

### Intermediate 80

### 1-Methyl-1H-pyrazolo[3,4-b]pyridin-5-ylamine

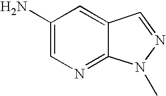

### Step 1

### Benzhydrylidene-(1-methyl-1H-pyrazolo[3,4-b]pyridin-5-yl)-amine

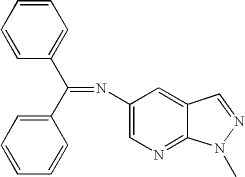

Prepared analogously to Intermediate 79 (Step 1) using 5-bromo-1-methyl-1H-pyrazolo[3,4-b]pyridine and benzophenone imine to provide the product as a yellow solid (916 mg, 92%). ¹H NMR (400 MHz, DMSO-d₆) δ ppm 3.96 (s, 3H), 7.20 (dd, J=7.56, 2.06 Hz, 2H), 7.25-7.38 (m, 3H), 7.39-7.59 (m, 4H), 7.70 (d, J=6.87 Hz, 2H), 7.96 (s, 1H), 8.03 (d, J=2.29 Hz, 1H); m/z (ES+APCI)⁺: 313 [M+H]⁺.

### Step 2

### 1-Methyl-1H-pyrazolo[3,4-b]pyridin-5-ylamine

Prepared analogously to Intermediate 79 (Step 2) using benzhydrylidene-(1-methyl-1H-pyrazolo[3,4-b]pyridin-5-yl)-amine to provide the product as an off-white solid (260 mg, 60%). ¹H NMR (400 MHz, DMSO-d₆) δ ppm 3.95 (s, 3H), 5.09 (s, 2H), 7.17 (d, J=2.75 Hz, 1H), 7.79 (s, 1H), 8.08 (d, J=2.75 Hz, 1H); m/z (ES+APCI)⁺: 149 [M+H]⁺.

### Intermediate 81

### 1-Cyclopropylmethyl-1H-pyrazol-4-ylamine

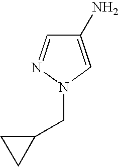

### Step 1

### 1-Cyclopropylmethyl-4-nitro-1H-pyrazole

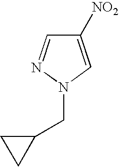

A mixture of 4-nitro-1H-pyrazole (1.0 g, 8.85 mmol), cyclopropylmethyl bromide (859 μl, 8.85 mmol) and potassium carbonate (1.22 g, 8.85 mmol) in DMF (25 ml) was stirred at 70° C. for 3 hours. On cooling to rt the mixture was diluted with EtOAc and washed with water (×4) and brine (×1), dried and concentrated. The residue was dissolved in EtOAc and washed with water (×3) and brine (×1), dried and concentrated to provide the product as a yellow oil (1.33 g, 90%). ¹H NMR (400 MHz, DMSO-d₆) δ ppm 0.35-0.48 (m, 2H), 0.48-0.62 (m, 2H), 1.22-1.34 (m, 1H), 4.03 (d, J=7.33 Hz, 2H), 8.27 (s, 1H), 8.91 (s, 1H).

### Step 2

### 1-Cyclopropylmethyl-1H-pyrazol-4-ylamine

Prepared analogously to Intermediate 70 (Step 2) using 1-cyclopropylmethyl-4-nitro-1H-pyrazole to provide the product as deep purple coloured oil (1.06 g, 97%). ¹H NMR (400 MHz, DMSO-d₆) δ ppm 0.23-0.34 (m, 2H), 0.43-0.51 (m, 2H), 1.03-1.17 (m, 1H), 3.69-3.90 (m, 4H), 6.87 (s, 1H), 7.05 (s, 1H); m/z (ES+APCI)⁺: 138 [M+H]⁺.

### Example 1

### N-{3-[5-Cyclopropyl-2-(3-fluoro-phenylamino)-pyrimidin-4-ylamino]-propyl}-acetamide trifluoroacetic acid salt

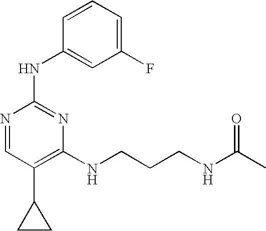

To a solution of intermediate 3 (35 mg, 0.10 mmol) in DMF (1 ml) was added glacial acetic acid (9 μl, 0.16 mmol), HATU (63.1 mg, 0.17 mmol) followed by N,N-diisopropylethylamine (108 μl, 0.62 mmol), and the resulting solution was left to stir at room temperature overnight. The volatiles were removed under reduced pressure and the crude product was re-dissolved in 1:9 methanol—DCM (1 ml) and eluted though an Isolute-NH₂ cartridge. The solvents were removed and the crude product purified by preparative LCMS (low pH buffer). ¹H NMR (400 MHz, DMSO-d₆) δ ppm 0.54-0.60 (m, 2H), 0.86-0.94 (m, 2H), 1.48-1.57 (m, 1H), 1.69-1.77 (m, 2H), 1.78 (s, 3H), 3.06-3.15 (m, 2H), 3.45-3.53 (m, 2H), 6.95 (td, J=8.4, 2.1 Hz, 1H), 7.29-7.35 (m, 1H), 7.37-7.45 (m, 1H), 7.56-7.65 (m, 2H), 7.93 (t, J=5.7 Hz, 1H), 8.46 (br. s, 1H), 10.25 (br. s, 1H); m/z (ES+APCI)⁺: 344 [M+H]⁺.

### Examples 2-10

Examples 2-10 were prepared analogously to Example 1 (the general structure is shown below followed by the tabulated examples).

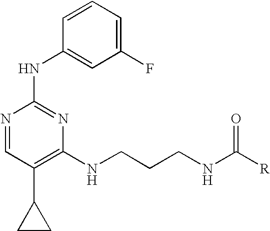

| | | | | HPLC |
|---|---|---|---|---|
| | | | | retention |
| | | | m/z | time |
| Example | R group | Name | (ES + APCI)⁺ | (min)* |
| 2 | 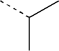 | N-{3-[5-Cyclopropyl-2-(3- fluoro-phenylamino)-pyrimidin- 4-ylamino]-propyl}- isobutyramide trifluoroacetic acid salt | 372 | 7.89 |
| 3 | 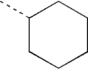 | Cyclohexanecarboxylic acid {3- [5-cyclopropyl-2-(3-fluoro- phenylamino)-pyrimidin-4- ylamino]-propyl}-amide trifluoroacetic acid salt | 412 | 8.42 |
| 4 | 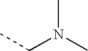 | N-{3-[5-Cyclopropyl-2-(3- fluoro-phenylamino)-pyrimidin- 4-ylamino]-propyl}-2- dimethylamino-acetamide trifluoroacetic acid salt | 387 | 6.84 |
| 5 | 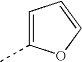 | Furan-2-carboxylic acid{3-[5- cyclopropyl-2-(3-fluoro- phenylamino)-pyrimidin-4- ylamino]-propyl}-amide trifluoroacetic acid salt | 396 | 7.79 |
| 6 | 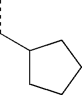 | 2-Cyclopentyl-N-{3-[5- cyclopropyl-2-(3-fluoro- phenylamino)-pyrimidin-4- ylamino]-propyl}-acetamide trifluoroacetic acid salt | 412 | 8.43 |
| 7 | 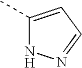 | 2H-Pyrazole-3-carboxylic acid {3-[5-cyclopropyl-2-(3-fluoro- phenylamino)-pyrimidin-4- ylamino]-propyl}-amide trifluoroacetic acid salt | 396 | 7.47 |
| 8 | 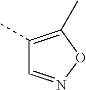 | 5-Methyl-isoxazole-4- carboxylic acid{3-[5- cyclopropyl-2-(3-fluoro- phenylamino)-pyrimidin-4- ylamino]-propyl}-amide trifluoroacetic acid salt | 411 | 8.14 |
| 9 | 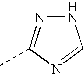 | 1H-1,2,4-Triazole-3-carboxylic acid {3-[5-cyclopropyl-2-(3- fluoro-phenylamino)-pyrimidin- 4-ylamino]-propyl}-amide trifluoroacetic acid salt | 397 | 7.35 |
| 10 | 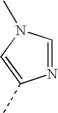 | 1-Methyl-1H-imidazole-4- carboxylic acid {3-[5- cyclopropyl-2-(3-fluoro- phenylamino)-pyrimidin-4- ylamino]-propyl}-amide trifluoroacetic acid salt | 410 | 7.08 |

*HPLC column: 21.2 × 100 mm (5 μm) C-18 Phenomenex Gemini; flow rate: 20 ml/min; run time: 10 min; gradient at start: 10% methanol and 90% water, gradient at finish: 100% methanol and 0% water; as buffer: 0.1% trifluoroacetic acid is added to the water.

### Example 11

### Thiazole-5-carboxylic acid {3-[5-cyclopropyl-2-(3-fluoro-phenylamino)-pyrimidin-4-ylamino]-propyl}-amide

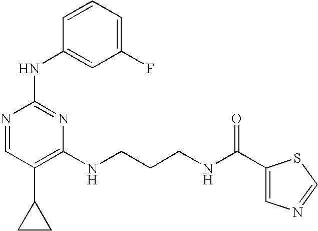

To a suspension of Intermediate 3 (35 mg, 0.10 mmol) in DMF (1 ml), was added thiazole-5-carboxylic acid (20.1 mg, 0.16 mmol), HATU (63.1 mg, 0.17 mmol) and N,N-diisopropylethylamine (108 μl, 0.62 mmol), and the resulting solution was stirred overnight. The volatiles were removed under reduced pressure and the crude product was re-dissolved in 1:9 methanol:DCM (1 ml) and eluted through an Isolute-NH₂ cartridge. After solvent removal, the crude product was purified by preparative LCMS (low pH buffer). After solvent removal, the product was re-dissolved in methanol/DCM (1:9) and eluted through an Isolute-NH₂ cartridge and solvents evaporated to give a solid (19 mg, 44%). ¹H NMR (400 MHz, CDCl₃) δ ppm 0.41-0.60 (m, 2H), 0.90-0.96 (m, 2H), 1.42-1.50 (m, 1H), 1.92-2.02 (m, 2H), 3.51-3.72 (m, 2H), 3.51-3.72 (m, 2H), 5.87 (t, J=6.2 Hz, 1H), 6.43 (t, J=5.7 Hz, 1H), 6.64 (td, J=8.1, 2.1 Hz, 1H), 6.92 (br. s, 1H), 7.03 (d, J=6.9 Hz, 1H), 7.16-7.24 (m, 1H), 7.77 (s, 1H), 7.82 (dt, J=12.4, 2.3 Hz, 1 H), 8.19 (s, 1H), 8.88 (s, 1H); m/z (ES+APCI)⁺: 413 [M+H]⁺.

### Examples 12-25

Examples 12-25 were prepared analogously to example 11 (the general structure is shown below followed by the tabulated examples).

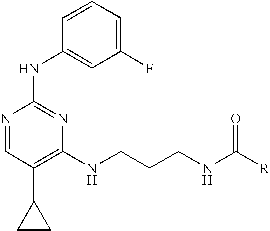

| | | | | HPLC |
|---|---|---|---|---|
| | | | m/z | retention |
| Example | R group | Name | (ES + APCI)⁺ | time (min)* |
| 12 | 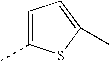 | 5-Methyl-thiophene-2- carboxylic acid {3-[5- cyclopropyl-2-(3-fluoro- phenylamino)-pyrimidin-4- ylamino]-propyl}-amide | 426 | 8.17 |
| 13 | 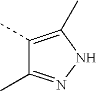 | 3,5-Dimethyl-1H-pyrazole- 4-carboxylic acid {3-[5- cyclopropyl-2-(3-fluoro- phenylamino)-pyrimidin-4- ylamino]-propyl}-amide | 424 | 7.85 |
| 14 | 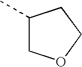 | Tetrahydro-furan-3- carboxylic acid {3-[5- cyclopropyl-2-(3-fluoro- phenylamino)-pyrimidin-4- ylamino]-propyl}-amide | 400 | 7.56 |
| 15 | 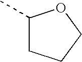 | Tetrahydro-furan-2- carboxylic acid {3-[5- cyclopropyl-2-(3-fluoro- phenylamino)-pyrimidin-4- ylamino]-propyl}-amide | 400 | 7.73 |
| 16 | 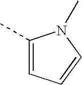 | 1-Methyl-1H-pyrrole-2- carboxylic acid {3-[5- cyclopropyl-2-(3-fluoro- phenylamino)-pyrimidin-4- ylamino]-propyl}-amide | 409 | 8.09 |
| 17 | 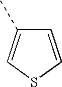 | Thiophene-3-carboxylic acid {3-[5-cyclopropyl-2-(3- fluoro-phenylamino)- pyrimidin-4-ylamino]- propyl}-amide | 412 | 8.06 |
| 18 | 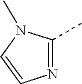 | 1-Methyl-1H-imidazole-2- carboxylic acid {3-[5- cyclopropyl-2-(3-fluoro- phenylamino)-pyrimidin-4- ylamino]-propyl}-amide | 410 | 7.36 |
| 19 | 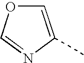 | Oxazole-4-carboxylic acid {3-[5-cyclopropyl-2-(3- fluoro-phenylamino)- pyrimidin-4-ylamino]- propyl}-amide | 397 | 7.55 |
| 20 | 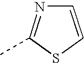 | Thiazole-2-carboxylic acid {3-[5-cyclopropyl-2-(3- fluoro-phenylamino)- pyrimidin-4-ylamino]- propyl}-amide | 413 | 7.84 |
| 21 | 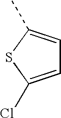 | 5-Chloro-thiophene-2- carboxylic acid {3-[5- cyclopropyl-2-(3-fluoro- phenylamino)-pyrimidin-4- ylamino]-propyl}-amide | 446 | 8.49 |
| 22 | 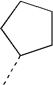 | Cyclopentanecarboxylic acid {3-[5-cyclopropyl-2-(3- fluoro-phenylamino) pyrimidin-4-ylamino]- propyl}-amide | 398 | 8.22 |
| 23 | 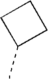 | Cyclobutanecarboxylic acid {3-[5-cyclopropyl-2-(3- fluoro-phenylamino)- pyrimidin-4-ylamino]- propyl}-amide | 384 | 8.00 |
| 24 | 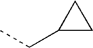 | 2-Cyclopropyl-N-{3-[5- cyclopropyl-2-(3-fluoro- phenylamino)-pyrimidin-4- ylamino]-propyl}-acetamide | 384 | 7.92 |
| 25 | 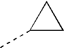 | Cyclopropanecarboxylic acid {3-[5-cyclopropyl-2-(3- fluoro-phenylamino)- pyrimidin-4-ylamino]- propyl}-amide | 370 | 7.74 |

*HPLC column: 21.2 × 100 mm (5 μm) C-18 Phenomenex Gemini; flow rate: 20 ml/min; run time: 10 min; gradient at start: 10% methanol and 90% water, gradient at finish: 100% methanol and 0% water; as buffer: 0.1% trifluoroacetic acid is added to the water.

### Example 26

### Thiophene-2-carboxylic acid {3-[5-cyclopropyl-2-(3-fluoro-phenylamino)-pyrimidin-4-ylamino]-propyl}-amide

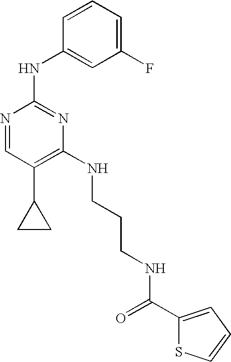

To a suspension of Intermediate 6 (110 mg, 0.326 mmol) and 3-fluoroaniline (0.425 mmol) in acetonitrile (1.27 ml) and water (0.128 ml) at room temperature was added, dropwise, 4M hydrogen chloride in dioxane (119 ml, 0.474 mmol). The reaction was stirred at 50° C. for 18 h and then quenched with water (10 ml). The pH was adjusted to 8 with saturated sodium bicarbonate solution and then extracted with DCM (3×60 ml). The combined organic layers were dried (MgSO₄) and evaporated to dryness. The crude product was purified by preparative LCMS (high pH buffer). The product was re-purified by flash chromatography on the Biotage SP4, eluting with 0 to 3.5% methanol/DCM. This gave the desired product as a white solid (20 mg, 15%). ¹H NMR (400 MHz, DMSO-d₆) δ ppm 0.44-0.51 (m, 2H), 0.78-0.86 (m, 2H), 1.42-1.51 (m, 1H), 1.80-1.90 (m, 2H), 3.28-3.39 (m, 2H), 3.46-3.54 (m, 2H), 6.57-6.65 (m, 1H), 6.89-6.96 (m, 1H), 7.13 (dd, J=5.0, 3.7 Hz, 1H), 7.16-7.24 (m, 1H), 7.41-7.46 (m, 1H), 7.64 (s, 1H), 7.71-7.77 (m, 2H), 7.84-7.91 (m, 1H), 8.52-8.57 (m, 1H), 9.16 (s, 1H); m/z (ES+APCI)⁺: 412 [M+H]⁺.

### Example 27

### Thiophene-2-carboxylic acid {3-[5-cyclopropyl-2-(3-trifluoromethyl-phenylamino)-pyrimidin-4-ylamino]-propyl}-amide

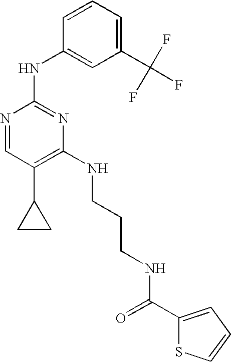

To a suspension of Intermediate 6 (40 mg, 0.119 mmol) and 3-trifluoromethyl-phenylamine (19 μl, 0.154 mmol) in acetonitrile (0.46 ml) and water (0.047 ml) at room temperature was added 4M HCl in dioxane (0.043 ml, 0.172 mmol) dropwise, and the resulting mixture was stirred at 50° C. for 18 h. After evaporating to dryness the crude product was subjected to flash chromatography on a Biotage SP4 (methanol/DCM gradient) followed by preparative LCMS (high pH buffer) to give a white solid (8 mg, 15%). ¹H NMR (400 MHz, DMSO-d₆) δ ppm 0.46-0.52 (m, 2H), 0.80-0.87 (m, 2H), 1.43-1.53 (m, 1H), 1.79-1.89 (m, 2H), 3.28-3.39 (m, 2H), 3.47-3.56 (m, 2H), 6.91-6.99 (m, 1H), 7.11-7.16 (m, 2H), 7.38-7.44 (m, 1H), 7.66 (s, 1H), 7.71-7.75 (m, 2H), 7.83-7.87 (m, 1H), 8.46 (s, 1H), 8.51-8.58 (m, 1H), 9.32 (br. s, 1H); m/z (ES+APCI)⁺: 462

### Example 28

### Thiophene-2-carboxylic acid {3-[2-(4-acetylamino-phenylamino)-5-cyclopropyl-pyrimidin-4-ylamino]-propyl}-amide. Hydrochloride salt

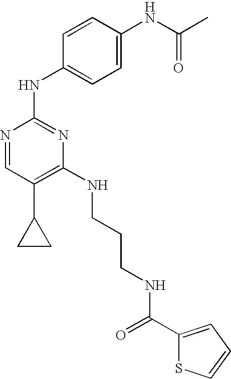

Intermediate 6 (60 mg, 0.178 mmol), N-(4-aminophenyl)acetamide (32 mg, 0.214 mmol), glacial acetic acid (5 μl, 0.071 mmol) and n-butanol (1 ml) were combined and heated at 100° C. for 18 h. The reaction mixture was filtered, and the residue washed with methanol and dried to give the title compound as a pale brown solid (19 mg, 24%). ¹H NMR (400 MHz, DMSO-d₆) δ ppm 0.43-0.49 (m, 2H), 0.79-0.85 (m, 2H), 1.42-1.50 (m, 1H), 1.78-1.89 (m, 2H), 2.00 (s, 3H), 3.29-3.38 (m, 2H), 3.45-3.53 (m, 2H), 7.02 (br. s, 1H), 7.10-7.15 (m, 1H), 7.42 (d, J=9.2 Hz, 2H), 7.58 (s, 1H), 7.62 (d, J=9.2 Hz, 2H), 7.71-7.75 (m, 2H), 8.50-8.61 (m, 1H), 8.92 (br. s, 1H), 9.75 (br. s, 1H); m/z (ES+APCI)⁺: 451 [M+H]⁺.

### Example 29

### Thiophene-2-carboxylic acid {3-[5-cyclopropyl-2-(3-hydroxy-phenylamino)-pyrimidin-4-ylamino]-propyl}-amide Trifluoroacetic acid salt

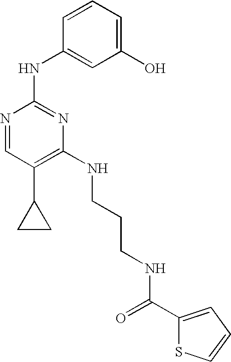

Intermediate 6 (50 mg, 0.148 mmol), 3-aminophenol (19 mg, 0.178 mmol), glacial acetic acid (2 μl, 0.03 mmol) and n-butanol (1 ml) were combined and irradiated at 150° C. for 20 minutes in a Biotage I-60 microwave reactor. The reaction mixture was evaporated and then purified by preparative LCMS (low pH buffer) to give the desired product as the TFA salt, white solid (22 mg, 28%). ¹H NMR (400 MHz, DMSO-d₆) δ ppm 0.53-0.60 (m, 2H), 0.89 (m, 2H), 1.52 (m, 1H), 1.86 (m, 2H), 3.33 (m, 2H), 3.39-3.61 (m, 2H), 6.55 (dd, J=7.8, 1.8 Hz, 1H), 7.01 (m, 2H), 7.10-7.17 (m, 2H), 7.55 (s, 1H), 7.73 (m, 2H), 8.53-8.62 (m, 2H), 9.60 (br. s, 1H), 10.11 (br. s, 1H); m/z (ES+APCI)⁺: 410 [M+H]⁺.

### Example 30

### Thiophene-2-carboxylic acid {3-[5-cyclopropyl-2-(6-methyl-pyridin-3-ylamino)-pyrimidin-4-ylamino]-propyl}-amide

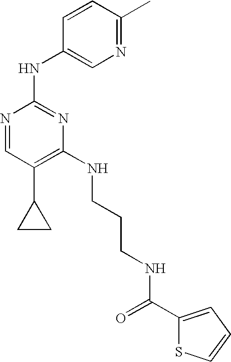

Intermediate 6 (60 mg, 0.178 mmol), 6-methyl-pyridin-3-ylamine (29 mg, 0.267 mmol), glacial acetic acid (5 μl, 0.071 mmol) and n-butanol (1 ml) were combined and heated at 100° C. for 18 h. More 6-methyl-pyridin-3-ylamine (10 mg, 0.089 mmol) was added and the reaction continued at 100° C. for 6 h. The reaction mixture was evaporated and then purified by preparative LCMS (low pH buffer), triturated with DCM, then eluted through a 1 g Isolute-NH₂ cartridge with 9:1 DCM: methanol to give a white solid (12 mg, 18%). ¹H NMR (400 MHz, DMSO-d₆) δ ppm 0.43-0.49 (m, 2H), 0.78-0.86 (m, 2H), 1.42-1.50 (m, 1H), 1.79-1.88 (m, 2H), 2.35 (s, 3H), 3.28-3.42 (m, 2H), 3.44-3.52 (m, 2H), 6.85-6.90 (m, 1H), 7.05 (d, J=8.7 Hz, 1H), 7.14 (dd, J=5.0, 3.7 Hz, 1H), 7.61 (s, 1H), 7.71-7.76 (m, 2H), 8.09 (dd, J=8.5, 2.5 Hz, 1H), 8.51-8.60 (m, 1H), 8.73 (d, J=2.7 Hz, 1H), 8.97 (br. s, 1H); m/z (ES+APCI)⁺: 409 [M+H]⁺.

### Example 31

### [3-(5-Cyclopropyl-4-{3-[(thiophene-2-carbonyl)-amino]-propylamino}-pyrimidin-2-ylamino)-phenyl]-carbamic acid tert-butyl ester

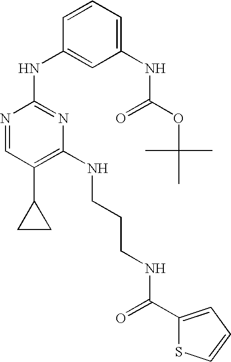

Intermediate 6 (50 mg, 0.148 mmol), (3-aminophenyl)carbamic acid tert-butyl ester (46 mg, 0.267 mmol), glacial acetic acid (2 μl, 0.03 mmol) and n-butanol (1 ml) were combined and irradiated at 150° C. for 20 minutes in a Biotage I-60 microwave reactor. The reaction mixture was evaporated and subjected to preparative LCMS (low pH buffer), subjected to anion exchange chromatography using a 1 g Isolute-NH₂ cartridge and then triturated with petroleum ether: diethyl ether (2:1) to give the desired product as a white solid (40 mg, 53%). ¹H NMR (400 MHz, DMSO-d₆) δ ppm 0.43-0.49 (m, 2H), 0.78-0.91 (m, 2H), 1.41-1.51 (m, 10H), 1.77-1.86 (m, 2H), 3.24-3.36 (m, 2H), 3.49-3.55 (m, 2H), 6.77-6.81 (m, 1H), 6.83 (dd, J=8.2, 0.9 Hz, 1H), 7.00-7.06 (m, 1H), 7.13 (dd, J=5.0, 3.7 Hz, 1H), 7.35 (dd, J=8.0, 1.1 Hz, 1H), 7.59 (s, 1H), 7.69-7.75 (m, 2H), 7.91 (s, 1H), 8.50-8.56 (m, 1H), 8.79 (s, 1H), 9.15 (br. s, 1H); m/z (ES+APCI)⁺: 409 [M−CO₂^{t}Bu+2H]⁺.

### Example 32

### Thiophene-2-carboxylic acid {3-[5-cyclopropyl-2-(3-methanesulfonylamino-phenylamino)-pyrimidin-4-ylamino]-propyl}-amide trifluoroacetic acid salt

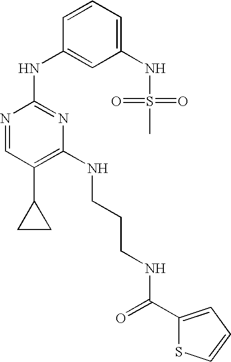

Intermediate 6 (41 mg, 0.121 mmol), N-(3-amino-phenyl)-methane sulfonamide (68 mg, 0.365 mmol), glacial acetic acid (8 μl, 0.121 mmol) and n-butanol (1.3 ml) were combined and irradiated at 150° C. for 40 minutes in a Biotage I-60 microwave reactor. The reaction mixture was evaporated and then purified by preparative LCMS (low pH buffer) to give a white solid (42 mg, 57%). ¹H NMR (400 MHz, DMSO-d₆) δ ppm 0.53-0.61 (m, 2H), 0.86-0.93 (m, 2H), 1.49-1.57 (m, 1H), 1.80-1.89 (m, 2H), 3.00 (s, 3H), 3.27-3.35 (m, 2H), 3.53-3.61 (m, 2H), 6.92-6.97 (m, 1H), 7.13 (dd, J=4.8, 3.9 Hz, 1H), 7.27-7.34 (m, 1H), 7.36-7.42 (m, 2H), 7.58 (s, 1H), 7.68-7.76 (m, 2H), 8.46-8.60 (m, 2H), 9.86 (s, 1H), 10.20 (br. s, 1H); m/z (ES+APCI)⁺: 487 [M+H]⁺.

### Examples 33-36

Examples 33-36 were prepared analogously to example 32 (the general structure is shown below followed by the tabulated examples).

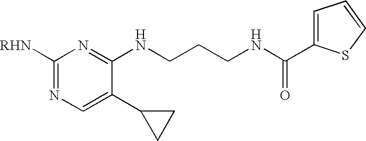

| | | | | HPLC |
|---|---|---|---|---|
| | | | m/z | retention |
| Example | R group | Name | (ES + APCI)⁺ | time (min)* |
| 33 | 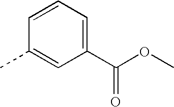 | 3-(5-Cyclopropyl-4-{3- [(thiophene-2-carbonyl)- amino]-propylamino}- pyrimidin-2-ylamino)-benzoic acid methyl ester trifluoroacetic acid salt | 452 | 8.16 |
| 34 | 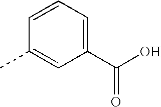 | 3-(5-Cyclopropyl-4-{3- [(thiophene-2-carbonyl)- amino]-propylamino}- pyrimidin-2-ylamino)-benzoic acid trifluoroacetic acid salt | 438 | 7.66 |
| 35 | 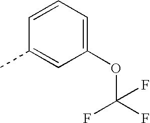 | Thiophene-2-carboxylic acid {3-[5-cyclopropyl-2-(3- trifluoromethoxy- phenylamino)-pyrimidin-4- ylamino]-propyl}-amide trifluoroacetic acid salt | 478 | 8.36 |
| 36 | 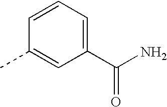 | Thiophene-2-carboxylic acid {3-[2-(3-carbamoyl- phenylamino)-5-cyclopropyl- pyrimidin-4-ylamino]-propyl}- amide trifluoroacetic acid salt | 437 | 7.33 |

*HPLC column: 21.2 × 100 mm (5 μm) C-18 Phenomenex Gemini; flow rate: 20 ml/min; run time: 10 min; gradient at start: 10% methanol and 90% water, gradient at finish: 100% methanol and 0% water; as buffer: 0.1% trifluoroacetic acid is added to the water.

### Example 37

### Thiophene-2-carboxylic acid {3-[2-(benzothiazol-6-ylamino)-5-cyclopropyl-pyrimidin-4-ylamino]-propyl}-amide. Hydrochloride salt

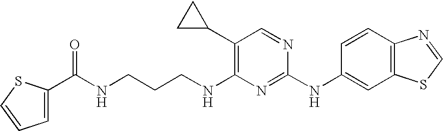

Intermediate 6 (41 mg, 0.121 mmol), benzothiazol-6-ylamine (55 mg, 0.365 mmol), glacial acetic acid (8 μl, 0.121 mmol) and n-butanol (1.3 ml) were combined and irradiated at 150° C. for 40 minutes in a Biotage I-60 microwave reactor. The reaction mixture was filtered, washed with methanol and the resultant solid was dried to give the desired product as a white solid (25 mg, 46%). ¹H NMR (400 MHz, DMSO-d₆) δ ppm 0.54-0.60 (m, 2H), 0.88-0.95 (m, 2H), 1.50-1.59 (m, 1H), 1.82-1.92 (m, 2H), 3.29-3.36 (m, 2H), 3.50-3.58 (m, 2H), 7.11 (dd, J=4.8, 3.9 Hz, 1H), 7.59 (s, 1H), 7.64 (dd, J=8.7, 1.8 Hz, 1H), 7.72-7.75 (m, 2H), 8.08 (d, J=9.2 Hz, 1H), 8.40 (d, J=1.8 Hz, 1H), 8.60-8.66 (m, 2H), 9.33 (s, 1H), 10.40 (br. s, 1H); m/z (ES+APCI)⁺: 451 [M+H]⁺.

### Example 38

### 1-{3-[5-Cyclopropyl-2-(3-fluoro-phenylamino)-pyrimidin-4-ylamino]-propyl}-pyrrolidin-2-one

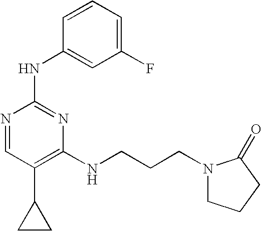

Intermediate 7 (97 mg, 0.33 mmol), 3-fluororaniline (94 μl, 0.99 mmol) and glacial acetic acid (60 μl, 1.06 mmol) were combined in n-butanol (1 ml) and heated in the microwave for 40 minutes at 140° C. The solvents were removed under reduced pressure and the crude product was purified by preparative LCMS (low pH buffer) and the resulting product subjected to anion exchange chromatography using an Isolute-NH₂ cartridge to give a white solid (46 mg, 38%). ¹H NMR (400 MHz, DMSO-d₆) δ ppm 0.46-0.51 (m, 2H), 0.78-0.90 (m, 2H), 1.44-1.52 (m, 1H), 1.79 (quin, J=6.9 Hz, 2H), 1.85-1.98 (m, 2H), 2.14-2.27 (m, 2H), 3.23-3.31 (m, 2H), 3.35 (t, J=7.1 Hz, 2H), 3.43 (q, J=6.4 Hz, 2H), 6.62 (td, J=8.2, 2.3 Hz, 1H), 6.84 (t, J=6.0 Hz, 1H), 7.18-7.25 (m, 1H), 7.44 (d, J=8.2 Hz, 1H), 7.65 (s, 1H), 7.79-7.86 (m, 1H), 9.08 (s, 1H); m/z (ES+APCI)⁺: 370 [M+H]⁺.

### Example 39

### N-(3-{5-Cyclopropyl-4-[3-(2-oxo-pyrrolidin-1-yl)-propylamino]-pyrimidin-2-ylamino}-phenyl)-acetamide trifluoroacetate salt

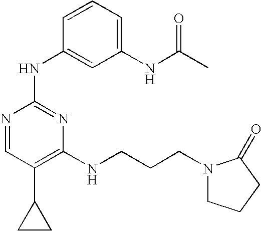

Intermediate 7 (97 mg, 0.33 mmol), 3-aminoacetanilide (148 mg, 0.99 mmol) and glacial acetic acid (60 μl, 1.06 mmol) were combined in n-butanol (1 ml) and heated in the microwave for 1 hour at 150° C. The solvents were removed under reduced pressure and the crude product was purified by preparative LCMS (low pH). The resulting TFA salt was subjected anion exchange chromatography using an Isolute-NH₂ cartridge to give a white solid (46 mg, 33%). ¹H NMR (400 MHz, DMSO-d₆) δ ppm 0.44-0.50 (m, 2H), 0.76-0.88 (m, 2H), 1.42-1.50 (m, 1H), 1.61-1.82 (m, 2H), 1.86-1.95 (m, 2H), 2.01 (s, 3H), 2.14-2.28 (m, 2H), 3.25 (t, J=6.9 Hz, 2H), 3.30-3.35 (m, 2H), 3.43 (q, J=6.4 Hz, 2H), 6.84 (t, J=5.7 Hz, 1H), 7.03-7.13 (m, 2H), 7.39 (d, J=7.3 Hz, 1H), 7.59 (s, 1H), 7.92 (s, 1H), 8.91 (s, 1H), 9.80 (s, 1H); m/z (ES+APCI)⁺: 409 [M+H]⁺.

### Example 40

### Cyclobutanecarboxylic acid {3-[2-(benzothiazol-6-ylamino)-5-cyclopropyl-pyrimidin-4-ylamino]-propyl}-amide. Hydrochloride salt

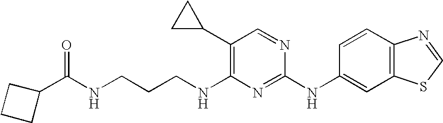

Intermediate 8 (50 mg, 0.162 mmol), benzothiazol-6-ylamine (73 mg, 0.486 mmol), glacial acetic acid (9 μl, 0.162 mmol) and n-butanol (1.5 ml) were combined and irradiated at 150° C. for 40 minutes in a Biotage I-60 microwave reactor. The reaction mixture was filtered, the residue was washed with methanol and dried to give the product as a white solid (52 mg, 76%). ¹H NMR (400 MHz, DMSO-d₆) δ ppm 0.54-0.61 (m, 2H), 0.87-0.95 (m, 2H), 1.51-1.60 (m, 1H), 1.65-1.78 (m, 3H), 1.78-1.90 (m, 1H), 1.90-2.00 (m, 2H), 2.02-2.15 (m, 2H), 2.88-2.99 (m, 1H), 3.08-3.15 (m, 2H), 3.39-3.55 (m, 2H), 7.61-7.70 (m, 2H), 7.75-7.82 (m, 1H), 8.11 (d, J=8.7 Hz, 1H), 8.41 (d, J=2.3 Hz, 1H), 8.64-8.73 (m, 1H), 9.34 (s, 1H), 10.68 (br. s, 1H); m/z (ES+APCI)⁺: 423 [M+H]⁺.

### Example 41

### Cyclobutanecarboxylic acid {3-[5-cyclopropyl-2-(3-morpholin-4-yl-phenylamino)-pyrimidin-4-ylamino]-propyl}-amide

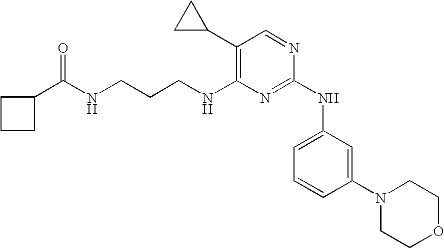

Intermediate 8 (50 mg, 0.162 mmol), 3-morpholin-4-yl-phenylamine (86 mg, 0.486 mmol), glacial acetic acid (9 μl, 0.162 mmol) and n-butanol (1.5 ml) were combined and irradiated at 150° C. for 40 minutes in a Biotage I-60 microwave reactor. The reaction mixture was evaporated and subjected to preparative LCMS (low pH buffer). The resulting TFA salt was subjected anion exchange chromatography using an Isolute-NH₂ cartridge to give a white solid (39 mg, 53%). ¹H NMR (400 MHz, DMSO-d₆) δ ppm 0.43-0.49 (m, 2H), 0.77-0.87 (m, 2H), 1.42-1.50 (m, 1H), 1.62-1.78 (m, 3H), 1.79-1.91 (m, 1H), 1.92-2.02 (m, 2H), 2.05-2.16 (m, 2H), 2.90-3.00 (m, 1H), 3.02-3.07 (m, 4H), 3.09-3.16 (m, 2H), 3.40-3.47 (m, 2H), 3.70-3.75 (m, 4H), 6.45 (dd, J=8.2, 1.8 Hz, 1H), 6.77-6.82 (m, 1H), 7.01-7.08 (m, 1H), 7.22 (dd, J=8.0, 1.1 Hz, 1H), 7.44-7.47 (m, 1H), 7.60 (s, 1H), 7.65-7.70 (m, 1H), 8.72 (br. s, 1H); m/z (ES+APCI)⁺: 451.

### Example 42

### Cyclobutanecarboxylic acid {3-[5-cyclopropyl-2-(3-imidazol-1-ylmethyl-phenylamino)-pyrimidin-4-ylamino]-propyl}-amide

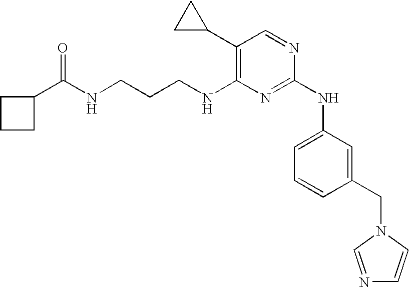

Prepared analogously to example 41 from Intermediate 8 and 3-(1H-imidazol-1-ylmethyl)aniline to give a white solid (7 mg, 10%). ¹H NMR (400 MHz, DMSO-d₆) δ ppm 0.44-0.48 (m, 2H), 0.79-0.85 (m, 2H), 1.42-1.49 (m, 1H), 1.64-1.76 (m, 3H), 1.80-1.89 (m, 1H), 1.92-2.01 (m, 2H), 2.04-2.15 (m, 2H), 2.90-3.00 (m, 1H), 3.09-3.15 (m, 2H), 3.36-3.42 (m, 2H), 5.10-5.14 (m, 2H), 6.64-6.68 (m, 1H), 6.82-6.86 (m, 1H), 6.89-6.90 (m, 1H), 7.11-7.14 (m, 1H), 7.15-7.20 (m, 1H), 7.60 (s, 1H), 7.63 (dd, J=8.2, 1.4 Hz, 1H), 7.67-7.76 (m, 3H), 8.96 (br. s, 1H); m/z (ES+APCI)⁺: 446.

### Example 43

### Cyclobutanecarboxylic acid (3-{5-cyclopropyl-2-[6-(4-methyl-piperazin-1-yl)-pyridin-3-ylamino]-pyrimidin-4-ylamino}-propyl)-amide

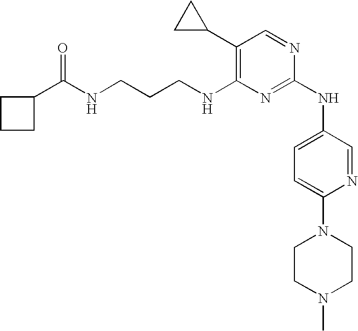

Intermediate 8 (50 mg, 0.162 mmol), 6-(4-methyl-piperazin-1-yl)-pyridin-3-ylamine (93 mg, 0.486 mmol), glacial acetic acid (9 μl, 0.162 mmol) and n-butanol (1.5 ml) were combined and irradiated at 150° C. for 40 minutes in a Biotage I-60 microwave reactor. Glacial acetic acid (27 μl, 0.486 mmol) was added and the reaction was irradiated at 150° C. for a further 40 minutes. The reaction mixture was evaporated and then purified by preparative LCMS (low pH buffer), and the resulting TFA salt was subjected anion exchange chromatography using an Isolute-NH₂ cartridge to give the product as a white solid (14.4 mg, 19%). ¹H NMR (400 MHz, DMSO-d₆) δ ppm 0.40-0.46 (m, 2H), 0.77-0.84 (m, 2H), 1.39-1.48 (m, 1H), 1.60-1.78 (m, 3H), 1.79-1.92 (m, 1H), 1.92-2.02 (m, 2H), 2.04-2.17 (m, 2H), 2.20 (s, 3H), 2.36-2.43 (m, 4H), 2.46-2.54 (m, 4H), 2.91-3.00 (m, 1H), 3.07-3.16 (m, 2H), 3.29-3.42 (m, 2H), 6.73-6.80 (m, 2H), 7.55 (s, 1H), 7.65-7.70 (m, 1H), 7.94 (dd, J=9.2, 2.7 Hz, 1H), 8.41 (d, J=2.3 Hz, 1H), 8.63 (br. s, 1H); m/z (ES+APCI)⁺: 465.

### Example 44

### Thiophene-2-carboxylic acid {3-[5-cyclopropyl-2-(3-dimethylamino-phenylamino)-pyrimidin-4-ylamino]-propyl}-amide trifluoroacetic acid salt

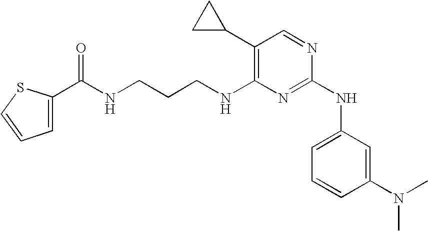

Intermediate 6 (41 mg, 0.122 mmol), N,N-dimethyl-benzene-1,3-diamine (61 mg, 0.365 mmol), glacial acetic acid (8 μl, 0.122 mmol) and n-butanol (1.3 ml) were combined and irradiated at 150° C. for 40 minutes in a Biotage I-60 microwave reactor. The reaction mixture was evaporated and subjected to preparative LCMS (low pH buffer) followed by flash chromatography on the Biotage SP4 (gradient elution from 0 to 8% methanol in DCM) gave a white solid (5 mg, 7%). ¹H NMR (400 MHz, DMSO-d₆) δ ppm 0.43-0.49 (m, 2H), 0.78-0.85 (m, 2H), 1.41-1.51 (m, 1H), 1.78-1.87 (m, 2H), 2.85 (s, 6H), 3.34 (s, 2H), 3.48-3.55 (m, 2H), 6.24 (dd, J=8.2, 2.3 Hz, 1H), 6.74-6.81 (m, 1H), 6.95-7.00 (m, 1H), 7.08-7.11 (m, 1H), 7.13 (dd, J=5.0, 3.7 Hz, 1H), 7.24-7.27 (m, 1H), 7.60 (s, 1H), 7.69-7.76 (m, 2H), 8.51-8.57 (m, 1H), 8.63 (br. s, 1H); m/z (ES+APCI)⁺: 437 [M+H]⁺.

### Example 45

### Thiophene-2-carboxylic acid (3-{5-cyclopropyl-2-[3-(4-methyl-piperazine-1-carbonyl)-phenylamino]-pyrimidin-4-ylamino}-propyl)-amide

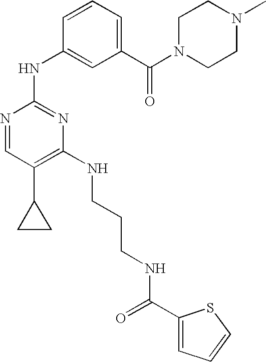

To a solution of Example 34 (20.1 mg, 0.036 mmol) in DMF (0.5 ml) was added 1-methylpiperazine (6 μl, 0.055 mmol), HATU (22 mg, 0.58 mmol) and N,N-diisopropylethylamine (38 μl, 0.219 mmol). The reaction was stirred at room temperature for 18 hours. The mixture was evaporated then filtered through a 1 g Isolute-NH₂ cartridge, eluting with 9:1 DCM:methanol. Purification by flash chromatography on the Biotage SP4 (gradient elution from 0 to 10% methanol in DCM) gave the desired product as a white solid (18.9 mg, 98%). ¹H NMR (400 MHz, DMSO-d₆, 85° C.) δ ppm 0.45-0.52 (m, 2H), 0.81-0.89 (m, 2H), 1.46-1.55 (m, 1H), 1.83-1.93 (m, 2H), 2.28 (s, 3H), 2.39-2.46 (m, 4H), 3.33-3.42 (m, 2H), 3.46-3.57 (m, 6H), 6.58-6.65 (m, 1H), 6.83 (ddd, J=7.6, 1.4, 1.1 Hz, 1H), 7.11 (dd, J=4.8, 3.9 Hz, 1H), 7.22-7.28 (m, 1H), 7.63 (s, 1H), 7.66-7.72 (m, 2H), 7.76 (ddd, J=8.2, 2.3, 0.9 Hz, 1H), 7.86-7.90 (m, 1H), 8.22-8.28 (m, 1H), 8.73 (br. s, 1H); m/z (ES+APCI)⁺: 520 [M+H]⁺.

### Example 46

### Thiophene-2-carboxylic acid {3-[5-cyclopropyl-2-(3-morpholin-4-yl-phenylamino)-pyrimidin-4-ylamino]-propyl}-amide trifluoroacetic acid salt

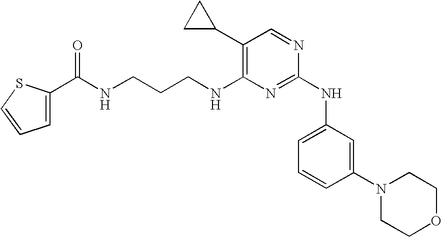

Prepared analogously to example 44 from Intermediate 6 and 3-morpholin-4-ylaniline. The product was isolated as a white foam (25 mg, 35%). ¹H NMR (400 MHz, DMSO-d₆) δ ppm 0.44-0.50 (m, 2H), 0.78-0.85 (m, 2H), 1.42-1.51 (m, 1H), 1.78-1.88 (m, 2H), 3.01-3.08 (m, 4H), 3.29-3.38 (m, 2H), 3.46-3.55 (m, 2H), 3.70-3.76 (m, 4H), 6.44 (dd, J=8.0, 2.1 Hz, 1H), 6.79-6.83 (m, 1H), 6.99-7.06 (m, 1H), 7.13 (dd, J=5.0, 3.7 Hz, 1H), 7.23 (dd, J=8.0, 1.1 Hz, 1H), 7.43-7.47 (m, 1H), 7.61 (s, 1H), 7.71-7.76 (m, 2H), 8.52-8.57 (m, 1H), 8.72 (br. s, 1H); m/z (ES+APCI)⁺: 479 [M+H]⁺.

### Example 47

### Thiophene-2-carboxylic acid (3-{5-cyclopropyl-2-[3-(2-oxo-pyrrolidin-1-yl)-phenylamino]-pyrimidin-4-ylamino}-propyl)-amide. Hydrochloride salt

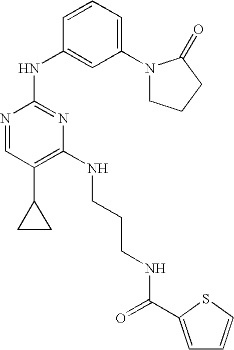

Intermediate 6 (40 mg, 0.119 mmol), 1-(3-amino-phenyl)-pyrrolidin-2-one (63 mg, 0.356 mmol), glacial acetic acid (8 μl, 0.119 mmol) and n-butanol (1 ml) were combined and irradiated at 150° C. for 40 minutes in a Biotage 1-60 microwave reactor. The reaction mixture was evaporated, the residue was triturated with methanol and dried to yield a white solid (12.4 mg, 22%). ¹H NMR (400 MHz, DMSO-d₆) δ ppm 0.52-0.60 (m, 2H), 0.87-0.94 (m, 2H), 1.50-1.60 (m, 1H), 1.78-1.89 (m, 2H), 2.00-2.12 (m, 2H), 3.26-3.33 (m, 2H), 3.33-3.46 (m, 2H), 3.52-3.63 (m, 2H), 3.78-3.88 (m, 2H), 7.12 (dd, J=4.8, 3.9 Hz, 1H), 7.23-7.40 (m, 3H), 7.60 (s, 1H), 7.70-7.77 (m, 2H), 8.04-8.11 (m, 1H), 8.57-8.70 (m, 2H), 10.29 (br. s, 1H); m/z (ES+APCI)⁺: 477 [M+H]⁺.

### Example 48

### Cyclobutanecarboxylic acid (3-{5-cyclopropyl-2-[3-(2-oxo-pyrrolidin-1-yl)-phenylamino]-pyrimidin-4-ylamino}-propyl)-amide

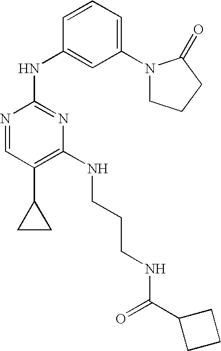

Intermediate 8 (50 mg, 0.162 mmol), 1-(3-aminophenyl)-pyrrolidin-2-one (86 mg, 0.485 mmol), glacial acetic acid (9 μl, 0.162 mmol) and n-butanol (1.5 ml) were combined and irradiated at 150° C. for 40 minutes in a Biotage 1-60 microwave reactor. The reaction mixture was evaporated, the residue was triturated with methanol and the resultant solid was dried to yield the title compound as a white solid (27 mg, 37%). ¹H NMR (400 MHz, DMSO-d₆) δ ppm 0.53-0.60 (m, 2H), 0.87-0.94 (m, 2H), 1.50-1.60 (m, 1H), 1.65-1.77 (m, 3H), 1.78-1.91 (m, 1H), 1.91-2.01 (m, 2H), 2.02-2.14 (m, 4H), 2.87-3.00 (m, 1H), 3.05-3.13 (m, 2H), 3.32-3.43 (m, 2H), 3.47-3.56 (m, 2H), 3.80-3.87 (m, 2H), 7.25-7.43 (m, 3H), 7.63 (s, 1H), 7.73-7.81 (m, 1H), 8.06-8.12 (m, 1H), 8.56-8.67 (m, 1H), 10.42 (br. s, 1H); m/z (ES+APCI)⁺: 449 [M+H]⁺.

### Example 49

### Pyrrolidine-1-carboxylic acid [3-(5-cyclopropyl-4-{3-[(thiophene-2-carbonyl)-amino]-propylamino}-pyrimidin-2-ylamino)-phenyl]-amide

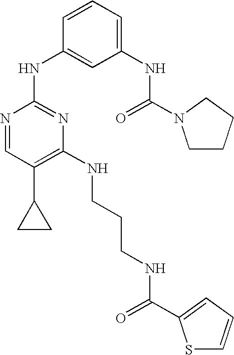

Intermediate 6 (50 mg, 0.148 mmol), Intermediate 10 (43 mg, 0.178 mmol), tris(dibenzylideneacetone)dipalladium(0) (8 mg, 0.009 mmol), xantphos (7 mg, 0.012 mmol) and sodium tert-butoxide (43 mg, 0.44 mmol) were combined with dioxane (3 ml), sealed and then purged with nitrogen gas. The reaction mixture was heated at 105° C. for 18 h. The reaction mixture was evaporated onto silica, then subjected to flash chromatography on the Biotage SP4 (gradient elution from 0 to 10% methanol in DCM). Further purification by cation exchange chromatography using an Isolute SCX cartridge gave a white solid (9 mg, 12%). ¹H NMR (400 MHz, DMSO-d₆) δ ppm 0.42-0.48 (m, 2H), 0.78-0.85 (m, 2H), 1.40-1.50 (m, 1H), 1.77-1.87 (m, 6H), 3.24-3.61 (m, 8H), 6.76-6.81 (m, 1H), 6.88-6.92 (m, 1H), 6.99-7.04 (m, 1H), 7.10-7.14 (m, 1H), 7.27-7.32 (m, 1H), 7.59 (s, 1H), 7.69-7.75 (m, 2H), 7.86-7.90 (m, 1H), 7.94 (s, 1H), 8.52-8.56 (m, 1H), 8.75 (br. s, 1H); m/z (ES+APCI)⁺: 506 [M+H]⁺.

### Example 50

### Thiophene-2-carboxylic acid {3-[5-cyclopropyl-2-(pyridin-3-ylamino)-pyrimidin-4-ylamino]-propyl}-amide

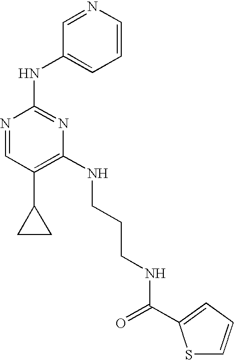

Intermediate 6 (60 mg, 0.179 mmol), 3-aminopyridine (20 mg, 0.213 mmol), tris-(dibenzylideneacetone)dipalladium(0) (9.8 mg, 0.011 mmol), xantphos (8 mg, 0.014 mmol) and sodium tert-butoxide (48 mg, 0.5 mmol) were combined with dioxane (3 ml), sealed and then purged with nitrogen gas. The reaction mixture was heated at 105° C. for 18 h. The reaction mixture was evaporated onto silica, then purified by flash chromatography on a Biotage SP4 (gradient elution from 0 to 10% methanol in DCM) to give an off-white solid (18 mg, 26%). ¹H NMR (400 MHz, DMSO-d₆) δ ppm 0.44-0.51 (m, 2H), 0.83 (m, 2H), 1.47 (m, 1H), 1.79-1.89 (m, 2H), 3.34 (m, 2H), 3.49 (m, 2H), 6.92 (m, 1H), 7.13 (dd, J=5.0, 3.7 Hz, 1H), 7.21 (dd, J=8.5, 4.8 Hz, 1H), 7.64 (s, 1H), 7.70-7.76 (m, 2H), 8.03 (dd, J=4.6, 1.4 Hz, 1H), 8.21-8.26 (m, 1H), 8.53-8.59 (m, 1H), 8.84-8.87 (m, 1H), 9.10 (s, 1H); m/z(ES+APCI)⁺: 395 [M+H]⁺.

### Example 51

### Thiophene-2-carboxylic acid {3-[2-(3-acetylamino-phenylamino)-5-cyclopropyl-pyrimidin-4-ylamino]-propyl}-amide

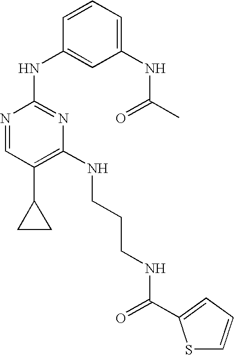

Intermediate 6 (60 mg, 0.179 mmol), N-(3-aminophenyl)acetamide (32 mg, 0.215 mmol), tris(dibenzylideneacetone)dipalladium(0) (9.8 mg, 0.011 mmol), xantphos (8 mg, 0.014 mmol) and sodium tert-butoxide (48 mg, 0.5 mmol) were suspended in dioxane (3 ml), sealed and then purged with nitrogen gas. The reaction mixture was heated at 105° C. for 18 h. The reaction mixture was evaporated onto silica subjected to flash chromatography on the Biotage SP4 (gradient elution from 0 to 10% methanol in DCM). Further purification by preparative LCMS (high pH buffer) gave the desired product as a white solid (7 mg, 9%). ¹H NMR (400 MHz, DMSO-d₆) δ ppm 0.43-0.49 (m, 2H), 0.78-0.85 (m, 2H), 1.42-1.51 (m, 1H), 1.78-1.87 (m, 2H), 2.01 (s, 3H), 3.29-3.37 (m, 2H), 3.46-3.56 (m, 2H), 6.76-6.82 (m, 1H), 7.02-7.11 (m, 2H), 7.13 (dd, J=4.8, 3.9 Hz, 1H), 7.39-7.43 (m, 1H), 7.60 (s, 1H), 7.70-7.76 (m, 2H), 7.93-7.95 (m, 1H), 8.51-8.57 (m, 1H), 8.85 (br. s, 1H), 9.77 (br. s, 1H); m/z (ES+APCI)⁺: 451 [M+H]⁺.

### Example 52

### Thiophene-2-carboxylic acid {3-[5-cyclopropyl-2-(2-fluoro-phenylamino)-pyrimidin-4-ylamino]-propyl}-amide

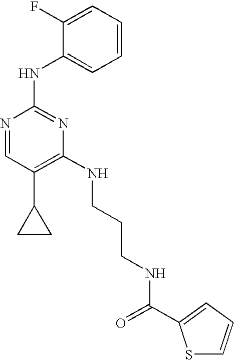

Intermediate 6 (60 mg, 0.179 mmol), 2-fluorophenylamine (21 mg, 0.215 mmol), palladium (II) acetate (1.6 mg, 0.007 mmol), xantphos (8 mg, 0.014 mmol) and cesium carbonate (174 mg, 0.5 mmol) were combined with dioxane (3 ml), sealed and then purged with nitrogen gas. The reaction mixture was heated at 105° C. for 18 h. The reaction mixture was evaporated onto silica then subjected to flash chromatography on the Biotage SP4 (gradient elution from 0 to 10% methanol in DCM). Further purification by preparative LCMS (high pH buffer) gave a white solid (4 mg, 5%). ¹H NMR (400 MHz, DMSO-d₆) δ ppm 0.41-0.50 (m, 2H), 0.77-0.85 (m, 2H), 1.40-1.49 (m, 1H), 1.73-1.85 (m, 2H), 3.22-3.39 (m, 2H), 3.39-3.48 (m, 2H), 6.82-6.90 (m, 1H), 6.91-6.98 (m, 1H), 7.05-7.10 (m, 1H), 7.11-7.19 (m, 2H), 7.58 (s, 1H), 7.69-7.77 (m, 2H), 8.02 (br. s, 1H), 8.04-8.10 (m, 1H), 8.45-8.60 (m, 1H); m/z (ES+APCI)⁺: 412 [M+H]⁺.

### Example 53

### Thiophene-2-carboxylic acid {3-[5-cyclopropyl-2-(isoquinolin-6-ylamino)-pyrimidin-4-ylamino]-propyl}-amide

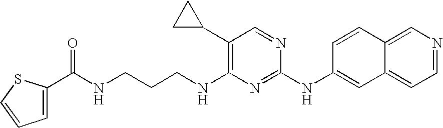

Intermediate 6 (50 mg, 0.148 mmol), Isoquinolin-6-ylamine (26 mg, 0.178 mmol), tris(dibenzylideneacetone)dipalladium(0) (8 mg, 0.009 mmol), xantphos (7 mg, 0.012 mmol) and sodium tert-butoxide (43 mg, 0.44 mmol) were combined with dioxane (3 ml), sealed and then purged with nitrogen gas. The reaction mixture was heated at 105° C. for 18 h, evaporated and purified through a silica plug, eluting with 0 to 10% methanol/DCM. Further purification by preparative LCMS (high pH buffer) gave the desired product as a white solid (15 mg, 23%). ¹H NMR (400 MHz, DMSO-d₆) δ ppm 0.42-0.50 (m, 2H), 0.78-0.86 (m, 2H), 1.41-1.52 (m, 1H), 1.71-1.81 (m, 2H), 3.24-3.31 (m, 2H), 3.36-3.45 (m, 2H), 6.81-6.89 (m, 1H), 7.13 (dd, J=4.8, 3.9 Hz, 1H), 7.54-7.63 (m, 2H), 7.70-7.76 (m, 3H), 8.02 (d, J=6.4 Hz, 1H), 8.24 (dd, J=7.8, 0.9 Hz, 1H), 8.43 (d, J=6.0 Hz, 1H), 8.48-8.55 (m, 1H), 8.90 (s, 1H), 9.23 (s, 1H); m/z (ES+APCI)⁺: 445 [M+H]⁺.

### Examples 54-55

Examples 54-55 were prepared analogously to Example 53 (the general structure is shown below followed by the tabulated examples).

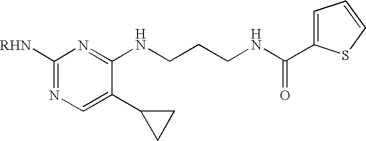

| | | | | HPLC |
|---|---|---|---|---|
| | | | | retention |
| | | | m/z | time |
| Example | R group | Name | (ES + APCI)⁺ | (min)* |
| 54 | 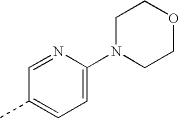 | Thiophene-2-carboxylic acid {3-[5-cyclopropyl-2-(6- morpholin-4-yl-pyridin-3- ylamino)-pyrimidin-4- ylamino]-propyl}-amide | 480 | 8.67 |
| 55 | 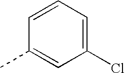 | Thiophene-2-carboxylic acid {3-[2-(3-chloro- phenylamino)-5- cyclopropyl-pyrimidin-4- ylamino]-propyl}-amide | 427 | 9.61 |

*HPLC column: 21.2 × 100 mm (10 μm) C-18 Phenomenex Gemini; flow rate: 20 ml/min; run time: 10 min; gradient at start: 10% methanol and 90% water, gradient at finish: 100% methanol and 0% water; as buffer: ammonium bicarbonate (10 mmol) and ammonium hydroxide is added to the water.

### Example 56

### Thiophene-2-carboxylic acid {3-[5-cyclopropyl-2-(quinoxalin-6-ylamino)-pyrimidin-4-ylamino]-propyl}-amide

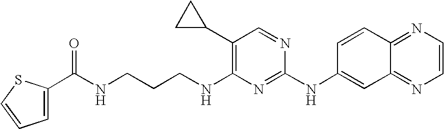

Prepared analogously to Example 50 from Intermediate 6 and quinoxalin-6-ylamine to give desired product as a yellow solid (25 mg, 38%). ¹H NMR (400 MHz, DMSO-d₆) δ ppm 0.49-0.55 (m, 2H), 0.82-0.89 (m, 2H), 1.46-1.56 (m, 1H), 1.87-1.97 (m, 2H), 3.36-3.45 (m, 2H), 3.55-3.64 (m, 2H), 6.99-7.05 (m, 1H), 7.11 (dd, J=4.8, 3.9 Hz, 1H), 7.69-7.75 (m, 3H), 7.91 (d, J=9.2 Hz, 1H), 8.04 (dd, J=9.2, 2.3 Hz, 1H), 8.52-8.58 (m, 1H), 8.66 (d, J=1.8 Hz, 1H), 8.77 (d, J=1.8 Hz, 1H), 8.82 (d, J=2.3 Hz, 1H), 9.58 (br. s, 1H); m/z (ES+APCI)⁺: 446 [M+H]⁺.

### Example 57

### Thiophene-2-carboxylic acid [3-(5-cyclopropyl-2-phenylamino-pyrimidin-4-ylamino)-propyl]-amide

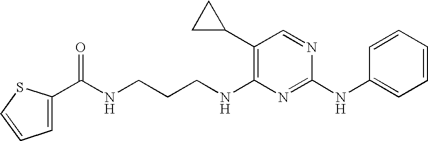

Prepared analogously to Example 50 from Intermediate 6 and phenylamine to give the desired product as a white solid (20 mg, 9%). ¹H NMR (400 MHz, DMSO-d₆) δ ppm 0.41-0.51 (m, 2H), 0.77-0.87 (m, 2H), 1.42-1.50 (m, 1H), 1.78-1.89 (m, 2H), 3.23-3.41 (m, 2H), 3.43-3.53 (m, 2H), 6.77-6.86 (m, 2H), 7.13 (dd, J=5.0, 3.7 Hz, 1H), 7.15-7.21 (m, 2H), 7.61 (s, 1H), 7.68-7.78 (m, 4H), 8.50-8.57 (m, 1H), 8.87 (br. s, 1H); m/z (ES+APCI)⁺: 394 [M+H]⁺.

### Example 58

### Thiophene-2-carboxylic acid {3-[5-cyclopropyl-2-(4-fluoro-phenylamino)-pyrimidin-4-ylamino]-propyl}-amide

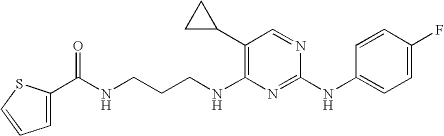

Prepared analogously to Example 50 from Intermediate 6 and 4-fluorophenylamine to give desired product as an off-white solid (25 mg, 41%). ¹H NMR (400 MHz, DMSO-d₆) δ ppm 0.36-0.53 (m, 2H), 0.67-0.91 (m, 2H), 1.38-1.51 (m, 1H), 1.67-1.91 (m, 2H), 3.19-3.43 (m, 2H), 3.43-3.61 (m, 2H), 6.73-6.92 (m, 1H), 6.92-7.08 (m, 2H), 7.13 (dd, J=4.8, 3.9 Hz, 1H), 7.60 (s, 1H), 7.67-7.76 (m, 4H), 8.43-8.61 (m, 1H), 8.85-8.99 (m, 1H); m/z (ES+APCI)⁺: 412 [M+H]⁺.

### Example 59

### Thiophene-2-carboxylic acid [3-(5-cyclopropyl-2-o-tolylamino-pyrimidin-4-ylamino)-propyl]-amide

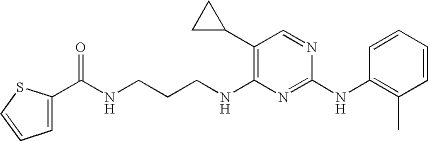

Prepared analogously to Example 50 from Intermediate 6 and o-tolylamine to give desired product as an off-white solid (10 mg, 17%). ¹H NMR (400 MHz, DMSO-d₆) δ ppm 0.41-0.46 (m, 2H), 0.77-0.83 (m, 2H), 1.40-1.47 (m, 1H), 1.73-1.81 (m, 2H), 2.21 (s, 3H), 3.26-3.36 (m, 2H), 3.37-3.45 (m, 2H), 6.71-6.76 (m, 1H), 6.86-6.91 (m, 1H), 7.04-7.16 (m, 3H), 7.54 (s, 1H), 7.69-7.76 (m, 4H), 8.46-8.58 (m, 1H); m/z (ES+APCI)⁺: 408 [M+H]⁺.

### Example 60

### Thiophene-2-carboxylic acid {3-[5-cyclopropyl-2-(2-methyl-benzothiazol-5-ylamino)-pyrimidin-4-ylamino]-propyl}-amide

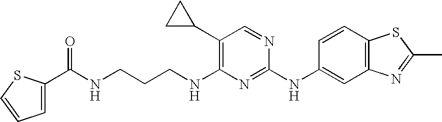

Prepared analogously to Example 50 from Intermediate 6 and 2-Methyl-benzothiazol-5-ylamine gave the desired product as a white solid (7 mg, 10%). ¹H NMR (400 MHz, DMSO-d₆, 85° C.) δ ppm 0.47-0.51 (m, 2H), 0.82-0.88 (m, 2H), 1.48-1.55 (m, 1H), 1.88-1.96 (m, 2H), 2.74 (s, 3H), 3.38-3.44 (m, 2H), 3.54-3.60 (m, 2H), 6.56-6.60 (m, 1H), 7.10 (dd, J=5.0, 3.7 Hz, 1H), 7.65 (s, 1H), 7.66-7.70 (m, 2H), 7.72-7.74 (m, 2H), 8.25-8.29 (m, 1H), 8.46-8.48 (m, 1H), 8.73 (br. s, 1H); m/z (ES+APCI)⁺: 465 [M+H]⁺.

### Example 61

### Thiophene-2-carboxylic acid {3-[2-(3-cyano-phenylamino)-5-cyclopropyl-pyrimidin-4-ylamino]-propyl}-amide

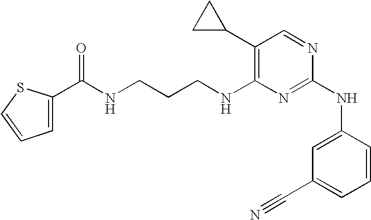

Prepared analogously to Example 50 from Intermediate 6 and 3-amino benzonitrile to give desired product as a white solid (15 mg, 24%). ¹H NMR (400 MHz, DMSO-d₆) δ ppm 0.46-0.51 (m, 2H), 0.80-0.87 (m, 2H), 1.44-1.51 (m, 1H), 1.81-1.89 (m, 2H), 3.26-3.41 (m, 2H), 3.45-3.59 (m, 2H), 6.95-7.00 (m, 1H), 7.13 (dd, J=4.8, 3.9 Hz, 1H), 7.23-7.28 (m, 1H), 7.38-7.43 (m, 1H), 7.67 (s, 1H), 7.71-7.76 (m, 2H), 7.92-7.96 (m, 1H), 8.38-8.41 (m, 1H), 8.52-8.57 (m, 1H), 9.33 (br. s, 1H); m/z (ES+APCI)⁺: 419 [M+H]⁺.

### Example 62

### Thiophene-2-carboxylic acid {3-[5-cyclopropyl-2-(2-trifluoromethyl-1H-benzimidazol-5-ylamino)-pyrimidin-4-ylamino]-propyl}-amide

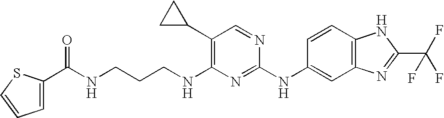

Prepared analogously to Example 28 from Intermediate 6 and 2-trifluoromethyl-1H-benzimidazol-5-ylamine to give the desired product as a white solid (10 mg, 13%). ¹H NMR (400 MHz, DMSO-d₆) δ ppm 0.46-0.52 (m, 2H), 0.80-0.87 (m, 2H), 1.45-1.52 (m, 1H), 1.84-1.92 (m, 2H), 3.21-3.45 (m, 2H), 3.46-3.58 (m, 2H), 6.84-6.89 (m, 1H), 7.12 (dd, J=5.0, 3.7 Hz, 1H), 7.42-7.63 (m, 2H), 7.65 (s, 1H), 7.68-7.75 (m, 2H), 8.43-8.47 (m, 1H), 8.49-8.64 (m, 2H), 9.13 (br. s, 1H); m/z (ES+APCI)⁺: 502 [M+H]⁺.

### Example 63

### Thiophene-2-carboxylic acid {3-[5-cyclopropyl-2-(pyridin-4-ylamino)-pyrimidin-4-ylamino]-propyl}-amide

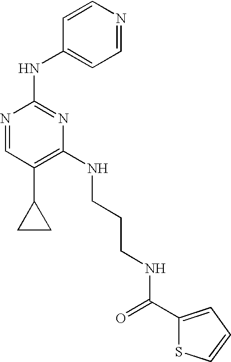

Prepared analogously to Example 51 from Intermediate 6 and 4-aminopyridine. m/z (ES+APCI)⁺: 395 [M+H]⁺.

### Example 64

### Cyclobutanecarboxylic acid {3-[5-cyclopropyl-2-(5-fluoro-pyridin-3-ylamino)-pyrimidin-4-ylamino]-propyl}-amide

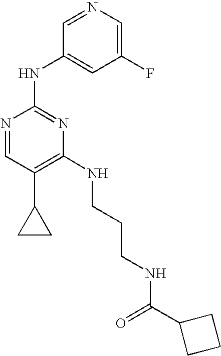

Intermediate 8 (45 mg, 0.146 mmol), 3-amino-5-fluoropyridine (20 mg, 0.175 mmol), tris(dibenzylideneacetone)dipalladium(0) (8 mg, 0.009 mmol), xantphos (7 mg, 0.012 mmol) and sodium tert-butoxide (42 mg, 0.437 mmol) were combined with dioxane (3 ml), sealed and then purged with nitrogen gas. The reaction mixture was heated at 100° C. for 18 hours, evaporated and purified through a silica plug, eluting with 0 to 10% methanol/DCM. Further purification by preparative LCMS (low pH buffer) followed by anion exchange chromatography of the resulting TFA salt using an Isolute-NH₂ cartridge to give the product as a white solid (28 mg, 50%). ¹H NMR (400 MHz, DMSO-d₆) δ ppm 0.46-0.51 (m, 2H), 0.81-0.88 (m, 2H), 1.40-1.53 (m, 1H), 1.64-1.78 (m, 3H), 1.79-1.92 (m, 1H), 1.92-2.03 (m, 2H), 2.03-2.18 (m, 2H), 2.91-3.02 (m, 1H), 3.08-3.20 (m, 2H), 3.38-3.47 (m, 2H), 6.97-7.07 (m, 1H), 7.67 (s, 1H), 7.68-7.74 (m, 1H), 7.97-8.04 (m, 1H), 8.29-8.40 (m, 1H), 8.60-8.65 (m, 1H), 9.47 (br. s, 1H); m/z (ES+APCI)⁺: 385 [M+H]⁺.

### Example 65

### Cyclobutanecarboxylic acid {3-[5-cyclopropyl-2-(pyridin-3-ylamino)-pyrimidin-4-ylamino]-propyl}-amide

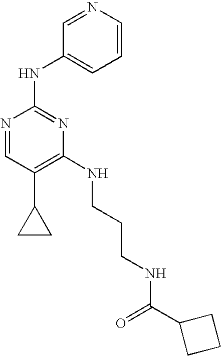

Prepared analogously to Example 64 from Intermediate 8 and 3-aminopyridine to give desired product as a white solid (22 mg, 31%). ¹H NMR (400 MHz, DMSO-d₆) δ ppm 0.44-0.50 (m, 2H), 0.80-0.86 (m, 2H), 1.43-1.51 (m, 1H), 1.65-1.77 (m, 3H), 1.78-1.92 (m, 1H), 1.93-2.03 (m, 2H), 2.04-2.17 (m, 2H), 2.91-3.02 (m, 1H), 3.10-3.18 (m, 2H), 3.39-3.45 (m, 2H), 6.89-6.94 (m, 1H), 7.24 (dd, J=8.5, 4.8 Hz, 1H), 7.63 (s, 1H), 7.67-7.73 (m, 1H), 8.04 (dd, J=4.6, 1.4 Hz, 1H), 8.23 (ddd, J=8.4, 2.6, 1.4 Hz, 1H), 8.86 (d, J=2.3 Hz, 1H), 9.10 (br. s, 1H); m/z (ES+APCI)⁺: 367 [M+H]⁺.

### Example 66

### Cyclopentanecarboxylic acid {3-[5-cyclopropyl-2-(pyridin-3-ylamino)-pyrimidin-4-ylamino]-propyl}-amide

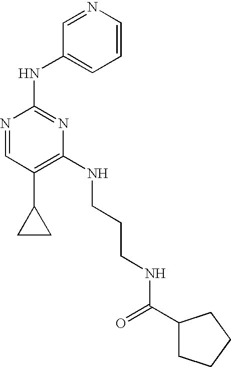

Intermediate 9 (63 mg, 0.195 mmol), 3-aminopyridine (22 mg, 0.234 mmol), tris(dibenzylideneacetone)dipalladium(0) (11 mg, 0.012 mmol), xantphos (9 mg, 0.016 mmol) and sodium tert-butoxide (56 mg, 0.585 mmol) were combined with dioxane (3 ml), sealed and then purged with nitrogen gas. The reaction mixture was heated at 100° C. for 18 hours, evaporated and eluted through a silica plug, then purified by preparative LCMS (low pH buffer). The resulting TFA salt was subjected to anion exchange chromatography using an Isolute-NH₂ cartridge to give the product as a white solid (15.5 mg, 21%). ¹H NMR (400 MHz, DMSO-d₆) δ ppm 0.43-0.50 (m, 2H), 0.79-0.86 (m, 2H), 1.42-1.51 (m, 3H), 1.54-1.65 (m, 4H), 1.65-1.75 (m, 4H), 2.46-2.55 (m, 1H), 3.09-3.18 (m, 2H), 3.39-3.47 (m, 2H), 6.87-6.96 (m, 1H), 7.24 (dd, J=8.2, 4.6 Hz, 1H), 7.63 (s, 1H), 7.77-7.88 (m, 1H), 8.04 (dd, J=4.6, 1.4 Hz, 1H), 8.23 (ddd, J=8.4, 1.4, 1.3 Hz, 1H), 8.86 (d, J=2.3 Hz, 1H), 9.12 (br. s, 1H); m/z (ES+APCI)⁺: 381 [M+H]⁺.

### Example 67

### Cyclopentanecarboxylic acid {3-[2-(3-acetylamino-phenylamino)-5-cyclopropyl-pyrimidin-4-ylamino]-propyl}-amide

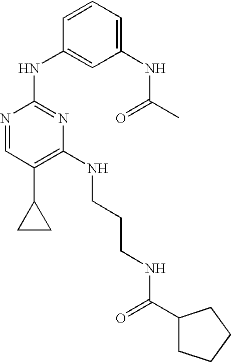

To a solution of Intermediate 5 (70 mg, 0.186 mmol) in DMF (2 ml) was added cyclopentanecarboxylic acid (30 μl, 0.279 mmol), then HATU (113 mg, 0.298 mmol) and N,N-diisopropylethylamine (194 μl, 1.17 mmol). The reaction was stirred at room temperature for 90 minutes. The mixture was evaporated then eluted through a 1 g Isolute-NH₂ cartridge with 9:1 DCM: methanol. The crude product was subjected to preparative LCMS (low pH buffer) followed by flash chromatography using a Biotage SP4 (gradient elution from 0-8% methanol in DCM). The resulting TFA salt was subjected to anion exchange chromatography using an Isolute-NH₂ cartridge to give a white solid (36 mg, 44%). ¹H NMR (400 MHz, DMSO-d₆, 85° C.) δ ppm 0.52-0.60 (m, 2H), 0.90-0.97 (m, 2H), 1.44-1.53 (m, 2H), 1.54-1.68 (m, 5H), 1.68-1.80 (m, 4H), 2.06 (s, 3H), 2.42-2.57 (m, 1H), 3.10-3.18 (m, 2H), 3.50-3.58 (m, 2H), 7.15-7.23 (m, 1H), 7.23-7.32 (m, 2H), 7.46-7.56 (m, 2H), 7.94-7.98 (m, 1H), 8.11-8.19 (m, 1H), 9.69-9.80 (m, 2H); m/z (ES+APCI)⁺: 437 [M+H]⁺.

### Example 68

### Cyclobutanecarboxylic acid {3-[2-(3-acetylamino-phenylamino)-5-cyclopropyl-pyrimidin-4-ylamino]-propyl}-amide

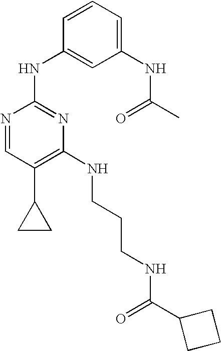

Prepared analogously to Example 69 from Intermediate 5 and cyclobutanecarboxylic acid to give the desired product as a white foam (12 mg, 13%). ¹H NMR (400 MHz, DMSO-d₆) δ ppm 0.42-0.50 (m, 2H), 0.78-0.87 (m, 2H), 1.42-1.51 (m, 1H), 1.61-1.78 (m, 3H), 1.80-1.91 (m, 1H), 1.92-2.04 (m, 5H), 2.03-2.17 (m, 2H), 2.89-3.00 (m, 1H), 3.08-3.15 (m, 2H), 3.40-3.49 (m, 2H), 6.79-6.85 (m, 1H), 7.04-7.13 (m, 2H), 7.37-7.41 (m, 1H), 7.59 (s, 1H), 7.64-7.70 (m, 1H), 7.93 (br. s, 1H), 8.87 (br. s, 1H), 9.78 (br. s, 1H); m/z (ES+APCI)⁺: 423 [M+H]⁺.

### Example 69

### Thiophene-2-carboxylic acid {3-[2-(3-amino-phenylamino)-5-cyclopropyl-pyrimidin-4-ylamino]-propyl}-amide

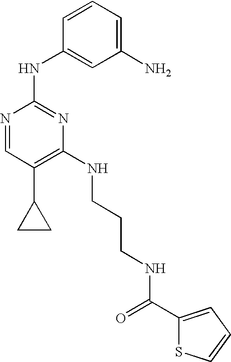

A suspension of Example 31 (32.8 mg, 0.064 mmol) in 4M hydrogen chloride in dioxane (1 ml, 4 mmol) was stirred vigorously for 4 h at room temperature The reaction mixture was evaporated to dryness and purified by preparative LCMS (high pH buffer) to give an off-white solid (17 mg, 65%). ¹H NMR (400 MHz, DMSO-d₆) δ ppm 0.42-0.50 (m, 2H), 0.79-0.86 (m, 2H), 1.41-1.51 (m, 1H), 1.78-1.89 (m, 2H), 3.25-3.37 (m, 2H), 3.45-3.54 (m, 2H), 6.11-6.16 (m, 1H), 6.81-6.89 (m, 2H), 6.97 (br. s, 1H), 7.01-7.16 (m, 2H), 7.55 (s, 1H), 7.69-7.77 (m, 2H), 8.52-8.59 (m, 1H), 8.76 (br. s, 1H); m/z (ES+APCI)⁺: 409 [M+H]⁺.

### Example 70

### Cyclobutanecarboxylic acid {3-[5-cyclopropyl-2-(3-hydroxymethyl-phenylamino)-pyrimidin-4-ylamino]-propyl}-amide. xHCl salt

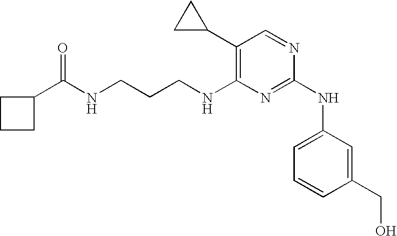

Example 70 was prepared analogously to Example 40 from Intermediate 8 and 3-(hydroxymethyl)aniline to give a white solid (26 mg, 37%). ¹H NMR (400 MHz, DMSO-d₆) δ ppm 0.53-0.60 (m, 2H), 0.82-0.94 (m, 2H), 1.50-1.59 (m, 1H), 1.65-1.78 (m, 3H), 1.78-1.91 (m, 1H), 1.92-2.02 (m, 2H), 2.02-2.15 (m, 2H), 2.89-3.01 (m, 1H), 3.06-3.16 (m, 2H), 3.44-3.54 (m, 2H), 4.51 (s, 2H), 7.06-7.10 (m, 1H), 7.31-7.36 (m, 1H), 7.39-7.45 (m, 1H), 7.58-7.65 (m, 2H), 7.71-7.84 (m, 1H), 8.61 (br. s, 1H), 10.35 (br. s, 1H), 11.98 (br. s, 1H); m/z (ES+APCI)⁺: 396 [M+H]⁺.

### Example 71

### 3-{4-[3-(Cyclobutanecarbonyl-amino)-propylamino]-5-cyclopropyl-pyrimidin-2-ylamino}-benzoic acid hydrochloride salt

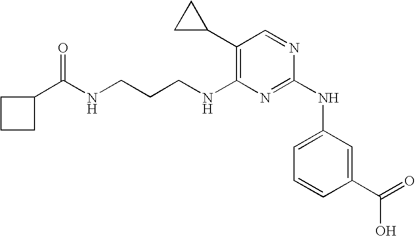

Example 71 was prepared analogously to Example 40 from Intermediate 8 and 3-aminobenzioc acid to give a white solid (139 mg, 22%). ¹H NMR (400 MHz, DMSO-d₆) δ ppm 0.43-0.50 (m, 2H), 0.78-0.85 (m, 2H), 1.42-1.50 (m, 1H), 1.64-1.76 (m, 3H), 1.78-1.90 (m, 1H), 1.91-2.01 (m, 2H), 2.03-2.15 (m, 2H), 2.89-2.99 (m, 1H), 3.08-3.15 (m, 2H), 3.43-3.49 (m, 2H), 6.83-6.90 (m, 1H), 7.27-7.33 (m, 1H), 7.39-7.43 (m, 1H), 7.62 (s, 1H), 7.64-7.70 (m, 1H), 7.86 (dd, 8.2, 1.4 Hz, 1H), 8.56-8.58 (m, 1H), 9.12 (br. s, 1H); m/z (ES+APCI)⁺: 410 [M+H]⁺.

### Example 72

### Cyclobutanecarboxylic acid {3-[5-cyclopropyl-2-(3,5-difluoro-phenylamino)-pyrimidin-4-ylamino]-propyl}-amide. Hydrochloride salt

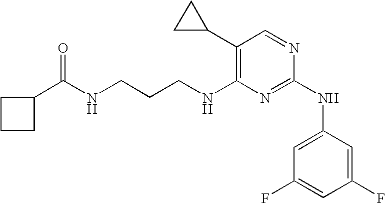

Example 72 was prepared analogously to Example 40 from Intermediate 8 and 3,5-difluoroaniline to give a white solid (16 mg, 22%). ¹H NMR (400 MHz, DMSO-d₆) δ ppm 0.55-0.62 (m, 2H), 0.89-0.96 (m, 2H), 1.52-1.61 (m, 1H), 1.66-1.80 (m, 3H), 1.81-1.91 (m, 1H), 1.92-2.02 (m, 2H), 2.03-2.15 (m, 2H), 2.90-3.01 (m, 1H), 3.08-3.16 (m, 2H), 3.46-3.53 (m, 2H), 6.95-7.04 (m, 1H), 7.33-7.42 (m, 2H), 7.69 (s, 1H), 7.76-7.83 (m, 1H), 8.72 (br. s, 1H), 10.83 (br. s, 1H); m/z (ES+APCI)⁺: 402.2 [M+H]⁺.

### Example 73

### 3-{4-[3-(Cyclobutanecarbonyl-amino)-propylamino]-5-cyclopropyl-pyrimidin-2-ylamino}-N-(2-methoxy-ethyl)-benzamide

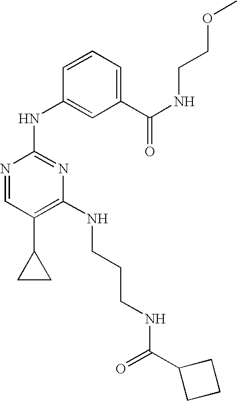

To a solution of 2-methoxyethylamine (9 μl, 0.1 mmol) in DMF (1 ml) was added Example 71 (30 mg, 0.067 mmol), HATU (41 mg, 0.11 mmol) and diisopropylethylamine (70 μl, 0.4 mmol) and the resulting mixture was stirred at room temperature for 72 hours. The mixture was evaporated then eluted through a 1 g Isolute-NH₂ cartridge with 9:1 DCM: methanol eluent, then subjected to preparative LCMS (low pH buffer). The resulting TFA salt was subjected anion exchange chromatography using an Isolute-NH₂ cartridge to give the title compound as a white solid (20 mg, 64%). ¹H NMR (400 MHz, DMSO-d₆) δ ppm 0.43-0.50 (m, 2H), 0.79-0.86 (m, 2H), 1.42-1.52 (m, 1H), 1.63-1.77 (m, 3H), 1.78-1.91 (m, 1H), 1.91-2.01 (m, 2H), 2.04-2.16 (m, 2H), 2.89-2.99 (m, 1H), 3.08-3.16 (m, 2H), 3.26 (s, 3H), 3.35-3.49 (m, 6H), 6.81-6.89 (m, 1H), 7.23-7.30 (m, 2H), 7.61 (s, 1H), 7.66-7.72 (m, 1H), 7.77-7.81 (m, 1H), 8.31-8.39 (m, 2H), 9.04 (br. s, 1H); m/z (ES+APCI)⁺: 467 [M+H]⁺.

### Examples 74-82

Examples 74-82 were prepared analogously to example M5 (the general structure is shown below followed by the tabulated examples).

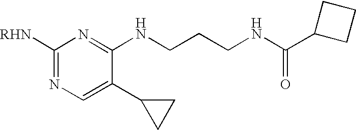

| | | | | HPLC |
|---|---|---|---|---|
| | | | m/z | retention |
| Example | R group | Name | (ES + APCI)⁺ | time (min)* |
| 74 | 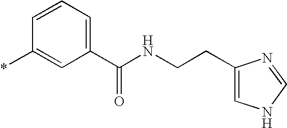 | 3-{4-[3- (Cyclobutanecarbonyl- amino)-propylamino]-5- cyclopropyl-pyrimidin-2- ylamino}-N-[2-(1H- imidazol-4-yl)-ethyl]- benzamide | 503 | 6.88 |
| 75 | 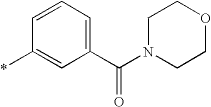 | Cyclobutanecarboxylic acid (3-{5-cyclopropyl-2- [3-(morpholine-4- carbonyl)-phenylamino]- pyrimidin-4-ylamino}- propyl)-amide | 479 | 7.33 |
| 76 | 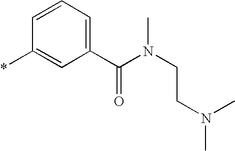 | 3-{4-[3- (Cyclobutanecarbonyl- amino)-propylamino]-5- cyclopropyl-pyrimidin-2- ylamino}-N-(2- dimethylamino-ethyl)-N- methyl-benzamide | 494 | 6.71 |
| 77 | 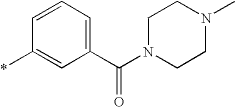 | Cyclobutanecarboxylic acid (3-{5-cyclopropyl-2- [3-(4-methyl-piperazine- 1-carbonyl)- phenylamino]-pyrimidin- 4-ylamino}-propyl)- amide | 492 | 6.63 |
| 78 | 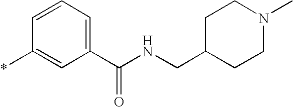 | 3-{4-[3- (Cyclobutanecarbonyl- amino)-propylamino]-5- cyclopropyl-pyrimidin-2- ylamino}-N-(1-methyl- piperidin-4-ylmethyl)- benzamide | 520 | 8.43 |
| 79 | 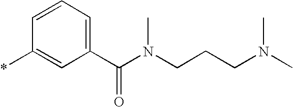 | 3-{4-[3- (Cyclobutanecarbonyl- amino)-propylamino]-5- cyclopropyl-pyrimidin-2- ylamino}-N-(3- dimethylamino-propyl)- N-methyl-benzamide | 508 | 6.69 |
| 80 | 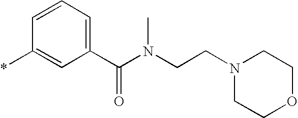 | 3-{4-[3- (Cyclobutanecarbonyl- amino)-propylamino]-5- cyclopropyl-pyrimidin-2- ylamino}-N-methyl-N-(2- morpholin-4-yl-ethyl)- benzamide | 536 | 6.73 |
| 81 | 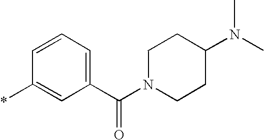 | Cyclobutanecarboxylic acid (3-{5-cyclopropyl-2- [3-(4-dimethylamino- piperidine-1-carbonyl)- phenylamino]-pyrimidin- 4-ylamino}-propyl)- amide | 520 | 6.64 |
| 82 | 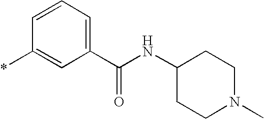 | 3-{4-[3- (Cyclobutanecarbonyl- amino)-propylamino]-5- cyclopropyl-pyrimidin-2- ylamino}-N-(1-methyl- piperidin-4-yl)- benzamide | 506 | 6.75 |

### Example 83

### Cyclobutanecarboxylic acid {3-[5-cyclopropyl-2-(3-1,2,4-triazol-1-ylmethyl-phenylamino)-pyrimidin-4-ylamino]-propyl}-amide

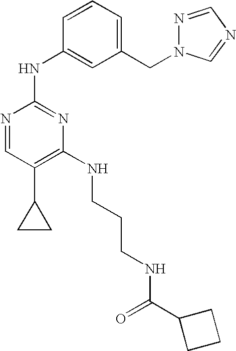

Example 83 was prepared analogously to Example 43 from Intermediate 8 and 3-(4H-1,2,4-triazol-4-ylmethyl)aniline to give the title compound as a white solid (45 mg, 62%). ¹H NMR (400 MHz, DMSO-d₆) δ ppm 0.43-0.49 (m, 2H), 0.79-0.85 (m, 2H), 1.42-1.50 (m, 1H), 1.65-1.77 (m, 3H), 1.78-1.91 (m, 1H), 1.92-2.01 (m, 2H), 2.04-2.15 (m, 2H), 2.90-2.99 (m, 1H), 3.09-3.16 (m, 2H), 3.38-3.45 (m, 2H), 5.34 (s, 2H), 6.69-6.73 (m, 1H), 6.80-6.86 (m, 1H), 7.15-7.21 (m, 1H), 7.60 (s, 1H), 7.62-7.71 (m, 2H), 7.74-7.78 (m, 1H), 7.97 (s, 1H), 8.62 (s, 1H), 8.96 (br. s, 1H); m/z (ES+APCI)⁺: 447 [M+H]⁺.

### Examples 84-87

Examples 84-87 were prepared in a similar manner to Example 83 (the general structure is shown below followed by the tabulated examples).

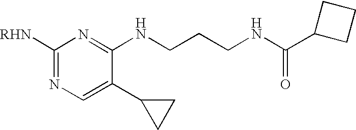

| | | | | HPLC |
|---|---|---|---|---|
| | | | m/z | retention |
| Example | R group | Name | (ES + APCI)⁺ | time (min)* |
| 84 | 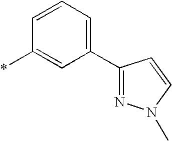 | Cyclobutanecarboxylic acid (3-{5-cyclopropyl-2- [3-(1-methyl-1H-pyrazol- 3-yl)-phenylamino]- pyrimidin-4-ylamino}- propyl)-amide | 446 | 10.31 |
| 85 | 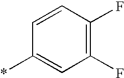 | Cyclobutanecarboxylic acid {3-[5-cyclopropyl-2- (3,4-difluoro- phenylamino)-pyrimidin- 4-ylamino]-propyl}- amide | 402 | 8.12 |
| 86 | 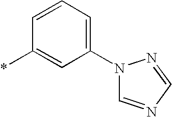 | Cyclobutanecarboxylic acid (3-{5-cyclopropyl-2- [3-(2H-1,2,4-triazol-1- yl)-phenylamino]- pyrimidin-4-ylamino}- propyl)-amide | 433 | 7.68 |
| 87 | 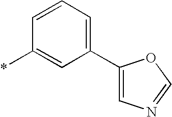 | Cyclobutanecarboxylic acid {3-[5-cyclopropyl-2- (3-oxazol-5-yl- phenylamino)-pyrimidin- 4-ylamino]-propyl}- amide | 433 | 10.36 |

### Example 88

### Cyclobutanecarboxylic acid {3-[5-cyclopropyl-2-(3-oxo-2,3-dihydro-1H-isoindol-5-ylamino)-pyrimidin-4-ylamino]-propyl}-amide hydrochloride salt

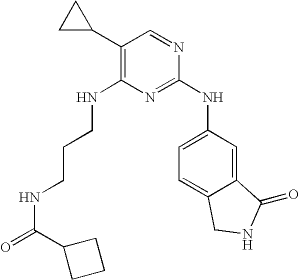

Example 88 analogously to Example 40 from Intermediate 8 and 6-amino-2,3-dihydro-1H-isoindol-1-one to give the desired product as a pale yellow solid (23 mg, 31%). ¹H NMR (400 MHz, DMSO-d₆) δ ppm 0.54-0.61 (m, 2H), 0.87-0.95 (m, 2H), 1.50-1.60 (m, 1H), 1.65-1.78 (m, 3H), 1.79-1.90 (m, 1H), 1.90-2.00 (m, 2H), 2.02-2.15 (m, 2H), 2.88-3.00 (m, 1H), 3.09-3.18 (m, 2H), 3.44-3.53 (m, 2H), 4.37 (s, 2H), 7.57-7.68 (m, 3H), 7.77-7.82 (m, 1H), 8.06 (d, J=1.4 Hz, 1H), 8.62-8.75 (m, 2H), 10.64 (br. s, 1H); m/z (ES+APCI)⁺: 421 [M+H]⁺.

### Example 89

### 3-{4-[3-(Cyclobutanecarbonyl-amino)-propylamino]-5-cyclopropyl-pyrimidin-2-ylamino}-N-(1H-imidazol-2-ylmethyl)-benzamide. Hydrochloride salt

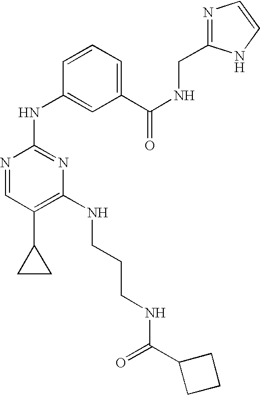

To a solution of C-(1H-Imidazol-2-yl)-methylamine (6 mg, 0.13 mmol) in DMF (1 ml) was added Example 71 (40 mg, 0.089 mmol), HATU (36 mg, 0.14 mmol) and diisopropylethylamine (62 μl, 0.54 mmol). The reaction was stirred at room temperature for 18 hours. The reaction mixture was evaporated, triturated with methanol, dried to give the title compound as a white solid (6 mg, 18%). ¹H NMR (400 MHz, DMSO-d₆) δ ppm 0.43-0.49 (m, 2H), 0.79-0.85 (m, 2H), 1.43-1.51 (m, 1H), 1.63-1.77 (m, 3H), 1.78-1.90 (m, 1H), 1.91-2.01 (m, 2H), 2.04-2.15 (m, 2H), 2.88-2.99 (m, 1H), 3.08-3.15 (m, 2H), 3.41-3.47 (m, 2H), 4.46 (d, J=5.5 Hz, 2H), 6.78-6.87 (m, 2H), 6.98-7.03 (m, 1H), 7.25-7.30 (m, 1H), 7.33-7.38 (m, 1H), 7.61 (s, 1H), 7.68-7.73 (m, 1H), 7.80-7.84 (m, 1H), 8.36-8.39 (m, 1H), 8.76-8.81 (m, 1H), 9.01-9.08 (m, 1H), 11.76 (br. s, 1H); m/z (ES+APCI)⁺: 489 [M+H]⁺.

### Example 90

### Cyclobutanecarboxylic acid {3-[5-cyclopropyl-2-(1-oxo-2,3-dihydro-1H-isoindol-5-ylamino)-pyrimidin-4-ylamino]-propyl}-amide. Trifluoroacetic acid salt

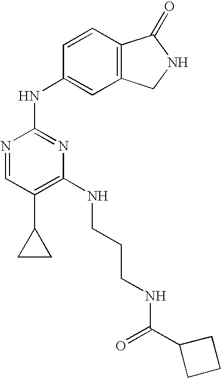

Intermediate 8 (50 mg, 0.162 mmol), 5-amino-2,3-dihydro-isoindol-1-one (72 mg, 0.485 mmol), glacial acetic acid (9 μl, 0.162 mmol) and n-butanol (1.5 ml) were combined and irradiated at 150° C. for 60 minutes in a Biotage I-60 microwave reactor. The reaction was evaporated, purified by LCMS (low pH buffer) triturated with methanol and dried to give the title compound as a white solid (6 mg, 7%). ¹H NMR (400 MHz, DMSO-d₆) δ ppm 0.55-0.61 (m, 2H), 0.87-0.93 (m, 2H), 1.50-1.57 (m, 1H), 1.64-1.77 (m, 3H), 1.78-1.90 (m, 1H), 1.91-2.00 (m, 2H), 2.02-2.13 (m, 2H), 2.88-2.98 (m, 1H), 3.09-3.16 (m, 2H), 3.28-3.56 (m, 2H), 4.38 (s, 2H), 7.61-7.69 (m, 3H), 7.71-7.77 (m, 1H), 7.86-7.91 (m, 1H), 8.37-8.53 (m, 2H), 10.39 (br. s, 1H); m/z (ES+APCI)⁺: 421 [M+H]⁺.

### Example 91

### Cyclobutanecarboxylic acid (3-{5-cyclopropyl-2-[3-(4-methyl-piperazin-1-yl)-phenylamino]-pyrimidin-4-ylamino}-propyl)-amide. Trifluoroacetic acid salt

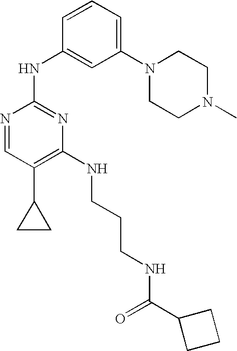

Intermediate 8 (40 mg, 0.130 mmol), 3-(4-methyl-piperazin-1-yl)-phenylamine (30 mg, 0.155 mmol), Pd₂(dba)₃ (7 mg, 0.008 mmol), xantphos (6 mg, 0.01 mmol) and sodium tert-butoxide (37 mg, 0.385 mmol) were combined with dioxane (1 ml), sealed and then purged with nitrogen gas. The reaction mixture was heated at 90° C. for 18 hours, evaporated and purified through a silica plug, eluting with 0 to 10% methanol/DCM. The crude product was further purified by LCMS to give the desired product as a white solid (24 mg, 32%). ¹H NMR (400 MHz, DMSO-d₆, 85° C.) δ ppm 0.54-0.61 (m, 2H), 0.90-0.97 (m, 2H), 1.53-1.63 (m, 1H), 1.70-1.94 (m, 4H), 1.98-2.19 (m, 4H), 2.88 (s, 3H), 2.92-3.02 (m, 1H), 3.11-3.18 (m, 2H), 3.24-3.55 (m, 10H), 6.80 (dd, J=8.0, 2.1 Hz, 1H), 7.11 (dd, J=8.0, 1.6 Hz, 1H), 7.16-7.20 (m, 1H), 7.24-7.31 (m, 1H), 7.44 (br. s, 1H), 7.55 (s, 1H), 8.12 (br. s, 1H), 9.82 (br. s, 1H); m/z (ES+APCI)⁺: 464 [M+H]⁺.

### Example 92

### Cyclobutanecarboxylic acid {3-[5-cyclopropyl-2-(3-morpholin-4-ylmethyl-phenylamino)-pyrimidin-4-ylamino]-propyl}-amide. Trifluoroacetic acid salt

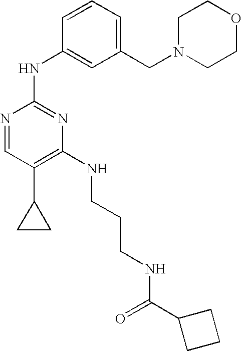

Example 92 was prepared in a similar manner to Example 91 from Intermediate 8 and 3-(morpholin-4-ylmethyl)aniline to give the desired product as a white solid (21 mg, 28%). ¹H NMR (400 MHz, DMSO-d₆, 85° C.) δ ppm 0.56-0.62 (m, 2H), 0.91-0.97 (m, 2H), 1.55-1.63 (m, 1H), 1.71-1.83 (m, 3H), 1.83-1.94 (m, 1H), 1.98-2.18 (m, 4H), 2.92-3.02 (m, 1H), 3.03-3.18 (m, 6H), 3.47-3.54 (m, 2H), 3.74-3.84 (m, 4H), 4.18-4.24 (m, 2H), 7.24-7.29 (m, 1H), 7.43-7.50 (m, 2H), 7.60 (s, 1H), 7.63-7.65 (m, 1H), 7.65-7.70 (m, 1H), 8.22 (br. s, 1H), 10.16 (br. s, 1H); m/z (ES+APCI)⁺: 465 [M+H]⁺.

### Example 93

### Cyclobutanecarboxylic acid {3-[5-cyclopropyl-2-(6-morpholin-4-yl-pyridin-3-ylamino)-pyrimidin-4-ylamino]-propyl}-amide

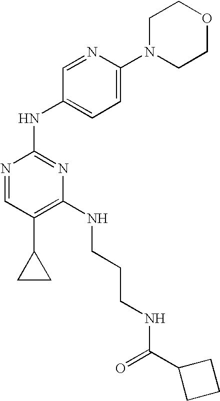

Intermediate 8 (40 mg, 0.130 mmol), 6-morpholin-4-yl-pyridin-3-ylamine (28 mg, 0.155 mmol), Pd₂(dba)₃ (7 mg, 0.008 mmol), xantphos (6 mg, 0.01 mmol) and sodium tert-butoxide (37 mg, 0.385 mmol) were combined with dioxane (1 ml), sealed and then purged with nitrogen gas. The reaction mixture was heated at 90° C. for 18 hours, evaporated and purified through a silica plug, eluting with 0 to 10% methanol/DCM. The crude product was triturated with diethyl ether, filtered and dried to give the desired product as a white solid (10 mg, 18%). ¹H NMR (400 MHz, DMSO-d₆) δ ppm 0.40-0.48 (m, 2H), 0.76-0.85 (m, 2H), 1.39-1.50 (m, 1H), 1.62-1.78 (m, 3H), 1.79-1.92 (m, 1H), 1.92-2.02 (m, 2H), 2.04-2.16 (m, 2H), 2.90-3.01 (m, 1H), 3.08-3.15 (m, 2H), 3.28-3.33 (m, 4H), 3.35-3.42 (m, 2H), 3.67-3.73 (m, 4H), 6.73-6.82 (m, 2H), 7.56 (s, 1H), 7.63-7.71 (m, 1H), 7.97 (dd, J=9.2, 2.7 Hz, 1H), 8.42-8.46 (m, 1H), 8.67 (br. s, 1H); m/z (ES+APCI)⁺: 452 [M+H]⁺.

### Example 94

### Cyclobutanecarboxylic acid (3-{5-cyclopropyl-2-[3-(2H-tetrazol-5-yl)-phenylamino]-pyrimidin-4-ylamino}-propyl)-amide

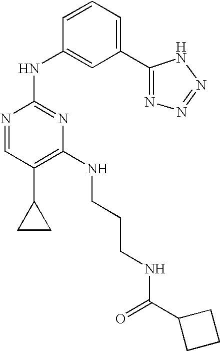

Intermediate 8 (50 mg, 0.162 mmol), 3-(2H-tetrazol-5-yl)-phenylamine (78 mg, 0.484 mmol), glacial acetic acid (9 μl, 0.162 mmol) and n-butanol (1.5 ml) were combined and irradiated at 150° C. for 40 minutes in a Biotage I-60 microwave reactor. The mixture was evaporated and purified by flash chromatography on the Biotage SP4, eluting with 0 to 12% methanol/DCM. The product was then eluted through a 0.5 g Isolute-NH₂ cartridge with 9:1 DCM:methanol to give the free base as a white solid (16 mg, 23%). ¹H NMR (400 MHz, DMSO-d₆, 85° C.) δ ppm 0.46-0.50 (m, 2H), 0.82-0.87 (m, 2H), 1.47-1.55 (m, 1H), 1.68-1.87 (m, 4H), 1.92-2.01 (m, 2H), 2.05-2.16 (m, 2H), 2.89-2.98 (m, 1H), 3.15-3.21 (m, 2H), 3.53-3.60 (m, 2H), 6.41-6.47 (m, 1H), 7.15-7.21 (m, 1H), 7.49 (dd, J=7.6, 1.6 Hz, 1H), 7.50-7.53 (m, 1H), 7.61 (s, 1H), 7.65-7.71 (m, 1H), 8.45 (br. s, 1H), 8.55-8.58 (m, 1H); m/z (ES+APCI)⁻: 432 [M−H]⁻.

### Example 95

### (3-{4-[3-(Cyclobutanecarbonyl-amino)-propylamino]-5-cyclopropyl-pyrimidin-2-ylamino}-phenyl)-acetic acid. Hydrochloride

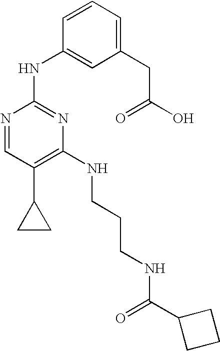

Intermediate 8 (50 mg, 0.162 mmol), (3-amino-phenyl)-acetic acid (74 mg, 0.484 mmol), glacial acetic acid (9 μl, 0.162 mmol) and n-butanol (1.5 ml) were combined and irradiated at 150° C. for 40 minutes in a Biotage I-60 microwave reactor. The mixture was evaporated and purified by flash chromatography on the Biotage SP4, eluting with 0 to 12% methanol/DCM to give the product as a white solid (54 mg, 73%). ¹H NMR (400 MHz, DMSO-d₆, 85° C.) δ ppm 0.53-0.59 (m, 2H), 0.88-0.96 (m, 2H), 1.53-1.63 (m, 1H), 1.72-1.94 (m, 4H), 1.99-2.08 (m, 2H), 2.09-2.19 (m, 2H), 2.90-3.21 (m, 3H), 3.47-3.59 (m, 4H), 6.94-7.02 (m, 1H), 7.24-7.32 (m, 1H), 7.42-7.52 (m, 2H), 7.56-7.64 (m, 2H), 7.86 (br. s, 1H), 9.75 (br. s, 1H); m/z (ES+APCI)⁺: 424 [M+H]⁺.

### Example 96

### Cyclobutanecarboxylic acid {3-[2-(3H-benzimidazol-5-ylamino)-5-cyclopropyl-pyrimidin-4-ylamino]-propyl}-amide

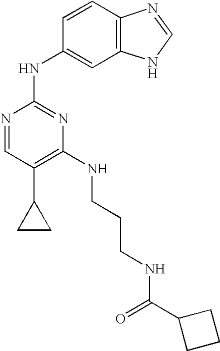

Intermediate 8 (50 mg, 0.162 mmol), 3H-benzimidazol-5-ylamine (65 mg, 0.484 mmol), glacial acetic acid (9 μl, 0.162 mmol) and n-butanol (1.5 ml) were combined and irradiated at 150° C. for 40 minutes in a Biotage I-60 microwave reactor. The mixture was evaporated and purified by flash chromatography on the Biotage SP4, eluting with 0 to 12% methanol/DCM. The product was then eluted through a 0.5 g Isolute-NH₂ cartridge with 9:1 DCM: methanol. Further purification by LCMS (low pH buffer) gave the title compound as a white solid (13 mg, 15%). ¹H NMR (400 MHz, DMSO-d₆) δ ppm 0.58-0.63 (m, 2H), 0.88-0.94 (m, 2H), 1.52-1.60 (m, 1H), 1.64-2.00 (m, 7H), 2.02-2.13 (m, 2H), 2.90-3.00 (m, 1H), 3.10-3.17 (m, 2H), 3.45-3.56 (m, 2H), 7.61 (dd, J=8.7, 1.8 Hz, 1H), 7.68 (s, 1H), 7.80-7.87 (m, 2H), 8.23 (d, J=1.4 Hz, 1H), 8.56-8.62 (m, 1H), 9.33 (s, 1H), 10.94 (br. s, 1H); m/z (ES+APCI)⁺: 406 [M+H]⁺.

### Example 97

### Cyclobutanecarboxylic acid {3-[2-(6-bromo-pyridin-3-ylamino)-5-cyclopropyl-pyrimidin-4-ylamino]-propyl}-amide

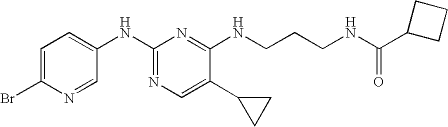

A mixture of Intermediate 8 (200 mg, 0.649 mmol), 2-bromo-5-aminopyridine (225 mg, 1.30 mmol) and AcOH (37 μl, 0.649 mmol) in nBuOH were charged into a microwave reactor vial and sealed. The vial was placed in the Biotage I-60 microwave reactor and irradiated at 150° C. for 40 minutes. After the reaction a precipitate was observed. The mixture was filtered and the collected solid was washed with diethyl ether. The solid was dispersed into DCM and enough MeOH was added to give a clear solution. The solution was then washed with saturated aqueous sodium carbonate solution. The organic phase was dried and concentrated to give an off-white solid (214 mg, 74%). ¹H NMR (400 MHz, DMSO-d₆) δ ppm 0.44-0.50 (2H, m), 0.79-0.86 (2H, m), 1.43-1.51 (1H, m), 1.63-1.77 (3H, m), 1.81-2.02 (3H, m), 2.04-2.16 (2H, m), 2.92-3.02 (1H, m), 3.05-3.17 (2H, m), 3.37-3.45 (2H, m), 6.96 (1H, t, J=5.72 Hz), 7.48 (1H, d, J=9.16 Hz), 7.62-7.72 (2H, m), 8.16 (1H, dd, J=8.70, 2.75 Hz), 8.76 (1H, d, J=2.75 Hz), 9.29 (1H, s); m/z (ES+APCI)⁺: 445/447 [M+H]⁺.

### Example 98

### Cyclobutanecarboxylic acid {3-[5-cyclopropyl-2-(6-iodo-pyridin-3-ylamino)-pyrimidin-4-ylamino]-propyl}-amide

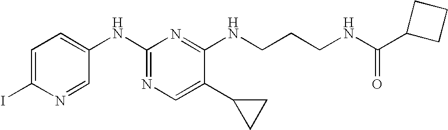

Prepared analogously to Example 97 from Intermediate 8 and 2-iodo-5-aminopyridine to give an off-white solid (209 mg, 65%). ¹H NMR (400 MHz, DMSO-d₆) δ ppm 0.42-0.50 (2H, m), 0.77-0.86 (2H, m), 1.43-1.51 (1H, m), 1.62-1.77 (3H, m), 1.82-2.02 (3H, m), 2.04-2.16 (2H, m), 2.97 (1H, m), 3.12 (2H, m), 3.37-3.45 (2H, m), 6.95 (1H, t, J=5.95 Hz), 7.57-7.72 (3H, m), 7.97 (1H, dd, J=8.70, 2.75 Hz), 8.76 (1H, d, J=2.75 Hz), 9.25 (1H, s); m/z (ES+APCI)⁺: 493 [M+H]⁺.

### Example 99

### Cyclobutanecarboxylic acid {3-[2-(6-chloro-pyridin-3-ylamino)-5-cyclopropyl-pyrimidin-4-ylamino]-propyl}-amide

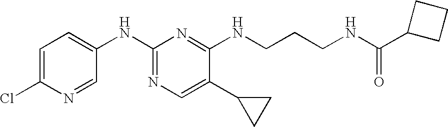

Prepared analogously to Example 97 from Intermediate 8 and 2-chloro-5-aminopyridine to give a mauve coloured solid (888 mg, 61%). ¹H NMR (400 MHz, DMSO-d₆) δ ppm 0.43-0.51 (2H, m), 0.78-0.88 (2H, m), 1.44-1.52 (1H, m), 1.64-1.78 (3H, m), 1.80-2.03 (3H, m), 2.05-2.17 (2H, m), 2.98 (1H, m), 3.07-3.24 (2H, m), 3.38-3.46 (2H, m), 6.97 (1H, t, J=5.72 Hz), 7.37 (1H, d, J=8.70 Hz), 7.61-7.74 (2H, m), 8.26 (1H, dd, J=8.93, 2.98 Hz), 8.77 (1H, d, J=2.75 Hz), 9.30 (1H, s); m/z (ES+APCI)⁺: 401/403 [M+H]⁺.

### Example 100

### Cyclobutanecarboxylic acid [3-(5-cyclopropyl-2-{6-[(2-dimethylamino-ethyl)-methyl-amino]-pyridin-3-ylamino}-pyrimidin-4-ylamino)-propyl]-amide

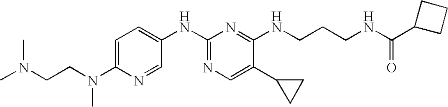

Prepared analogously to Example 97 from intermediate 8 and Intermediate 13 to give a purple coloured foamy solid (18 mg, 24%). ¹H NMR (400 MHz, DMSO-d₆) δ ppm 0.39-0.49 (2H, m), 0.76-0.84 (2H, m), 1.40-1.47 (1H, m), 1.60-1.77 (3H, m), 1.82-1.90 (1H, m), 1.91-2.02 (2H, m), 2.04-2.20 (8H, m), 2.36 (2H, t, J=7.10 Hz), 2.81-3.05 (4H, m), 3.07-3.16 (2H, m), 3.33-3.42 (2H, m), 3.55 (2H, t, J=6.87 Hz), 6.52 (1H, d, J=9.16 Hz), 6.74 (1H, t, J=5.72 Hz), 7.53 (1H, s), 7.69 (1H, t, J=5.72 Hz), 7.86 (1H, dd, J=9.16, 2.75 Hz), 8.30 (1H, d, J=2.29 Hz), 8.50 (1H, s); m/z (ES+APCI)⁺: 467 [M+H]⁺.

### Example 101

### Cyclobutanecarboxylic acid [3-(5-cyclopropyl-2-{4-[(2-hydroxy-ethyl)-methyl-amino]-phenylamino}-pyrimidin-4-ylamino)-propyl]-amide

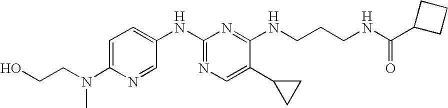

Prepared analogously to Example 97 from Intermediate 8 and Intermediate 15 to give a purple coloured foamy solid (36 mg, 50%). ¹H NMR (400 MHz, DMSO-d₆) δ ppm 0.40-0.48 (2H, m), 0.77-0.86 (2H, m), 1.39-1.47 (1H, m), 1.61-1.77 (3H, m), 1.79-1.91 (1H, m), 1.92-2.02 (2H, m), 2.05-2.16 (2H, m), 2.91-3.06 (4H, m), 3.06-3.15 (2H, m), 3.30-3.42 (2H, m), 3.47-3.58 (4H, m), 4.66-4.71 (1H, m), 6.54 (1H, d, J=9.16 Hz), 6.73 (1H, t, J=5.95 Hz), 7.53 (1H, s), 7.67 (1H, t, J=5.72 Hz), 7.85 (1H, dd, J=9.16, 2.75 Hz), 8.29 (1H, d, J=2.75 Hz), 8.48 (1H, s); m/z (ES+APCI)⁺: 441 [M+H]⁺.

### Example 102

### Cyclobutanecarboxylic acid [3-(5-cyclopropyl-2-{6-[methyl-(2-morpholin-4-yl-ethyl)-amino]-pyridin-3-ylamino}-pyrimidin-4-ylamino)-propyl]-amide

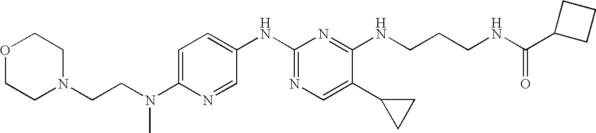

Prepared analogously to Example 97 from Intermediate 8 and Intermediate 17 to give a purple coloured foamy solid (38 mg, 46%). ¹H NMR (400 MHz, DMSO-d₆) δ ppm 0.40-0.48 (2H, m), 0.76-0.86 (2H, m), 1.39-1.47 (1H, m), 1.59-1.77 (3H, m), 1.79-2.02 (3H, m), 2.04-2.16 (2H, m), 2.37-2.47 (6H, m), 2.89-3.00 (4H, m), 3.06-3.15 (2H, m), 3.34-3.43 (2H, m), 3.48-3.65 (6H, m), 6.53 (1H, d, J=9.16 Hz), 6.72 (1H, t, J=5.95 Hz), 7.53 (1H, s), 7.66 (1H, t, J=5.72 Hz), 7.86 (1H, dd, J=9.16, 2.75 Hz), 8.30 (1H, d, J=2.29 Hz), 8.48 (1H, s); m/z (ES+APCI)⁺: 510 [M+H]⁺.

### Example 103

### Cyclobutanecarboxylic acid {3-[5-cyclopropyl-2-(6-hydroxymethyl-pyridin-3-ylamino)-pyrimidin-4-ylamino]-propyl}-amide

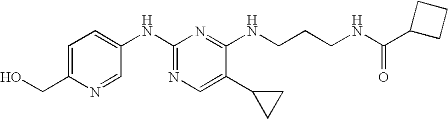

A solution of Intermediate 21 (590 mg, 1.16 mmol) and tetrabutylammonium fluoride trihydrate (1.1 g, 3.47 mmol) in THF (40 ml) was stirred at rt for 90 minutes. The mixture was then concentrated to dryness and the residue was diluted with DCM. The DCM extract was washed with water, dried and concentrated. The residue was purified by trituration with diethyl ether twice, successively to afford a pale brown coloured solid (446 mg, 97%). ¹H NMR (400 MHz, DMSO-d₆) δ ppm 0.44-0.50 (2H, m), 0.79-0.87 (2H, m), 1.43-1.51 (1H, m), 1.65-1.78 (3H, m), 1.82-1.92 (1H, m), 1.93-2.03 (2H, m), 2.06-2.16 (2H, m), 2.93-3.02 (1H, m), 3.09-3.19 (2H, m), 3.42 (2H, q, J=6.41 Hz), 4.47 (2H, d, J=5.50 Hz), 5.23 (1H, t, J=5.72 Hz), 6.88 (1H, t, J=5.95 Hz), 7.31 (1H, d, J=8.70 Hz), 7.62 (1H, s), 7.69 (1H, t, J=5.72 Hz), 8.18 (1H, dd, J=8.47, 2.52 Hz), 8.80 (1H, d, J=2.29 Hz), 9.04 (1H, s); m/z (ES+APCI)⁺: 397 [M+H]⁺.

### Example 104

### Cyclobutanecarboxylic acid {3-[5-cyclopropyl-2-(6-morpholin-4-ylmethyl-pyridin-3-ylamino)-pyrimidin-4-ylamino]-propyl}-amide

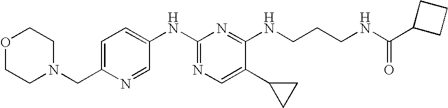

A mixture of Intermediate 22 (30 mg), morpholine (8.2 μl, 0.095 mmol) and DIPEA (66 μl, 0.379 mmol) in DCM (2 ml) were stirred at rt over the weekend. The mixture was then diluted with DCM and washed with saturated aqueous sodium hydrogen carbonate solution. The organic phase was dried and concentrated. The crude residue was purified by flash column chromatography on silica gel (3 g) eluting with 10:1 DCM:MeOH to give a white solid (14 mg). ¹H NMR (400 MHz, DMSO-d₆) δ ppm 0.44-0.49 (2H, m), 0.80-0.86 (2H, m), 1.43-1.50 (1H, m), 1.65-1.77 (3H, m), 1.81-1.89 (1H, m), 1.93-2.02 (2H, m), 2.05-2.16 (2H, m), 2.32-2.43 (4H, m), 2.93-3.02 (1H, m), 3.09-3.17 (2H, m), 3.37-3.50 (4H, m), 3.52-3.60 (4H, m), 6.88 (1H, t, J=5.95 Hz), 7.27 (1H, d, J=8.24 Hz), 7.62 (1H, s), 7.69 (1H, t, J=5.72 Hz), 8.18 (1H, dd, J=8.47, 2.52 Hz), 8.78 (1H, d, J=2.29 Hz), 9.05 (1H, s); m/z (ES+APCI)⁺: 466 [M+H]⁺.

### Example 105

### {3-[5-Cyclopropyl-2-(3-fluoro-phenylamino)-pyrimidin-4-yloxy]-propyl}-carbamic acid tert-butyl ester

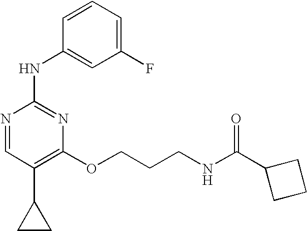

To solution of Intermediate 25 (24 mg, 0.071 mmol) in DMF (1 ml) was sequentially added added cyclobutanecarboxylic acid (10.2 μl, 0.11 mmol), HATU (43 mg, 0.11 mmol) and N,N-diisopropylethylamine (74 μl, 0.43 mmol) and the reaction mixture left to stir at room temperature overnight. The solvents were removed under reduced pressure and the crude material re-dissolved 1:9 methanol—DCM and eluted through an Isolute-NH₂ cartridge. The solvents were removed and the crude product purified by preparative LCMS (high pH buffer) to give the product as a white solid (7 mg, 26%). ¹H NMR (400 MHz, DMSO-d₆) δ ppm 0.65-0.70 (m, 2H), 0.75-0.85 (m, 2H), 1.69-1.81 (m, 2H), 1.83-2.01 (m, 5H), 2.03-2.14 (m, 2H), 2.91-3.01 (m, 1H), 3.15-3.29 (m, 2H), 4.37 (t, J=6.18 Hz, 2H), 6.70 (td, J=8.36, 2.52 Hz, 1H), 7.23-7.30 (m, 1H), 7.43-7.48 (m, 1H), 7.70-7.79 (m, 2H), 7.94 (s, 1H), 9.61 (s, 1H); m/z (ES+APCI)⁺: 407 [M+Na]⁺.

### Example 106

### Cyclobutanecarboxylic acid (3-{[5-cyclopropyl-2-(3-fluoro-phenylamino)-pyrimidin-4-yl]-methyl-amino}-propyl)-amide

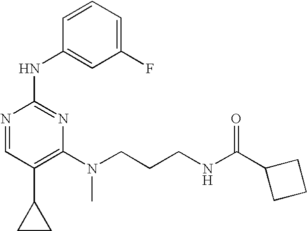

Intermediate 27 (40 mg, 0.12 mmol), 3-fluoroaniline (14 μl, 0.15 mmol), Pd₂(dba)₃ (6.8 mg, 0.007 mmol), xantphos (5.8 mg, 0.01 mmol) and sodium tert-butoxide (35.8 mg, 0.37 mmol) were combined in dioxane (2 ml), under nitrogen and the reaction mixture stirred at 90° C. for 2 days. The solvents were removed and the crude material re-dissolved in 1:9 methanol—DCM and eluted through a silica plug. The solvents were removed and the crude product purified by preparative LCMS (low pH buffer). After solvent removal, the product was re-dissolved in methanol/DCM (1:9) and eluted through an Isolute-NH₂ cartridge and solvents evaporated to give an off-white solid (11 mg, 22%). ¹H NMR (400 MHz, DMSO-d₆) δ ppm 0.57-0.62 (m, 2H), 0.78-0.87 (m, 2H), 1.67-1.77 (m, 3H), 1.79-1.90 (m, 2H), 1.91-2.00 (m, 2H), 2.02-2.14 (m, 2H), 2.89-2.96 (m, 1H), 3.01-3.13 (m, 2H), 3.20 (s, 3H), 3.67 (t, J=7.33 Hz, 2H), 6.64 (td, J=8.36, 2.52 Hz, 1H), 7.19-7.25 (m, 1H), 7.41 (d, J=8.24 Hz, 1H), 7.68 (t, J=5.50 Hz, 1H), 7.79 (dt, J=12.82, 2.29 Hz, 1H), 7.83 (s, 1H), 9.24 (s, 1H); m/z (ES+APCI)⁺: 398 [M+H]⁺.

### Example 107

### Cyclobutanecarboxylic acid {3-[5-cyclopropyl-2-(3-fluoro-phenylamino)-pyrimidin-4-ylamino]-propyl}-methyl-amide

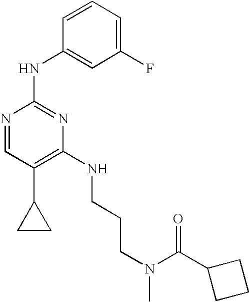

To a stirred solution of Intermediate 29 (21 mg, 0.06 mmol) in DMF (1 ml) was added sequentially, cyclobutanecarboxylic acid (8.6 μl, 0.09 mmol), HATU (36 mg, 0.1 mmol) and N,N-diisopropylethylamine (62 μl, 0.4 mmol), and the reaction mixture was left to stir overnight at room temperature. The solvent was evaporated and the crude material was re-dissolved in 1:9 methanol/DCM and eluted through an Isolute-NH₂ cartridge. The crude material was purified by preparative LCMS (low pH buffer) and solvents removed. The product as its TFA salt was then eluted through an Isolute-NH₂ cartridge using DCM:methanol (9:1), to give the final product as a white solid, (8 mg, 33%). ¹H NMR (400 MHz, DMSO-d₆) δ ppm 0.47-0.58 (m, 2H), 0.82-0.95 (m, 2H), 1.48-1.57 (m, 1H), 1.67-1.83 (m, 3H), 1.85-2.00 (m, 2H), 2.07-2.23 (m, 4H), 2.83 (s, 3H), 3.20-3.36 (m, 1H), 3.39-3.52 (m, 3H), 6.63-6.70 (m, 1H), 6.94-7.01 (m, 1H), 7.22-7.29 (m, 1H), 7.43-7.48 (m, 1H), 7.69 (s, 1H), 7.88-7.95 (m, 1H), 9.21 (s, 1H); m/z (ES+APCI)⁺: 398 [M+H]⁺.

### Example 108

### N-{3-[5-Cyclopropyl-2-(3-fluoro-phenylamino-pyrimidin-4-ylamino]-propyl}-butyramide

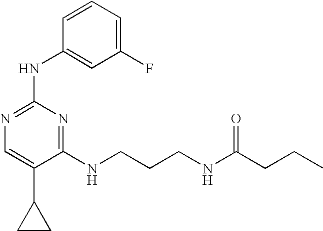

Prepared analogously to Example 107 from

Intermediate 3 (35 mg, 0.10 mmol) and butyric acid (14 μl, 0.16 mmol) to give a white solid (26 mg, 67%). ¹H NMR (400 MHz, DMSO-d₆) δ ppm 0.43-0.65 (m, 2H), 0.85-0.92 (m, 5H), 1.48-1.60 (m, 3H), 1.72-1.80 (m, 2H), 2.08 (t, J=7.3 Hz, 2H), 3.19 (q, J=6.6 Hz, 2H), 3.40-3.52 (m, 2H), 6.67 (td, J=8.5, 2.3 Hz, 1H), 6.96 (t, J=5.7 Hz, 1H), 7.22-7.29 (m, 1H), 7.47 (d, J=8.2 Hz, 1H), 7.69 (s, 1H), 7.85-7.94 (m, 2H), 9.20 (s, 1H); m/z (ES+APCI)⁺: 372 [M+H]⁺.

### Example 109

### Cyclobutanecarboxylic acid {3-[5-cyclopropyl-2-(6-morpholin-4-yl-pyridin-3-ylamino)-pyrimidin-4-ylamino]-propyl}-methyl-amide

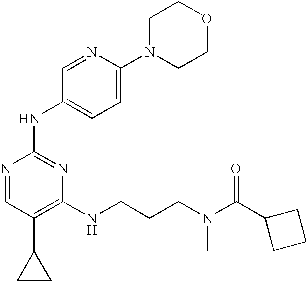

To a solution of Intermediate 40 (27 mg, 0.06 mmol) in DMF (1 ml) was added butyric acid (9 μL, 0.1 mmol), followed by HATU (39 mg, 0.1 mmol) then N,N-diisopropylethylamine (67 μl, 0.4 mmol) and the resulting solution was left to stir at room temperature overnight. The volatiles were removed under reduced pressure and the crude product was re-dissolved in 1:9 methanol:DCM (1 ml) and eluted though an Isolute-NH₂ cartridge. The solvents were removed and the crude product purified by preparative LCMS (low pH buffer) and eluted through an Isolute-NH₂ cartridge to give a solid (10 mg, 33%). ¹H NMR (400 MHz, DMSO-d₆, 40° C.) δ ppm 0.41-0.58 (m, 2H), 0.83-0.92 (m, 2H), 1.47-1.54 (m, 1H), 1.69-1.90 (m, 4H), 1.91-2.04 (m, 1H), 2.09-2.23 (m, 3H), 2.82 (s, 1H), 2.89 (s, 2H), 3.24-3.28 (m, 1H), 3.32-3.49 (m, 8H), 3.72-3.77 (m, 4H), 6.73-6.83 (m, 2H), 7.60-7.62 (m, 1H), 7.95-8.00 (m, 1H), 8.49-8.52 (m, 1H), 8.60 (s, 1H); m/z (ES+APCI)⁺: 466 [M+H]⁺.

### Example 110

### 1-{(3-[5-Cyclopropyl-2-(3-fluoro-phenylamino)-pyrimidin-4-ylamino]-propyl}-piperidin-2-one

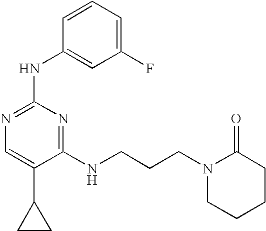

Intermediate 31 (71 mg, 0.2 mmol), 3-fluoroaniline (66.5 μl, 0.7 mmol) and glacial acetic acid (42.2 μl, 0.7 mmol) were mixed together in n-butanol (1 ml) and heated in the microwave at 150° C. for 1 hour. The solvents were evaporated and the crude material was purified by preparative LCMS (low pH buffer). The product as its TFA salt was then eluted through an Isolute-NH₂ cartridge using DCM:methanol 9:1 to give the final product as a white solid (31 mg, 35%). ¹H NMR (400 MHz, DMSO-d₆) δ ppm 0.44-0.59 (m, 2H), 0.83-0.91 (m, 2H), 1.47-1.54 (m, 1H), 1.68-1.85 (m, 6H), 2.27 (t, J=6.2 Hz, 2H), 3.23-3.35 (m, 2H), 3.39-3.49 (m, 4H), 6.67 (td, J=8.2, 2.3 Hz, 1H), 7.05 (t, J=6.0 Hz, 1H), 7.22-7.29 (m, 1H), 7.43-7.48 (m, 1H), 7.68 (s, 1H), 7.90 (dt, J=12.8, 2.3 Hz, 1H), 9.23 (s, 1H); m/z (ES+APCI)⁺: 384 [M+H]⁺.

### Example 111

### 1-{3-[5-Cyclopropyl-2-(3-fluoro-phenylamino)-pyrimidin-4-ylamino]-propyl}-3-methyl-H-pyridin-2-one

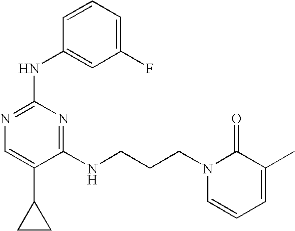

Prepared analogously to Example 110 from Intermediate 33 (49 mg, 0.15 mmol) and 3-fluoroaniline (44 μl, 0.46 mmol) to give a white solid (35 mg, 57%). ¹H NMR (400 MHz, DMSO-d₆) δ ppm 0.44-0.64 (m, 2H), 0.84-0.94 (m, 2H), 1.49-1.57 (m, 1H), 1.97-2.07 (m, 5H), 3.48 (q, J=6.4 Hz, 2H), 4.04 (t, J=6.9 Hz, 2H), 6.18 (t, J=−6.6 Hz, 1H), 6.67 (td, J=8.5, 2.3 Hz, 1H), 7.02 (t, J=5.7 Hz, 1H), 7.22-7.30 (m, 1H), 7.34 (d, J=5.5 Hz, 1H), 7.45-7.49 (m, 1H), 7.56-7.60 (m, 1H), 7.69 (s, 1H), 7.87-7.92 (m, 1H), 9.21 (s, 1H); m/z (ES+APCI)⁺: 394 [M+H]⁺.

### Example 112

### 1-{3-[5-Cyclopropyl-2-(3-fluoro-phenylamino)-pyrimidin-4-ylamino]-propyl}-3-methyl-imidazolidin-2-one

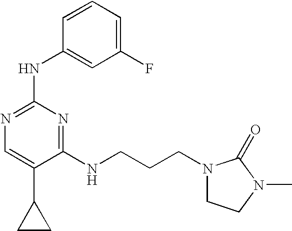

Prepared analogously to Example 110 from Intermediate 35 (80 mg, 0.26 mmol) and 3-fluoroaniline (75 μl, 0.78 mmol) to give a white solid (26 mg, 26%). ¹H NMR (400 MHz, DMSO-d₆) δ ppm 0.42-0.58 (m, 2H), 0.82-0.90 (m, 2H), 1.46-1.54 (m, 1H), 1.75-1.84 (m, 2H), 2.67 (s, 3H), 3.17-3.36 (m, 6H), 3.42-3.51 (m, 2H), 6.65 (td, J=8.1, 2.1 Hz, 1H), 6.93 (t, J=6.0 Hz, 1H), 7.21-7.28 (m, 1H), 7.46 (d, J=8.2 Hz, 1H), 7.67 (s, 1H), 7.90 (dt, J=13.3, 2.3 Hz, 1H), 9.20 (s, 1H); m/z (ES+APCI)⁺: 385 [M+H]⁺.

### Example 113

### 1-{3-[5-Cyclopropyl-2-(3-fluoro-phenylamino)-pyrimidin-4-ylamino]-propyl}-3,3-dimethyl-pyrrolidin-2-one

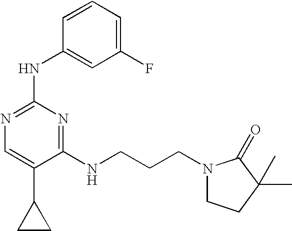

Prepared analogously to Example 110 from Intermediate 37 (78 mg, 0.24 mmol) and 3-fluoroaniline (70 μl, 0.73 mmol) to give a white solid (34 mg, 27%). ¹H NMR (400 MHz, DMSO-d₆) δ ppm 0.57-0.72 (m, 2H), 0.91-1.01 (m, 2H), 1.05 (s, 6H), 1.55-1.62 (m, 1H), 1.79-1.90 (m, 4H), 3.30 (t, J=6.9 Hz, 4H), 3.39-3.69 (m, 2H), 6.97-7.03 (m, 1H), 7.37 (d, J=9.2 Hz, 1H), 7.43-7.50 (m, 1H), 7.63-7.68 (m, 1H), 7.68 (s, 1H), 8.44 (br. s, 1H), 10.37 (br. s, 1H); m/z (ES+APCI)⁺: 398 [M+H]⁺.

### Example 114

### 1-{3-[5-Cyclopropyl-2-(3-fluoro-phenylamino)-pyrimidin-4-ylamino]-propyl}-3-trifluoromethyl-1H-pyridin-2-one

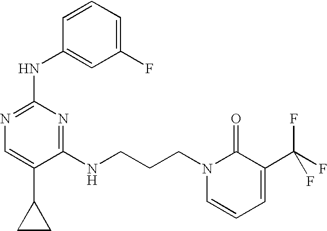

Intermediate 39 (22 mg, 0.06 mmol), 3-fluoroaniline (17 μl, 0.18 mmol) and glacial acetic acid (10.8 μl, 0.19 mmol) were mixed together in n-butanol (0.5 ml) and heated in the microwave at 150° C. for 1 hour. The solvents were evaporated and the crude material was purified by preparative LCMS (high pH buffer) to give a white solid (13 mg, 50%). ¹H NMR (400 MHz, DMSO-d₆) δ ppm 0.42-0.60 (m, 2H), 0.84-0.90 (m, 2H), 1.48-1.55 (m, 1H), 2.02-2.10 (m, 2H), 3.48-3.54 (m, 2H), 4.10 (t, J=7.1 Hz, 2H), 6.40 (t, J=6.9 Hz, 1H), 6.66 (td, J=8.2, 2.3 Hz, 1H), 6.96 (t, J=5.7 Hz, 1H), 7.22-7.28 (m, 1H), 7.47 (d, J=9.6 Hz, 1H), 7.69 (s, 1H), 7.88 (dt, J=12.8, 2.3 Hz, 1H), 7.96 (d, J=6.0 Hz, 1H), 8.09 (dd, J=6.6, 2.1 Hz, 1H), 9.20 (s, 1H); m/z (ES+APCI)⁺: 448 [M+H]⁺.

### Example 115

### Cyclobutanecarboxylic acid (3-{5-cyclopropyl-2-[6-(1-methyl-1H-pyrazol-4-yl)-pyridin-3-ylamino]-pyrimidin-4-ylamino}-propyl)-amide

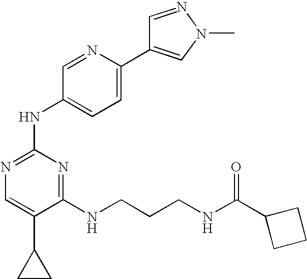

Prepared analogously to Example 114 from Intermediate 8 (25 mg, 0.08 mmol) and Intermediate 41 (42 mg, 0.24 mmol) to give the title compound (2.1 mg, 6%). ¹H NMR (400 MHz, DMSO-d₆) δ ppm 0.43-0.64 (m, 2H), 0.81-0.96 (m, 2H), 1.46-1.60 (m, 1H), 1.62-1.81 (m, 3H), 1.81-1.96 (m, 1H), 1.96-2.09 (m, 2H), 2.09-2.27 (m, 2H), 2.89-3.10 (m, 1H), 3.10-3.29 (m, 2H), 3.44-3.56 (m, 2H), 3.82-3.99 (m, 3H), 6.93 (t, J=5.3 Hz, 1H), 7.55 (d, J=8.2 Hz, 1H), 7.67 (s, 1H), 7.73 (t, J=5.5 Hz, 1H), 7.91 (s, 1H), 8.17 (s, 1H), 8.25 (d, J=8.7 Hz, 1H), 8.84 (s, 1H), 9.11 (s, 1H); m/z (ES+APCI)⁺: 447 [M+H]⁺.

### Example 116

### Cyclobutanecarboxylic acid {3-[5-cyclopropyl-2-(6-pyrimidin-5-yl-pyridin-3-ylamino)-pyrimidin-4-ylamino]-propyl}-amide

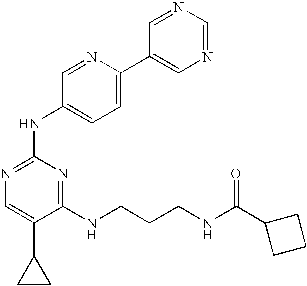

Example 97 (109 mg, 0.24 mmol), pyrimidine-5-boronic acid (33 mg, 0.27 mmol), Pd(dppf)Cl₂ (10 mg, 0.01 mmol) and 2M sodium carbonate (267 μl) were mixed together in dioxane (2 ml) and the solution degassed for 5 min. The reaction mixture was then stirred under nitrogen at 100° C. for 18 h. The reaction mixture was partitioned between water and ethyl acetate, extracted three times with ethyl acetate, the combined organic extracts washed with brine, dried (MgSO₄) and solvents removed. The crude product was purified by flash chromatography using a Biotage SP4 (DCM/methanol gradient) to give a white solid (35 mg, 32%). ¹H NMR (400 MHz, DMSO-d₆) δ ppm 0.46-0.62 (m, 2H), 0.86-0.91 (m, 2H), 1.49-1.57 (m, 1H), 1.71-1.81 (m, 3H), 1.83-1.91 (m, 1H), 1.96-2.05 (m, 2H), 2.09-2.19 (m, 2H), 2.98-3.03 (m, 1H), 3.20 (q, J=6.4 Hz, 2H), 3.45-3.52 (m, 2H), 7.01 (t, J=6.0 Hz, 1H), 7.70-7.76 (m, 2H), 8.08 (d, J=8.7 Hz, 1H), 8.47 (dd, J=8.7, 2.3 Hz, 1H), 9.07 (d, J=2.3 Hz, 1H), 9.19 (s, 1H), 9.40-9.44 (m, 3H); m/z (ES+APCI)⁺: 445 [M+H]⁺.

### Example 117

### Cyclobutanecarboxylic acid {3-[5-cyclopropyl-2-(3-pyrimidin-5-yl-phenylamino)-pyrimidin-4-ylamino]-propyl}-amide

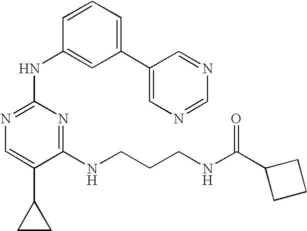

Example 141 below (100 mg, 0.23 mmol), pyrimidine-5-boronic acid (31 mg, 0.25 mmol), Pd(dppf)Cl₂ (9.2 mg, 0.01 mmol) and 2M sodium carbonate (248 μl, 0.50 mmol) were mixed together in dioxane (2 ml) and the solution degassed for 5 min. The reaction mixture was then stirred at 100° C. under nitrogen overnight. The solvents were evaporated and the crude product was purified by flash chromatography using a Biotage SP4 (DCM/methanol gradient) to give a beige solid (50 mg, 50%). ¹H NMR (400 MHz, DMSO-d₆) δ ppm 0.42-0.62 (m, 2H), 0.84-0.90 (m, 2 H), 1.48-1.56 (m, 1H), 1.66-1.81 (m, 3H), 1.85-1.95 (m, 1H), 1.97-2.06 (m, 2H), 2.09-2.19 (m, 2H), 2.95-3.04 (m, 1H), 3.09-3.18 (m, 2H), 3.47 (q, J=6.4 Hz, 2H), 6.92 (t, J=6.0 Hz, 1H), 7.29 (d, J=8.2 Hz, 1H), 7.43 (t, J=8.0 Hz, 1H), 7.69 (s, 1H), 7.72 (t, J=5.7 Hz, 1H), 7.85 (d, J=9.6 Hz, 1H), 8.31 (s, 1H), 9.09 (s, 2H), 9.12 (s, 1H), 9.23 (s, 1H); m/z (ES+APCI)⁺: 444 [M+H]⁺.

### Example 118

### Cyclobutanecarboxylic acid (3-{5-cyclopropyl-2-[6-(1H-pyrazol-4-yl)-pyridin-3-ylamino]-pyrimidin-4-ylamino}-propyl)-amide

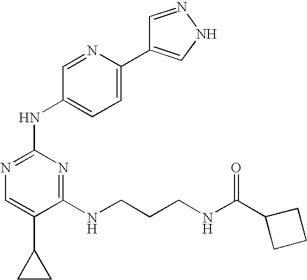

Example 97 (80 mg, 0.18 mmol), 4,4,5,5-tetramethyl-2-(1H-pyrazol-4-yl)-1,3,2-dioxaborolane (38 mg, 0.20 mmol), Pd(dppf)Cl₂ (7 mg, 0.01 mmol) and 2M sodium carbonate (198 μl) were mixed together in dioxane (1 ml) and the solution degassed. The reaction mixture was then stirred at 100° C. under nitrogen overnight. The reaction mixture was cooled under nitrogen, and volatiles evaporated. The crude material was re-dissolved in methanol, eluted through a plug of silica (DCM/methanol) and solvents evaporated. The crude material was purified by preparative LCMS (low pH buffer) to give the final product as a white solid (7 mg, 9%). ¹H NMR (400 MHz, DMSO-d₆) δ ppm 0.50-0.70 (m, 2H), 0.93-0.98 (m, 2H), 1.55-1.62 (m, 1H), 1.70-1.81 (m, 3H), 1.82-1.90 (m, 1H), 1.94-2.03 (m, 2H), 2.07-2.17 (m, 2H), 2.94-3.03 (m, 1H), 3.11-3.34 (m, 2H), 3.34-3.63 (m, 2H, obscured by water peak), 7.63 (s, 1H), 7.76 (t, J=5.7 Hz, 1H), 7.81 (d, J=8.7 Hz, 1H), 8.04-8.10 (m, 1H), 8.23 (br. s, 2H), 8.45 (br. s, 1H), 8.73 (br. s, 1H), 10.21 (br. s, 1H); m/z (ES+APCI)⁺: 433 [M+H]⁺.

### Example 119

### Cyclobutanecarboxylic acid {3-[2-([2,4′]bipyridinyl-5-ylamino)-5-cyclopropyl-pyrimidin-4-ylamino]-propyl}-amide

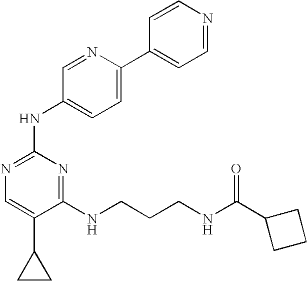

Example 97 (80 mg, 0.18 mmol), 4-pyridineboronic acid pinacol ester (41 mg, 0.20 mmol), Pd(dppf)Cl₂ (7 mg, 0.01 mmol) and 2M sodium carbonate (198 μl) were mixed together in dioxane (1 ml) and the solution degassed. The reaction mixture was then stirred under nitrogen at 100° C. overnight. The reaction mixture was cooled under nitrogen, and volatiles evaporated. The crude material was re-dissolved in methanol, eluted through a plug of silica (DCM/methanol) and solvents evaporated. The crude material was purified by preparative LCMS (low pH buffer). The product, as its TFA salt was eluted through an Isolute-NH₂ cartridge using DCM:methanol 9:1 to give a yellow solid (16 mg, 20%). ¹H NMR (400 MHz, DMSO-d₆) δ ppm 0.46-0.61 (m, 2H), 0.82-0.94 (m, 2H), 1.50-1.58 (m, 1H), 1.69-1.94 (m, 4H), 1.96-2.06 (m, 2H), 2.10-2.21 (m, 2H), 2.97-3.07 (m, 1H), 3.15-3.25 (m, 2H), 3.40-3.55 (m, 2H), 7.03 (t, J=6.0 Hz, 1H), 7.72 (s, 1H), 7.76 (t, 1H), 8.02-8.05 (m, 2H), 8.09 (d, J=9.2 Hz, 1H), 8.48 (dd, J=8.9, 2.5 Hz, 1H), 8.64-8.69 (m, 2H), 9.06 (d, J=2.3 Hz, 1H), 9.47 (s, 1H); m/z (ES+APCI)⁺: 444 [M+H]⁺.

### Example 120

### Cyclobutanecarboxylic acid {3-[2-([2,3′]bipyridinyl-5-ylamino)-5-cyclopropyl-pyrimidin-4-ylamino]-propyl}-amide

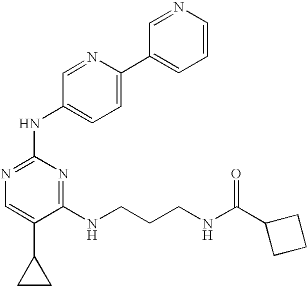

Example 97 (80 mg, 0.18 mmol), 3-pyridineboronic acid (24 mg, 0.20 mmol), Pd(dppf)Cl₂ (7 mg, 0.01 mmol) and 2M sodium carbonate (198 μl) were mixed together in dioxane (1 ml) and the solution degassed. The reaction mixture was then stirred under nitrogen at 100° C. overnight. The reaction mixture was cooled under nitrogen, and volatiles evaporated. The crude material was re-dissolved in methanol, eluted through a plug of silica (DCM/methanol) and solvents evaporated. The crude material was purified by preparative LCMS (low pH buffer). The product, as its TFA salt was eluted through an Isolute-NH₂ cartridge using DCM:methanol 9:1 to give a white solid (34 mg, 43%). ¹H NMR (400 MHz, DMSO-d₆) δ ppm 0.47-0.62 (m, 2H), 0.84-0.91 (m, 2H), 1.49-1.57 (m, 1H), 1.70-1.91 (m, 4H), 1.95-2.05 (m, 2H), 2.09-2.20 (m, 2H), 3.01 (quin, J=8.5 Hz, 1H), 3.20 (q, J=6.4 Hz, 2H), 3.45-3.53 (m, 2H), 7.00 (t, J=6.0 Hz, 1H), 7.51 (dd, J=8.0, 4.8 Hz, 1H), 7.70 (s, 1H), 7.75 (t, J=5.7 Hz, 1H), 8.00 (d, J=9.2 Hz, 1H), 8.38-8.45 (m, 2H), 8.57-8.59 (m, 1H), 9.04 (d, J=2.7 Hz, 1H), 9.25 (d, J=1.8 Hz, 1H), 9.36 (s, 1H); m/z (ES+APCI)⁺: 444 [M+H]⁺.

### Example 121

### Cyclobutanecarboxylic acid {3-[5-cyclopropyl-2-(3-pyridin-4-yl-phenylamino)-pyrimidin-4-ylamino]-propyl}-amide

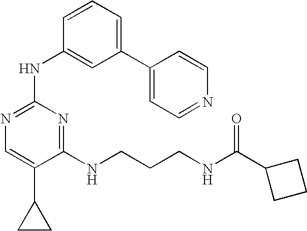

Example 141 (70 mg, 0.16 mmol), pyridine-4-boronic acid (22 mg, 0.17 mmol), Pd(dppf)Cl₂ (6.4 mg, 0.01 mmol) and 2M sodium carbonate (173 μl, 0.35 mmol) were mixed together in dioxane (1.5 ml) and the solution degassed. The reaction mixture was then stirred at 100° C. under nitrogen overnight. The volatiles were evaporated and the crude product eluted through a plug of silica, followed by purification by preparative LCMS (low pH buffer). The product as its TFA salt was then eluted through an Isolute-NH₂ cartridge using DCM:methanol 9:1, to give a white solid (37 mg, 53%). ¹H NMR (400 MHz, DMSO-d₆) δ ppm 0.40-0.61 (m, 2H), 0.81-0.91 (m, 2H), 1.47-1.55 (m, 1H), 1.66-1.80 (m, 3H), 1.82-1.94 (m, 1H), 1.95-2.05 (m, 2H), 2.07-2.18 (m, 2H), 2.93-3.05 (m, 1H), 3.13 (q, J=6.4 Hz, 2H), 3.39-3.52 (m, 2H), 6.90 (t, J=6.0 Hz, 1H), 7.27 (d, J=7.8 Hz, 1H), 7.39 (t, J=7.8 Hz, 1H), 7.63-7.66 (m, 2H), 7.68 (s, 1H), 7.70 (t, J=5.7 Hz, 1H), 7.84 (d, J=8.2 Hz, 1H), 8.31 (s, 1H), 8.64-8.68 (m, 2H), 9.10 (s, 1H); m/z (ES+APCI)⁺: 443 [M+H]⁺.

### Example 122

### Cyclobutanecarboxylic acid (3-{5-cyclopropyl-2-[3-(4-methyl-piperazin-1-ylmethyl)-phenylamino]-pyrimidin-4-ylamino}-propyl)-amide

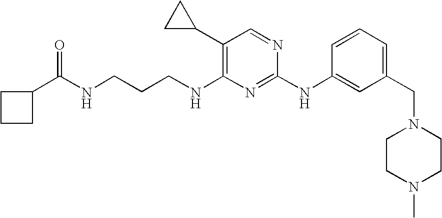

Intermediate 42 (125 mg, 0.27 mmol) and N-methyl piperazine (300 μl, 27 mmol) were combined in THF (2 ml) and stirred at room temperature for 18 h. The reaction mixture was diluted with EtOAc (20 ml) and partitioned with water (20 ml). The organic phase was washed with brine, dried (MgSO₄) and evaporated. The crude material was purified by flash chromatography on the Biotage SP4, eluting with 0 to 10% Methanol/DCM gradient. Further purification by elution through a 1 g Isolute-NH₂ cartridge with 9:1 DCM: methanol eluent gave the desired product as a white solid (23%). ¹H NMR (400 MHz, DMSO-d₆) δ ppm 0.43-0.49 (m, 2H), 0.79-0.86 (m, 2H), 1.42-1.52 (m, 1H), 1.65-1.78 (m, 3H), 1.78-1.92 (m, 1H), 1.92-2.02 (m, 2H), 2.04-2.17 (m, 5H), 2.17-2.47 (m, 6H), 2.89-3.00 (m, 1H), 3.09-3.18 (m, 2H), 3.32-3.38 (m, 4H), 3.42-3.49 (m, 2H), 6.73-6.78 (m, 1H), 6.81-6.87 (m, 1H), 7.08-7.17 (m, 1H), 7.51-7.57 (m, 1H), 7.60 (d, J=0.9 Hz, 1H), 7.66-7.72 (m, 1H), 7.80-7.84 (m, 1H), 8.86 (br. s, 1H); m/z (ES+APCI)⁺: 478 [M+H]⁺.

### Example 123

### Cyclobutanecarboxylic acid (3-{5-cyclopropyl-2-[3-(1-morpholin-4-yl-ethyl)-phenylamino]-pyrimidin-4-ylamino}-propyl)-amide

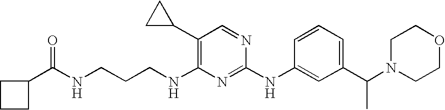

Intermediate 8 (121 mg, 0.39 mmol), Intermediate 43 (122 mg, 0.59 mmol) and trifluoroacetic acid (29 μl, 0.39 mmol) were combined with n-butanol (1.5 ml) and heated at 75° C. for 1.5 h The reaction mixture was evaporated and then purified by LCMS (low pH buffer). The product was then eluted through a 1 g Isolute-NH₂ cartridge with 9:1 DCM: methanol to give a white solid (61%). ¹H NMR (400 MHz, DMSO-d₆) δ ppm 0.44-0.51 (m, 2H), 0.81-0.87 (m, 2H), 1.26-1.39 (m, 3H), 1.44-1.52 (m, 1H), 1.66-1.77 (m, 3H), 1.78-1.92 (m, 1H), 1.93-2.03 (m, 2H), 2.04-2.16 (m, 2H), 2.30-2.45 (m, 2H), 2.90-3.01 (m, 1H), 3.09-3.17 (m, 2H), 3.21-3.63 (m, 9H), 6.81-6.89 (m, 1H), 7.05 (br. s, 1H), 7.16-7.23 (m, 1H), 7.54-7.58 (m, 1H), 7.60 (d, J=0.9 Hz, 1H), 7.65-7.72 (m, 1H), 7.77-7.81 (m, 1H), 9.03 (br. s, 1H); m/z (ES+APCI)⁺: 479 [M+H]⁺.

### Example 124

### Cyclobutanecarboxylic acid (3-{5-cyclopropyl-2-[3-(pyrrolidine-1-carbonyl)-phenylamino]-pyrimidin-4-ylamino}-propyl)-amide

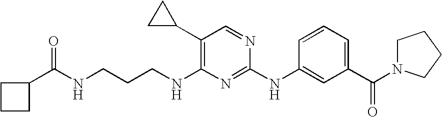

Prepared in a similar manner to Example 73 from Example 71 and pyrrolidine, to give the desired product as a white solid (24 mg, 88%). ¹H NMR (400 MHz, DMSO-d₆) δ ppm 0.44-0.50 (m, 2H), 0.78-0.87 (m, 2H), 1.42-1.51 (m, 1H), 1.62-1.91 (m, 8H), 1.92-2.02 (m, 2H), 2.04-2.16 (m, 2H), 2.90-3.01 (m, 1H), 3.07-3.16 (m, 2H), 3.31-3.48 (m, 6H), 6.85-6.90 (m, 1H), 6.91-6.96 (m, 1H), 7.22-7.28 (m, 1H), 7.62 (s, 1H), 7.68-7.73 (m, 1H), 7.73-7.78 (m, 1H), 8.00-8.04 (m, 1H), 9.05 (br. s, 1H); m/z (ES+APCI)⁺: 463 [M+H]⁺.

### Examples 125-126

Examples 125-126 were prepared analogously to Example 124 (the general structure is shown below followed by the tabulated examples).

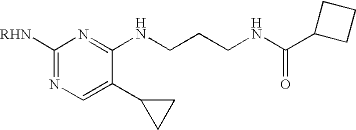

| | | | | HPLC |
|---|---|---|---|---|
| | | | m/z | retention |
| Example | R group | Name | (ES + APCI)⁺ | time (min)* |
| 125 | 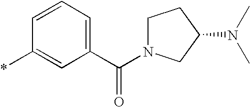 | Cyclobutanecarboxylic acid (3-{5-cyclopropyl-2- [3-((S)-3-dimethylamino- pyrrolidine-1-carbonyl)- phenylamino]-pyrimidin- 4-ylamino}-propyl)- amide | 506 | 6.66 |
| 126 | 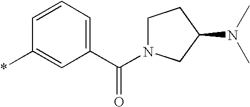 | Cyclobutanecarboxylic acid (3-{5-cyclopropyl-2- [3-((R)-3-dimethylamino- pyrrolidine-1-carbonyl)- phenylamino]-pyrimidin- 4-ylamino}-propyl)- amide | 506 | 6.70 |

*HPLC column: 21.2 × 100 mm (5 μm) C-18 Phenomenex Gemini; flow rate: 20 ml/min; run time: 10 min; gradient at start: 10% methanol and 90% water, gradient at finish: 100% methanol and 0% water; as buffer: 0.1% trifluoroacetic acid is added to the water.

### Example 127

### Cyclobutanecarboxylic acid [3-(5-cyclopropyl-2-{3-[2-(4-methyl-piperazin-1-yl)-2-oxo-ethyl]-phenylamino}-pyrimidin-4-ylamino)-propyl]-amide

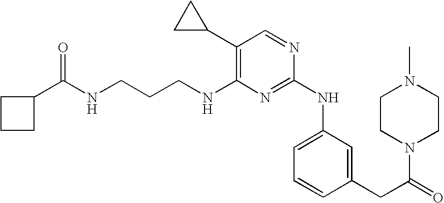

To a solution of N-methylpiperazine (8 μL, 0.07 mmol) in DMF (1 ml) was added Example 95 (50 mg, 0.11 mmol), HATU (44 mg, 0.12 mmol) and diisopropylethylamine (76 μl, 0.43 mmol), and the reaction was stirred at room temperature for 18 hours. The mixture was evaporated then eluted through a 0.5 g Isolute-NH₂ cartridge with 9:1 DCM:methanol eluent, then purified by LCMS (low pH buffer). The product was then eluted through a 0.5 g Isolute-NH₂ cartridge with 9:1 DCM:methanol to liberate the free base as a white solid (23 mg, 42%). ¹H NMR (400 MHz, DMSO-d₆) δ ppm 0.43-0.49 (m, 2H), 0.78-0.86 (m, 2H), 1.42-1.50 (m, 1H), 1.64-1.77 (m, 3H), 1.79-1.91 (m, 1H), 1.92-2.02 (m, 2H), 2.04-2.16 (m, 5H), 2.17-2.26 (m, 4H), 2.89-3.01 (m, 1H), 3.08-3.16 (m, 2H), 3.39-3.49 (m, 6H), 3.63 (s, 2H), 6.65-6.71 (m, 1H), 6.78-6.85 (m, 1H), 7.08-7.16 (m, 1H), 7.53-7.58 (m, 1H), 7.60 (s, 1H), 7.65-7.73 (m, 2H), 8.88 (br. s, 1H); m/z (ES+APCI)⁺: 506 [M+H]⁺.

### Example 128

### Cyclobutanecarboxylic acid [3-(5-cyclopropyl-2-{3-[2-(4-dimethylamino-piperidin-1-yl)-2-oxo-ethyl]-phenylamino}-pyrimidin-4-ylamino)-propyl]-amide

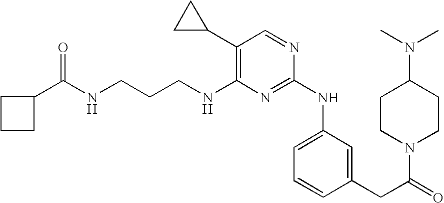

Example 128 was prepared in a similar manner to Example 127 from Example 95 and 4-(dimethylamino)piperidine to give the desired product as an off-white solid (20 mg, 56%). ¹H NMR (400 MHz, DMSO-d₆) δ ppm 0.45-0.50 (m, 2H), 0.82-0.88 (m, 2H), 1.14-1.25 (m, 2H), 1.46-1.54 (m, 1H), 1.63-1.72 (m, 2H), 1.72-1.93 (m, 4H), 1.98-2.08 (m, 2H), 2.08-2.19 (m, 8H), 2.23-2.31 (m, 1H), 2.92-3.02 (m, 1H), 3.02-3.08 (m, 4H), 3.14-3.22 (m, 2H), 3.44-3.51 (m, 2H), 3.64 (s, 2H), 6.45-6.55 (m, 1H), 6.68-6.76 (m, 1H), 7.09-7.17 (m, 1H), 7.38 (br. s, 1H), 7.53-7.57 (m, 1H), 7.61 (d, J=0.9 Hz, 1H), 7.68-7.71 (m, 1H), 8.47 (br. s, 1H); m/z (ES+APCI)⁺: 534 [M+H]⁺.

### Example 129

### Cyclobutanecarboxylic acid {3-[5-cyclopropyl-2-(1H-indazol-6-ylamino)-pyrimidin-4-ylamino]-propyl}-amide. Hydrochloride

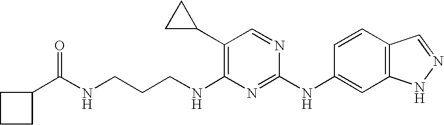

Prepared analogously to Example 40 from Intermediate 8 and 6-aminoindazole to give the desired product as a white solid (25 mg, 35%). ¹H NMR (400 MHz, DMSO-d₆) δ ppm 0.55-0.61 (m, 2H), 0.88-0.94 (m, 2H), 1.53-1.62 (m, 1H), 1.64-1.74 (m, 1H), 1.75-1.88 (m, 3H), 1.89-1.99 (m, 2H), 2.03-2.14 (m, 2H), 2.91-3.01 (m, 1H), 3.13-3.20 (m, 2H), 3.50-3.57 (m, 2H), 7.10-7.15 (m, 1H), 7.66 (s, 1H), 7.75 (d, J=8.7 Hz, 1H), 7.88-7.94 (m, 1H), 8.01-8.06 (m, 2H), 8.61-8.68 (m, 1H), 10.64 (s, 1H), 12.01 (br. s, 1H); m/z (ES+APCI)⁺: 406 [M+H]⁺.

### Example 130

### Cyclobutanecarboxylic acid {3-[5-cyclopropyl-2-(1H-indazol-5-ylamino)-pyrimidin-4-ylamino]-propyl}-amide

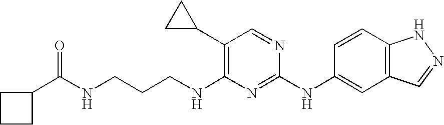

Prepared in a similar manner to Example 43 from Intermediate 8 and 5-aminoindazole. The product was isolated as a white solid (41 mg, 62%). ¹H NMR (400 MHz, DMSO-d₆) δ ppm 0.43-0.49 (m, 2H), 0.78-0.86 (m, 2H), 1.41-1.51 (m, 1H), 1.67-1.78 (m, 3H), 1.78-1.91 (m, 1H), 1.91-2.02 (m, 2H), 2.05-2.17 (m, 2H), 2.89-3.01 (m, 1H), 3.12-3.20 (m, 2H), 3.40-3.49 (m, 2H), 6.78-6.85 (m, 1H), 7.38 (d, J=9.2 Hz, 1H), 7.54 (dd, J=9.2, 1.8 Hz, 1H), 7.61 (s, 1H), 7.65-7.73 (m, 1H), 7.93 (s, 1H), 8.30 (d, J=1.4 Hz, 1H), 8.85 (s, 1H); m/z (ES+APCI)⁺: 406 [M+H]⁺.

### Example 131

### Cyclobutanecarboxylic acid {3-[5-cyclopropyl-2-(3-pyrrolidin-1-ylmethyl-phenylamino)-pyrimidin-4-ylamino]-propyl}-amide

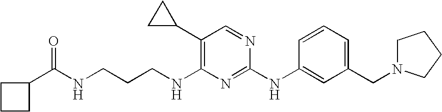

Intermediate 44 (40 mg, 0.089 mmol), pyrrolidine (0.13 mmol) and diisopropylethylamine (0.53 mmol) were combined in DCM (2 ml) and stirred at room temperature for 18 h and then heated at 35° C. for 30 min. The mixture was evaporated and the residue was eluted through a 0.5 g Isolute-NH₂ cartridge with 9:1 DCM:methanol, and then purified by flash chromatography on the Biotage SP4, eluting with 0 to 15% methanol/DCM gradient, to give the desired product as a white foam (12 mg, 29%). ¹H NMR (400 MHz, DMSO-d₆) δ ppm 0.39-0.46 (m, 2H), 0.75-0.83 (m, 2H), 1.38-1.47 (m, 1H), 1.59-1.73 (m, 7H), 1.74-1.88 (m, 1H), 1.89-1.99 (m, 2H), 2.01-2.13 (m, 2H), 2.32-2.41 (m, 4H), 2.85-2.96 (m, 1H), 3.06-3.15 (m, 2H), 3.37-3.48 (m, 4H), 6.71-6.75 (m, 1H), 6.76-6.83 (m, 1H), 7.05-7.13 (m, 1H), 7.48-7.54 (m, 1H), 7.57 (d, J=0.9 Hz, 1H), 7.60-7.67 (m, 1H), 7.79 (s, 1H), 8.78-8.83 (m, 1H); m/z (ES+APCI)⁺: 449 [M+H]⁺.

### Example 132

### Cyclobutanecarboxylic acid [3-(2-{3-[(cyclopentyl-methyl-amino)-methyl]-phenylamino}-5-cyclopropyl-pyrimidin-4-ylamino)-propyl]-amide

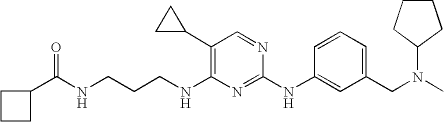

Intermediate 44 (40 mg, 0.089 mmol), cyclopentylmethylamine (0.13 mmol) and diisopropylethylamine (0.53 mmol) were combined in DCM (2 ml) and stirred at room temperature for 18 h and then heated at 35° C. for 30 min. The mixture was evaporated and purified LCMS (low pH buffer). The product was then eluted through a 0.5 g Isolute-NH₂ cartridge with 9:1 DCM:methanol to give the a white foam (12%). ¹H NMR (400 MHz, DMSO-d₆) δ ppm 0.43-0.49 (m, 2H), 0.79-0.85 (m, 2H), 1.35-1.56 (m, 5H), 1.57-1.91 (m, 8H), 1.92-2.04 (m, 5H), 2.04-2.16 (m, 2H), 2.65-2.74 (m, 1H), 2.88-2.99 (m, 1H), 3.09-3.16 (m, 2H), 3.39 (s, 2H), 3.42-3.49 (m, 2H), 6.74-6.79 (m, 1H), 6.79-6.84 (m, 1H), 7.10-7.15 (m, 1H), 7.53-7.57 (m, 1H), 7.60 (d, J=0.9 Hz, 1H), 7.64-7.69 (m, 1H), 7.78-7.81 (m, 1H), 8.84 (br. s, 1H); m/z (ES+APCI)⁺: 477 [M+H]⁺.

### Example 133

### Cyclobutanecarboxylic acid [3-(5-cyclopropyl-2-{3-[(isobutyl-methyl-amino)-methyl]-phenylamino}-pyrimidin-4-ylamino)-propyl]-amide

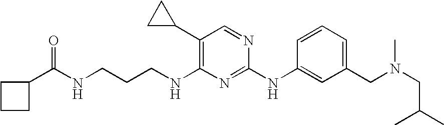

Prepared analogously to Example 131 from Intermediate 44 and isobutylmethylamine to give the desired product as a white foam (5.2 mg, 12%). ¹H NMR (400 MHz, DMSO-d₆, 85° C.) δ ppm 0.43-0.51 (m, 2H), 0.80-0.94 (m, 8H), 1.46-1.55 (m, 1H), 1.70-1.94 (m, 5H), 1.98-2.08 (m, 2H), 2.08-2.21 (m, 7H), 2.91-3.13 (m, 1H), 3.14-3.21 (m, 2H), 3.40 (s, 2H), 3.44-3.52 (m, 2H), 6.46-6.53 (m, 1H), 6.78-6.85 (m, 1H), 7.10-7.18 (m, 1H), 7.36 (br. s, 1H), 7.56-7.63 (m, 2H), 7.68-7.74 (m, 1H), 8.44 (br. s, 1H); m/z (ES+APCI)⁺: 465 [M+H]⁺.

### Example 134

### Cyclobulanecarboxylic acid {3-[5-cyclopropyl-2-(3-piperidin-1-ylmethyl-phenylamino)-pyrimidin-4-ylamino]-propyl}-amide

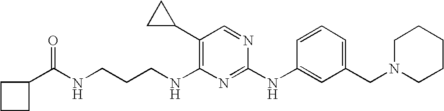

Example 134 was prepared analogously to Example 131 from Intermediate 44 and piperidine to give the desired product as a white solid (21 mg, 51%). ¹H NMR (400 MHz, DMSO-d₆, 85° C.) δ ppm 0.44-0.50 (m, 2H), 0.82-0.89 (m, 2H), 1.35-1.57 (m, 7H), 1.68-1.93 (m, 4H), 1.97-2.20 (m, 4H), 2.37-2.45 (m, 4H), 2.90-3.12 (m, 1H), 3.14-3.21 (m, 2H), 3.37-3.55 (m, 4H), 6.48-6.55 (m, 1H), 6.79-6.84 (m, 1H), 7.11-7.18 (m, 1H), 7.36 (br. s, 1H), 7.57-7.62 (m, 2H), 7.72-7.78 (m, 1H), 8.47 (br. s, 1H); m/z (ES+APCI)⁺: 463 [M+H]⁺.

### Example 135

### Cyclobutanecarboxylic acid (3-{5-cyclopropyl-2-[3-(4,4-difluoro-piperidin-1-ylmethyl)-phenylamino]-pyrimidin-4-ylamino}-propyl)-amide

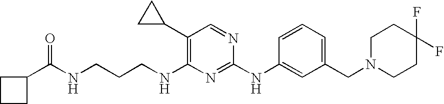

Example 135 was prepared analogously to Example 131 from Intermediate 44 and 4,4-difluoropiperidine to give the desired product as an off-white solid (33 mg, 75%). ¹H NMR (400 MHz, DMSO-d₆) δ ppm 0.43-0.49 (m, 2H), 0.79-0.86 (m, 2H), 1.42-1.51 (m, 1H), 1.65-1.77 (m, 3H), 1.78-2.03 (m, 7H), 2.05-2.16 (m, 2H), 2.44-2.48 (m, 4H), 2.88-3.01 (m, 1H), 3.10-3.17 (m, 2H), 3.42-3.49 (m, 4H), 6.76-6.80 (m, 1H), 6.81-6.86 (m, 1H), 7.10-7.18 (m, 1H), 7.57-7.59 (m, 1H), 7.60 (d, J=0.9 Hz, 1H), 7.65-7.70 (m, 1H), 7.78-7.83 (m, 1H), 8.87 (br. s, 1H); m/z (ES+APCI)⁺: 499 [M+H]⁺.

### Example 136

### Cyclobutanecarboxylic acid {3-[2-(3-cyanomethyl-phenylamino)-5-cyclopropyl-pyrimidin-4-ylamino]-propyl}-amide

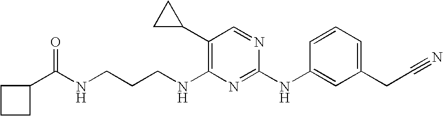

Intermediate 8 (324 mg, 1.05 mmol), Intermediate 45 (208 mg, 1.57 mmol) and trifluoroacetic acid (78 μl, 1.05 mmol) were heated in n-butanol (3 ml) at 75° C. for 3 h. The resultant white precipitate was filtered, washing with a minimum amount of methanol. The solid was dissolved in 9:1 DCM; methanol and eluted through a 1 g Isolute-NH₂ cartridge with 9:1 DCM:methanol to give the product as a white solid (0.33 g, 78%). ¹H NMR (400 MHz, DMSO-d₆) δ ppm 0.46-0.52 (m, 2H), 0.81-0.88 (m, 2H), 1.45-1.54 (m, 1H), 1.66-1.78 (m, 3H), 1.79-1.92 (m, 1H), 1.93-2.02 (m, 2H), 2.04-2.17 (m, 2H), 2.90-3.02 (m, 1H), 3.08-3.18 (m, 2H), 3.42-3.50 (m, 2H), 3.99 (s, 2H), 6.84-6.88 (m, 1H), 7.18-7.22 (m, 1H), 7.23-7.28 (m, 1H), 7.57-7.61 (m, 1H), 7.62 (d, J=0.9 Hz, 1H), 7.67-7.74 (m, 1H), 7.83-7.88 (m, 1H), 9.28 (br. s, 1H); m/z (ES+APCI)⁺: 405 [M+H]⁺.

### Example 137

### Cyclobutanecarboxylic acid {3-[5-cyclopropyl-2-(1-methyl-1H-indazol-5-ylamino)-pyrimidin-4-ylamino]-propyl}-amide

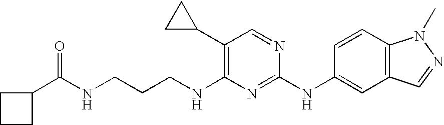

Example 137 was prepared analogously to Example 94 to from Intermediate 8 and 1-methyl-1H-indazol-5-ylamine to give the desired product as a pale pink solid (40 mg, 59%). ¹H NMR (400 MHz, DMSO-d₆) δ ppm 0.43-0.49 (m, 2H), 0.78-0.86 (m, 2H), 1.42-1.50 (m, 1H), 1.67-1.77 (m, 3H), 1.78-1.90 (m, 1H), 1.91-2.02 (m, 2H), 2.05-2.17 (m, 2H), 2.89-3.00 (m, 1H), 3.10-3.21 (m, 2H), 3.40-3.50 (m, 2H), 3.98 (s, 3H), 6.77-6.84 (m, 1H), 7.47 (d, J=8.7 Hz, 1H), 7.57-7.62 (m, 2H), 7.65-7.72 (m, 1H), 7.88-7.92 (m, 1H), 8.30 (s, 1H), 8.86 (s, 1H); m/z (ES+APCI)⁺: 420 [M+H]⁺.

### Example 138

### Cyclobutanecarboxylic acid {3-[5-cyclopropyl-2-(2-methyl-2H-indazol-6-ylamino)-pyrimidin-4-ylamino]-propyl}-amide

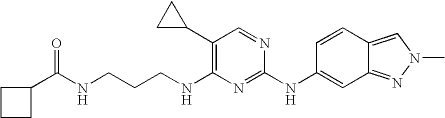

Example 138 was prepared analogously to Example 94 to from Intermediate 8 and 2-methyl-2H-indazol-6-ylamine to give the desired product as a pale brown solid (39 mg, 57%). ¹H NMR (400 MHz, DMSO-d₆) δ ppm 0.45-0.51 (m, 2H), 0.80-0.86 (m, 2H), 1.43-1.51 (m, 1H), 1.66-1.89 (m, 4H), 1.90-2.00 (m, 2H), 2.05-2.16 (m, 2H), 2.89-3.01 (m, 1H), 3.13-3.21 (m, 2H), 3.43-3.52 (m, 2H), 4.07 (s, 3H), 6.83-6.89 (m, 1H), 7.16 (dd, J=9.2, 1.8 Hz, 1H), 7.48 (d, J=8.7 Hz, 1H), 7.64 (d, J=0.9 Hz, 1H), 7.74-7.81 (m, 1H), 8.11-8.14 (m, 1H), 8.32 (s, 1H), 8.90 (s, 1H); m/z (ES+APCI)⁺: 420 [M+H]⁺.

### Example 139

### Cyclobutanecarboxylic acid {3-[5-cyclopropyl-2-(1-methyl-1H-indazol-6-ylamino)-pyrimidin-4-ylamino]-propyl}-amide

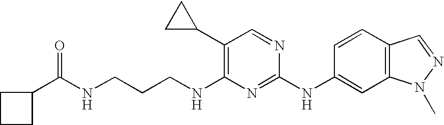

Example 139 was prepared analogously to Example 94 to from Intermediate 8 and 1-methyl-1H-indazol-6-ylamine to give the desired product as an off-white solid (42 mg, 61%). ¹H NMR (400 MHz, DMSO-d₆) δ ppm 0.46-0.53 (m, 2H), 0.82-0.88 (m, 2H), 1.46-1.54 (m, 1H), 1.66-1.90 (m, 4H), 1.91-2.01 (m, 2H), 2.03-2.15 (m, 2H), 2.88-2.99 (m, 1H), 3.10-3.19 (m, 2H), 3.50-3.58 (m, 2H), 3.93 (s, 3H), 6.87-6.94 (m, 1H), 7.19 (dd, J=8.7, 1.8 Hz, 1H), 7.53 (d, J=8.7 Hz, 1H), 7.65-7.71 (m, 2H), 7.82-7.86 (m, 1H), 8.42 (s, 1H), 9.17 (s, 1H); m/z (ES+APCI)⁺: 420 [M+H]⁺.

### Example 140

### Cyclobutanecarboxylic acid (3-{5-cyclopropyl-2-[3-(2-oxo-imidazolidin-1-yl)-phenylamino]-pyrimidin-4-ylamino}-propyl)-amide

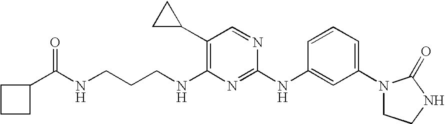

Example 140 was prepared analogously to Example 94 from Intermediate 8 and 1-(3-amino-phenyl)-imidazolidin-2-one to give the desired product as a white solid (50 mg, 69%). ¹H NMR (400 MHz, DMSO-d₆) δ ppm 0.44-0.48 (m, 2H), 0.80-0.85 (m, 2H), 1.43-1.50 (m, 1H), 1.64-1.77 (m, 3H), 1.79-1.91 (m, 1H), 1.92-2.02 (m, 2H), 2.05-2.16 (m, 2H), 2.89-2.99 (m, 1H), 3.08-3.14 (m, 2H), 3.36-3.42 (m, 2H), 3.42-3.48 (m, 2H), 3.78-3.83 (m, 2H), 6.76-6.81 (m, 1H), 6.86 (br. s, 1H), 7.08-7.13 (m, 1H), 7.18-7.22 (m, 1H), 7.38-7.41 (m, 1H), 7.60 (d, J=0.9 Hz, 1H), 7.64-7.69 (m, 1H), 7.80-7.82 (m, 1H), 8.81 (br. s, 1H); m/z (ES+APCI)⁺: 450 [M+H]⁺.

### Example 141

### Cyclobutanecarboxylic acid {3-[2-(3-bromo-phenylamino)-5-cyclopropyl-pyrimidin-4-ylamino]-propyl}-amide

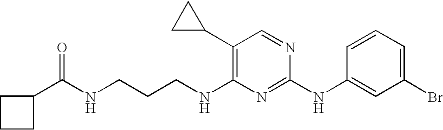

Example 141 was prepared analogously to Example 94 to from Intermediate 8 and 3-bromo-phenylamine to give the desired product as an off-white foam (0.26 g, 92%). ¹H NMR (400 MHz, DMSO-d₆) δ ppm 0.45-0.50 (m, 2H), 0.80-0.86 (m, 2H), 1.43-1.51 (m, 1H), 1.67-1.78 (m, 3H), 1.79-1.92 (m, 1H), 1.94-2.03 (m, 2H), 2.04-2.17 (m, 2H), 2.91-3.02 (m, 1H), 3.09-3.20 (m, 2H), 3.39-3.48 (m, 2H), 6.90-6.95 (m, 1H), 6.99 (ddd, J=7.8, 1.8, 0.9 Hz, 1H), 7.12-7.19 (m, 1H), 7.57-7.61 (m, 1H), 7.64 (d, J=0.9 Hz, 1H), 7.66-7.71 (m, 1H), 8.26-8.31 (m, 1H), 9.14 (s, 1H); m/z (ES+APCI)⁺: 444/446 [M+H]⁺.

### Example 142

### Cyclobutanecarboxylic acid (3-{5-cyclopropyl-2-[3-(piperazine-1-carbonyl)-phenylamino]-pyrimidin-4-ylamino}-propyl)-amide. Hydrochloride

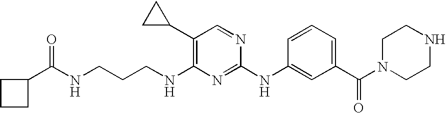

### Step 1

To a solution of piperazine-1-carboxylic acid tert-butyl ester (11 mg, 0.06 mmol) in DMF (1 ml) was added Example 71 (40 mg, 0.09 mmol), HATU (36 mg, 0.09 mmol) and diisopropylethylamine (63 μl, 0.35 mmol), and the reaction was stirred at room temperature for 18 hours. The mixture was evaporated, taken up in 9:1 DCM:MeOH and filtered through a 0.5 g Isolute-NH₂ cartridge, eluting with 9:1 DCM:methanol. Flash chromatography on the Biotage SP4 (0 to 10% methanol/DCM gradient), gave a colourless oil (34 mg, 100%), which was used in the next step without further purification.

### Step 2

The crude product from Step 1 (50 mg, 0.09 mmol) and 4M hydrogen chloride solution in dioxan (5 ml) were combined and stirred at room temperature for 18 h and evaporated. Purification by flash chromatography using a Biotage SP4 (gradient elution from 0-20% methanol in DCM), gave the desired product as a white solid (7 mg, 22%). ¹H NMR (400 MHz, DMSO-d₆) δ ppm 0.43-0.50 (m, 2H), 0.77-0.90 (m, 2H), 1.42-1.54 (m, 1H), 1.61-1.77 (m, 3H), 1.78-2.03 (m, 3H), 2.04-2.17 (m, 2H), 2.65-2.87 (m, 4H), 2.92-3.04 (m, 1H), 3.09-3.15 (m, 2H), 3.28-3.49 (m, 2H), 3.51-3.67 (m, 4H), 4.15 (br. s, 1H), 6.80-6.85 (m, 1H), 6.91-6.96 (m, 1H), 7.23-7.30 (m, 1H), 7.62 (s, 1H), 7.74-7.78 (m, 1H), 7.78-7.83 (m, 1H), 7.89-7.93 (m, 1H), 9.08 (br. s, 1H): m/z (ES+APCI)⁺: 478 [M+H]⁺.

### Example 143

### Cyclobutanecarboxylic acid (3-{5-cyclopropyl-2-[3-(2-morpholin-4-yl-ethoxy)-phenylamino]-pyrimidin-4-ylamino}-propyl)-amide

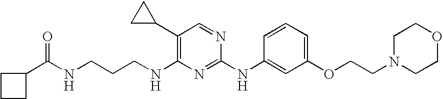

Example 143 was prepared analogously to Example 64 from Intermediate 8 and 3-(2-morpholin-4-yl-ethoxy)-phenylamine to give the desired product as a white foam (15 mg, 23%). ¹H NMR (400 MHz, DMSO-d₆) δ ppm 0.44-0.49 (m, 2H), 0.80-0.86 (m, 2H), 1.42-1.50 (m, 1H), 1.64-1.77 (m, 3H), 1.79-1.92 (m, 1H), 1.93-2.02 (m, 2H), 2.05-2.16 (m, 2H), 2.44-2.48 (m, 4H), 2.67 (t, J=5.7 Hz, 2H), 2.90-3.00 (m, 1H), 3.10-3.17 (m, 2H), 3.40-3.47 (m, 2H), 3.53-3.60 (m, 4H), 4.02 (t, J=5.7 Hz, 2H), 6.39-6.46 (m, 1H), 6.82-6.90 (m, 1H), 7.03-7.12 (m, 1H), 7.21-7.25 (m, 1H), 7.57-7.59 (m, 1H), 7.61 (d, J=0.9 Hz, 1H), 7.65-7.72 (m, 1H), 8.87 (br. s, 1H): m/z (ES+APCI)⁺: 495 [M+H]⁺.

### Example 144

### Pyrrolidine-1-carboxylic acid (3-{4-[3-(cyclobutanecarbonyl-amino)-propylamino]-5-cyclopropyl-pyrimidin-2-ylamino}-phenyl)-amide

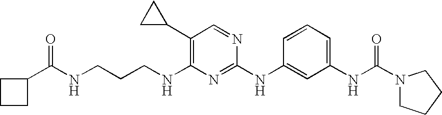

Example 144 was prepared analogously to Example 64 from Intermediate 8 and Intermediate 10 to give the desired product as a white foam (5 mg, 8%). ¹H NMR (400 MHz, DMSO-d₆) δ ppm 0.42-0.48 (m, 2H), 0.79-0.85 (m, 2H), 1.42-1.50 (m, 1H), 1.63-1.77 (m, 3H), 1.80-1.91 (m, 5H), 1.92-2.01 (m, 2H), 2.04-2.15 (m, 2H), 2.89-2.99 (m, 1H), 3.08-3.14 (m, 2H), 3.32-3.38 (m, 4H), 3.42-3.48 (m, 2H), 6.73-6.78 (m, 1H), 6.90-6.94 (m, 1H), 7.00-7.06 (m, 1H), 7.27-7.30 (m, 1H), 7.58 (d, J=0.9 Hz, 1H), 7.64-7.69 (m, 1H), 7.87-7.90 (m, 1H), 7.94 (br. s, 1H), 8.73 (br. s, 1H); m/z (ES+APCI)⁺: 478 [M+H]⁺.

### Example 145

### Cyclobutanecarboxylic acid (3-{5-cyclopropyl-2-[3-(1-methyl-1-morpholin-4-yl-ethyl)-phenylamino]-pyrimidin-4-ylamino}-propyl)-amide

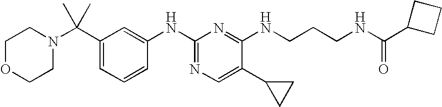

Prepared analogously to Example 91 using Intermediate 8 and 3-(1-methyl-1-morpholin-4-yl-ethyl)-phenylamine to provide the product as a white solid (24 mg, 38%). ¹H NMR (400 MHz, DMSO-d₆) δ ppm 0.43-0.48 (m, 2H), 0.79-0.85 (m, 2H), 1.27 (s, 6H), 1.42-1.49 (m, 1H), 1.64-1.76 (m, 3H), 1.80-1.88 (m, 1H), 1.92-2.01 (m, 2H), 2.04-2.15 (m, 2H), 2.31-2.40 (m, 4H), 2.90-2.97 (m, 1H), 3.08-3.15 (m, 2H), 3.43-3.51 (m, 2H), 3.51-3.57 (m, 4H), 6.79 (t, J=5.95 Hz, 1H), 6.99 (d, J=8.24 Hz, 1H), 7.13 (t, J=8.01 Hz, 1H), 7.59 (s, 1H), 7.61-7.70 (m, 2H), 7.85 (s, 1H), 8.80 (s, 1H); m/z (ES+APCI)⁺: 493 [M+H]⁺.

### Example 146

### Cyclobutanecarboxylic acid (3-{5-cyclopropyl-2-[6-((S)-3-dimethylamino-pyrrolidin-1-ylmethyl)-pyridin-3-ylamino]-pyrimidin-4-ylamino}-propyl)-amide

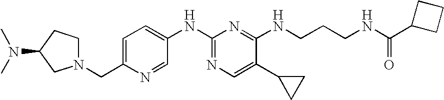

A solution of (S)-(−)-3-dimethylaminopyrrolidine (14.4 μl, 0.114 mmol) and DIPEA (79 μl, 0.455 mmol) in DCM (1 ml) was added to a stirred suspension of Intermediate 22 in DCM (1 ml), and the resultant solution was stirred at rt for 3 days. The mixture was concentrated to dryness and the resulting residue was dissolved in 10:1 DCM-MeOH, and the resulting solution was passed through a plug of silica gel. The filtrate was concentrated to dryness and the residue was purified by mass-triggered preparative HPLC (low pH buffer). The purified material was passed through a SCX cartridge to provide the product as the free base (1.4 mg). ¹H NMR (400 MHz, CHLOROFORM-d) δ ppm 0.49-0.54 (m, 2H), 0.91-0.97 (m, 2H), 1.45-1.52 (m, 1H), 1.61-2.05 (m, 8H), 2.07-2.16 (m, 2H), 2.20-2.42 (m, 7H), 2.54-2.61 (m, 1H), 2.76-2.85 (m, 2H), 2.88-3.02 (m, 2H), 3.37 (q, J=6.41 Hz, 2H), 3.56 (q, J=6.26 Hz, 2H), 3.69-3.79 (m, 2H), 5.87 (t, J=6.18 Hz, 1H), 6.04 (t, J=6.18 Hz, 1H), 6.90 (br. s, 1H), 7.31 (d, J=7.56 Hz, 1H), 7.73 (s, 1H), 7.96 (dd, J=8.47, 2.52 Hz, 1H), 8.84 (d, J=2.29 Hz, 1H); m/z (ES+APCI)⁺: 493 [M+H]⁺.

### Example 147

### Cyclobutanecarboxylic acid (3-{5-cyclopropyl-2-[6-(4-methyl-piperazin-1-ylmethyl)-pyridin-3-ylamino]-pyrimidin-4-ylamino}-propyl)-amide

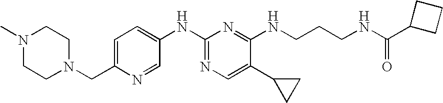

Prepared analogously to Example 146 using Intermediate 22 and N-methylpiperazine to provide the product as the free base (1.4 mg). ¹H NMR (400 MHz, CHLOROFORM-d) δ ppm 0.49-0.54 (m, 2H), 0.88-0.97 (m, 2H), 1.45-1.60 (m, 1H), 1.64-2.00 (m, 4H), 2.07-2.17 (m, 2H), 2.20-2.41 (m, 5H), 2.41-2.71 (m, 8H), 2.93-3.00 (m, 1H), 3.38 (q, J=6.41 Hz, 2H), 3.53-3.64 (m, 4H), 5.85 (t, J=6.18 Hz, 1H), 6.06 (t, J=5.95 Hz, 1H), 6.93 (br. s, 1H), 7.31 (m, 1H), 7.72 (m, 1H), 7.97 (dd, J=8.47, 2.52 Hz, 1H), 8.85 (m, 1H); m/z (ES+APCI)⁺: 479 [M+H]⁺.

### Example 148

### Cyclobutanecarboxylic acid (3-{5-cyclopropyl-2-[6-(4,4-difluoro-piperidin-1-ylmethyl)-pyridin-3-ylamino]-pyrimidin-4-ylamino}-propyl)-amide

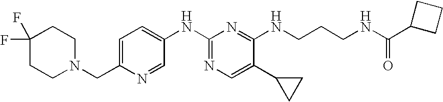

Prepared analogously to Example 146 using Intermediate 22 and 4,4-difluoropiperidine HCl to provide the product as the free base (14 mg). ¹H NMR (400 MHz, CHLOROFORM-d) δ ppm 0.49-0.57 (m, 2H), 0.88-0.99 (m, 2H), 1.45-1.54 (m, 1H), 1.75-2.17 (m, 10H), 2.19-2.33 (m, 2H), 2.57-2.65 (m, 4H), 2.97 (quin, J=8.70 Hz, 1H), 3.38 (q, J=6.11 Hz, 2H), 3.48-3.70 (m, 4H), 5.79 (br. s, 1H), 6.11 (t, J=5.95 Hz, 1H), 7.05-7.14 (m, 1H), 7.29 (d, J=8.24 Hz, 1H), 7.71 (s, 1H), 8.01 (dd, J=8.47, 2.52 Hz, 1H), 8.81-8.86 (m, 1H), m/z (ES+APCI)⁺: 500 [M+H]⁺.

### Example 149

### Cyclobutanecarboxylic acid {3-[5-cyclopropyl-2-(pyrimidin-5-ylamino)-pyrimidin-4-ylamino]-propyl}-amide

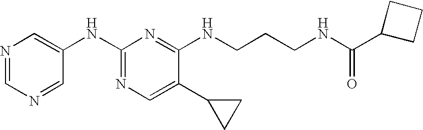

Intermediate 8 (40 mg, 0.130 mmol), 5-aminopyrimidine (14.8 mg, 0.156 mmol), xantphos (6.0 mg, 0.010 mmol), sodium t-butoxide (37 mg, 0.390 mmol) and Pd₂(dba)₃ (7.1 mg, 0.008 mmol) were placed in a sealed tube. The mixture was degassed, placed under an atmosphere of nitrogen and heated at 100° C. overnight. The mixture was diluted with EtOAc and water. The organic phase was washed with brine, dried and concentrated. The residue was purified by mass-triggered preparative HPLC (low pH buffer) to afford a colourless oil. This material was passed through a SCX cartridge to provide the product as the free base (2.4 mg, 5%). ¹H NMR (400 MHz, DMSO-d₆) δ ppm 0.46-0.54 (m, 2H), 0.80-0.90 (m, 2H), 1.45-1.52 (m, 1H), 1.65-1.77 (m, 3H), 1.81-1.90 (m, 1H), 1.93-2.16 (m, 4H), 2.97 (quin, J=8.24 Hz, 1H), 3.09-3.16 (m, 2H), 3.35-3.46 (m, 2H), 6.99 (t, J=5.72 Hz, 1H), 7.63-7.71 (m, 2H), 8.67 (s, 1H), 9.15 (s, 2H), 9.29 (s, 1H); m/z (ES+APCI)⁺: 368 [M+H]⁺.

### Example 150

### Cyclobutanecarboxylic acid {3-[5-cyclopropyl-2-(3,4,5,6-tetrahydro-2H-[1,2′]bipyridinyl-5′-ylamino)-pyrimidin-4-ylamino]-propyl}-amide

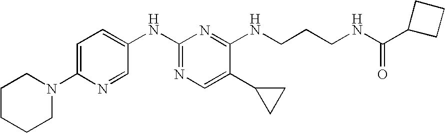

Prepared analogously to Example 97 using Intermediate 8 and 3,4,5,6-tetrahydro-2H-[1,2′]bipyridinyl-5′-ylamine to provide product as the free base (31 mg, 53%). ¹H NMR (400 MHz, DMSO-d₆) δ ppm 0.40-0.45 (m, 2H), 0.76-0.84 (m, 2H), 1.39-1.47 (m, 1H), 1.48-1.59 (m, 6H), 1.62-1.76 (m, 3H), 1.80-1.90 (m, 1H), 1.92-2.01 (m, 2H), 2.04-2.15 (m, 2H), 2.90-3.00 (m, 1H), 3.07-3.17 (m, 2H), 3.33-3.42 (m, 6H), 6.71-6.78 (m, 2H), 7.53 (s, 1H), 7.65 (t, J=5.72 Hz, 1H), 7.89 (dd, J=9.16, 2.75 Hz, 1H), 8.37 (d, J=2.75 Hz, 1H), 8.58 (s, 1H); m/z (ES+APCI)⁺: 450 [M+H]⁺.

### Example 151

### Cyclobutanecarboxylic acid {3-[5-cyclopropyl-2-(6-pyrrolidin-1-yl-pyridin-3-ylamino)-pyrimidin-4-ylamino]-propyl}-amide

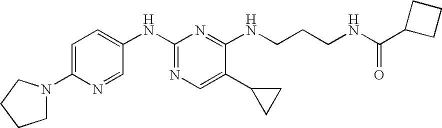

Prepared analogously to Example 97 using Intermediate 8 and 6-pyrrolidin-1-yl-pyridin-3-ylamine to provide product as the free base (31 mg, 55%). ¹H NMR (400 MHz, DMSO-d₆) δ ppm 0.39-0.46 (m, 2H), 0.77-0.85 (m, 2H), 1.40-1.47 (m, 1H), 1.62-1.78 (m, 3H), 1.81-2.02 (m, 7H), 2.05-2.16 (m, 2H), 2.96 (quin, J=8.24 Hz, 1H), 3.07-3.18 (m, 2H), 3.26-3.43 (m, 6H), 6.38 (d, J=9.16 Hz, 1H), 6.75 (t, J=5.72 Hz, 1H), 7.53 (s, 1H), 7.66 (t, J=5.95 Hz, 1H), 7.84 (dd, J=8.70, 2.75 Hz, 1H), 8.31 (d, J=2.29 Hz, 1H), 8.48 (s, 1H); m/z (ES+APCI)⁺: 436 [M+H]⁺.

### Example 152

### Cyclobutanecarboxylic acid {3-[5-cyclopropyl-2-(2-morpholin-4-yl-pyrimidin-5-ylamino)-pyrimidin-4-ylamino]-propyl}-amide

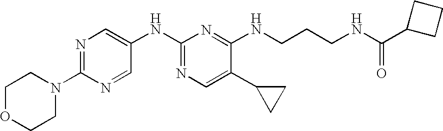

Prepared and analogously to Example 149 using Intermediate 8 and Intermediate 47 to give a yellow solid (9 mg, 15%). ¹H NMR (400 MHz, DMSO-d₆) δ ppm 0.41-0.48 (m, 2H), 0.78-0.86 (m, 2H), 1.40-1.48 (m, 1H), 1.61-1.77 (m, 3H), 1.81-1.91 (m, 1H), 1.92-2.16 (m, 4H), 2.91-3.02 (m, 1H), 3.06-3.17 (m, 2H), 3.29-3.42 (m, 2H), 3.55-3.69 (m, 8H), 6.86 (br. s, 1H), 7.53-7.57 (m, 1H), 7.66 (d, J=5.95 Hz, 1H), 8.63-8.74 (m, 3H); m/z (ES+APCI)⁺: 453 [M+H]⁺.

### Example 153

### Cyclobutanecarboxylic acid {3-[5-cyclopropyl-2-(1-methyl-1H-pyrazol-4-ylamino)-pyrimidin-4-ylamino]-propyl}-amide

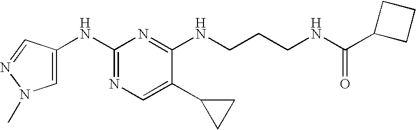

Prepared and analogously to Example 149 using Intermediate 8 and 1-methyl-1H-pyrazol-4-ylamine to provide a white solid (17 mg, 35%). ¹H NMR (400 MHz, DMSO-d₆) δ ppm 0.40-0.47 (m, 2H), 0.76-0.85 (m, 2H), 1.39-1.47 (m, 1H), 1.60-1.78 (m, 3H), 1.82-1.92 (m, 1H), 1.94-2.03 (m, 2H), 2.05-2.16 (m, 2H), 2.97 (quin, J=8.36 Hz, 1H), 3.06-3.16 (m, 2H), 3.40 (q, J=6.41 Hz, 2H), 3.76 (s, 3H), 6.71 (t, J=5.72 Hz, 1H), 7.40 (s, 1H), 7.54 (s, 1H), 7.69 (t, J=5.14 Hz, 1H), 7.76 (s, 1H), 8.65 (br. s, 1H); m/z (ES+APCI)⁺: 370 [M+H]⁺.

### Example 154

### Cyclobutanecarboxylic acid (3-{5-cyclopropyl-2-[6-(2-morpholin-4-yl-ethoxy)-pyridin-3-ylamino]-pyrimidin-4-ylamino}-propyl)-amide

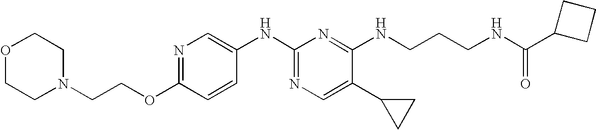

Prepared analogously to Example 97 using Intermediate 8 and Intermediate 49 to give an off-white solid (20 mg, 25%). ¹H NMR (400 MHz, DMSO-d₆) δ ppm 0.42-0.50 (m, 2H), 0.79-0.87 (m, 2H), 1.41-1.48 (m, 1H), 1.63-1.77 (m, 3H), 1.81-1.89 (m, 1H), 1.93-2.02 (m, 2H), 2.05-2.16 (m, 2H), 2.45 (br. s, 4H), 2.66 (t, J=5.72 Hz, 2H), 2.92-2.99 (m, 1H), 3.08-3.15 (m, 2H), 3.35-3.42 (m, 2H), 3.53-3.59 (m, 4H), 4.27-4.34 (m, 2H), 6.72 (d, J=9.16 Hz, 1H), 6.82 (t, J=5.95 Hz, 1H), 7.58 (s, 1H), 7.64-7.69 (m, 1H), 8.04 (dd, J=8.70, 2.75 Hz, 1H), 8.44 (d, J=2.29 Hz, 1H), 8.80 (s, 1H); m/z (ES+APCI)⁺: 496 [M+H]⁺.

### Example 155

### Cyclobutanecarboxylic acid (3-{5-cyclopropyl-2-[1-(2-morpholin-4-yl-ethyl)-1H-pyrazol-4-ylamino]-pyrimidin-4-ylamino}-propyl)-amide

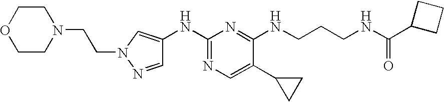

Prepared and analogously to Example 149 using Intermediate 8 and Intermediate 51 to give the product as an off-white coloured solid (17 mg, 28%). ¹H NMR (400 MHz, DMSO-d₆) δ ppm 0.41-0.47 (m, 2H), 0.78-0.85 (m, 2H), 1.40-1.47 (m, 1H), 1.61-1.78 (m, 3H), 1.82-1.90 (m, 1H), 1.94-2.03 (m, 2H), 2.06-2.16 (m, 2H), 2.33-2.44 (m, 4H), 2.64-2.72 (m, 2H), 2.93-3.00 (m, 1H), 3.06-3.16 (m, 2H), 3.35-3.45 (m, 2H), 3.51-3.57 (m, 4H), 4.12-4.19 (m, 2H), 6.71 (t, J=5.95 Hz, 1H), 7.43 (s, 1H), 7.55 (s, 1H), 7.65-7.72 (m, 1H), 7.82 (s, 1H), 8.66 (br. s, 1H), m/z (ES+APCI)⁺: 469 M+H]⁺.

### Example 156

### Cyclobutanecarboxylic acid {3-[5-cyclopropyl-2-(6-piperidin-1-ylmethyl-pyridin-3-ylamino)-pyrimidin-4-ylamino]-propyl}-amide

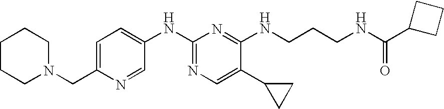

Prepared analogously to Example 146 using Intermediate 22 and piperidine to provide the product as the free base (7.5 mg). ¹H NMR (400 MHz, DMSO-d₆) δ ppm 0.44-0.49 (m, 2H), 0.80-0.86 (m, 2H), 1.34-1.53 (m, 7H), 1.65-1.77 (m, 3H), 1.81-1.89 (m, 1H), 1.93-2.02 (m, 2H), 2.05-2.16 (m, 2H), 2.34 (br. s, 4H), 2.93-3.00 (m, 1H), 3.09-3.16 (m, 2H), 3.36-3.49 (m, 4H), 6.87 (t, J=5.95 Hz, 1H), 7.25 (d, J=8.24 Hz, 1H), 7.61 (s, 1H), 7.69 (t, J=5.72 Hz, 1H), 8.16 (dd, J=8.47, 2.52 Hz, 1H), 8.77 (d, J=2.29 Hz, 1H), 9.04 (s, 1H); m/z (ES+APCI)⁺: 464 [M+H]⁺.

### Example 157

### Cyclobutanecarboxylic acid (3-{5-cyclopropyl-2-[6-(3,3-difluoro-piperidin-1-ylmethyl)-pyridin-3-ylamino]-pyrimidin-4-ylamino}-propyl)-amide

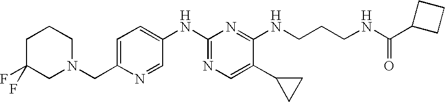

Prepared analogously to Example 146 using Intermediate 22 and 3,3-difluoropiperidine HCl to provide the product as the free base (16 mg). ¹H NMR (400 MHz, DMSO-d₆) δ ppm 0.44-0.49 (m, 2H), 0.80-0.86 (m, 2H), 1.43-1.51 (m, 1H), 1.60-1.77 (m, 5H), 1.79-2.02 (m, 5H), 2.05-2.16 (m, 2H), 2.42 (t, J=4.81 Hz, 2H), 2.61-2.69 (m, 2H), 2.92-3.00 (m, 1H), 3.09-3.17 (m, 2H), 3.42 (q, J=6.41 Hz, 2H), 3.59 (s, 2H), 6.88 (t, J=5.72 Hz, 1H), 7.25 (d, J=8.24 Hz, 1H), 7.62 (s, 1H), 7.69 (t, J=5.72 Hz, 1H), 8.19 (dd, J=8.70, 2.75 Hz, 1H), 8.80 (d, J=2.75 Hz, 1H), 9.08 (s, 1H); m/z (ES+APCI)⁺: 500 [M+H]⁺.

### Example 158

### Cyclobutanecarboxylic acid (3-{5-cyclopropyl-2-[6-(2-morpholin-4-yl-ethylamino)-pyridin-3-ylamino]-pyrimidin-4-ylamino}-propyl)-amide

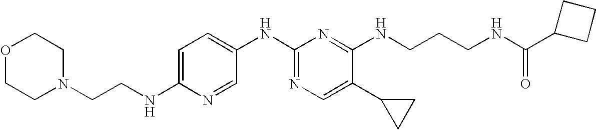

Prepared analogously to Example 97 using Intermediate 8 and N2-(2-morpholin-4-yl-ethyl)-pyridine-2,5-diamine to give an off-white coloured solid (20 mg, 31%). ¹H NMR (400 MHz, DMSO-d₆) δ ppm 0.39-0.44 (m, 2H), 0.76-0.82 (m, 2H), 1.38-1.46 (m, 1H), 1.61-1.77 (m, 3H), 1.81-1.91 (m, 1H), 1.92-2.02 (m, 2H), 2.05-2.15 (m, 2H), 2.36-2.48 (m, 6H), 2.95 (quin, J=8.24 Hz, 1H), 3.06-3.14 (m, 2H), 3.25-3.31 (m, 2H), 3.35-3.41 (m, 2H), 3.53-3.60 (m, 4H), 5.88 (t, J=5.72 Hz, 1H), 6.41 (d, J=8.70 Hz, 1H), 6.71 (t, J=5.72 Hz, 1H), 7.51 (s, 1H), 7.63-7.73 (m, 2H), 8.21 (d, J=2.75 Hz, 1H), 8.40 (s, 1H); m/z (ES+APCI)⁺: 495 [M+H]⁺.

### Example 159

### Cyclobutanecarboxylic acid (3-{5-cyclopropyl-2-[6-((R)-3-dimethylamino-pyrrolidin-1-ylmethyl)-pyridin-3-ylamino]-pyrimidin-4-ylamino}-propyl)-amide

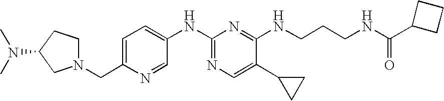

Prepared analogously to Example 146 using Intermediate 22 and (R)-(+)-3-dimethylaminopyrrolidine to provide the product as the free base (8 mg). ¹H NMR (400 MHz, DMSO-d₆) δ ppm 0.44-0.49 (m, 2H), 0.80-0.86 (m, 2H), 1.43-1.50 (m, 1H), 1.54-1.63 (m, 1H), 1.65-1.91 (m, 5H), 1.93-2.16 (m, 10H), 2.22-2.30 (m, 1H), 2.44 (td, J=8.82, 6.18 Hz, 1H), 2.52-2.71 (m, 3H), 2.97 (quin, J=8.24 Hz, 1H), 3.13 (q, J=6.41 Hz, 2H), 3.37-3.47 (m, 2H), 3.52 (d, J=13.28 Hz, 1H), 3.61 (d, J=13.28 Hz, 1H), 6.88 (t, J=5.95 Hz, 1H), 7.23 (d, J=8.70 Hz, 1H), 7.61 (s, 1H), 7.69 (t, J=5.72 Hz, 1H), 8.14-8.18 (m, 1H), 8.77 (d, J=2.75 Hz, 1H), 9.04 (s, 1H); m/z (ES+APCI)⁺: 493 [M+H]⁺.

### Example 160

### Cyclobutanecarboxylic acid (3-{5-cyclopropyl-2-[1-(2-piperidin-1-yl-ethyl)-1H-pyrazol-4-ylamino]-pyrimidin-4-ylamino}-propyl)-amide

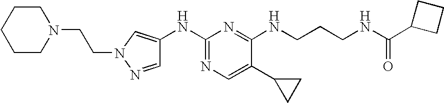

Prepared and analogously to Example 149 using Intermediate 8 and Intermediate 53 to give the product as the free base (19 mg, 31%). ¹H NMR (400 MHz, DMSO-d₆) δ ppm 0.38-0.47 (m, 2H), 0.75-0.85 (m, 2H), 1.29-1.49 (m, 7H), 1.60-1.77 (m, 3H), 1.79-1.91 (m, 1H), 1.92-2.02 (m, 2H), 2.04-2.15 (m, 2H), 2.34 (br. s, 4H), 2.63 (t, J=6.87 Hz, 2H), 2.96 (quin, J=8.24 Hz, 1H), 3.04-3.16 (m, 2H), 3.40 (q, J=−6.11 Hz, 2H), 4.10 (t, J=6.87 Hz, 2H), 6.70 (t, J=5.50 Hz, 1H), 7.41 (s, 1H), 7.53 (s, 1H), 7.69 (t, J=5.72 Hz, 1H), 7.80 (s, 1H), 8.65 (br. s, 1H); m/z (ES+APCI)⁺: 467 [M+H]⁺.

### Example 161

### Cyclobutanecarboxylic acid {3-[5-cyclopropyl-2-(1-isopropyl-1H-pyrazol-4-ylamino)-pyrimidin-4-ylamino]-propyl}-amide

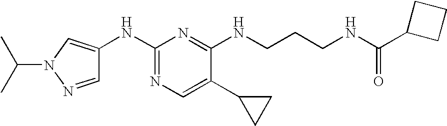

Prepared and analogously to Example 149 using Intermediate 8 and 1-isopropyl-1H-pyrazol-4-ylamine to give the product as a white foamy solid (22 mg, 34%). ¹H NMR (400 MHz, DMSO-d₆) δ ppm 0.36-0.45 (m, 2H), 0.75-0.84 (m, 2H), 1.32-1.47 (m, 7H), 1.60-1.77 (m, 3H), 1.79-1.91 (m, 1H), 1.93-2.02 (m, 2H), 2.05-2.16 (m, 2H), 2.96 (quin, J=8.24 Hz, 1H), 3.05-3.17 (m, 2H), 3.35-3.47 (m, 2H), 4.39 (quin, J=6.64 Hz, 1H), 6.71 (t, J=5.50 Hz, 1H), 7.41 (s, 1H), 7.54 (s, 1H), 7.68 (t, J=5.72 Hz, 1H), 7.82 (s, 1H), 8.65 (br. s, 1H); m/z (ES+APCI)⁺: 398 [M+H]⁺.

### Example 162

### Cyclobutyl-(3-{[5-cyclopropyl-2-(3-fluoro-phenylamino)-pyrimidin-4-ylamino]-methyl}-azetidin-1-yl)-methanone

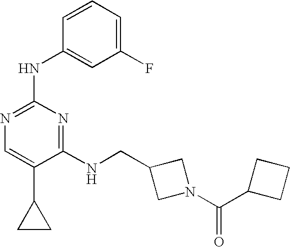

A solution of Intermediate 56 (37 mg, 0.1 mmol), 3-fluoroaniline (50 mg, 0.46 mmol) and glacial AcOH (3 drops) in n-butanol (2 ml) was heated at 150° C. in the microwave for 35 min. The crude mixture was concentrated under vacuum and purified by LCMS (low pH buffer). ¹H NMR (400 MHz, DMSO-d₆) δ ppm 0.48-0.65 (m, 2H), 0.80-0.97 (m, 2H), 1.60-1.80 (m, 2H), 1.81-2.29 (m, 6H), 2.85-3.08 (m, 1H), 3.11-3.57 (m, 3H), 3.57-3.75 (m, 2H), 3.76-3.92 (m, 1H), 4.47-4.72 (m, 1H), 6.83-7.01 (m, 1H), 7.20-7.44 (m, 2H), 7.51-7.70 (m, 2H); m/z (ES+APCI)⁺: 396 [M+H]⁺.

### Example 163

### Cyclobutanecarboxylic acid (3-{5-cyclopropyl-2-[4-(pyrimidin-2-ylsulfamoyl)-phenylamino]-pyrimidin-4-ylamino}-propyl)-amide

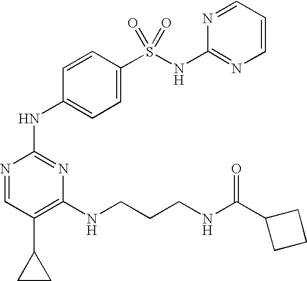

A solution of Intermediate 8 (50 mg, 0.16 mmol), 4-amino-N-pyrimidin-2-yl-benzenesulfonamide (162 mg, 0.65 mmol) and glacial AcOH (37 μl) in n-Butanol was heated at 150° C. in the microwave for 35 min. The precipitate was filtered and the filtrate was concentrated under vacuum and purified by preparative LCMS (low pH buffer). The resulting product was run through an amino propyl cartridge to give the free base (9.4 mg, 11%). ¹H NMR (400 MHz, CHLOROFORM-d) δ ppm 0.46-0.63 (m, 2H), 1.00 (d, J=6.87 Hz, 2H), 1.54 (d, J=4.12 Hz, 1H), 1.68-1.80 (m, 2H), 1.80-1.89 (m, 1H), 1.90-2.00 (m, 1H), 2.07-2.19 (m, 2H), 2.19-2.32 (m, 2H), 2.63 (s, 1H), 2.91-3.11 (m, 1H), 3.36 (q, J=5.95 Hz, 2H), 3.52-3.63 (m, 2H), 5.87 (t, J=6.41 Hz, 1H), 6.83 (br. s, 1H), 6.94 (t, J=4.81 Hz, 1H), 7.77 (d, J=9.16 Hz, 3H), 8.01 (d, J=8.70 Hz, 2H), 8.59 (d, J=4.58 Hz, 2H), 8.73 (br. s, 1H); m/z (ES+APCI)⁺: 523 [M+H]⁺.

### Examples 164-170

Examples 164-170 were prepared analogously to Example 163 (the general structure is shown below followed by the tabulated examples).

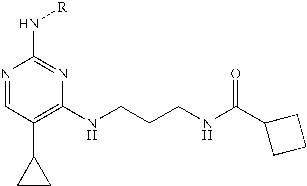

| | | | | HPLC |
|---|---|---|---|---|
| | | | m/z | retention |
| Example | Rgroup | Name | (ES + APCI)⁺ | time (min)* |
| 164 | 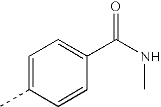 | 4-{4-[3-(Cyclobutanecarbonyl- amino)-propylamino]-5- cyclopropyl-pyrimidin-2- ylamino}-N-methyl-benzamide | 423 | 7.33^{a} |
| 165 | 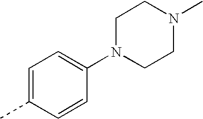 | Cyclobutanecarboxylic acid (3-{5- cyclopropyl-2-[4-(4-methyl- piperazin-1-yl)-phenylamino]- pyrimidin-4-ylamino}-propyl)- amide | 464 | 6.61^{a} |
| 166 | 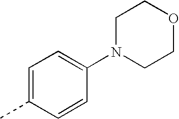 | Cyclobutanecarboxylic acid {3-[5- cyclopropyl-2-(4-morpholin-4-yl- phenylamino)-pyrimidin-4- ylamino]-propyl}-amide | 451 | 7.36^{a} |
| 167 | 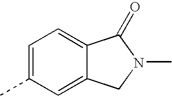 | Cyclobutanecarboxylic acid {3-[5- cyclopropyl-2-(2-methyl-1-oxo- 2,3-dihydro-1H-isoindol-5- ylamino)-pyrimidin-4-ylamino]- propyl}-amide | 435 | 6.31^{a} |
| 168 | 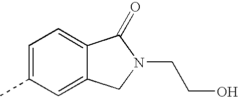 | Cyclobutanecarboxylic acid (3-{5- cyclopropyl-2-[2-(2-hydroxy- ethyl)-1-oxo-2,3-dihydro-1H- isoindol-5-ylamino]-pyrimidin-4- ylamino}propyl)-amide | 465 | 5.96^{a} |
| 169 | 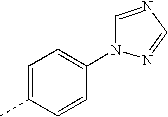 | Cyclobutanecarboxylic acid (3-{5- cyclopropyl-2-[4-(2H-1,2,4- triazol-1-yl)-phenylamino]- pyrimidin-4-ylamino}-propyl)- amide | 433 | 3.95^{a} |
| 170 | 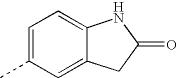 | Cyclobutanecarboxylic acid {3-[5- cyclopropyl-2-(2-oxo-2,3- dihydro-1H-indol-5-ylamino)- pyrimidin-4-ylamino]-propyl}- amide | 421 | 5.91^{a} |

^{a}: HPLC column: 21.2 × 100 mm (5 μm) C-18 Phenomenex Gemini-NX; flow rate: 30 ml/min; Run time: 12.0 min: Solvent A: 0.1% Trifluoro acetic acid in water, Solvent B: Methanol; Gradient - 10-100% B; Gradient time: 9.0 min.
^{b}: HPLC column: 4.6 × 50 mm (5 μm) C-18 Phenomenex Gemini-NX; flow rate: 2 ml/min; Run time: 4.6 min: Solvent A: 0.1% Ammonium Hydroxide in water, Solvent B: Methanol; Gradient - 10-100% B; Gradient time: 3.5 min.

### Example 171

### Cyclobutanecarboxylic acid (3-{5-cyclopropyl-2-[4-(1-methyl-piperidin-4-yl)-phenylamino]-pyrimidin-4-ylamino}-propyl)-amide

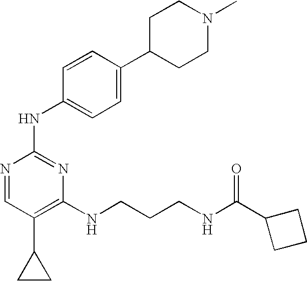

A solution of Intermediate 8 (45 mg, 0.15 mmol), 4-(1-methyl-piperidin-4-yl)-phenylamine (33 mg, 0.18 mmol), Pd₂(dba)₃ (8 mg, 0.009 mmol), xantphos (7 mg, 0.012 mmol) and NaO^{t}Bu (42 mg, 0.45 mmol) in dioxane was degassed and heated at 90° C. under N₂ overnight. The reaction mixture was concentrated under vacuum and purified by silica gel chromatography followed by preparative LCMS, to give the desired product (14 mg, 20%). ¹H NMR (400 MHz, CHLOROFORM-d) δ ppm 0.44-0.58 (m, 2H), 0.85-0.95 (m, 2H), 1.39-1.54 (m, 1H), 1.69-1.95 (m, 7H), 1.97-2.11 (m, 4H), 2.13-2.29 (m, 2H), 2.33 (s, 3H), 2.37-2.50 (m, 1H), 2.67-2.82 (m, 1H), 2.97 (d, J=10.53 Hz, 2H), 3.32 (q, J=6.41 Hz, 2H), 3.48 (s, 1H), 3.60 (q, J=5.95 Hz, 2H), 5.76 (t, J=6.18 Hz, 1H), 5.82 (t, J=6.18 Hz, 1H), 6.90 (s, 1H), 7.16 (d, J=8.70 Hz, 2H), 7.52 (d, J=8.70 Hz, 2H), 7.72 (s, 1H); m/z (ES+APCI)⁺: 463 [M+H]⁺.

### Example 172

### Cyclobutanecarboxylic acid (3-{5-cyclopropyl-2-[4-(2-dimethylamino-ethyl)-phenylamino]-pyrimidin-4-ylamino}-propyl)-amide

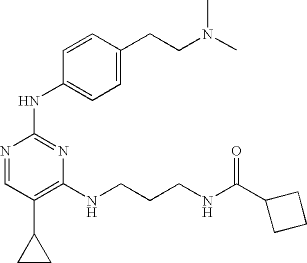

A solution of Intermediate 8 (45 mg, 0.15 mmol), 4-(2-dimethylamino-ethyl)-phenylamine (30 mg, 0.18 mmol), Pd₂(dba)₃ (8 mg, 0.009 mmol), xantphos (7 mg, 0.012 mmol) and NaO^{t}Bu (42 mg, 0.45 mmol) in dioxane was degassed and heated at 90° C. under N₂ overnight. The reaction mixture was purified silica gel chromatography followed by preparative LCMS. The resulting residues were run through an Isolute-NH₂ cartridge, eluting with MeOH, and concentrated under vacuum to give the desired product (10.2 mg, 16%). ¹H NMR (400 MHz, CHLOROFORM-d) δ ppm 0.45-0.55 (m, 2H), 0.85-0.97 (m, 2H), 1.39-1.53 (m, 1H), 1.68-1.96 (m, 4H), 1.96-2.10 (m, 2H), 2.15-2.28 (m, 2H), 2.30 (s, 6H), 2.47-2.56 (m, 2H), 2.68-2.78 (m, 3H), 3.31 (q, J=6.41 Hz, 2H), 3.60 (q, J=6.41 Hz, 2H), 5.79 (q, J=6.26 Hz, 2H), 7.00 (s, 1H), 7.14 (d, J=8.70 Hz, 2H), 7.51 (d, J=8.24 Hz, 2H), 7.73 (s, 1H); m/z (ES+APCI)⁺: 437 [M+H]⁺.

### Example 173

### Cyclobutanecarboxylic acid (3-{5-cyclopropyl-2-[4-(2-dimethylamino-methyl)-phenylamino]-pyrimidin-4-ylamino}-propyl)-amide

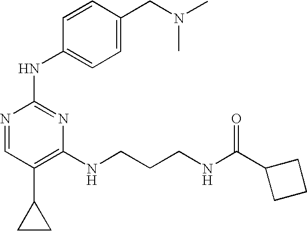

Prepared analogously to Example 172 from Intermediate 8 (45 mg, 0.15 mmol) to give the product (9.7 mg, 15%). ¹H NMR (400 MHz, CHLOROFORM-d) δ ppm 0.46-0.53 (m, 2H), 0.86-0.96 (m, 2H), 1.40-1.52 (m, 1H), 1.71-1.96 (m, 4H), 1.99-2.12 (m, 2H), 2.17-2.30 (m, 8H), 2.78 (quin, J=8.36 Hz, 1H), 3.24-3.36 (m, 2H), 3.37 (s, 2H), 3.59 (q, J=6.41 Hz, 2H), 5.78 (t, J=6.18 Hz, 1H), 5.87 (t, J=6.41 Hz, 1H), 7.08 (s, 1H), 7.22 (d, J=8.70 Hz, 2H), 7.56 (d, J=8.70 Hz, 2H), 7.73 (s, 1H); m/z (ES+APCI)⁺: 423 [M+H]⁺.

### Example 174

### Cyclobutanecarboxylic acid (3-{5-cyclopropyl-2-[4-(morpholine-4-carbonyl)-phenylamino]-pyrimidin-4-ylamino}-propyl)-amide

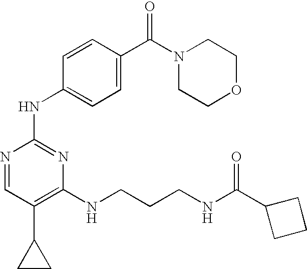

A solution of Intermediate 57 (40 mg, 0.1 mmol), morpholine (9 mg, 0.1 mmol), DIPEA (29 μL, 0.3 mmol) and HATU (42 mg, 0.11 mmol) in DMF was stirred at RT for 18 h. The reaction was concentrated under vacuum and purified by preparative LCMS (low pH buffer). The resulting fractions were combined and run through a carbonate cartridge, eluting with MeOH, then concentrated under vacuum to give the product (4.4 mg, 9%). ¹H NMR (400 MHz, DMSO-d6) δ ppm 0.43-0.51 (m, 2H), 0.78-0.87 (m, 2H), 1.41-1.53 (m, 1H), 1.65-1.78 (m, 3H), 1.80-1.92 (m, 1H), 1.93-2.03 (m, 2H), 2.03-2.18 (m, 2H), 2.97 (t, J=8.47 Hz, 1H), 3.09-3.19 (m, 2H), 3.43 (q, J=6.41 Hz, 3H), 3.50 (br. s, 3H), 3.55-3.65 (m, 4H), 6.89 (t, J=5.72 Hz, 1H), 7.31 (d, J=8.70 Hz, 2H), 7.63 (s, 1H), 7.70 (t, J=5.72 Hz, 1H), 7.83 (d, J=8.70 Hz, 2H), 9.17 (s, 1H); m/z (ES+APCI)+: 479 [M+H]⁺.

### Examples 175-176

Prepared analogously to Example 174 (the general structure is shown below followed by the tabulated examples).

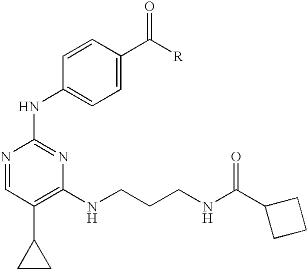

| | | | | HPLC |
|---|---|---|---|---|
| | | | | retention |
| | | | m/z | time |
| Example | R group | Name | (ES + APCI)⁺ | (min)* |
| 175 | 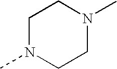 | Cyclobutanecarboxylic acid (3- {5-cyclopropyl-2-[4-(4-methyl- piperazine-1-carbonyl)- phenylamino]-pyrimidin-4- ylamino}-propyl)-amide | 492 | 9.29 |
| 176 | 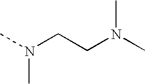 | 4-{4-[3-(Cyclobutanecarbonyl- amino)-propylamino]-5- cyclopropyl-pyrimidin-2- ylamino}-N-(2-dimethylamino- ethyl)-N-methyl-benzamide | 494 | 9.36 |

*HPLC column: 21.2 × 100 mm (5 μm) C-18 Phenomenex Gemini; flow rate: 20 ml/min; run time: 10 min; gradient at start: 10% methanol and 90% water, gradient at finish: 100% methanol and 0% water; as buffer: 0.1% trifluoroacetic acid is added to the water.

### Example 177

### Cyclobutanecarboxylic acid (3-{5-cyclopropyl-2-[4-(2-hydroxy-ethyl)-phenylamino]-pyrimidin-4-ylamino}-propyl)-amide

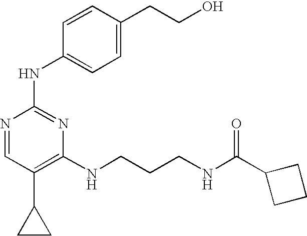

A solution of Intermediate 8 (40 mg, 0.13 mmol), 2-(4-amino-phenyl)-ethanol (55 mg, 0.4 mmol) and glacial AcOH (2 mg) in n-butanol heated at 150° C. in the microwave for 30 min. The resulting precipitate was filtered and washed with MeOH followed by petroleum ether to give the desired product (16 mg, 30%). ¹H NMR (400 MHz, DMSO-d₆) δ ppm 0.51-0.60 (m, 2H), 0.87-0.94 (m, 2H), 1.48-1.59 (m, 1H), 1.67-1.78 (m, 3H), 1.79-1.92 (m, 1H), 1.93-2.02 (m, 2H), 2.04-2.16 (m, 2H), 2.71 (t, J=7.10 Hz, 2H), 2.95 (t, J=8.47 Hz, 1H), 3.07-3.17 (m, 2H), 3.46 (q, J=6.41 Hz, 2H), 3.60 (t, J=7.10 Hz, 2H), 4.65 (br. s, 1H), 7.24 (d, J=8.70 Hz, 2H), 7.45 (d, J=8.24 Hz, 2H), 7.56 (s, 1H), 7.76 (t, J=5.72 Hz, 1H), 8.56 (br. s, 1H), 10.18 (s, 1H); m/z (ES+APCI)⁺: 409 [M+H]⁺.

### Example 178

### Cyclobutanecarboxylic acid {3-[5-cyclopropyl-2-(2-oxo-2,3-dihydro-1H-benzimidazol-5-ylamino)-pyrimidin-4-ylamino]-propyl}-amide

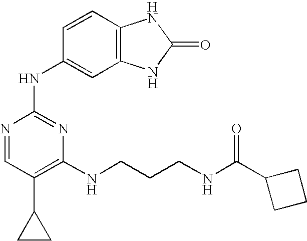

A solution of Intermediate 8 (80 mg 0.26 mmol), 5-amino-1,3-dihydro-benzimidazol-2-one (77 mg, 0.52 mmol) and glacial AcOH (cat.) in n-butanol was heated at 150° C. in the microwave for 35 min. The precipitate was removed and the filtrate was concentrated under vacuum and triturated in EtOAc yielding a second precipitate. This was purified by preparative LCMS (low pH buffer) to give the product (11 mg, 10%). ¹H NMR (400 MHz, DMSO-d₆) δ ppm 0.52-0.60 (m, 2H), 1.47-1.58 (m, 1H), 1.67-1.78 (m, 3H), 1.79-1.93 (m, 1H), 1.93-2.03 (m, 2H), 2.03-2.16 (m, 2H), 2.88-3.02 (m, 1H), 3.05-3.15 (m, 2H), 3.45 (q, J=6.41 Hz, 2H), 6.90-7.02 (m, 2H), 7.24 (br. s, 1H), 7.47 (br. s, 1H), 7.75 (t, J=5.72 Hz, 1H), 8.48 (t, J=5.50 Hz, 1H), 10.16 (s, 1H), 10.66 (s, 1H), 10.77 (s, 1H); m/z (ES+APCI)⁺: 422 [M+H]⁺.

### Example 179

### Cyclobutanecarboxylic acid {3-[5-cyclopropyl-2-(1,3-dimethyl-2-oxo-2,3-dihydro-1H-benzimidazol-5-ylamino)-pyrimidin-4-ylamino]-propyl}-amide

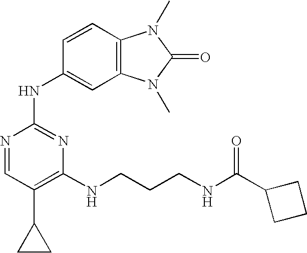

Prepared analogously to Example 178 from Intermediate 8 and 5-amino-1,3-dimethyl-1,3-dihydro-2H-benzimidazol-2-one to give the desired product (32 mg, 27%). ¹H NMR (400 MHz, DMSO-d₆) δ ppm 0.51-0.60 (m, 2H), 0.88-0.94 (m, 2H), 1.13-1.20 (m, 4H), 1.50-1.59 (m, 1H), 1.66-1.77 (m, 3H), 1.83-1.89 (m, 1H), 1.93-2.01 (m, 4H), 2.02-2.13 (m, 2H), 2.93 (t, J=8.24 Hz, 1H), 3.04-3.13 (m, 2H), 3.47 (q, J=6.26 Hz, 2H), 4.03 (q, J=6.87 Hz, 2H), 7.06-7.13 (m, 1H), 7.15-7.20 (m, 1H), 7.45 (s, 1H), 7.51 (br. s, 1H), 7.73 (t, J=5.50 Hz, 1H); m/z (ES+APCI)⁺: 450 [M+H]⁺.

### Example 180

### 4-{4-[3-(Cyclobutanecarbonyl-amino)-propylamino]-5-cyclopropyl-pyrimidin-2-ylamino}-N,N-dimethyl-benzamide

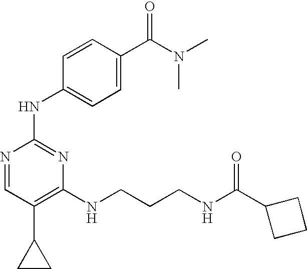

A solution of Intermediate 8 (50 mg, 0.16 mmol), 4-amino-N,N-dimethyl-benzamide (78 mg, 0.49 mmol) and glacial AcOH (cat.) in n-butanol was heated at 150° C. in the microwave for 35 min. The crude reaction mixture was concentrated under vacuum and purified by prep LCMS (high pH buffer) to give the product (40 mg, 57%). ¹H NMR (400 MHz, DMSO-d₆) δ ppm 0.41-0.54 (m, 2H), 0.73-0.89 (m, 2H), 1.40-1.54 (m, 1H), 1.63-1.78 (m, 3H), 1.78-1.92 (m, 1H), 1.91-2.04 (m, 2H), 2.04-2.20 (m, 2H), 2.87-3.04 (m, 7H), 3.08-3.19 (m, 2H), 3.43 (q, J=6.41 Hz, 2H), 6.89 (t, J=5.95 Hz, 1H), 7.30 (d, J=8.70 Hz, 2H), 7.63 (s, 1H), 7.70 (t, J=5.72 Hz, 1H), 7.81 (d, J=8.70 Hz, 2H), 9.14 (s, 1H) 437 [M+H]⁺.

### Examples 181-183

The following examples were prepared analogously to Example 180 (the general structure is shown below followed by the tabulated examples).

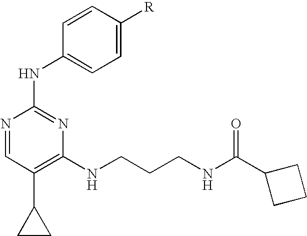

| | | | | |
|---|---|---|---|---|
| 181 | 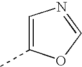 | Cyclobutanecarboxylic acid {3- [5-cyclopropyl-2-(4- oxazol-5-yl- phenylamino)-pyrimidin-4- ylamino]-propyl}-amide | 433 | 2.70^{a} |
| 182 | 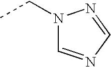 | Cyclobutanecarboxylic acid {3- [5-cyclopropyl-2- (4-1,2,4-triazol- 1-ylmethyl-phenylamino)- pyrimidin-4-ylamino]- propyl}- amide | 447 | 7.97^{b} |
| 183 | 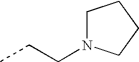 | Cyclobutanecarboxylic acid (3- {5-cyclopropyl-2-[4-(2- pyrrolidin-1-yl-ethyl)- phenylamino]-pyrimidin-4- ylamino}-propyl)-amide | 463 | 9.64^{b} |

^{a}: HPLC column: 4.6 × 50 mm (5 μm) C-18 Phenomenex Gemini-NX; flow rate: 2 ml/min; Run time: 4.6 min: Solvent A: 0.1% Ammonium Hydroxide in water, Solvent B: Methanol; Gradient - 10-100% B; Gradient time: 3.5 min:
^{b}: HPLC column: 21.2 × 100 mm (5 μm) C-18 Phenomenex Gemini-NX; flow rate: 40 ml/min: gradient time: 7.5 min, run time: 9 min; gradient at start: 10% methanol and 90% water, gradient at finish: 100% methanol and 0% water; as buffer: 0.1% ammonium hydroxide is added to the water

### Example 184

### Cyclobutanecarboxylic acid (3-{5-cyclopropyl-2-[4-(2-oxo-pyrrolidin-1-yl)-phenylamino]-pyrimidin-4-ylamino}-propyl)-amide. xHCl

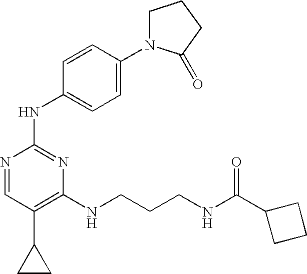

A solution of Intermediate 8 (50 mg, 0.16 mmol), 1-(4-amino-phenyl)-pyrrolidin-2-one (80 mg, 0.49 mmol) and glacial AcOH (cat.) in n-butanol was heated at 150° C. in the microwave for 35 min. The resulting precipitate was filtered and recrystallised from 1:1 MeOH:H₂O to give the desired product (6.2 mg, 9%). ¹H NMR (400 MHz, DMSO-d₆) δ ppm 0.53-0.60 (m, 2H), 0.86-0.94 (m, 2H), 1.49-1.59 (m, 1H), 1.66-1.78 (m, 3H), 1.78-1.91 (m, 1H), 1.91-2.01 (m, 2H), 2.02-2.14 (m, 4H), 2.95 (quin, J=8.24 Hz, 1H), 3.05-3.15 (m, 2H), 3.46 (q, J=6.41 Hz, 2H), 3.84 (t, J=7.10 Hz, 2H), 7.50-7.61 (m, 3H), 7.65-7.72 (m, 2H), 7.77 (t, J=5.72 Hz, 1H), 8.59 (br. s, 1H), 10.28 (s, 1H), 11.91 (br. s, 1H); m/z (ES+APCI)⁺: 449 [M+H]⁺.

### Example 185

### Cyclobutanecarboxylic acid {3-[5-cyclopropyl-2-(6-oxazol-5-yl-pyridin-3-ylamino)-pyrimidin-4-ylamino]-propyl}-amide

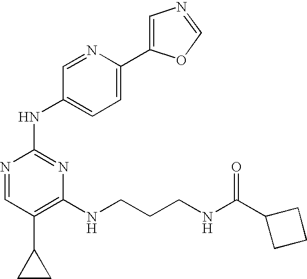

Prepared analogously to Example 172 from Intermediate 8 (70 mg, 0.23 mmol) and Intermediate 64 (35 mg, 0.23 mmol) to give the desired product as a white solid (9 mg, 9%). ¹H NMR (400 MHz, DMSO-d₆) δ ppm 0.41-0.56 (m, 2H), 0.76-0.92 (m, 2H), 1.48 (br. s, 1H), 1.66-1.76 (m, 3H), 1.83 (d, J=10.07 Hz, 1H), 1.97 (dd, J=8.70, 3.21 Hz, 2H), 2.03-2.17 (m, 2H), 2.97 (t, J=8.24 Hz, 1H), 3.09-3.20 (m, 2H), 3.44 (d, J=5.95 Hz, 2H), 6.97 (s, 1H), 7.58 (s, 1H), 7.63-7.69 (m, 2H), 7.71 (s, 1H), 8.38 (dd, J=8.93, 2.52 Hz, 1H), 8.44 (s, 1H), 8.93 (d, J=2.29 Hz, 1H), 9.38 (s, 1H); m/z (ES+APCI)⁺: 434 [M+H]⁺.

### Examples 186-191

Examples 186-191 were prepared analogously to Example 172 (the general structure is shown below followed by the tabulated examples).

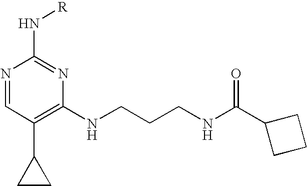

| | | | | HPLC |
|---|---|---|---|---|
| | | | m/z | retention |
| | | | (ES + | time |
| Example | R group | Name | APCI)⁺ | (min)* |
| 186 | 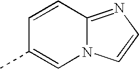 | Cyclobutanecarboxylic acid {3-[5-cyclopropyl-2- (imidazo[1,2-a]pyridin-6- ylamino)-pyrimidin-4- ylamino]-propyl}-amide | 406 | 3.36 |
| 187 | 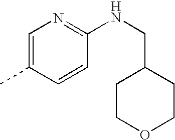 | Cyclobutanecarboxylic acid [3-(5-cyclopropyl-2-{(6- [(tetrahydro-pyran-4- ylmethyl)-amino]-pyridin- 3-ylamino}-pyrimidin-4- ylamino)-propyl]-amide | 478 | 3.39 |
| 188 | 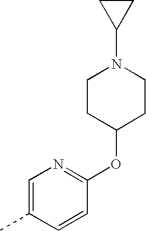 | Cyclobutanecarboxylic acid (3-{5-cyclopropyl-2-[6-(1- cyclopropyl-piperidin-4- yloxy)-pyridin-3-ylamino]- pyrimidin-4-ylamino}- propyl)-amide | 506 | 3.81 |
| 189 | 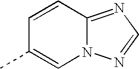 | Cyclobutanecarboxylic acid {3-[5-cyclopropyl-2- ([1,2,4]triazolo[1,5- a]pyridin-6-ylamino)- pyrimidin-4-ylamino]- propyl}-amide | 407 | 3.39 |
| 190 | 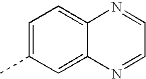 | Cyclobutanecarboxylic acid {3-[5-cyclopropyl-2- (quinoxalin-6-ylamino)- pyrimidin-4-ylamino]- propyl}-amide | 418 | 3.47 |
| 191 | 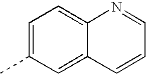 | Cyclobutanecarboxylic acid {3-[5-cyclopropyl-2- (quinolin-6-ylamino)- pyrimidin-4-ylamino]- propyl}-amide | 417 | 3.54 |

*HPLC column: 21.2 × 100 mm (10 μm) C-18 Phenomenex Gemini; flow rate: 20 ml/min; run time: 10 min; gradient at start: 10% methanol and 90% water, gradient at finish: 100% methanol and 0% water; as buffer: ammonium bicarbonate (10 mmol) and ammonium hydroxide is added to the water.

### Example 192

### Cyclobutanecarboxylic acid (3-{5-cyclopropyl-2-[1-(tetrahydro-pyran-4-yl)-1H-pyrazol-4-ylamino]-pyrimidin-4-ylamino}-propyl)-amide

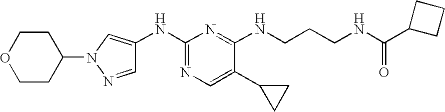

Prepared analogously to Example 91 using Intermediate 8 and 1-(tetrahydro-pyran-4-yl)-1H-pyrazol-4-ylamine to provide the product as an off-white solid (18 mg, 32%). ¹H NMR (400 MHz, DMSO-d₆) δ ppm 0.40-0.46 (m, 2H), 0.77-0.84 (m, 2H), 1.40-1.47 (m, 1H), 1.65-1.78 (m, 3H), 1.80-2.03 (m, 7H), 2.06-2.17 (m, 2H), 2.98 (quin, J=8.36 Hz, 1H), 3.09-3.18 (m, 2H), 3.37-3.49 (m, 4H), 3.94 (dd, J=11.22, 3.43 Hz, 2H), 4.27-4.36 (m, 1H), 6.70-6.77 (m, 1H), 7.45 (s, 1H), 7.55 (s, 1H), 7.71 (t, J=5.72 Hz, 1H), 7.86 (s, 1H), 8.69 (br. s, 1H); m/z (ES+APCI)⁺: 440 [M+H]⁺.

### Example 193

### Cyclobutanecarboxylic acid {3-[5-cyclopropyl-2-(1-cyclopropylmethyl-1H-indazol-6-ylamino)-pyrimidin-4-ylamino]-propyl}-amide

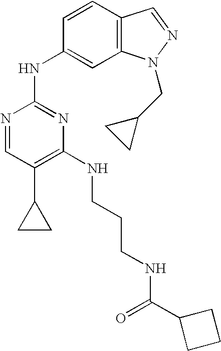

Prepared analogously to Example 90 using Intermediate 8 and Intermediate 73 to provide the product as a white foamy solid (35 mg, 59%). ¹H NMR (400 MHz, DMSO-d₆) δ ppm 0.32-0.37 (m, 2H), 0.43-0.52 (m, 4H), 0.81-0.87 (m, 2H), 1.19-1.29 (m, 1H), 1.46-1.53 (m, 1H), 1.66-1.87 (m, 4H), 1.90-1.99 (m, 2H), 2.03-2.14 (m, 2H), 2.88-2.97 (m, 1H), 3.15 (q, J=6.41 Hz, 2H), 3.53 (q, J=6.26 Hz, 2H), 4.15 (d, J=6.87 Hz, 2H), 6.89 (t, J=5.72 Hz, 1H), 7.19-7.23 (m, 1H), 7.53 (d, J=9.16 Hz, 1H), 7.65-7.71 (m, 2H), 7.84 (s, 1H), 8.42 (s, 1H), 9.16 (s, 1H); m/z (ES+APCI)⁺: 460 [M+H]⁺.

### Example 194

### Cyclobutanecarboxylic acid {3-[5-cyclopropyl-2-(2-cyclopropylmethyl-2H-indazol-6-ylamino)-pyrimidin-4-ylamino]-propyl}-amide

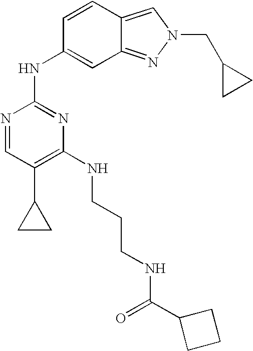

Prepared analogously to Example 90 using Intermediate 8 and Intermediate 74 to provide the product as an off-white solid (28 mg, 47%). ¹H NMR (400 MHz, DMSO-d₆) δ ppm 0.40-0.58 (m, 6H), 0.80-0.86 (m, 2H), 1.31-1.41 (m, 1H), 1.44-1.51 (m, 1H), 1.66-1.87 (m, 4H), 1.88-1.99 (m, 2H), 2.05-2.16 (m, 2H), 2.90-2.99 (m, 1H), 3.18 (q, J=6.41 Hz, 2H), 3.43-3.51 (m, 2H), 4.19 (d, J=7.33 Hz, 2H), 6.88 (t, J=5.95 Hz, 1H), 7.16-7.20 (m, 1H), 7.49 (d, J=9.16 Hz, 1H), 7.64 (s, 1H), 7.80 (t, J=5.95 Hz, 1H), 8.21 (s, 1H), 8.33 (s, 1H), 8.91 (s, 1H); m/z (ES+APCI)⁺: 460 [M+H]⁺.

### Examples 195-208

Examples 195-208 were prepared analogously to Example 91 (the general structure is shown below followed by the tabulated examples) using intermediate 8 and the appropriate amines. The amines for Examples 202, 203 and 205 were prepared analogously to Intermediate 70.

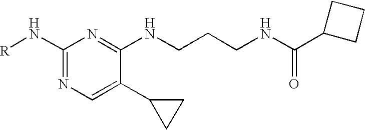

| | | | | HPLC |
|---|---|---|---|---|
| | | | m/z | retention |
| | | | (ES + | time |
| Example | Rgroup | Name | APCI)⁺ | (min) |
| 195 | 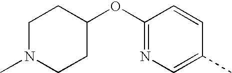 | Cyclobutanecarboxylic acid (3-{5- cyclopropyl-2-[6-(1- methyl-piperidin-4- yloxy)-pyridin-3- ylamino]-pyrimidin-4- ylamino}-propyl)- amide | 480 | 5.12^{a} |
| 196 | 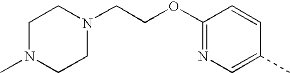 | Cyclobutanecarboxylic acid [3-(5- cyclopropyl-2-{6-[2- (4-methyl-piperazin- 1-yl)-ethoxy]-pyridin- 3-ylamino}- pyrimidin-4-ylamino)- propyl]-amide | 509 | 4.77^{a} |
| 197 | 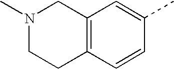 | Cyclobutanecarboxylic acid {3-[5- cyclopropyl-2-(2- methyl-1,2,3,4- tetrahydro- isoquinolin-7- ylamino)-pyrimidin-4- ylamino]-propyl}- amide | 435 | 2.23^{b} |
| 198 | 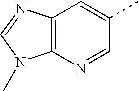 | Cyclobutanecarboxylic acid {3-[5- cyclopropyl-2-(3- methyl-3H- imidazo[4,5- b]pyridin-6-ylamino)- pyrimidin-4-ylamino]- propyl}-amide | 421 | 2.36^{b} |
| 199 | 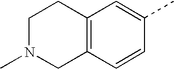 | Cyclobutanecarboxylic acid {3-[5- cyclopropyl-2-(2- methyl-1,2,3,4- tetrahydro- isoquinolin-6- ylamino)-pyrimidin-4- ylamino]-propyl}- amide | 435 | 2.19^{b} |
| 200 | 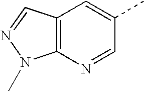 | Cyclobutanecarboxylic acid {3-[5- cyclopropyl-2-(1- methyl-1H- pyrazolo[3,4- b]pyridin-5-ylamino)- pyrimidin-4-ylamino]- propyl}-amide | 421 | 2.64^{b} |
| 201 | 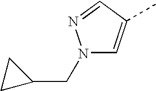 | Cyclobutanecarboxylic acid {3-[5- cyclopropyl-2-(1- cyclopropylmethyl- 1H-pyrazol-4- ylamino)-pyrimidin-4- ylamino]-propyl}- amide | 410 | 2.27^{c} |
| 202 | 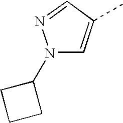 | Cyclobutanecarboxylic acid {3-[2-(1- cyclobutyl-1H- pyrazol-4-ylamino)-5- cyclopropyl- pyrimidin-4-ylamino]- propyl}-amide | 410 | 3.48^{d} |
| 203 | 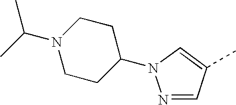 | Cyclobutanecarboxylic acid (3-{5- cyclopropyl-2-[1-(1- isopropyl-piperidin-4- yl)-1H-pyrazol-4- ylamino]-pyrimidin-4- ylamino}-propyl)- amide | 481 | 3.6^{d} |
| 204 | 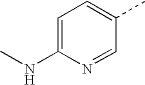 | Cyclobutanecarboxylic acid {3-[5- cyclopropyl-2-(6- methylamino-pyridin- 3-ylamino)-pyrimidin- 4-ylamino]-propyl}- amide | 396 | 3.21^{d} |
| 205 | 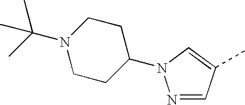 | Cyclobutanecarboxylic acid (3-{2-[1-(1-tert- butyl-piperidin-4-yl)- 1H-pyrazol-4- ylamino]-5- cyclopropyl- pyrimidin-4- ylamino}-propyl)- amide | 495 | 3.67^{d} |
| 206 | 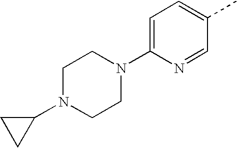 | Cyclobutanecarboxylic acid (3-{5- cyclopropyl-2-[6-(4- cyclopropyl- piperazin-1-yl)- pyridin-3-ylamino]- pyrimidin-4- ylamino}-propyl)- amide | 491 | 1.62^{c} |
| 207 | 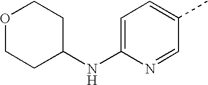 | Cyclobutanecarboxylic acid (3-{5- cyclopropyl-2-[6- (tetrahydro-pyran-4- ylamino)-pyridin-3- ylamino]-pyrimidin-4- ylamino}-propyl)- amide | 466 | 1.75^{c} |
| 208 | 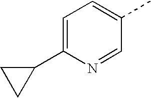 | Cyclobutanecarboxylic acid {3-[5- cyclopropyl-2-(6- cyclopropyl-pyridin- 3-ylamino)-pyrimidin- 4-ylamino]-propyl}- amide | 407 | 1.87^{e} |

^{a}HPLC column: 21.2 × 100 mm (5 μm) C-18 Phenomenex Gemini-NX; flow rate: 40 ml/min; Run time: 9.0 min: Solvent A: 0.1% Trifluoro acetic acid in water, Solvent B: Methanol; Gradient-10-100% B; Gradient time: 7.5 min.
^{b}HPLC column: 4.6 × 50 mm (5 μm) C-18 Phenomenex Gemini-NX; flow rate: 2 ml/min; Run time: 4.6 min: Solvent A: 0.1% Trifluoro acetic acid in water, Solvent B: Methanol; Gradient-10-100% B; Gradient time: 3.5 min.
^{c}HPLC column: 4.6 × 50 mm (5 μm) C-18 Phenomenex Gemini-NX; flow rate: 2 ml/min; Run time: 4.6 min: Solvent A: 0.1% Formic acid in water, Solvent B: Methanol; Gradient-10-100% B; Gradient time: 3.5 min.
^{d}HPLC column: 4.6 × 50 mm (5 μm) C-18 Phenomenex Gemini-NX; flow rate: 2 ml/min; Run time: 4.6 min: Solvent A: 0.1% Ammonium Hydroxide in water, Solvent B: Methanol; Gradient-10-100% B; Gradient time: 3.5 min.
^{e}HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient-10-100% B; Gradient time: 2.35 min.

### Examples 209-214

Examples 209-214 were prepared analogously to Example 90 (the general structure is shown below followed by the tabulated examples) using Intermediate 8 and the appropriate amines

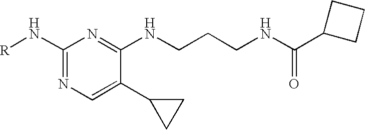

| | | | | HPLC |
|---|---|---|---|---|
| | | | m/z | Retention |
| | | | (ES + | time |
| Example | R group | Name | APCI)⁺ | (min) |
| 209 | 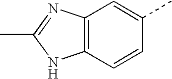 | Cyclobutanecarboxylicacid{3- [5-cyclopropyl-2-(2-methyl- 3H-benzimidazol-5-ylamino)- pyrimidin-4-ylamino]- propyl}-amide | 420 | 2.35^{a} |
| 210 | 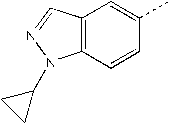 | Cyclobutanecarboxylicacid{3- [5-cyclopropyl-2-(1- cyclopropyl-1H-indazol-5- ylamino)-pyrimidin-4- ylamino]-propyl}-amide | 446 | 2.44^{b} |
| 211 | 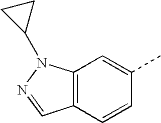 | Cyclobutanecarboxylicacid{3- [5-cyclopropyl-2-(1- cyclopropyl-1H-indazol-6- ylamino)-pyrimidin-4- ylamino]-propyl}-amide | 446 | 2.67^{b} |
| 212 | 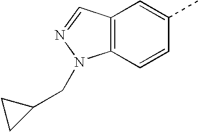 | Cyclobutanecarboxylicacid{3- [5-cyclopropyl-2-(1- cyclopropylmethyl-1H- indazol-5-ylamino)- pyrimidin-4-ylamino]- propyl}-amide | 460 | 2.77^{b} |
| 213 | 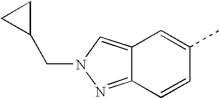 | Cyclobutanecarboxylicacid{3- [5-cyclopropyl-2-(2- cyclopropylmethyl-2H- indazol-5-ylamino)- pyrimidin-4-ylamino]- propyl}-amide | 460 | 2.71^{b} |
| 214 | 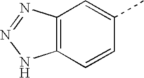 | Cyclobutanecarboxylicacid{3- [2-(1H-benzotriazol-5- ylamino)-5-cyclopropyl- pyrimidin-4-ylamino]- propyl}-amide | 407 | 1.06^{c} |

^{a}HPLC column: 4.6 × 50 mm (5 μm) C-18 Phenomenex Gemini-NX; flow rate: 2 ml/min; Run time: 4.6 min: Solvent A: 0.1% Trifluoro acetic acid in water, Solvent B: Methanol; Gradient-10-100% B; Gradient time: 3.5 min.
^{b}HPLC column: 4.6 × 50 mm (5 μm) C-18 Phenomenex Gemini-NX; flow rate: 2 ml/min; Run time: 4.6 min: Solvent A: 0.1% Formic acid in water, Solvent B: Methanol; Gradient-10-100% B; Gradient time: 3.5 min.
^{c}HPLC column: 4.6 × 50 mm (5 μm) C-18 Xbridge; flow rate: 3 ml/min; Run time: 3.2 min: Solvent A: 0.1% Ammonium Hydroxide in water Solvent B: Acetonitrile; Gradient-10-100% B; Gradient time: 2.35 min.

### Results

All compounds exemplified below in Table 1 have IC₅₀ values against TBK1 of 10 μM or better. The tables below shows a potency score for each compound (***=TBK1 IC₅₀<100 nM; **=TBK1 IC₅₀ between 100 nM and 1 μM; *=TBK1 IC₅₀ between 1 μM and 10 μM):

TABLE 1

| Example # | Potency |
|---|---|
| Example 1 | ** |
| Example 2 | *** |
| Example 3 | *** |
| Example 4 | ** |
| Example 5 | ** |
| Example 6 | *** |
| Example 7 | ** |
| Example 8 | *** |
| Example 9 | ** |
| Example 10 | * |
| Example 11 | *** |
| Example 12 | ** |
| Example 13 | * |
| Example 14 | *** |
| Example 15 | ** |
| Example 16 | ** |
| Example 17 | *** |
| Example 18 | * |
| Example 19 | ** |
| Example 20 | *** |
| Example 21 | ** |
| Example 22 | *** |
| Example 23 | *** |
| Example 24 | *** |
| Example 25 | ** |
| Example 26 | ** |
| Example 27 | ** |
| Example 28 | *** |
| Example 29 | * |
| Example 30 | * |
| Example 31 | * |
| Example 32 | *** |
| Example 33 | ** |
| Example 34 | ** |
| Example 35 | ** |
| Example 36 | ** |
| Example 37 | *** |
| Example 38 | ** |
| Example 39 | ** |
| Example 40 | *** |
| Example 41 | *** |
| Example 42 | *** |
| Example 43 | *** |
| Example 44 | ** |
| Example 45 | ** |
| Example 46 | ** |
| Example 47 | ** |
| Example 48 | *** |
| Example 49 | *** |
| Example 50 | ** |
| Example 51 | *** |
| Example 52 | ** |
| Example 53 | * |
| Example 54 | *** |
| Example 55 | ** |
| Example 56 | ** |
| Example 57 | ** |
| Example 58 | ** |
| Example 59 | * |
| Example 60 | ** |
| Example 61 | ** |
| Example 62 | * |
| Example 63 | ** |
| Example 64 | *** |
| Example 65 | *** |
| Example 66 | *** |
| Example 67 | *** |
| Example 68 | *** |
| Example 69 | *** |
| Example 70 | *** |
| Example 71 | *** |
| Example 72 | *** |
| Example 73 | *** |
| Example 74 | *** |
| Example 75 | *** |
| Example 76 | *** |
| Example 77 | *** |
| Example 78 | *** |
| Example 79 | *** |
| Example 80 | *** |
| Example 81 | *** |
| Example 82 | ** |
| Example 83 | *** |
| Example 84 | *** |
| Example 85 | *** |
| Example 86 | *** |
| Example 87 | *** |
| Example 88 | *** |
| Example 89 | *** |
| Example 90 | *** |
| Example 91 | *** |
| Example 92 | *** |
| Example 93 | *** |
| Example 94 | ** |
| Example 95 | *** |
| Example 96 | *** |
| Example 97 | *** |
| Example 98 | *** |
| Example 99 | *** |
| Example 100 | *** |
| Example 101 | *** |
| Example 102 | *** |
| Example 103 | *** |
| Example 104 | *** |
| Example 105 | ** |
| Example 106 | ** |
| Example 107 | *** |
| Example 108 | *** |
| Example 109 | *** |
| Example 110 | ** |
| Example 111 | ** |
| Example 112 | ** |
| Example 113 | ** |
| Example 114 | ** |
| Example 115 | *** |
| Example 116 | *** |
| Example 117 | *** |
| Example 118 | *** |
| Example 119 | *** |
| Example 120 | *** |
| Example 121 | *** |
| Example 122 | *** |
| Example 123 | *** |
| Example 124 | *** |
| Example 125 | *** |
| Example 126 | *** |
| Example 127 | *** |
| Example 128 | *** |
| Example 129 | *** |
| Example 130 | *** |
| Example 131 | *** |
| Example 132 | *** |
| Example 133 | *** |
| Example 134 | *** |
| Example 135 | *** |
| Example 136 | *** |
| Example 137 | *** |
| Example 138 | *** |
| Example 139 | *** |
| Example 140 | *** |
| Example 141 | *** |
| Example 142 | *** |
| Example 143 | *** |
| Example 144 | *** |
| Example 145 | *** |
| Example 146 | *** |
| Example 147 | *** |
| Example 148 | *** |
| Example 149 | * |
| Example 150 | *** |
| Example 151 | *** |
| Example 152 | *** |
| Example 153 | *** |
| Example 154 | *** |
| Example 155 | *** |
| Example 156 | *** |
| Example 157 | *** |
| Example 158 | *** |
| Example 159 | *** |
| Example 160 | *** |
| Example 161 | *** |
| Example 162 | ** |
| Example 163 | *** |
| Example 164 | *** |
| Example 165 | *** |
| Example 166 | *** |
| Example 167 | *** |
| Example 168 | *** |
| Example 169 | *** |
| Example 170 | *** |
| Example 171 | *** |
| Example 172 | *** |
| Example 173 | *** |
| Example 174 | *** |
| Example 175 | *** |
| Example 176 | *** |
| Example 177 | *** |
| Example 178 | *** |
| Example 179 | *** |
| Example 180 | *** |
| Example 181 | *** |
| Example 182 | *** |
| Example 183 | *** |
| Example 184 | *** |
| Example 185 | *** |
| Example 186 | *** |
| Example 187 | *** |
| Example 188 | *** |
| Example 189 | *** |
| Example 190 | *** |
| Example 191 | *** |
| Example 192 | *** |
| Example 193 | *** |
| Example 194 | *** |
| Example 195 | *** |
| Example 196 | *** |
| Example 197 | *** |
| Example 198 | *** |
| Example 199 | *** |
| Example 200 | *** |
| Example 201 | *** |
| Example 202 | *** |
| Example 203 | *** |
| Example 204 | *** |
| Example 205 | *** |
| Example 206 | *** |
| Example 207 | *** |
| Example 208 | *** |
| Example 209 | *** |
| Example 210 | *** |
| Example 211 | *** |
| Example 212 | *** |
| Example 213 | *** |
| Example 214 | *** |

Table 2 below shows data for selected examples against a panel of different kinases, where the residual kinase activity, in %, at two concentrations of the compounds is expressed

TABLE 2

| | Example 23 | | Example 50 | | Example 47 | |
|---|---|---|---|---|---|---|
| | 1 μM | 10 μM | 1 μM | 10 μM | 1 μM | 10 μM |
| MKK1 | 52 | 25 | 44 | 24 | 33 | 17 |
| ERK1 | 88 | 75 | 99 | 65 | 89 | 81 |
| ERK2 | 107 | 108 | 106 | 62 | 90 | 66 |
| JNK1 | 91 | 73 | 94 | 87 | 92 | 86 |
| JNK2 | 78 | 55 | 102 | 66 | 98 | 89 |
| p38a MAPK | 72 | 86 | 106 | 90 | 105 | 100 |
| P38b MAPK | 86 | 92 | 99 | 89 | 99 | 90 |
| p38g MAPK | 81 | 91 | 93 | 80 | 97 | 77 |
| p38s MAPK | 77 | 72 | 79 | 67 | 84 | 84 |
| ERK8 | 14 | 6 | 13 | 5 | 33 | 10 |
| RSK1 | 47 | 24 | 49 | 20 | 45 | 10 |
| RSK2 | 44 | 14 | 69 | 28 | 65 | 28 |
| PDK1 | 44 | 10 | 22 | 4 | 9 | 2 |
| PKBa | 100 | 91 | 109 | 97 | 101 | 90 |
| PKBb | 106 | 114 | 116 | 107 | 117 | 97 |
| SGK1 | 100 | 64 | 86 | 57 | 71 | 59 |
| S6K1 | 38 | 30 | 63 | 30 | 63 | 30 |
| PKA | 80 | 73 | 91 | 80 | 96 | 80 |
| ROCK 2 | 77 | 40 | 77 | 51 | 85 | 48 |
| PRK2 | 67 | 60 | 68 | 58 | 76 | 68 |
| PKCa | 81 | 87 | 85 | 70 | 83 | 69 |
| PKC zeta | 99 | 89 | 84 | 73 | 80 | 78 |
| PKD1 | 68 | 53 | 60 | 36 | 57 | 52 |
| MSK1 | 74 | 34 | 77 | 70 | 61 | 54 |
| MNK1 | 85 | 62 | 94 | 81 | 93 | 73 |
| MNK2 | 39 | 10 | 56 | 21 | 36 | 16 |
| MAPKAP-K2 | 77 | 86 | 88 | 85 | 75 | 95 |
| PRAK | 93 | 96 | 64 | 64 | 70 | 76 |
| CAMKKb | 43 | 8 | 72 | 40 | 61 | 36 |
| CAMK1 | 77 | 66 | 94 | 71 | 91 | 73 |
| SmMLCK | 72 | 74 | 88 | 75 | 102 | 81 |
| PHK | 28 | 3 | 64 | 15 | 78 | 24 |
| CHK1 | 51 | 14 | 65 | 21 | 70 | 30 |
| CHK2 | 50 | 11 | 52 | 16 | 71 | 28 |
| GSK3b | 53 | 20 | 52 | 19 | 73 | 31 |
| CDK2-Cyclin A | 17 | 2 | 33 | 5 | 70 | 29 |
| PLK1 | 90 | 62 | 101 | 66 | 88 | 75 |
| PLK1 (Okadaic | 66 | 46 | 82 | 60 | 90 | 64 |
| Acid) | | | | | | |
| AMPK | 45 | 8 | 76 | 38 | 77 | 30 |
| MARK3 | 0 | 1 | 13 | 2 | 15 | 4 |
| BRSK2 | 64 | 26 | 87 | 42 | 91 | 36 |
| MELK | 23 | 3 | 29 | 17 | 14 | 27 |
| CK1 | 93 | 64 | 107 | 82 | 102 | 106 |
| CK2 | 111 | 107 | 72 | 80 | 80 | 78 |
| DYRK1A | 78 | 56 | 88 | 57 | 84 | 78 |
| DYRK2 | 67 | 50 | 76 | 38 | 81 | 63 |
| DYRK3 | 78 | 36 | 59 | 22 | 73 | 33 |
| NEK2a | 91 | 65 | 93 | 71 | 104 | 79 |
| NEK6 | 88 | 83 | 107 | 103 | 65 | 65 |
| IKKb | 75 | 80 | 90 | 88 | 91 | 86 |
| PIM1 | 78 | 46 | 63 | 92 | 82 | 57 |
| PIM2 | 74 | 67 | 100 | 89 | 101 | 93 |
| PIM3 | 73 | 37 | 102 | 50 | 99 | 78 |
| SRPK1 | 75 | 40 | 84 | 57 | 81 | 78 |
| MST2 | 73 | 33 | 73 | 33 | 84 | 70 |
| EF2K | 105 | 125 | 108 | 101 | 105 | 104 |
| HIPK2 | 59 | 26 | 83 | 54 | 88 | 67 |
| PAK4 | 72 | 31 | 75 | 35 | 80 | 57 |
| PAK5 | 74 | 56 | 73 | 47 | 69 | 68 |
| PAK6 | 82 | 50 | 78 | 50 | 77 | 85 |
| Src | 77 | 50 | 84 | 50 | 80 | 67 |
| Lck | 86 | 37 | 91 | 50 | 105 | 69 |
| CSK | 77 | 69 | 79 | 60 | 77 | 71 |
| FGF-R1 | 89 | 36 | 77 | 31 | 78 | 31 |
| IRR | 44 | 17 | 39 | 17 | 64 | 25 |
| EPH A2 | 71 | 71 | 88 | 73 | 47 | 76 |
| MST4 | 94 | 56 | 164 | 120 | 124 | 111 |
| SYK | 87 | 46 | 83 | 60 | 82 | 49 |
| YES1 | 77 | 22 | 82 | 29 | 94 | 74 |
| IKKe | 15 | 5 | 28 | 7 | 14 | 6 |
| IGF-1R | 86 | 87 | 47 | 32 | 39 | 35 |
| VEG-FR | 81 | 27 | 78 | 20 | 12 | 34 |
| BTK | 74 | 25 | 93 | 66 | 93 | 61 |
| IR-HIS | 105 | 86 | 109 | 109 | 105 | 85 |
| EPH-B3 | 120 | 95 | 100 | 97 | 48 | 93 |

| | | | |
|---|---|---|---|
| TBK1 IC₅₀ (μM) | 0.004 | 0.108 | 0.029 |

### Comparative Data

### Effect of 5-Substituent on the Pyrimidine Ring on TBK1 Activity and Selectivity Over Other Protein Kinases.

### SUMMARY

The following comparative data demonstrate that the 5-cyclopropyl pyrimidine derivatives exemplified in our patent show a unique and optimal balance of activity against the target enzyme versus selectivity over other protein kinases. The effect of substituting the 5-halo substituent on the pyrimidine ring with a cyclopropyl group was investigated and the 5-cyclopropyl derivatives showed improved kinase selectivity compared to their 5-halo counterparts. In addition to the 5-cyclopropyl derivatives, a range of linear and branched alkyl, as well as cyclopropylmethyl analogues were investigated (Table) and were found to have significantly reduced activity against the target enzyme (TBK1) compared to Example 23. Without wishing to be bound by theory, it is believed that the unique combination of steric and electronic properties of the cyclopropyl group account for these surprising and unpredictable results.

Comparison of a 5-Halo Pyrimidine with a Cyclopropyl Derivative

Table compares the activity of Example 23 with cyclobutanecarboxylic acid {3-[5-bromo-2-(3-fluoro-phenylamino)-pyrimidin-4-ylamino]-propyl}-amide (Compound A)¹³, against a panel of different kinases, where the residual kinase activity, in %, at two concentrations of the compounds is expressed. The higher the percentage residual activity, the lower the degree of inhibition of the kinase. The Gini coefficient (value between 0 and 1) has recently been used to quantify the selectivity profile of compounds against large numbers of kinases, with higher values indicating a higher degree of selectivity.¹² The data suggest that whilst both compounds show a similar inhibition of TBK1, replacement of the 5-bromo substituent on the pyrimidine ring with a cyclopropyl group (Example 23) results in a significant improvement of selectivity across this kinase panel. This is also reflected in the Gini coefficients, with Compound A giving a significantly lower value than Example 23.

TABLE 3
Selectivity data against a panel of different kinases

| | Example 23 | | Compound A | |
|---|---|---|---|---|
| | 1 μm | 10 μM | 1 μm | 10 μM |
| MKK1 | 52 | 25 | 15 | 2 |
| ERK1 | 88 | 75 | 28 | 7 |
| ERK2 | 107 | 108 | 49 | 10 |
| JNK1 | 91 | 73 | 46 | 9 |
| JNK2 | 78 | 55 | 40 | 5 |
| p38a MAPK | 72 | 86 | 99 | 78 |
| P38b MAPK | 86 | 92 | 96 | 80 |
| p38g MAPK | 81 | 91 | 65 | 17 |
| p38s MAPK | 77 | 72 | 55 | 15 |
| ERK8 | 14 | 6 | 9 | 3 |
| RSK1 | 47 | 24 | 27 | 9 |
| RSK2 | 44 | 14 | 38 | 9 |
| PDK1 | 44 | 10 | 6 | 1 |
| PKBa | 100 | 91 | 107 | 92 |
| PKBb | 106 | 114 | 91 | 93 |
| SGK1 | 100 | 64 | 59 | 18 |
| S6K1 | 38 | 30 | 49 | 13 |
| PKA | 80 | 73 | 75 | 22 |
| ROCK 2 | 77 | 40 | 33 | 11 |
| PRK2 | 67 | 60 | 24 | 4 |
| PKCa | 81 | 87 | 94 | 49 |
| PKC zeta | 99 | 89 | 90 | 42 |
| PKD1 | 68 | 53 | 7 | 1 |
| MSK1 | 74 | 34 | 62 | 24 |
| MNK1 | 85 | 62 | 49 | 15 |
| MNK2 | 39 | 10 | 6 | 7 |
| MAPKAP-K2 | 77 | 86 | 82 | 48 |
| PRAK | 93 | 96 | 65 | 31 |
| CAMKKb | 43 | 8 | 27 | 22 |
| CAMK1 | 77 | 66 | 54 | 25 |
| SmMLCK | 72 | 74 | 101 | 59 |
| PHK | 28 | 3 | 7 | 0 |
| CHK1 | 51 | 14 | 9 | 0 |
| CHK2 | 50 | 11 | 13 | 12 |
| GSK3b | 53 | 20 | 23 | 4 |
| CDK2-Cyclin A | 17 | 2 | 1 | 0 |
| PLK1 | 90 | 62 | 82 | 45 |
| PLK1 (Okadaic Acid) | 66 | 46 | 95 | 39 |
| AMPK | 45 | 8 | 4 | 2 |
| MARK3 | 0 | 1 | 3 | 2 |
| BRSK2 | 64 | 26 | 23 | 6 |
| MELK | 23 | 3 | 15 | 14 |
| CK1 | 93 | 64 | 91 | 50 |
| CK2 | 111 | 107 | 77 | 44 |
| DYRK1A | 78 | 56 | 74 | 38 |
| DYRK2 | 67 | 50 | 36 | 6 |
| DYRK3 | 78 | 36 | 33 | 7 |
| NEK2a | 91 | 65 | 95 | 57 |
| NEK6 | 88 | 83 | 88 | 93 |
| IKKb | 75 | 80 | 93 | 66 |
| PIM1 | 78 | 46 | 52 | 50 |
| PIM2 | 74 | 67 | 90 | 75 |
| PIM3 | 73 | 37 | 78 | 21 |
| SRPK1 | 75 | 40 | 69 | 31 |
| MST2 | 73 | 33 | 29 | 6 |
| EF2K | 105 | 125 | 96 | 88 |
| HIPK2 | 59 | 26 | 58 | 12 |
| PAK4 | 72 | 31 | 32 | 6 |
| PAK5 | 74 | 56 | 46 | 14 |
| PAK6 | 82 | 50 | 45 | 14 |
| Src | 77 | 50 | 53 | 12 |
| Lck | 86 | 37 | 57 | 10 |
| CSK | 77 | 69 | 86 | 51 |
| FGF-R1 | 89 | 36 | 17 | 1 |
| IRR | 44 | 17 | 32 | 15 |
| EPH A2 | 71 | 71 | 87 | 60 |
| MST4 | 94 | 56 | 90 | 47 |
| SYK | 87 | 46 | 25 | 4 |
| YES1 | 77 | 22 | 36 | 8 |
| IKKe | 15 | 5 | 13 | 3 |
| IGF-1R | 86 | 87 | 78 | 21 |
| VEG-FR | 81 | 27 | 4 | 4 |
| BTK | 74 | 25 | 94 | 33 |
| IR-HIS | 105 | 86 | 91 | 69 |
| EPH-B3 | 120 | 95 | 92 | 69 |

| | | |
|---|---|---|
| GINI Coefficient (@ 10 μM) | 0.35 | 0.19 |
| TBK1 IC50 (μM) | 0.004 | 0.002 |

TABLE 4
Structures of Example 23 and compound A

| | |
|---|---|
| 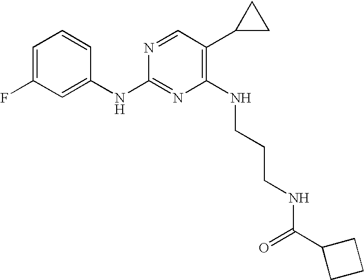 | Example 23 |
| 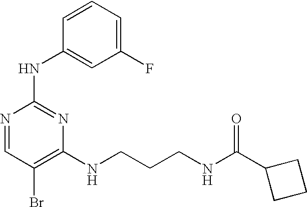 | Cyclobutanecarboxylic acid {3-[5-bromo-2-(3- fluoro-phenylamino)- pyrimidin-4-ylamino]- propyl}-amide (Compound A) |

Comparison of a 5-Cyclopropylpyrimidine with Isopropyl, Cyclopropylmethyl and Ethyl Derivatives

Table 5 compares the IC₅₀ values against TBK1 of Example 23 with cyclobutanecarboxylic acid {3-[2-(3-fluoro-phenylamino)-5-isopropyl-pyrimidin-4-ylamino]-propyl}-amide (Compound B), cyclobutanecarboxylic acid {3-[5-cyclopropylmethyl-2-(3-fluoro-phenylamino)-pyrimidin-4-ylamino]-propyl}-amide (Compound C) and cyclobutanecarboxylic acid {3-[5-ethyl-2-(3-fluoro-phenylamino)-pyrimidin-4-ylamino]-propyl}-amide (Compound D). These data indicate that the 5-cyclopropyl derivative (Example 23) was found to be >250-fold more active than both the isopropyl derivative (Compound B) or cyclopropylmethyl derivative (Compound C). The ethyl derivative (Example D) shows a 10-fold loss in activity compared to Example 23.

TABLE 5
IC50 values against TBK1 of Example 23, Compound B13 and Compound C13

| Structure | Example # | TBK1 IC₅₀ |
|---|---|---|
| 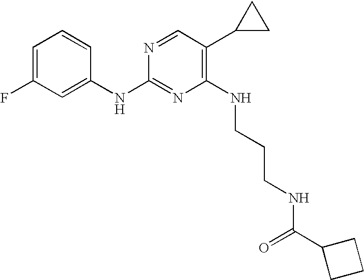 | Example 23 | 4 nM |
| 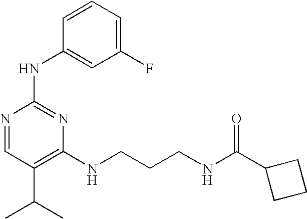 | Cyclobutanecarboxylic acid {3- [2-(3-fluoro-phenylamino)-5- isopropyl-pyrimidin-4-ylamino]- propyl}-amide (Compound B) | >1000 nM |
| 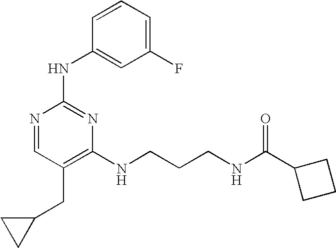 | Cyclobutanecarboxylic acid {3- [5-cyclopropylmethyl-2-(3- fluoro-phenylamino)-pyrimidin- 4-ylamino]-propyl}-amide (Compound C) | >1000 nM |
| 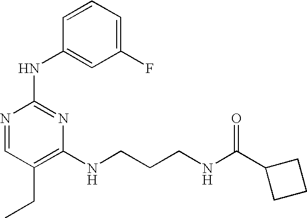 | Cyclobutanecarboxylic acid {3- [5-ethyl-2-(3-fluoro- phenylamino)-pyrimidin-4- ylamino]-propyl}-amide (Compound D) | 42 nM |

### REFERENCES & NOTES

1. Rezaie, T., Child, A., Hitchings, R., Brice, G., Miller, L., Coca-Prados, M., Heon, E., Krupin, T., Ritch, R., Kreutzer, D., Crick, R. P. and Sarfarazi, M. (2002) Adult-onset primary open-angle glaucoma caused by mutations in optineurin. Science 295, 1077-1079.
2. Sarfarazi, M. and Rezaie, T. (2003) Optineurin in primary open angle glaucoma. Opthalmol Clin North Am 16, 529-541.
3. Tezel, G. and Wax, M. B. (2000) Increased production of tumour necrosis factor-alpha by glial cells exposed to stimulated ischemia or elevated hydrostatic pressure induces apoptosis in cocultured retinal ganglion cells. J Neurosci 20, 8693-8700.
4. Yuan, L. and Neufeld, A. H. (2000) Tumor necrosis factor-alpha: a potentially neurodestructive cytokine produced by glia in the human glaucomatous optic nerve head. Glia 32, 42-50.
5. Perry et al (J Exp Med 199, 1651-1658, 2004) compared the role of TBK1 in interferon responses induced by a number of stimuli. TBK−/− mice were deficient in their ability to up regulate IFN beta production.
6. McWhirter et al (PNAS101, 233 238, 2004) Demonstrate that induction of type I interferon and related genes depends on TBK1. They also show that IKKepsilon and TBK1 directly phosphorylate serine residues that are critical for IRF3 activation.
7. Hemmi et al (J Exp Med 199, 1641-1650, 2004) indicate that TBK1 and IKK are essential for the activation of IFN beta and IFN inducible genes.
8. Davies, S. P., Reddy, H., Caivano, M. and Cohen, P. (2000) Specificity and mechanism of action of some commonly used protein kinase inhibitors. Biochem J 351, 95-105.
9. Bain, J., McLauchlan, H., Elliott, M. and Cohen, P. (2003) The specificities of protein kinase inhibitors: an update. Biochem J 371, 199-204.
10. Schwamborn, K., Weil, R., Courtois, G., Whiteside, S. T. and Israel, A. (2000) Phorbol esters and cytokines regulate the expression of the NEMO-related protein, a molecule involved in a NF-kappa B-independent pathway. J Biol Chem 275, 22780-22789.
11. Morton, S., Hesson, L., Peggie, M. and Cohen, P. (2008) Enhanced binding of TBK1 by an optineurin mutant that causes a familial form of primary open angle glaucoma. FEBS Letters 582, 997-1002.
12. P. P. Graczyk, J. Med. Chem. 50 (2007), p. 5773
13. Compounds A, B and D are generically claimed in WO 2004/048343 and can be prepared analogously to procedures outlined in this patent application; compound C is generically claimed in WO 2002/064586. Spectrocopic data for compound C: ¹H NMR (400 MHz, DMSO-d₆) δ ppm 0.12-0.25 (m, 2H), 0.35-0.59 (m, 2H), 0.94-1.12 (m, 1H), 1.69-1.81 (m, 3H), 1.85-1.95 (m, 1H), 1.96-2.06 (m, 2H), 2.08-2.19 (m, 2H), 2.29 (d, J=6.9 Hz, 2H), 2.99 (quin, J=8.2 Hz, 1H), 3.15 (q, J=6.6 Hz, 2H), 3.40-3.55 (m, 2H), 6.66 (td, J=8.5, 2.3 Hz, 1H), 6.82 (t, J=5.7 Hz, 1H), 7.22-7.32 (m, 1H), 7.48 (d, J=9.6 Hz, 1H), 7.70 (t, J=5.7 Hz, 1 H), 7.81 (s, 1H), 7.91 (dt, J=13.3, 2.3 Hz, 1H), 9.17 (s, 1H); m/z (ES+APCI)⁺: 398 [M+H]⁺.

1. A compound of formula (I), or a pharmaceutically acceptable salt or ester thereof, 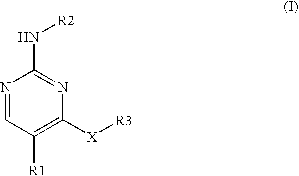
wherein:
R¹ is C₃₋₈-cycloalkyl;
X is O, NR⁷ or C₃₋₆-heterocycloalkyl;
R² is aryl, heteroaryl, fused or unfused aryl-C₃₋₆-heterocycloalkyl or fused or unfused heteroaryl-C₃₋₆-heterocycloalkyl, each of which is optionally substituted by one or more substitutents selected from aryl, heteroaryl, C₁₋₆-alkyl, C₃₋₇-cycloalkyl and a group A, wherein said C₁₋₆-alkyl group is optionally substituted by one or more substituents selected from aryl, heteroaryl, R¹⁰ and a group A, said heteroaryl group is optionally substituted by one or more R¹⁰ groups; and wherein said C₃₋₆-heterocycloalkyl group optionally contains one or more groups selected from oxygen, sulfur, nitrogen and CO;
R³ is C₁₋₆-alkyl optionally substituted by one or more substituents selected from aryl, heteroaryl, —NR⁴R⁵, —OR⁶, —NR⁷(CO)R⁶, —NR⁷(CO)NR⁴R⁵, —NR⁷SO₂R⁶, —NR⁷COOR⁷, —CONR⁴R⁵, C₃₋₆-heterocycloalkyl and 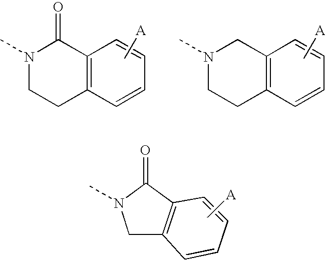
wherein said aryl, heteroaryl and C₃₋₆-heterocycloalkyl groups are each optionally substituted by one or more substituents selected from —C₁₋₆-alkyl and a group A, wherein said —C₁₋₆-alkyl group is optionally substituted by one or more substituents selected from aryl, heteroaryl and a group A;
A is selected from halogen, hydroxyl, cyano, trifluoromethyl, alkoxy, —NO₂, —NH₂, —NR⁴R⁵, —OR⁶, —NR⁷(CO)R⁶, —NR⁷(CO)NR⁴R⁵, —NR⁷COOR⁷, —NR⁷(SO₂)R⁶, —CO₂H, —NR⁷(SO₂)NR⁴R⁵, —COOR⁷, CONR⁴R⁵, COR⁶, SO₂NR⁴R⁵ and —SO₂CH₃;
each R⁴ and R⁵ is independently selected from hydrogen, C₃₋₇-cycloalkyl, aryl, heteroaryl, C₁₋₆-alkyl and a C₃₋₆-heterocycloalkyl ring optionally further containing one or more groups selected from oxygen, sulfur, nitrogen and CO and optionally substituted by one or more R¹⁰ groups, wherein said C₁₋₆-alkyl is optionally substituted by one or more substituents selected from halogen, cyano, hydroxyl, aryl, heteroaryl, —NR⁸R⁹, —NR⁷(CO)R⁶, —NR⁷COOR⁶, —NR⁷(SO₂)R⁶, —COOR⁶, —CONR⁸R⁹, OR¹⁰, —SO₂R⁶ and a C₃₋₆-heterocycloalkyl ring optionally further containing one or more groups selected from oxygen, sulfur, nitrogen and CO and optionally substituted by one or more or R¹⁰ groups; or
R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring may be saturated or unsaturated and is optionally substituted with one or more groups selected from NR⁸R⁹ and R¹⁰ groups;
each R⁶ is independently selected from C₁₋₆-alkyl, C₃₋₇ cycloalkyl, C₄₋₇-heterocycloalkyl, aryl and heteroaryl, each of which may be optionally substituted by one or more substituents selected from halogen, R¹⁰ and —NR⁸R⁹;
each R⁷ is selected from hydrogen, C₁₋₆-alkyl and C₃₋₇-cycloalkyl, wherein said C₁₋₆-alkyl is optionally substituted by one or more halogens;
each of R⁸ and R⁹ is independently selected from hydrogen and C₁₋₆-alkyl, wherein said C₁₋₆-alkyl group is optionally substituted by one or more halogens; or
R⁸ and R⁹ together with the N to which they are attached form a C₄₋₆-heterocycloalkyl ring optionally further containing one or more heteroatoms selected from oxygen and sulfur, wherein said C₄₋₆-heterocycloalkyl ring is optionally substituted by one or more R¹⁰ groups; and
each R¹⁰ is selected from halogen, C₃₋₇-cycloalkyl and C₁₋₆-alkyl optionally substituted by one or more halogens, wherein where R¹⁰ is C₁₋₆-alkyl and two or more R¹⁰ groups are attached to the same carbon atom, the R¹⁰ groups may be linked to form a spiroalkyl group.

2. A compound according to claim 1 wherein R¹ is cyclopropyl or cyclobutyl.

3. A compound according to claim 1 wherein R¹ is cyclopropyl.

4. A compound according to claim 1 wherein X is NH.

5. A compound according claim 1 wherein R² is an optionally substituted aryl or heteroaryl group selected from the following:

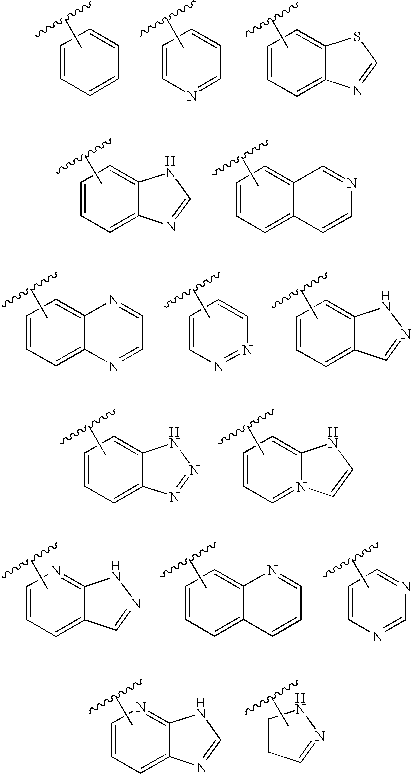

6. A compound according to claim 1 wherein R² is an aryl or heteroaryl group each of which is optionally substituted by one or more substituents selected from C₁₋₆-alkyl and a group A, wherein said C₁₋₆-alkyl group is optionally substituted by one or more substituents selected from aryl, heteroaryl and a group A, and wherein A is selected from halogen, OH, CN, CF₃, —NH₂, —NR⁴R⁵, —OR⁶, NR⁷(CO)R⁶, —NR⁷COOR⁷, —NR⁷(SO₂)R⁶, —COOH, —COOR⁷ and CONR⁴R⁵.

7. A compound according to claim 1 wherein R² is an aryl or heteroaryl group each of which is optionally substituted by one or more substituents selected from C₁₋₆-alkyl, halogen, CN, NHCO—C₁₋₆-alkyl, CF₃, COOH, CONH₂, OH, NH₂, NHSO₂—C₁₋₆-alkyl, O—CF₃, —NHCOO—C₁₋₆-alkyl, —CO₂—C₁₋₆-alkyl, —N(C₁₋₆-alkyl)₂, 4-methylpiperazin-1-yl, (4-methylpiperazin-1-yl)-CO—, (N-morpholinyl)-(CH₂)ₚ(O)_{q}—, (imidazol-1-yl)-(CH₂)ₚ— where q is 0, 1, 2 or 3 and each p is independently 1, 2 or 3 and NR⁴R⁵, wherein R⁴ and R⁵ and the nitrogen to which they are attached form a C₃₋₆-heterocycloalkyl ring optionally containing a CO group.

8. A compound according to claim 1 wherein R² is an aryl or heteroaryl group each of which is optionally substituted by one or more substituents selected from Me, Cl, F, CN, NHCOMe, CF₃, COOH, CONH₂, OH, NH₂, NHSO₂Me, OCF₃, —NHCOO^{t}Bu, —CO₂Me, —NMe₂, 4-methylpiperazin-1-yl, N-morpholinyl, (4-methylpiperazin-1-yl)-CO—, (N-morpholinyl)-CH₂CH₂O—, (imidazol-1-yl)-CH₂— and

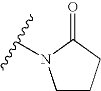

9. A compound according to claim 1 wherein R² is:
(a) a phenyl group substituted by one or more A groups, wherein A is preferably selected from CF₃, halogen, CN, NHSO₂Me, SO₂NR⁴R⁵, NR⁴R⁵, OR⁶, COOR⁷, NR⁷COOR⁷, NR⁷COR⁶, CONR⁴R⁵, NR⁷CONR⁴R⁵ and NR⁷SO₂R⁶;
(b) a phenyl group substituted by one or more C₁₋₆-alkyl groups, each of which in turn is optionally substituted by one or more groups selected from heteroaryl and A, wherein the heteroaryl group is preferably selected from imidazolyl and triazolyl, and the A group is preferably selected from CONR⁴R⁵, NR⁴R⁵, OR⁶, COOR⁷ and CN;
(c) a phenyl group substituted by one or more heteroaryl groups, wherein the heteroaryl group is preferably selected from pyrimidinyl, tetrazolyl, pyridinyl, pyrazolyl, oxazolyl and triazolyl;
(d) a pyridyl group substituted by one or more A groups, wherein the A group is preferably selected from NR⁴R⁵, halo and OR⁶;
(e) a pyridyl group substituted by a heteroaryl group, wherein the heteroaryl group is preferably selected from pyrazolyl, pyrimidinyl and pyridinyl;
(f) a pyridyl group substituted by a C₁₋₆-alkyl group, wherein said C₁₋₆-alkyl group is in turn optionally substituted with one or more substituents selected from NR⁴R⁵ and OR⁶;
(g) an optionally substituted fused aryl-C₃₋₆-heterocycloalkyl or fused heteroaryl-C₃₋₆-heterocycloalkyl, preferably selected from the following:

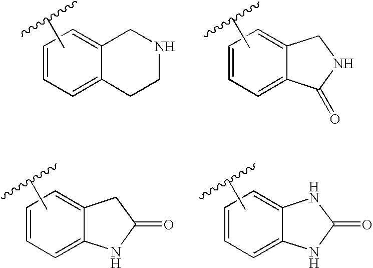

10. A compound according to claim 1 wherein R³ is C₁₋₄-alkyl optionally substituted by one or more substituents selected from heteroaryl, —NR⁴R⁵, —NR⁷(CO)R⁶, —NR⁷COOR⁷, C₃₋₆-heterocycloalkyl and

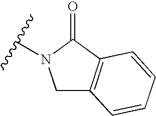

11. A compound according to claim 1 wherein R³ is C₁₋₄-alkyl substituted by —NR⁷(CO)R⁶.

12. A compound according to claim 11 wherein R⁷ is H and R⁶ is selected from C₁₋₆-alkyl, C₃₋₇ cycloalkyl, C₄₋₇-heterocycloalkyl, heteroaryl, each of which may be optionally substituted by one or more substituents selected from halogen, R¹⁰ and —NR⁸R⁹.

13. A compound according to claim 12 wherein R⁶ is selected from thienyl, cyclopentyl, CH₂-cyclopentyl, isopropyl, pyrazolyl, cyclohexyl, thiazolyl, oxazolyl, furanyl, CF₃, imidazolyl, cyclopropyl, CH₂-cyclopropyl, cyclobutyl, triazolyl, pyrrolyl, tetrahydrofuranyl, CH₂NMe₂ and iso-oxazolyl.

14. A compound according to claim 1 wherein R³ is C₁₋₄-alkyl substituted by a heteroaryl selected from pyrazolyl, tetrazolyl and triazolyl.

15. A compound according to claim 1 wherein R³ is C₁₋₄-alkyl substituted by a —NR⁴R⁵ group, wherein R⁴ and R⁵ together with the N to which they are attached form a C₃₋₆-heterocycloalkyl ring optionally further containing one or more CO groups, wherein said C₃₋₆-heterocycloalkyl ring is optionally substituted with one or more R¹⁰ groups.

16. A compound according to claim 1 wherein R³ is C₁₋₄-alkyl substituted by one of the following groups: 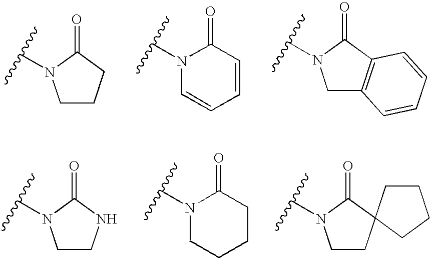
each of which may be optionally substituted with one or more R¹⁰ groups.

17. A compound according to claim 1 wherein R³ is C₁₋₄-alkyl substituted by a C₃₋₆-heterocycloalkyl group, wherein said C₃₋₆-heterocycloalkyl group is optionally substituted by one or more A groups.

18. A compound according to claim 1 wherein R³ is an optionally substituted C₃-alkyl group.

19. A compound according to claim 1 which is selected from the following:

| Structure | Example |
|---|---|
| 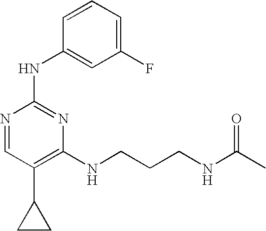 | Example 1 |
| 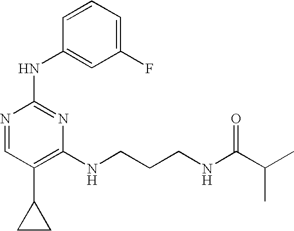 | Example 2 |
| 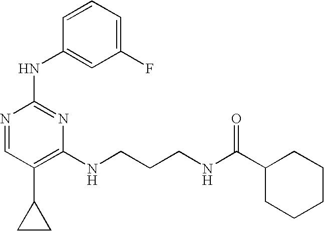 | Example 3 |
| 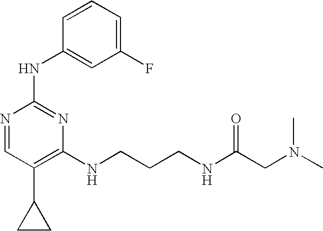 | Example 4 |
| 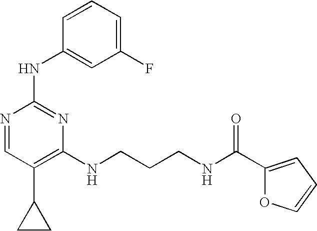 | Example 5 |
| 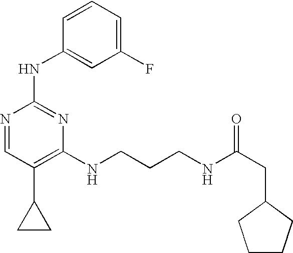 | Example 6 |
| 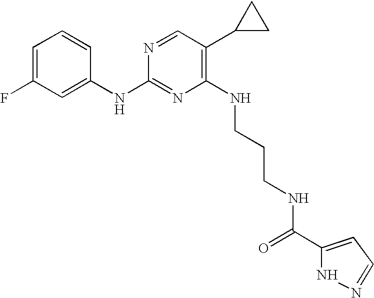 | Example 7 |
| 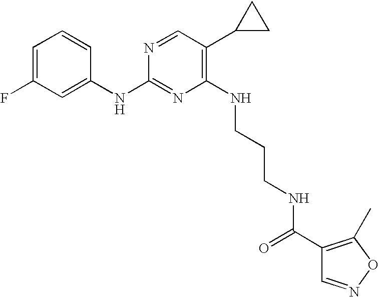 | Example 8 |
| 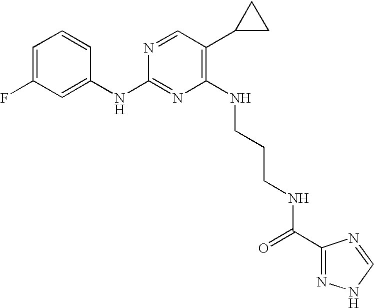 | Example 9 |
| 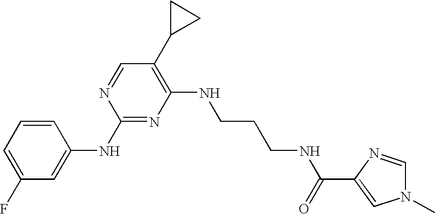 | Example 10 |
| 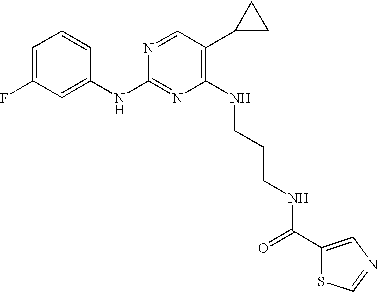 | Example 11 |
| 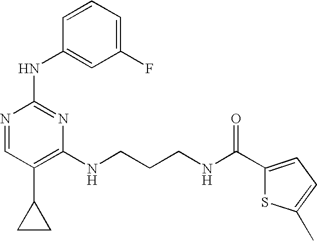 | Example 12 |
| 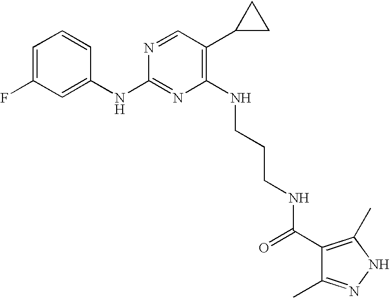 | Example 13 |
| 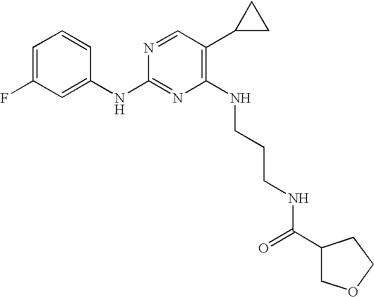 | Example 14 |
| 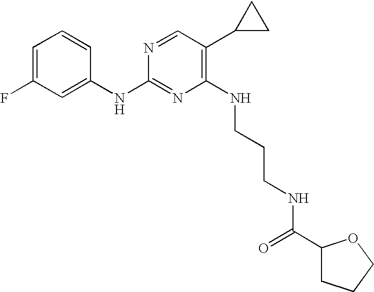 | Example 15 |
| 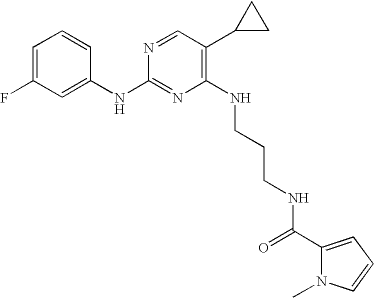 | Example 16 |
| 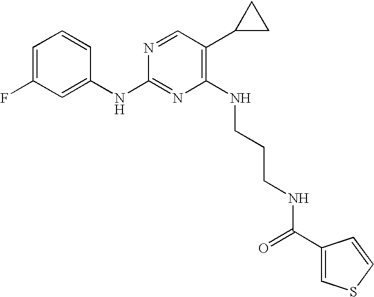 | Example 17 |
| 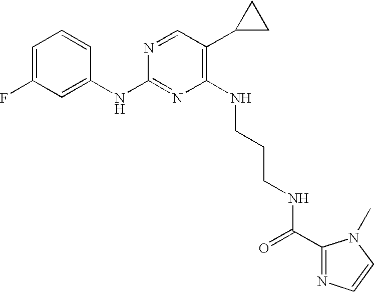 | Example 18 |
| 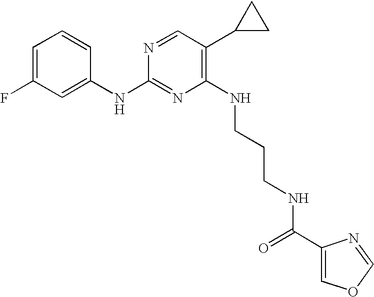 | Example 19 |
| 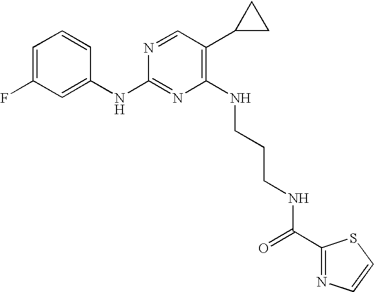 | Example 20 |
| 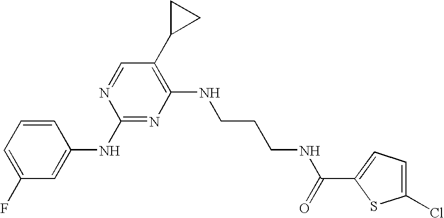 | Example 21 |
| 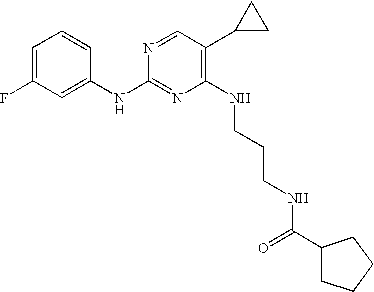 | Example 22 |
| 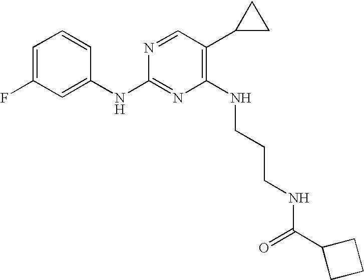 | Example 23 |
| 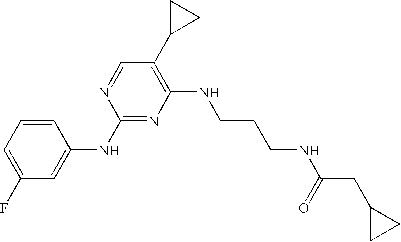 | Example 24 |
| 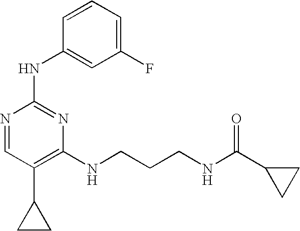 | Example 25 |
| 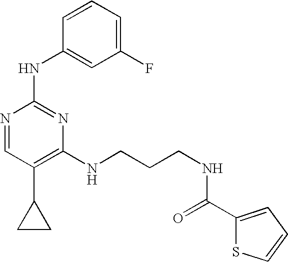 | Example 26 |
| 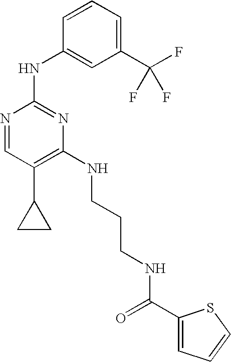 | Example 27 |
| 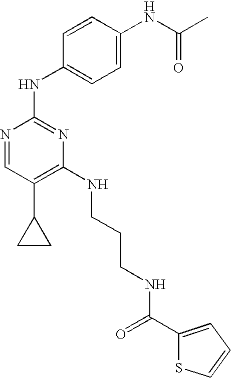 | Example 28 |
| 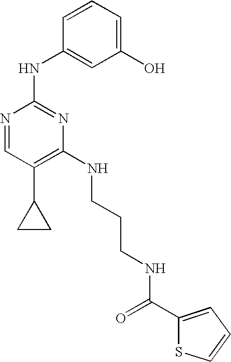 | Example 29 |
| 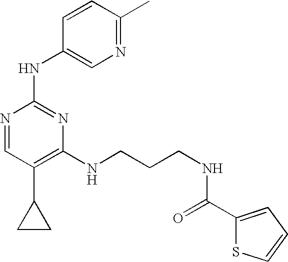 | Example 30 |
| 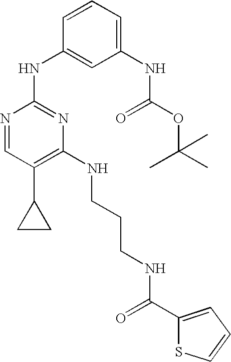 | Example 31 |
| 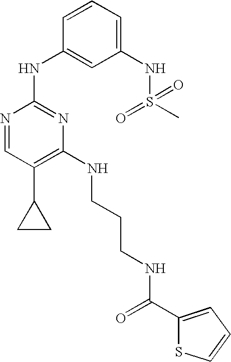 | Example 32 |
| 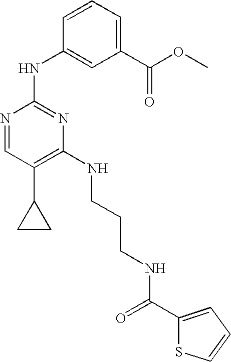 | Example 33 |
| 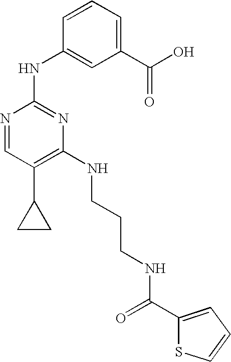 | Example 34 |
| 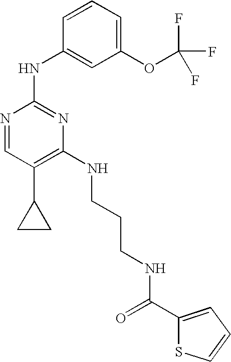 | Example 35 |
| 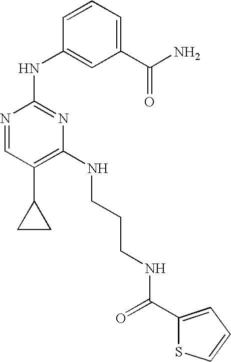 | Example 36 |
| 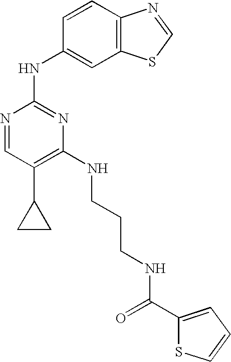 | Example 37 |
| 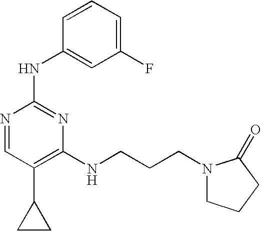 | Example 38 |
| 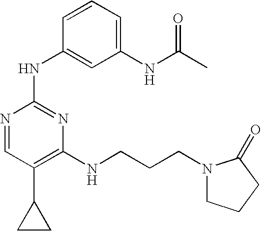 | Example 39 |
| 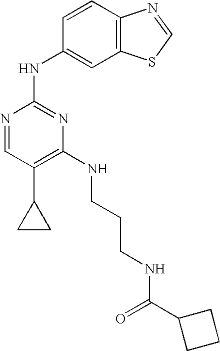 | Example 40 |
| 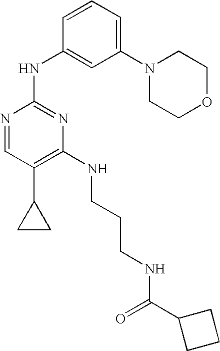 | Example 41 |
| 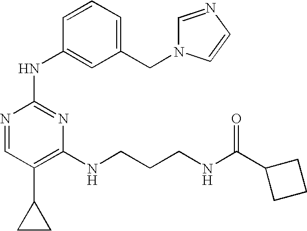 | Example 42 |
| 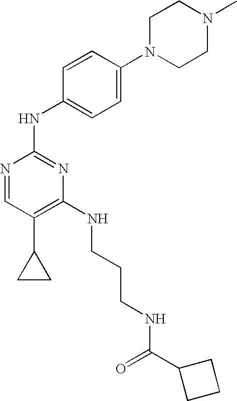 | Example 43 |
| 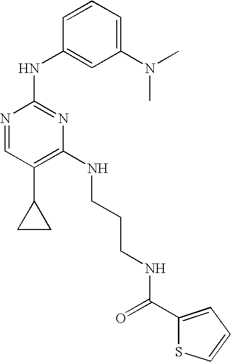 | Example 44 |
| 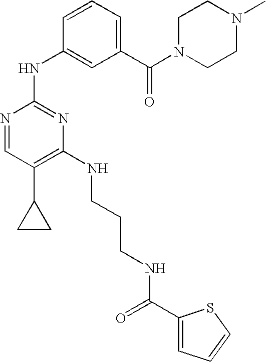 | Example 45 |
| 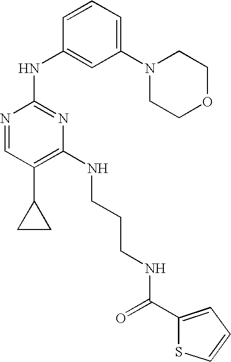 | Example 46 |
| 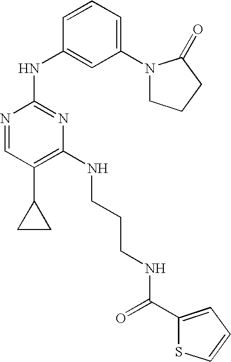 | Example 47 |
| 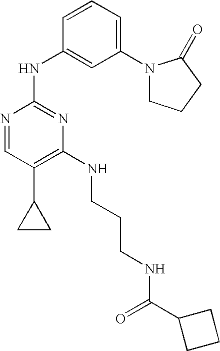 | Example 48 |
| 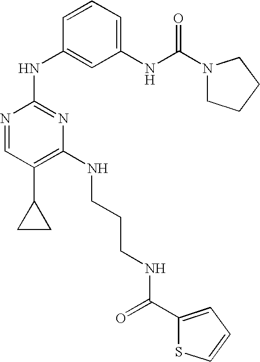 | Example 49 |
| 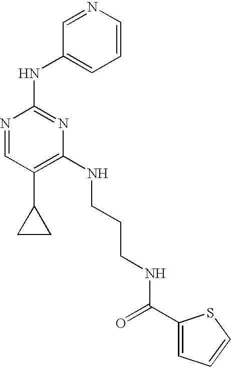 | Example 50 |
| 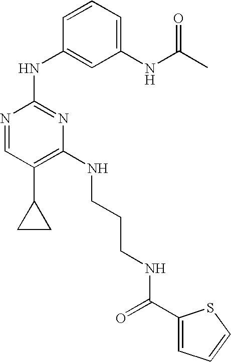 | Example 51 |
| 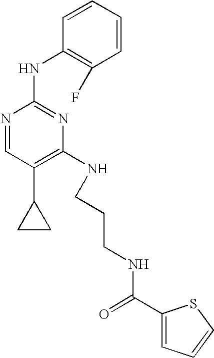 | Example 52 |
| 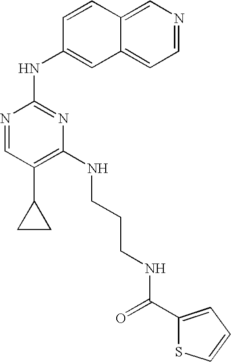 | Example 53 |
| 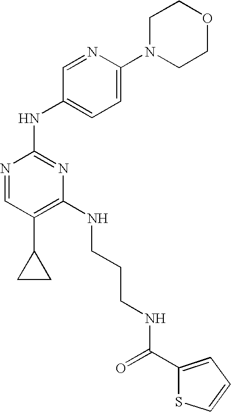 | Example 54 |
| 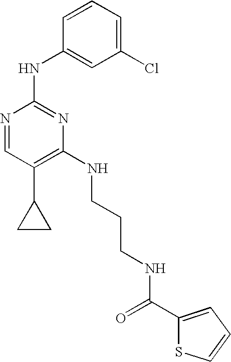 | Example 55 |
| 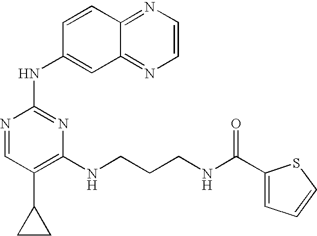 | Example 56 |
| 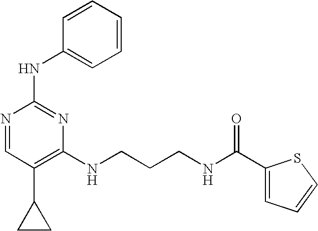 | Example 57 |
| 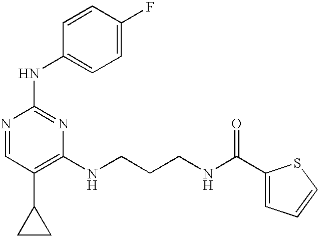 | Example 58 |
| 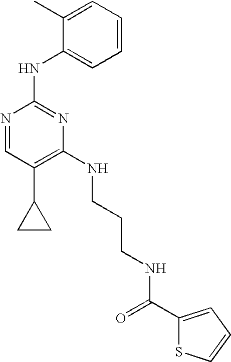 | Example 59 |
| 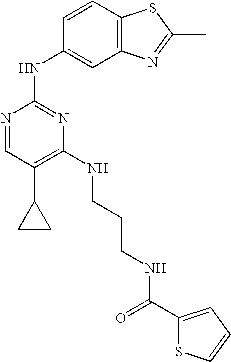 | Example 60 |
| 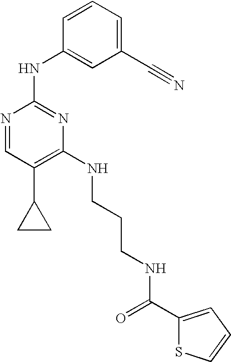 | Example 61 |
| 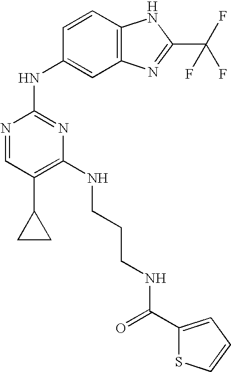 | Example 62 |
| 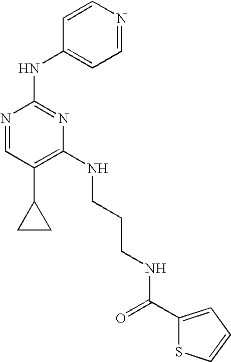 | Example 63 |
| 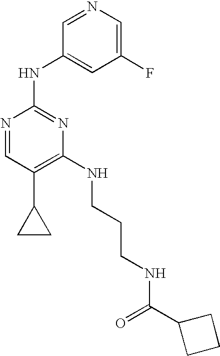 | Example 64 |
| 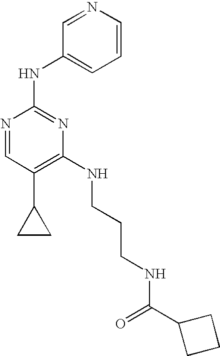 | Example 65 |
| 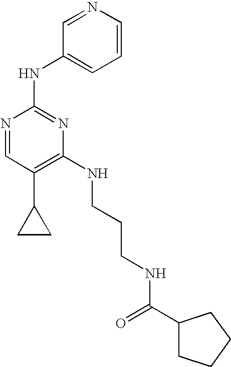 | Example 66 |
| 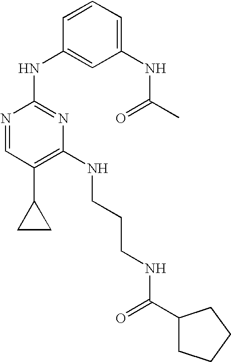 | Example 67 |
| 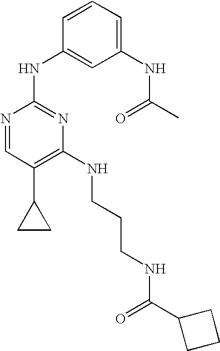 | Example 68 |
| 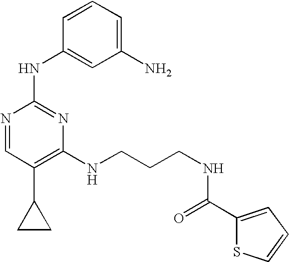 | Example 69 |
| 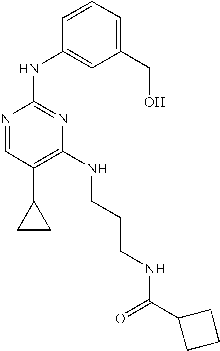 | Example 70 |
| 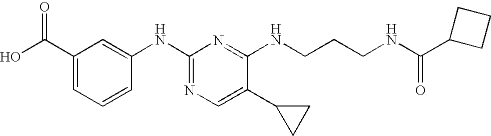 | Example 71 |
| 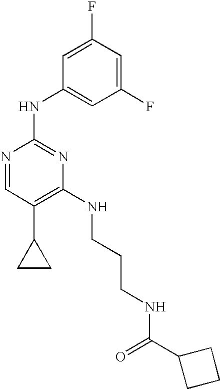 | Example 72 |
| 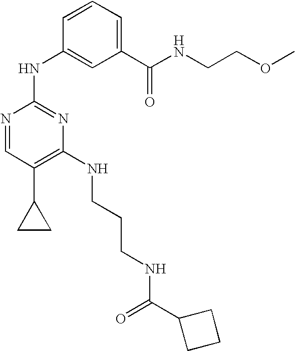 | Example 73 |
| 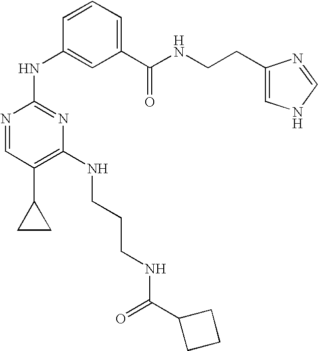 | Example 74 |
| 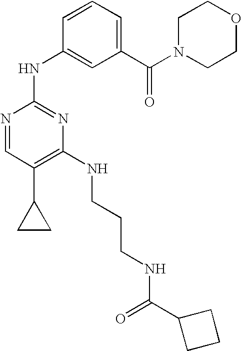 | Example 75 |
| 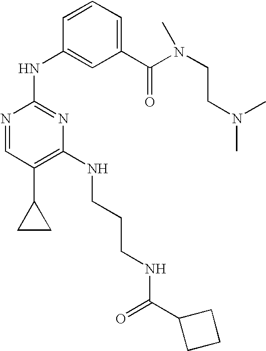 | Example 76 |
| 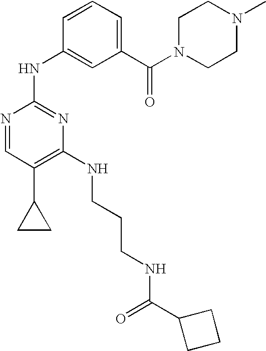 | Example 77 |
| 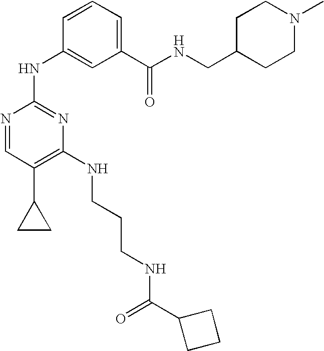 | Example 78 |
| 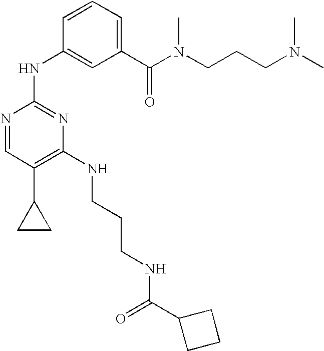 | Example 79 |
| 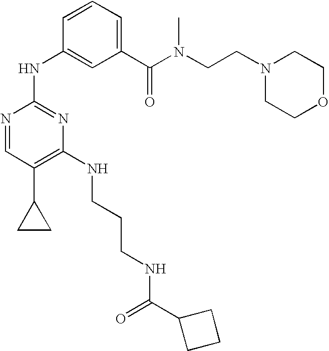 | Example 80 |
| 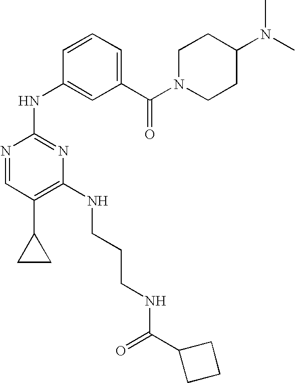 | Example 81 |
| 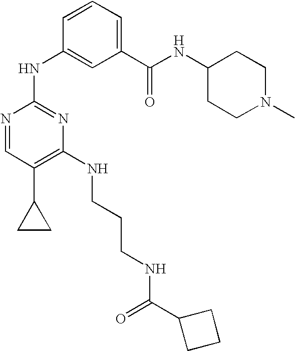 | Example 82 |
| 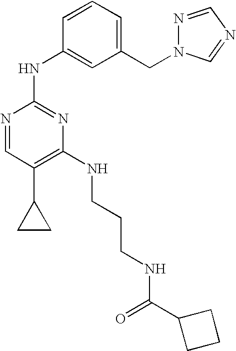 | Example 83 |
| 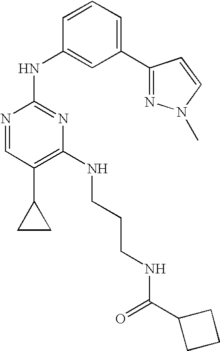 | Example 84 |
| 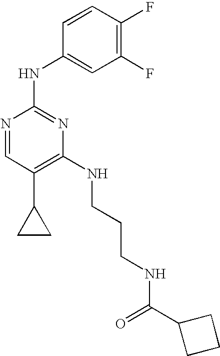 | Example 85 |
| 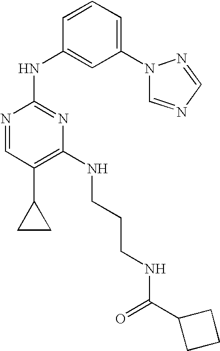 | Example 86 |
| 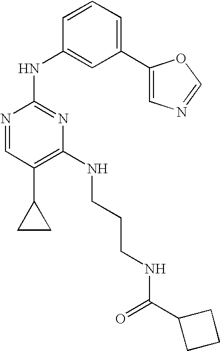 | Example 87 |
| 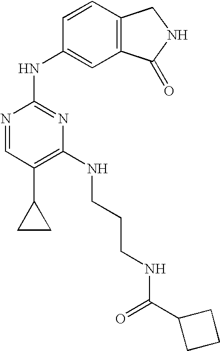 | Example 88 |
| 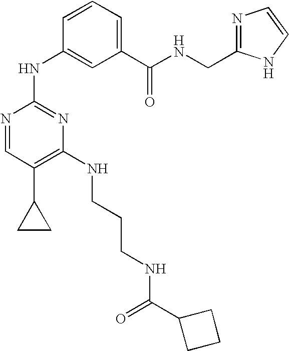 | Example 89 |
| 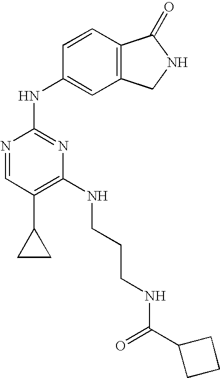 | Example 90 |
| 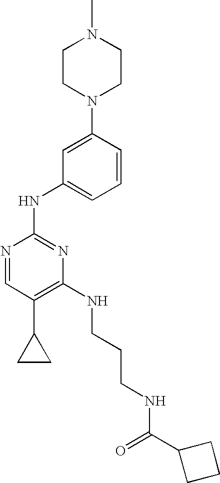 | Example 91 |
| 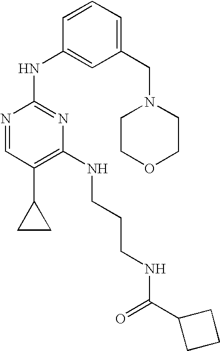 | Example 92 |
| 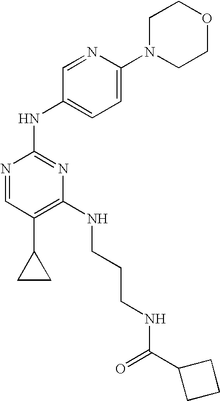 | Example 93 |
| 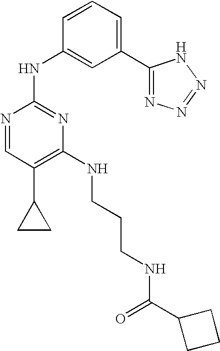 | Example 94 |
| 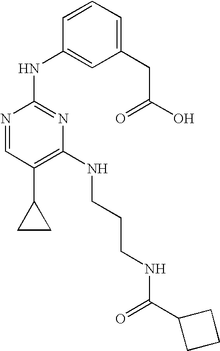 | Example 95 |
| 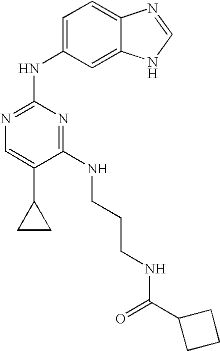 | Example 96 |
| 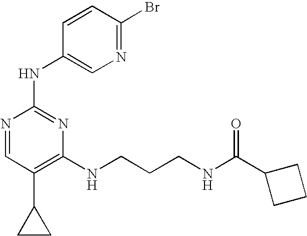 | Example 97 |
| 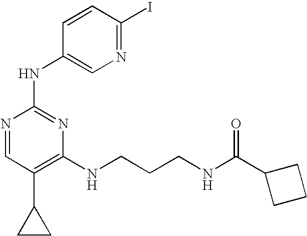 | Example 98 |
| 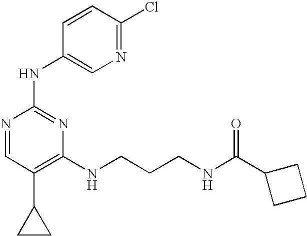 | Example 99 |
| 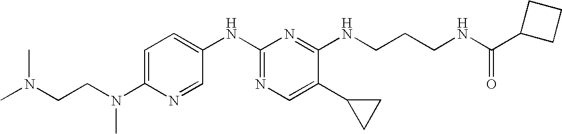 | Example 100 |
| 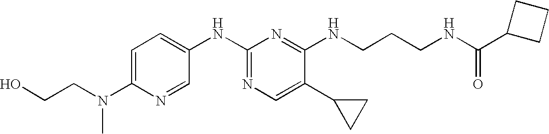 | Example 101 |
| 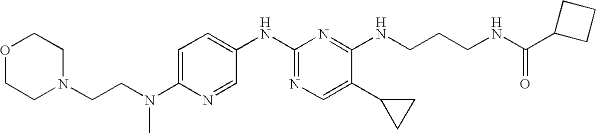 | Example 102 |
| 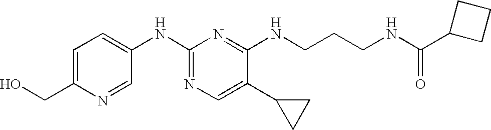 | Example 103 |
| 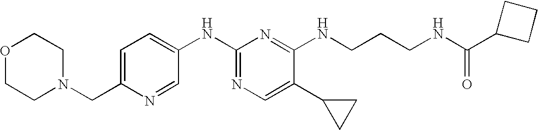 | Example 104 |
| 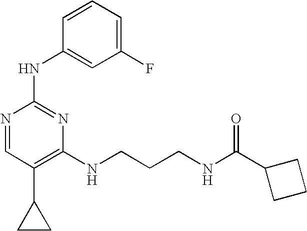 | Example 105 |
| 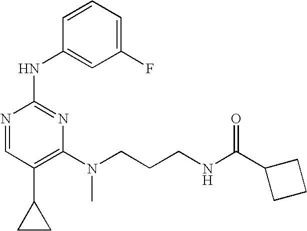 | Example 106 |
| 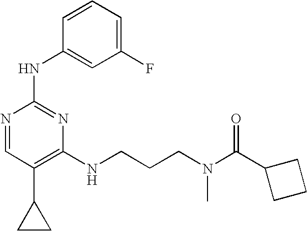 | Example 107 |
| 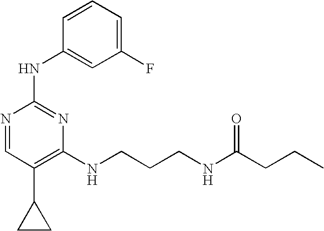 | Example 108 |
| 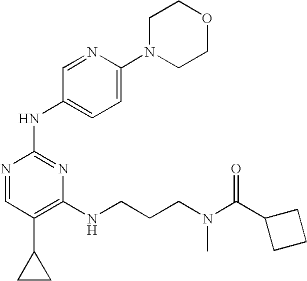 | Example 109 |
| 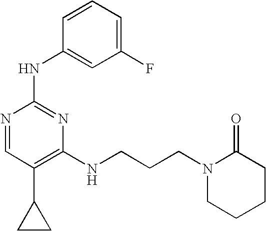 | Example 110 |
| 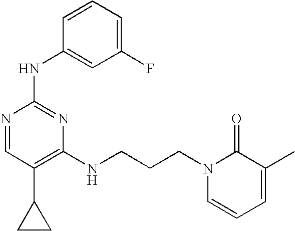 | Example 111 |
| 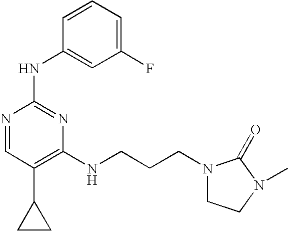 | Example 112 |
| 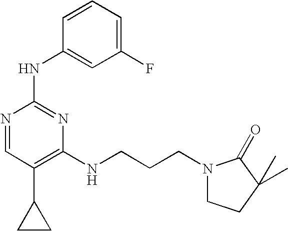 | Example 113 |
| 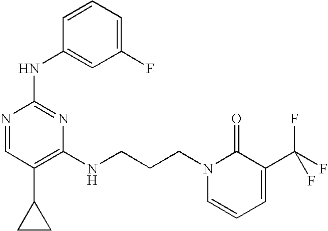 | Example 114 |
| 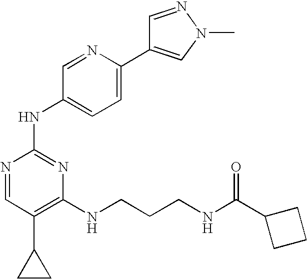 | Example 115 |
| 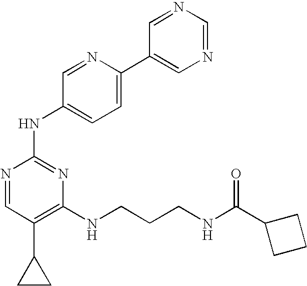 | Example 116 |
| 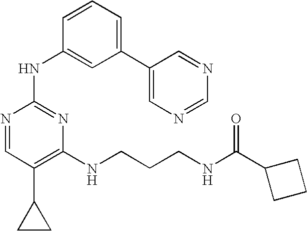 | Example 117 |
| 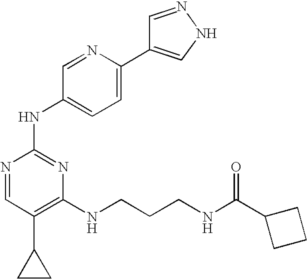 | Example 118 |
| 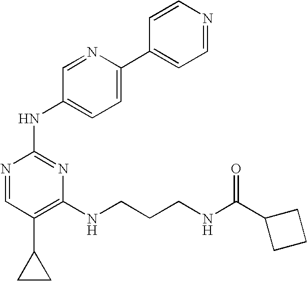 | Example 119 |
| 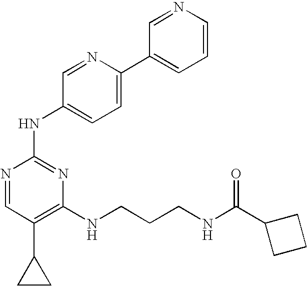 | Example 120 |
| 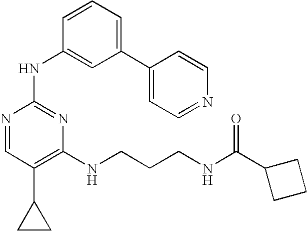 | Example 121 |
| 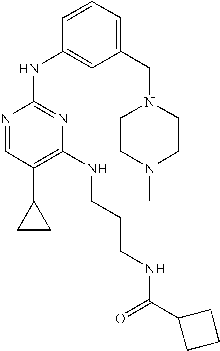 | Example 122 |
| 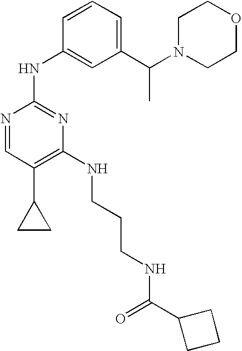 | Example 123 |
| 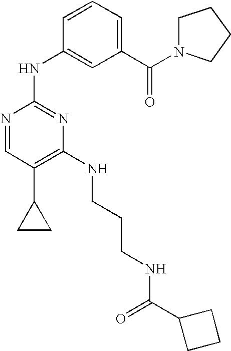 | Example 124 |
| 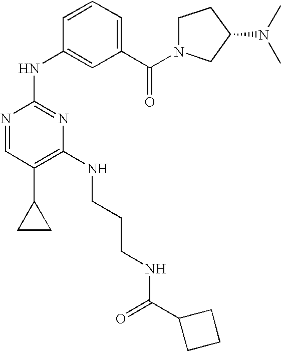 | Example 125 |
| 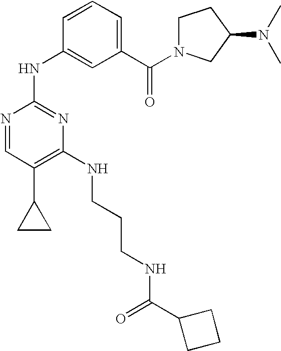 | Example 126 |
| 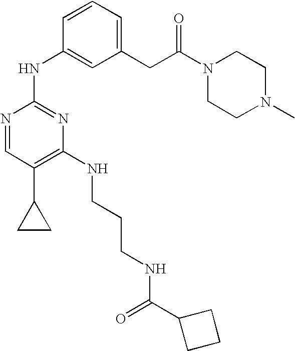 | Example 127 |
| 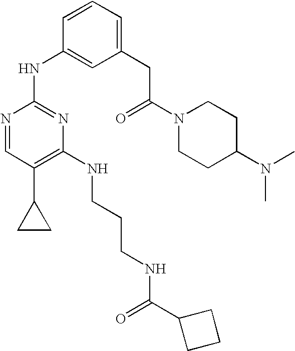 | Example 128 |
| 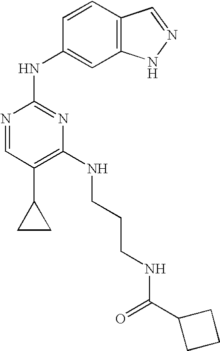 | Example 129 |
| 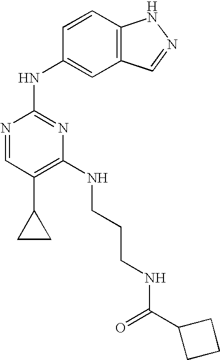 | Example 130 |
| 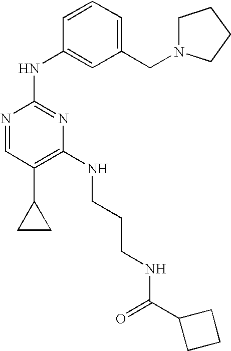 | Example 131 |
| 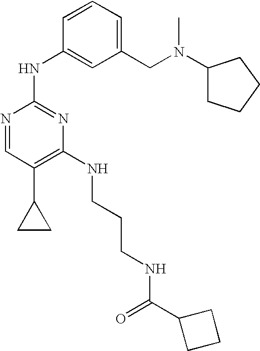 | Example 132 |
| 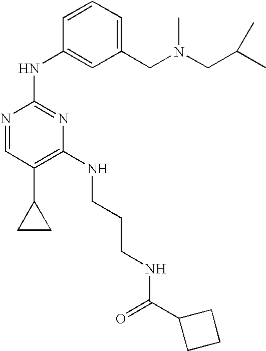 | Example 133 |
| 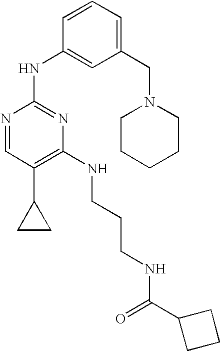 | Example 134 |
| 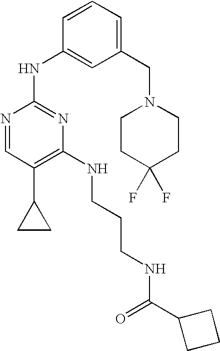 | Example 135 |
| 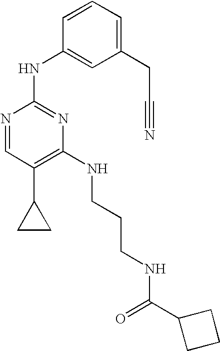 | Example 136 |
| 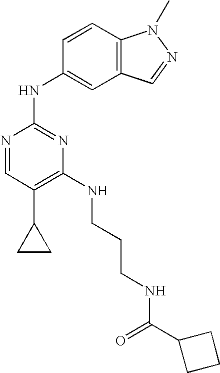 | Example 137 |
| 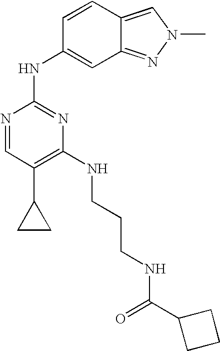 | Example 138 |
| 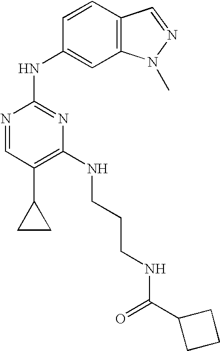 | Example 139 |
| 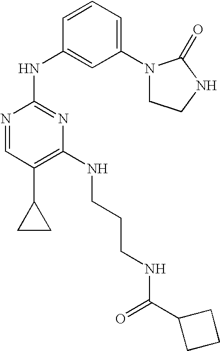 | Example 140 |
| 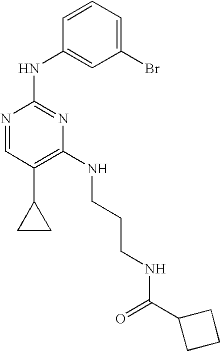 | Example 141 |
| 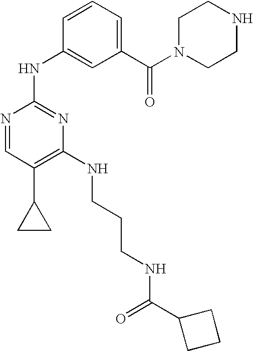 | Example 142 |
| 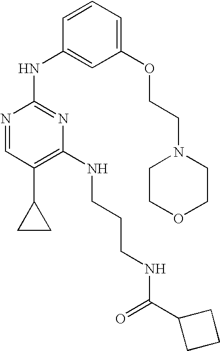 | Example 143 |
| 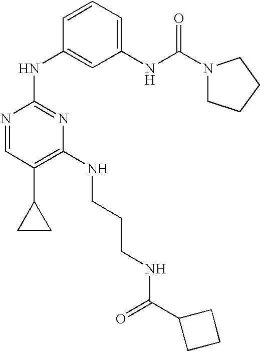 | Example 144 |
| 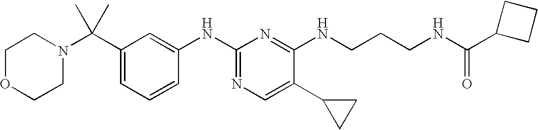 | Example 145 |
| 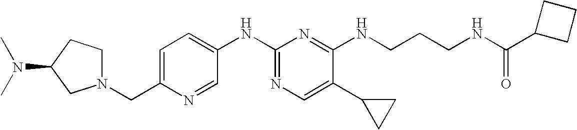 | Example 146 |
| 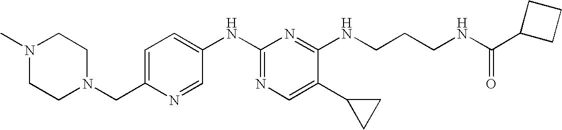 | Example 147 |
| 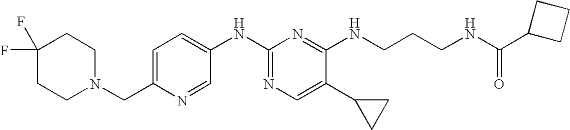 | Example 148 |
| 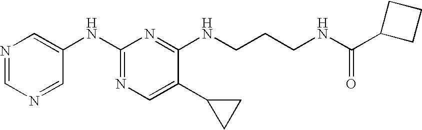 | Example 149 |
| 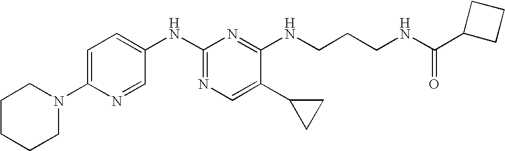 | Example 150 |
| 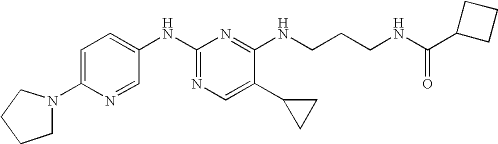 | Example 151 |
| 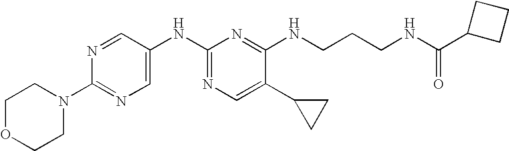 | Example 152 |
| 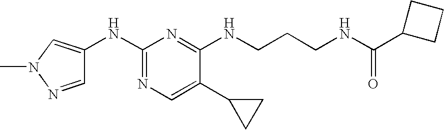 | Example 153 |
| 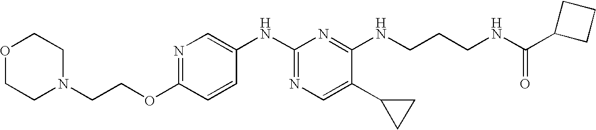 | Example 154 |
| 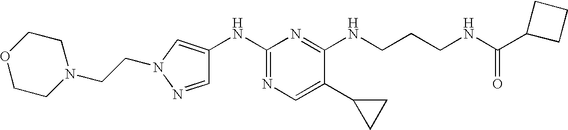 | Example 155 |
| 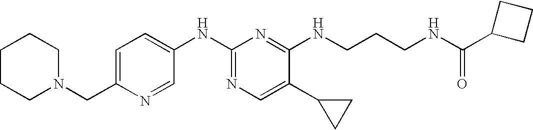 | Example 156 |
| 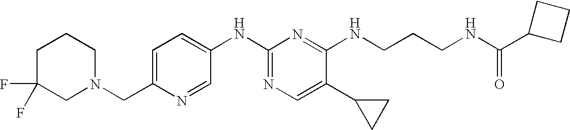 | Example 157 |
| 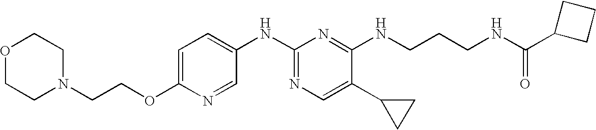 | Example 158 |
| 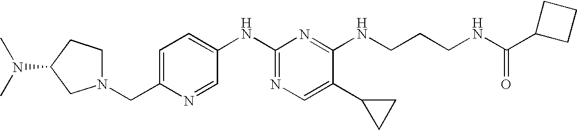 | Example 159 |
| 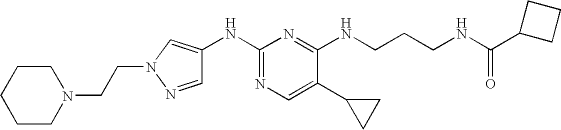 | Example 160 |
| 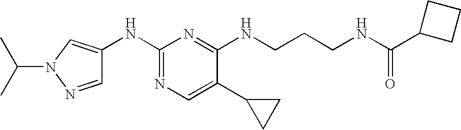 | Example 161 |
| 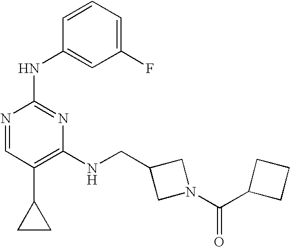 | Example 162 |
| 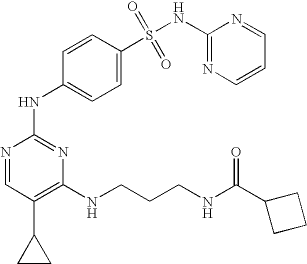 | Example 163 |
| 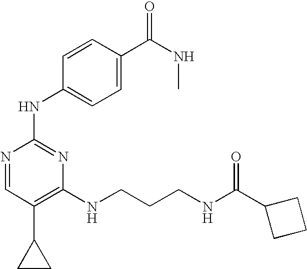 | Example 164 |
| 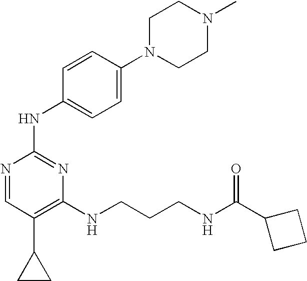 | Example 165 |
| 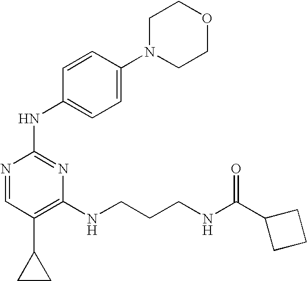 | Example 166 |
| 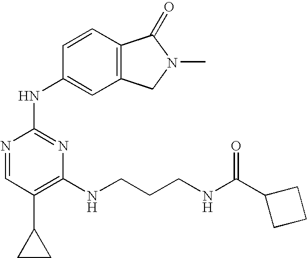 | Example 167 |
| 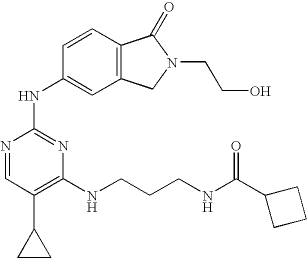 | Example 168 |
| 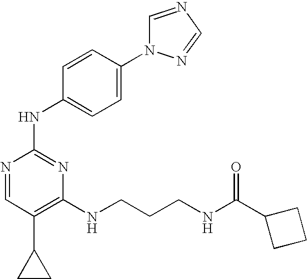 | Example 169 |
| 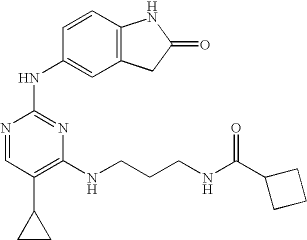 | Example 170 |
| 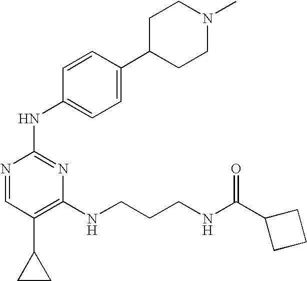 | Example 171 |
| 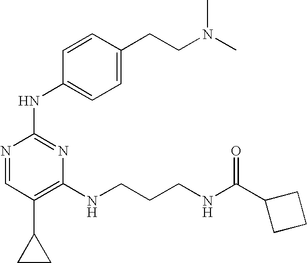 | Example 172 |
| 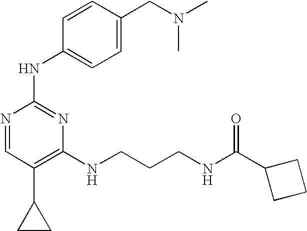 | Example 173 |
| 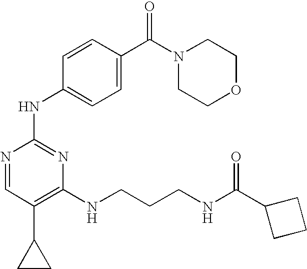 | Example 174 |
| 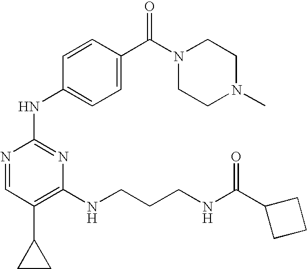 | Example 175 |
| 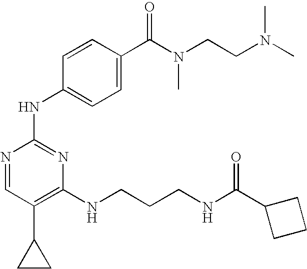 | Example 176 |
| 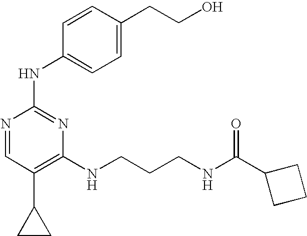 | Example 177 |
| 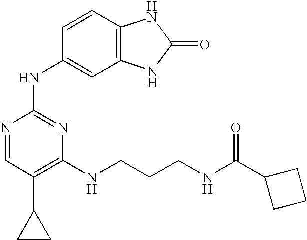 | Example 178 |
| 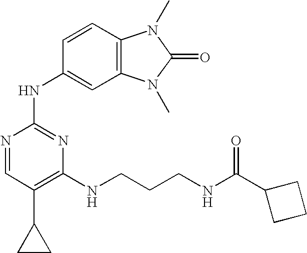 | Example 179 |
| 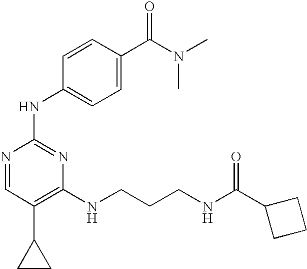 | Example 180 |
| 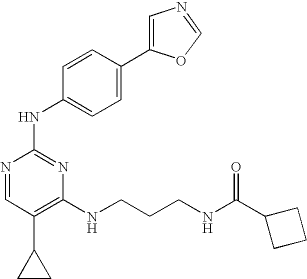 | Example 181 |
| 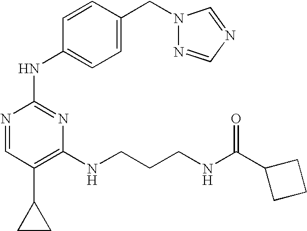 | Example 182 |
| 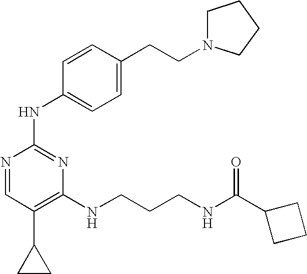 | Example 183 |
| 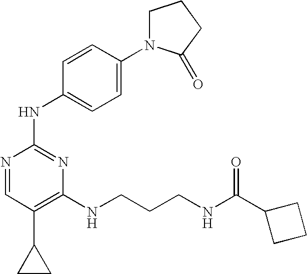 | Example 184 |
| 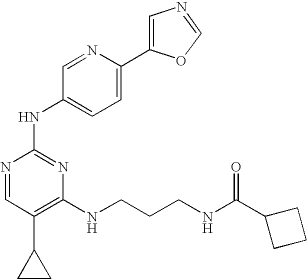 | Example 185 |
| 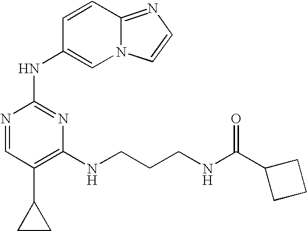 | Example 186 |
| 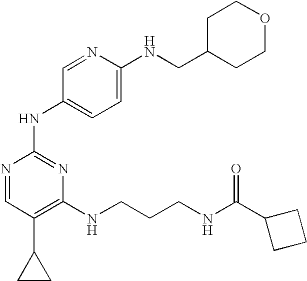 | Example 187 |
| 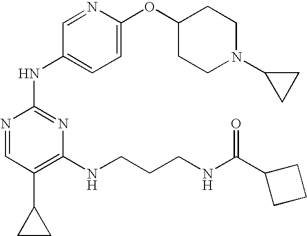 | Example 188 |
| 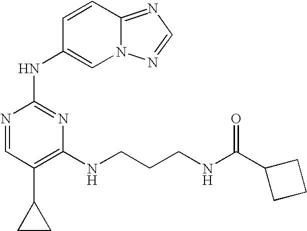 | Example 189 |
| 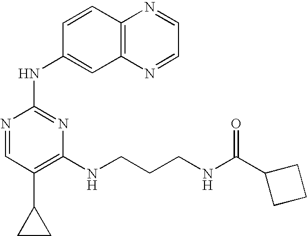 | Example 190 |
| 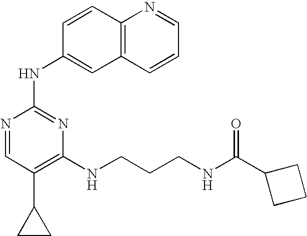 | Example 191 |
| 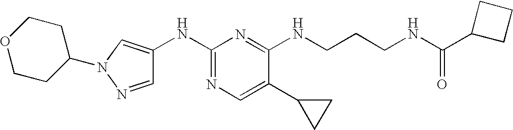 | Example 192 |
| 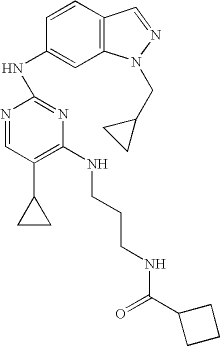 | Example 193 |
| 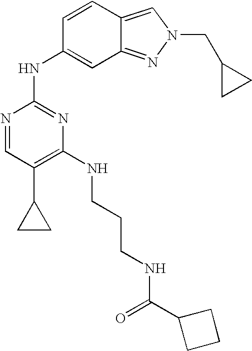 | Example 194 |
| 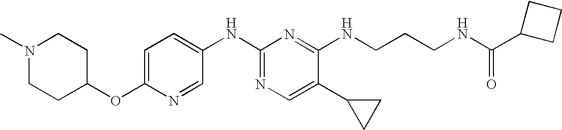 | Example 195 |
| 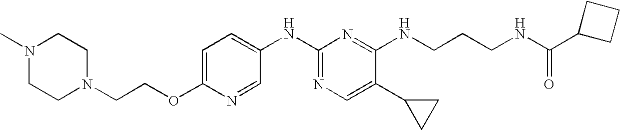 | Example 196 |
| 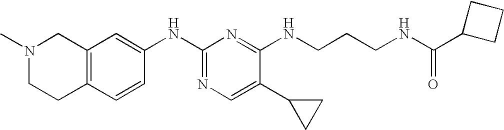 | Example 197 |
| 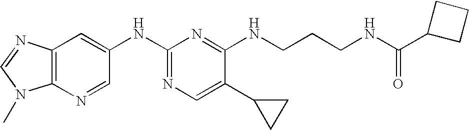 | Example 198 |
| 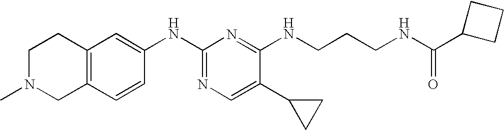 | Example 199 |
| 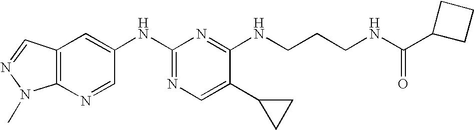 | Example 200 |
| 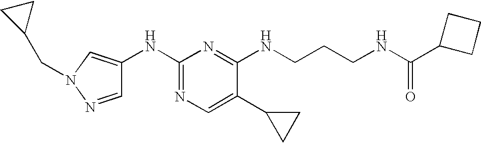 | Example 201 |
| 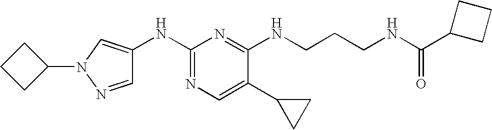 | Example 202 |
| 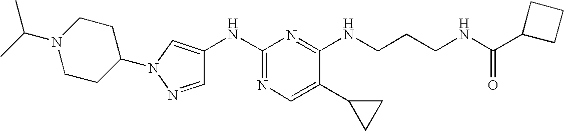 | Example 203 |
| 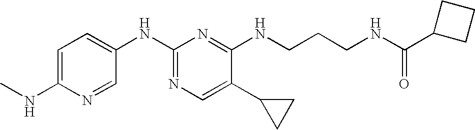 | Example 204 |
| 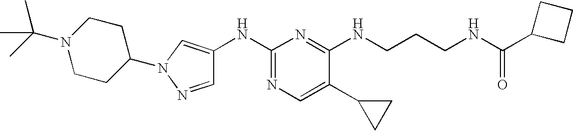 | Example 205 |
| 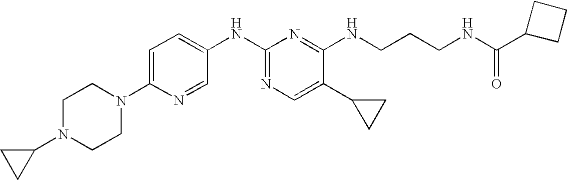 | Example 206 |
| 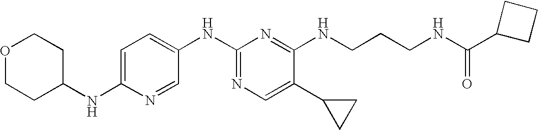 | Example 207 |
| 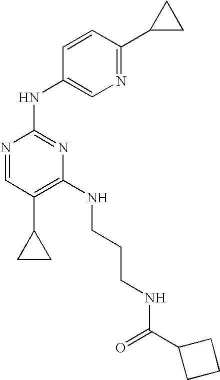 | Example 208 |
| 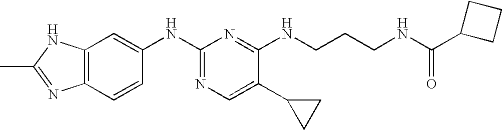 | Example 209 |
| 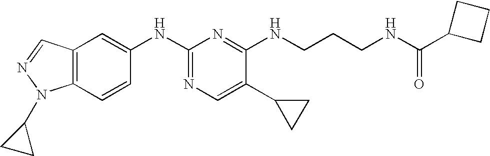 | Example 210 |
| 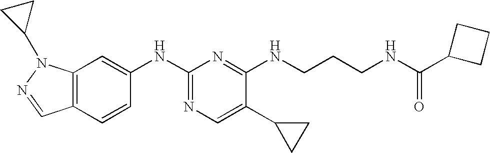 | Example 211 |
| 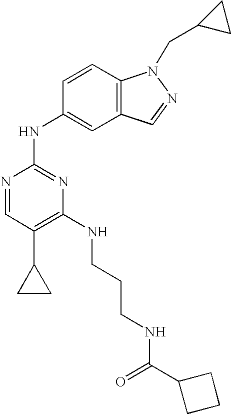 | Example 212 |
| 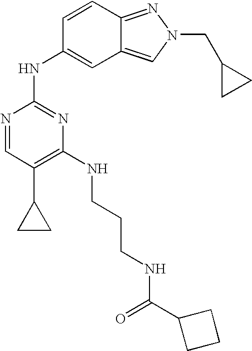 | Example 213 |
| 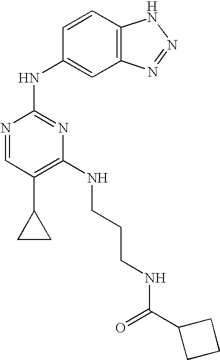 | Example 214 |

20. A pharmaceutical composition comprising at least one compound according to claim 1 and a pharmaceutically acceptable carrier, diluent or excipient.

21. A compound according to claim 1 for use in medicine.

22. A compound according to claim 1 for use in treating a disorder selected from cancer, septic shock, neurodegenerative diseases, Alzheimer's disease, Primary open Angle Glaucoma (POAG), hyperplasia, rheumatoid arthritis, psoriasis, artherosclerosis, retinopathy, osteoarthritis, endometriosis and chronic inflammation.

23. Use of a compound according to claim 1 in the preparation of a medicament for treating or preventing a disorder selected from cancer, septic shock, neurodegenerative diseases, Alzheimer's disease, Primary open Angle Glaucoma (POAG), hyperplasia, rheumatoid arthritis, psoriasis, artherosclerosis, retinopathy, osteoarthritis, endometriosis and chronic inflammation.

24. Use according to claim 23 wherein the compound is administered in an amount sufficient to inhibit a kinase selected from TBK1, MKK1, ERK8, RSK1, RSK2, PDK1, S6K1, MNK2, PHK, CHK1, CHK2, GSK3beta, CDK2, MARK3, MELK, IRR, VEG-FR and IKKepsilon.

25. Use of a compound according to claim 1 in the preparation of a medicament for the prevention or treatment of a disorder caused by, associated with or accompanied by any abnormal kinase activity, wherein the kinase is selected from TBK1, MKK1, ERK8, RSK1, RSK2, PDK1, S6K1, MNK2, PHK, CHK1, CHK2, GSK3beta, CDK2, MARK3, MELK, IRR, VEG-FR, IKKepsilon and combinations thereof.

26. Use according to claim 25 wherein the kinase is selected from TBK1, PDK1, ERK8, MARK3, and IKKepsilon and combinations thereof.

27. Use according to claim 25 wherein the kinase is selected from TBK1 and PDK1.

28. Use according to claim 25 wherein the kinase is TBK1.

29. Use according to claim 25 wherein the disorder is selected from cancer, septic shock, neurodegenerative diseases, Alzheimer's disease, diseases of the eye, including Primary open Angle Glaucoma (POAG), hyperplasia, rheumatoid arthritis, autoimmune diseases, artherosclerosis, retinopathy, osteoarthritis, fibrotic diseases, endometriosis and chronic inflammation.

30. Use according to claim 23 wherein the cancer is selected from papilloma, blastoglioma, Kaposi's sarcoma, melanoma, lung cancer, ovarian cancer, prostate cancer, squamous cell carcinoma, astrocytoma, head cancer, neck cancer, skin cancer, liver cancer, bladder cancer, breast cancer, lung cancer, uterus cancer, prostate cancer, testis carcinoma, colorectal cancer, thyroid cancer, pancreatic cancer, gastric cancer, hepatocellular carcinoma, leukemia, lymphoma, Hodgkin's disease and Burkitt's disease; the disease of the eye is selected from glaucoma, primary open angle glaucoma (POAG), normal tension glaucoma (NTG) and low tension laucoma (LTG); the auto-immune disease is selected from psoriasis, alopecia and multiple sclerosis; and the fibrotic disease is cirrhosis of the liver.

31. A method of treating a mammal having a disease state alleviated by the inhibition of a kinase selected from TBK1, MKK1, ERK8, RSK1, RSK2, PDK1, S6K1, MNK2, PHK, CHK1, CHK2, GSK3beta, CDK2, MARK3, MELK, IRR, VEG-FR, and IKKepsilon, wherein the method comprises administering to a mammal a therapeutically effective amount of a compound according to claim 1.

32. A method according to claim 31 wherein the disease state is alleviated by the inhibition of a kinase selected from TBK1, PDK1, ERK8, MARK3, and IKKepsilon.

33. A method according to claim 31 wherein the disease state is alleviated by the inhibition of TBK1 or PDK1.

34. A method according to claim 31 wherein the disease state is alleviated by the inhibition of TBK1.

35. A method according to claim 31 wherein the mammal is a human.

36. Use of a compound according to claim 1 in an assay for identifying further candidate compounds capable of inhibiting one or more kinases selected from TBK1, MKK1, ERK8, RSK1, RSK2, PDK1, S6K1, MNK2, PHK, CHK1, CHK2, GSK3beta, CDK2, MARK3, MELK, IRR, VEG-FR, and IKKepsilon.

37. Use according to claim 36 wherein said assay is a competitive binding assay.

38. Use according to claim 37 wherein said competitive binding assay comprises contacting a compound according to any one of claims 1 to 19 with a kinase selected from TBK1, MKK1, ERK8, RSK1, RSK2, PDK1, S6K1, MNK2, PHK, CHK1, CHK2, GSK3beta, CDK2, MARK3, MELK, IRR, VEG-FR, and IKKepsilon, and a candidate compound and detecting any change in the interaction between the compound according to any one of claims 1 to 19 and the kinase.

39. A process for preparing a compound of formula IV, wherein R¹, R², R³ and R⁷ are as defined in claim 1, said process comprising the steps of: 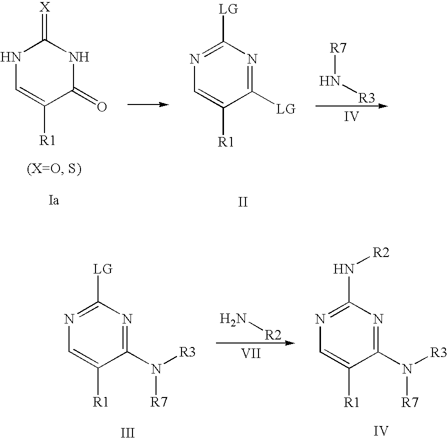
(i) converting a compound of formula Ia to a compound of formula II, where each LG is independently a leaving group;
(ii) reacting said compound of formula II with an amine of formula IV to form a compound of formula III;
(iii) reacting said compound of formula III with an amine of formula VII to form a compound of formula IV.

40. A process for preparing a compound of formula XIX, wherein R¹, R² and R³ are as defined in claim 1, said process comprising the steps of: 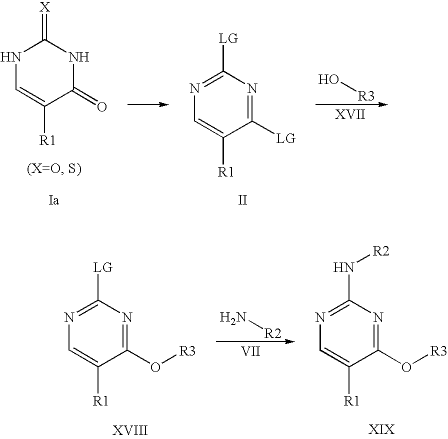
(i) converting a compound of formula Ia to a compound of formula II, where each LG is independently a leaving group;
(ii) reacting said compound of formula II with an amine of formula XVII to form a compound of formula XVIII;
(iii) reacting said compound of formula XVIII with an amine of formula VII to form a compound of formula XIX.

41. A combination comprising a compound according to claim 1 and a further therapeutic agent.

42. A pharmaceutical composition according to claim 20 which further comprises a second therapeutic agent.
